(19) United States
(12) Patent Application Publication
(10) Pub. No.: US 2005/0101644 A1
(43) Pub. Date: May 12, 2005

(54) Benzylether amine compounds useful as CCR-5 antagonists

(75) Inventors: David Davey, El Sobrante, CA (US); Wheeseong Lee, Orinda, CA (US); Shou-Fu Lu, San Ramon, CA (US); Gary Phillips, Pleasant Hill, CA (US); Guo Ping Wei, San Ramon, CA (US); Bin Ye, Moraga, CA (US)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 10/984,430

(22) Filed: Nov. 8, 2004

Related U.S. Application Data
(60) Provisional application No. 60/519,002, filed on Nov. 10, 2003.

(51) Int. Cl. A61K 31/44; A61K 31/40; A61K 31/198; A61K 31/165; A61K 31/138; A61K 31/137

(52) U.S. Cl. 514/357; 514/408; 514/567; 514/649; 514/620; 514/651; 546/334; 548/571; 562/439; 562/440; 564/60; 564/164; 564/338

(57) ABSTRACT

The present invention relates to compounds which are CCR-5 receptor antagonists of the general formula I: 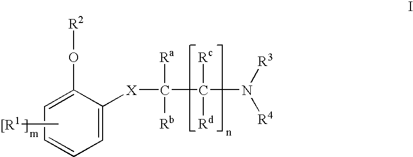
wherein R¹, R², R³, R⁴, R^{a}, R^{b}, R^{c}, R^{d}, X, m and n are as defined herein. The invention further comprises pharmaeceutical compositions comprising such compounds, as well as the use of such compounds to treat CCR-5 mediated disorders.

This application claims priority to U.S. Provisional Application Ser. No. 60/519,002 filed Nov. 10, 2003, the entirety of which is incorporated herein by reference.

### BACKGROUND OF THE INVENTION

Chemoattractant cytokines or chemokines are a family of proinflammatory mediators that promote recruitment and activation of leukocytes (e.g., monocytes, lymphocytes, and granulocytes). They can be released by many kinds of tissue cells after activation. Continuous release of chemokines at sites of inflammation mediates the ongoing migration of effector cells in chronic inflammation. The chemokines characterized to date are related in primary structure. They share four conserved cysteines, which form disulfide bonds. Based upon this conserved cysteine motif, the family is divided into two main branches, designated as the C—X—C chemokines (α-chemokines), and the C—C chemokines (β-chemokines), in which the first two conserved cysteines are separated by an intervening residue, or adjacent, respectively (Baggiolini, M. and Dahinden, C. A., Immunology Today, 15:127-133 (1994)).

The C—C chemokines include RANTES (Regulated on Activation, Normal T Expressed and Secreted), the macrophage inflammatory proteins 1α and 1β (MIP-1α and MIP-1β), and human monocyte chemotactic proteins 1-3 (MCP-1, MCP-2, MCP-3), which have been characterized as chemoattractants and activators of monocytes or lymphocytes. Chemokines, such as RANTES and MIP-1α have been implicated in a wide range of human acute and chronic inflammatory diseases including rheumatoid arthritis, and respiratory diseases, such as asthma and allergic disorders. In particular a number of laboratories have implicated chemokines in the pathophysiology of RA (rheumatoid arthritis). Several studies involving human arthritic patients have demonstrated an increase in the expression levels of the CCR-5 ligands RANTES, MIP-1β, and MIP-1α in diseased synovium and an increased selective accumulation of CCR-5⁺ lymphocytes in diseased synovium fluid. (Rathanaswami P. et al., Journal of Biological Chemistry 268: 5834-9 (1993) and Rot A. et al. Journal of Experimental Medicine 176: 1489-95 (1992)).

The chemokine receptors are members of a superfamily of G protein-coupled receptors (GPCR) which share structural features that reflect a common mechanism of action of signal transduction (Gerard, C. and Gerard, N. P., Annu Rev. Immunol., 12:775-808 (1994); Gerard, C. and Gerard, N. P., Curr. Opin. Immunol., 6:140-145 (1994)). The first receptor for the C—C chemokines that was cloned and expressed binds the chemokines MIP-1α and RANTES. Accordingly, this MIP-1α/RANTES receptor was designated C—C chemokine receptor 1 (also referred to as CCR-1; Neote, K., et al., Cell, 72:415-425 (1993); Horuk, R. et al., WO 94/11504, May 26, 1994; Gao, J.-I. et al., J. Exp. Med., 177:1421-1427 (1993)). Three other receptors have been characterized which bind and/or signal in response to RANTES: CCR-3 mediates binding and signaling of chemokines including eotaxin, RANTES, and MCP-3 (Ponath et al., J. Exp. Med., 183:2437 (1996)), CCR-4 binds chemokines including RANTES, MIP-1α, and MCP-1 (Power, et al., J. Biol. Chem., 270:19495 (1995)), and CCR-5 binds chemokines including MIP-1α, RANTES, and MIP-1β. (Samson, et al., Biochem. 35: 3362-3367 (1996)).

RANTES is a chemotactic chemokine for a variety of cell types, including monocytes, eosinophils, and a subset of T-cells. The ability of RANTES to induce the directed migration of monocytes and a memory population of circulating T-cells (Schall, T. et al., Nature, 347:669-71 (1990)) suggests that this chemokine and its receptor(s) play an important role in chronic inflammatory diseases, since these diseases are characterized by destructive infiltrates of T cells and monocytes.

### SUMMARY OF THE INVENTION

The present invention relates to compounds of the following formula I 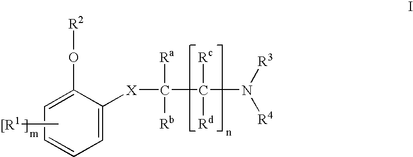
enantiomers, diastereomers, salts and solvates thereof wherein
X is a bond or oxygen;
m is 0, 1, 2, 3 or 4;
n is 0, 1 or 2;
R¹ is an optional substituent independently selected at each occurrence from halogen, alkyl, haloalkyl, nitro, or —NR⁵R⁶;
R² is
   a) hydrogen or
   b) alkyl, cycloalkyl, alkenyl, aryl or heteroaryl any of which may be optionally substituted with a group Y;
Y is
   a) aryl or heteroaryl either of which may be optionally substituted with one or more Z¹, Z², Z³;
   b) cycloalkyl or heterocyclo either of which optionally substituted with one or more Z¹, Z², Z³;
   c) —COOR⁷;
   d) —NR⁸R⁹;
   e) —CHR¹⁰(OR¹¹);
   f) —C(═O)—NR⁸R⁹;
   g) —NR¹²—(C═O)—NR⁸R⁹;
   h) —CN;
   i) —C(═N—OR¹³);
   j) alkoxy;
R³ and R⁴ are independently selected from
   a) hydrogen;
   b) alkyl, cycloalkyl, (cycloalkyl)alkyl, aryl, (aryl)alkyl, heterocyclo, (heterocyclo)alkyl, heteroaryl, or (heteroaryl)alkyl any of which may be optionally substituted with one or more Z¹, Z², Z³; or
   c) —C(O)R*, —C(O)OR*, —C(O)NHR* or —SO₂R*; or R³ and R⁴ together with the nitrogen atom to which they bonded may combine to form a
   heterocylo or heteroaryl ring optionally substituted with one or more Z¹, Z², Z³; R⁵ and R⁶ are independently H, —C(O)R*, —SO₂R*, or —C(O)NR^{8a}R^{9a};
R⁷, R⁸, R^{8a}, R⁹, and R^{9a} are independently
   a) hydrogen or
   b) alkyl, cycloalkyl, (cycloalkyl)alkyl, aryl, (aryl)alkyl, heterocyclo, (heterocyclo)alkyl, heteroaryl, or (heteroaryl)alkyl any of which may be optionally substituted with one or more Z¹, Z², Z³;
R¹⁰ is H, alkyl or —OR*;
R¹¹ and R¹² are independently H or alkyl;
R¹³ is alkyl;
R* at each occurrence is independently alkyl, cycloalkyl, (cycloalkyl)alkyl, aryl, (aryl)alkyl, heterocyclo, (heterocyclo)alkyl, heteroaryl, or (heteroaryl)alkyl any of which may be optionally substituted with one or more Z¹, Z², Z³;
R^{a} and R^{b} are independently hydrogen, —OR^{10a}, alkyl, hydroxyalkyl, or haloalkyl;
or R^{a} and R^{b} may combine to form oxo;
R^{c} and R^{d} at each occurrence are independently H, —OR^{10b}, alkyl or haloalkyl
R^{10a} and R^{10b} are independently hydrogen, alkyl, haloalkyl, aryl, or heteroaryl; Z¹, Z² and Z³ are optional substituents independently selected from
   (1) V, where V is
      (i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;
      (ii) a group (i) which is itself substituted by one or more of the same or different groups (i); or
      (iii) a group (i) or (ii) which is independently substituted by one or more (preferably 1 to 3) of the following groups (2) to (13) of the definition of
   (2) —OH or —OV,
   (3) —SH or —SV,
   (4) —C(O)H, —C(O)OH, —C(O)V, —C(O)OV or —O—C(O)V,
   (5) —SO₃H, —S(O)ₜV, or S(O)ₜN(V¹)V, where t is 1 or 2,
   (6) halo,
   (7) cyano,
   (8) nitro,
   (9) —U¹—NV²V³,
   (10) —U¹—N(V¹)—U²—NV²V³,
   (11) —U¹—N(V⁴)—U²—V,
   (12) —U¹—N(V⁴)—U²—H,
   (13) oxo;
U¹ and U² are each independently
   (1) a single bond,
   (2) —U³—S(O)ₜ—U⁴—,
   (3) —U³—C(O)—U⁴—,
   (4) —U³—C(S)—U⁴—,
   (5) —U³—O—U⁴—,
   (6) —U³—S—U⁴—,
   (7) —U³—O—C(O)—U⁴—,
   (8) —U³—C(O)—OU⁴—,
   (9) —U³—C(═NV^{1a})—U⁴—, or
   (10) —U³—C(O)—C(O)—U⁴—;
V¹, V^{1a}, V², V³ and V⁴
   (1) are each independently hydrogen or a group provided in the definition of Z¹; or
   (2) V² and V³ may together be alkylene or alkenylene, completing a 3- to 8-membered saturated or unsaturated ring together with the atoms to which they are attached, which ring is unsubstituted or substituted with one or more groups listed in the definition of Z¹, or
   (3) V² or V³, together with V¹, may be alkylene or alkenylene completing a 3- to 8-membered saturated or unsaturated ring together with the nitrogen atoms to which they are attached, which ring is unsubstituted or substituted with one or more groups listed in the definition of Z¹; and
U³ and U⁴ are each independently
   (1) a single bond,
   (2) alkylene,
   (3) alkenylene, or
   (4) alkynylene.

The above formula includes separated chiral species, e.g., diastereomers and enantiomers, as well as all mixtures thereof, e.g., racemates, etc.

The compounds of the present invention are useful in the prevention and treatment of a wide variety of inflammatory and immunoregulatory disorders and diseases, allergic conditions, atopic conditions, as well as autoimmune and immunodeficiency pathologies.

Also included in the invention are methods of using the compounds as agents for the treatment of CCR-5 mediated disease states, in particular for the treatment of inflammatory diseases or conditions, autoimmune disorders, and immune deficiency disorders such as HIV infection.

In another aspect, the instant invention may be used to evaluate specific antagonists of CCR-5 receptors. Accordingly, the present invention is directed to the use of these compounds in the preparation and execution of screening assays for compounds which modulate the activity of CCR-5 receptors. For example, the compounds of this invention are useful for isolating receptor mutants, which are excellent screening tools for more potent compounds. Furthermore, the compounds of this invention are useful in establishing or determining the binding site of other compounds to CCR-5 receptors, e.g., by competitive inhibition.

The compounds of the invention can be used in the treatment of mammals, preferably humans, comprising administering to such mammal in need thereof, an effective amount of a compound of formula (I), or a pharmaceutically acceptable salt thereof, optionally in the form of a separated diastereomer or enantiomer, e.g., less than 5%, 2%, or less of the other chiral entity(ies).

Preferred R² groups include alkyl (especially methyl) substituted with Y where Y is aryl (especially phenyl), cycloalkyl (especially cyclopropyl), —CHR¹⁰(OR¹¹), or heterocyclo (especially 1,3 dioxolanyl) any of which may be optionally substituted with one or more Z¹, Z², Z³. Preferred R² groups include the following:

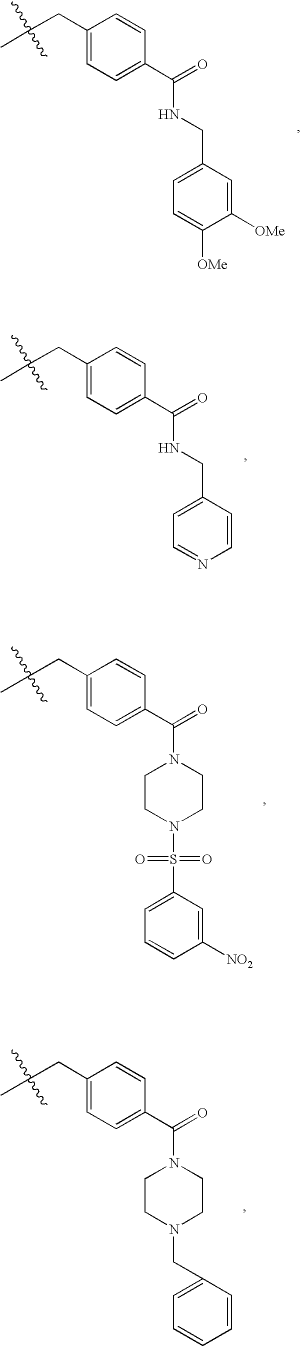

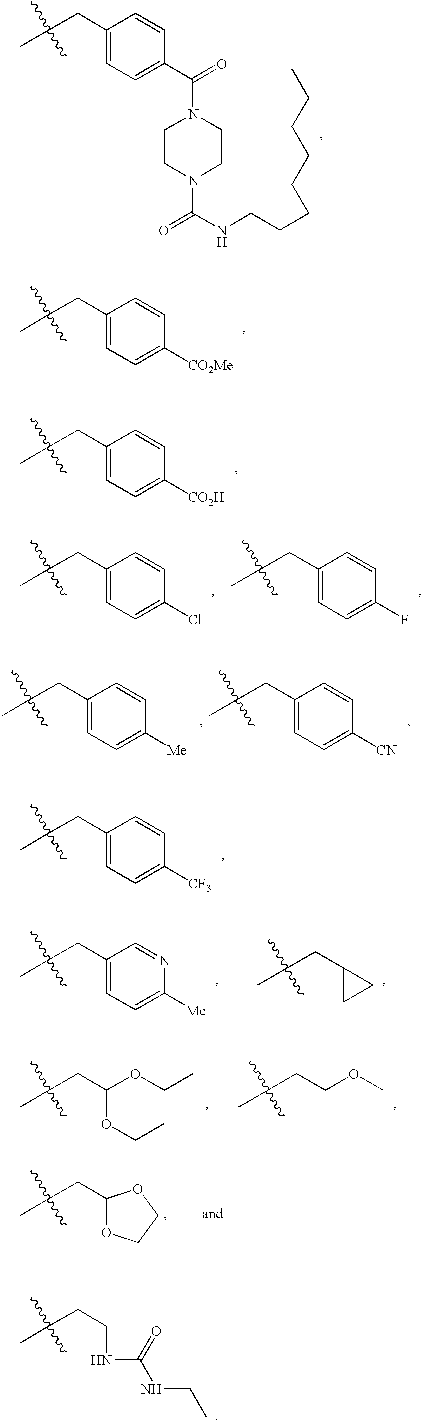

Preferred —NR³R⁴ groups include those where R³ and R⁴ are independently H, alkyl, (hydroxy)alkyl, (heteroaryl)alkyl (especially (pyridyl)alkyl), (heterocyclo)alkyl (especially (morpholinyl)alkyl) or —C(O)NHR* any of which may be optionally substituted with one or more Z¹, Z², Z³. Preferred —NR³R⁴ groups further include groups where R³ and R⁴ together with the nitrogen atom to which they are bonded, combine to form a heterocylo or heteroaryl ring optionally substituted with one or more Z¹, Z², Z³ such as

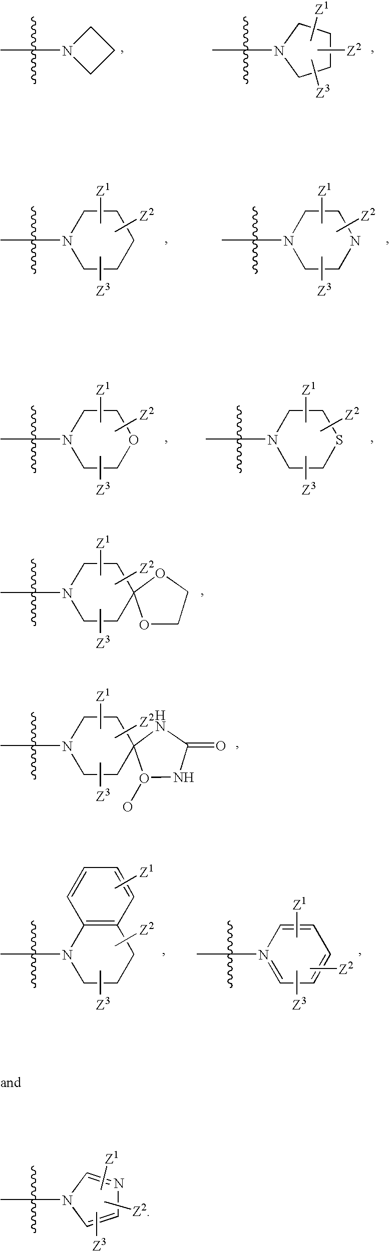

Preferred —NR³R⁴ groups include the following:

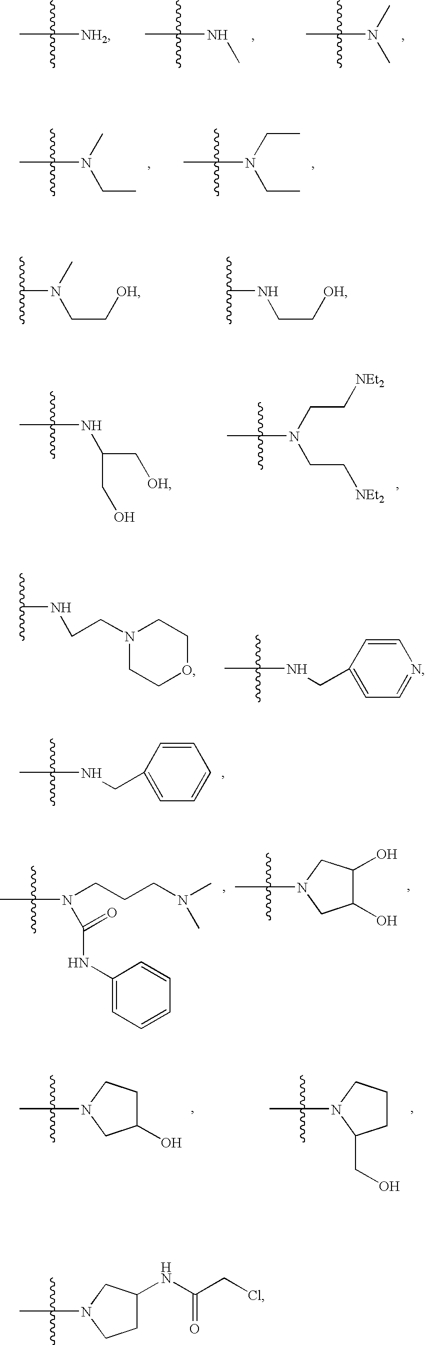

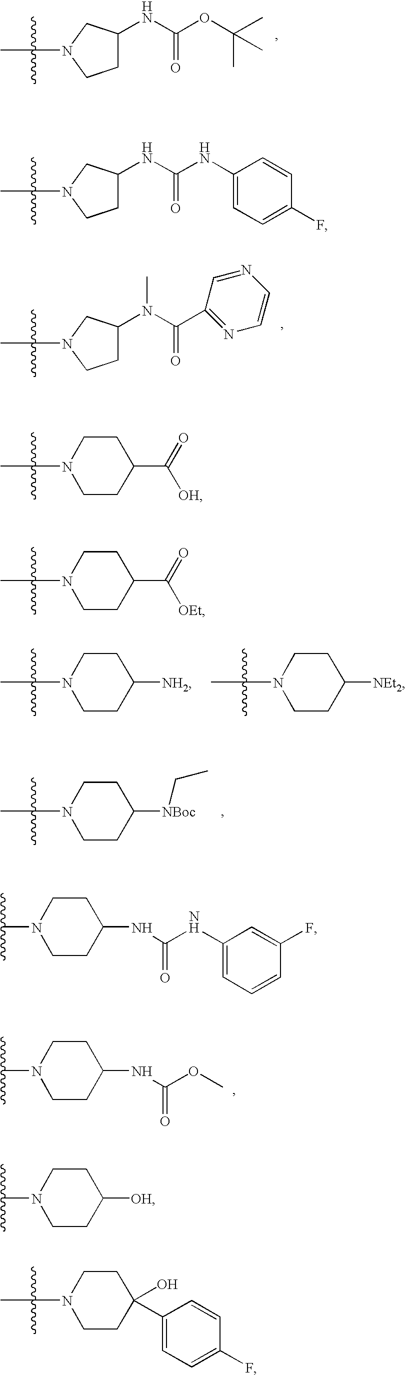

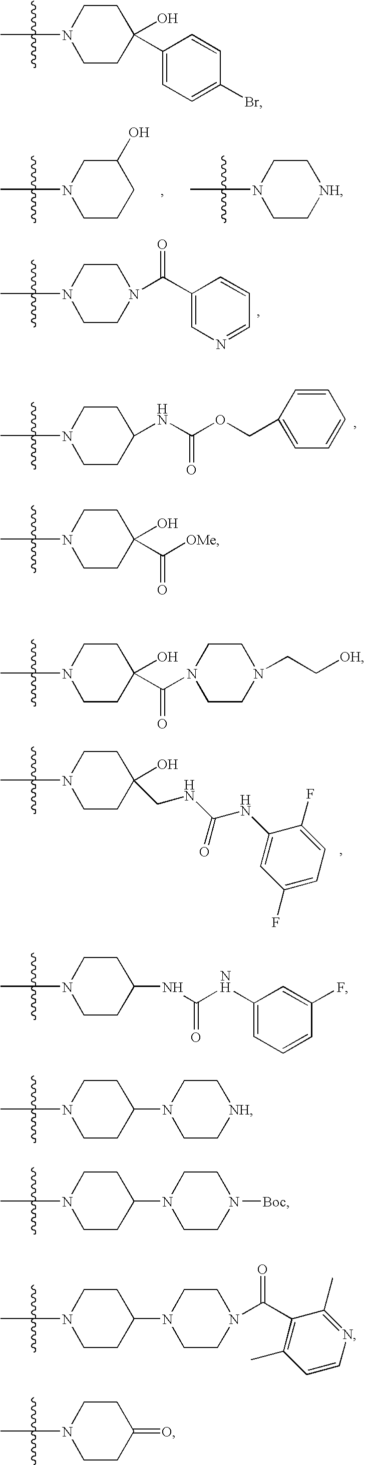

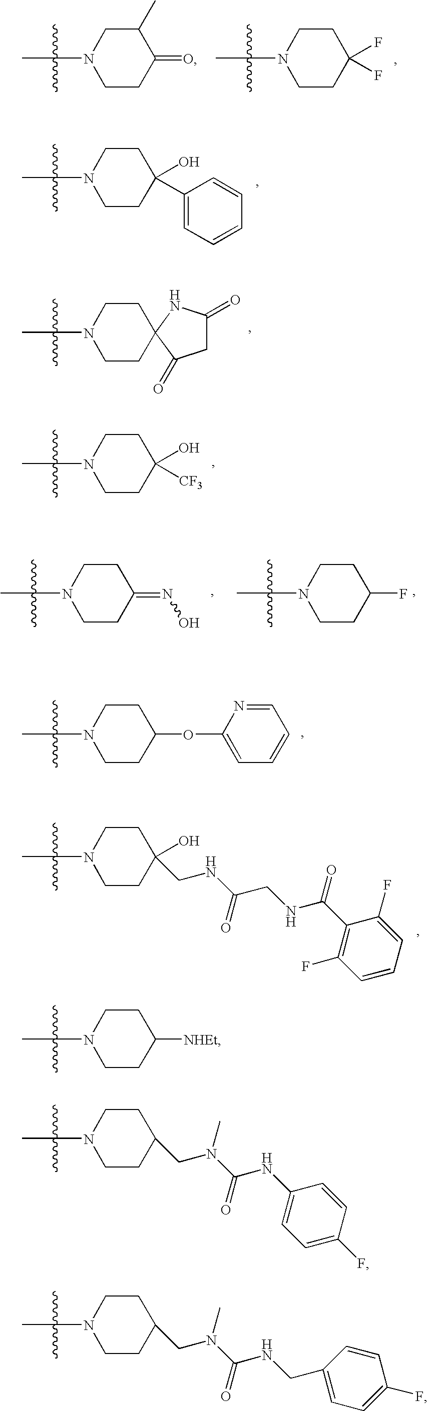

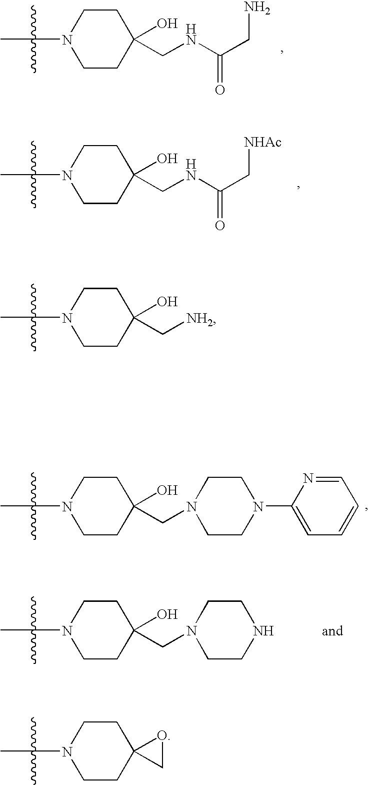

Preferred compounds of formula I include compounds of the following formula II 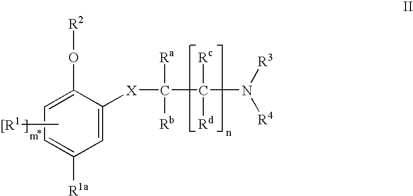
enantiomers, diastereomers, salts and solvates thereof
wherein
m is 0, 1, 2, or 3;
R^{1a} is halo (especially bromo); and
X, R¹, R², R³, R⁴, R^{a}, R^{b}, R^{c}, R^{d} and n are as defined above in formula I (including preferred groups).

Preferred compounds of formula II include compounds of the following formula III 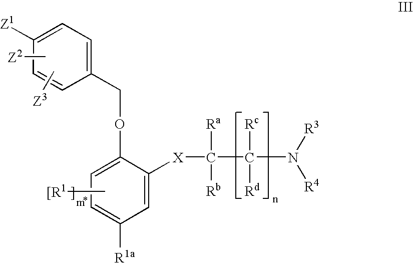
enantiomers, diastereomers, salts and solvates thereof
wherein
Z¹ is halo (especially chloro), cyano, alkyl, haloalkyl, aryl, —C(O)OH, —C(O)V, —C(O)OV, or —U¹—NV²V³ (especially where U¹ is —C(O)—);
Z² and Z³ are optional substituents as defined above in formula I; and X, R¹, R^{1a}, R², R³, R⁴, R^{a}, R^{b}, R^{c}, R^{d} and n and m* are as defined above in formula II (including preferred groups).

Other preferred embodiments of the present invention include:
a) A pharmaceutical composition comprising a compound of formula I in admixture with a pharmaceutically acceptable excipient, diluent, or carrier;
b) A method for modulation of chemokine receptor activity in a patient (e.g.,mammal, e.g., human) which comprises administering an effective amount of a compound of formula I;
c) A method for the prevention or treatment of an inflammatory or immunoregulatory disorder or disease which comprises administering to a patient an effective amount of a compound of formula I;
d) A method for the prevention or treatment of asthma, allergic rhinitis, dermatitis, conjunctivitis, or atherosclerosis which comprises administering to a patient an effective amount of a compound of formula I;
e) A method for the prevention or treatment of rheumatoid arthritis which comprises administering to a patient an effective amount of a compound of formula I;
f) A method for preventing infection by HIV, treating infection by HIV, delaying the onset of AIDS, or treating AIDS comprising administering to a patient an effective amount of a compound of formula I;
g) A method for the prevention or treatment of multiple sclerosis or psoriasis which comprises administering to a patient an effective amount of a compound of formula I;
h) A method of inhibiting the binding of MIP-1α or MIP-1β to a receptor comprising administering a therapeutically effective amount of a compound of formula I to a mammal in need thereof;
i) A method of inhibiting the binding of RANTES to a receptor comprising administering a therapeutically effective amount of a compound of formula I to a mammal in need thereof; and
j) A method of assaying compounds which modulate the activity of a CCR-5 receptor comprising screening against a compound of formula (I);

Preferred compounds of formula (I) are:
N-[[5-bromo-2-(4-chlorophenylmethoxy)phenyl]methyl]morpholineethanamine, dihydrochloride
5-bromo-2-(4-chlorophenylmethoxy)-N,N-diethylbenzenemethanamine, hydrochloride;
1-[[[5-bromo-2-(4-chlorophenylmethoxy)phenyl]methyl]amino]-2-propanol, Hydrochloride
1-[[5-bromo-2-(4-chlorophenylmethoxy)phenyl]methyl]-4-ethylpiperazine, dihydrochloride
N-[[5-bromo-2-(4-chlorophenylmethoxy)phenyl]methyl]-N′,N′-dimethylpropanediamine, dihydrochloride;
3-[[4-bromo-2-[(diethylamino)methyl]phenoxy]methyl]benzoic acid, methyl ester, hydrochloride;
4-[[5-bromo-2-[(4-chlorophenyl)methoxy]phenyl]methyl]thiomorpholine;
5-bromo-2-[(4-chlorophenyl)methoxy]-N-methyl-N-(phenylmethyl)benzenemethanamine;
5-bromo-2-[(4-chlorophenyl)methoxy]-N-ethylbenzenemethanamine;
4-[[5-bromo-2-[(4-chlorophenyl)methoxy]phenyl]methyl]morpholine;
5-bromo-2-[(4-chlorophenyl)methoxy]-N-(phenylmethyl)benzenemethanamine;
5-bromo-2-[(4-chlorophenyl)methoxy]-N,N-dimethylbenzenemethanamine;
[1-[[5-bromo-2-[(4-chlorophenyl)methoxy]phenyl]methyl]-3-pyrrolidinyl]-carbamic acid-1,1-dimethylethyl ester;
3-[[4-bromo-2-[(diethylamino)methyl]phenoxy]methyl]benzoic acid, methyl ester, hydrochloride;
1-[[5-bromo-2-[(4-iodophenyl)methoxy]phenyl]methyl]-4-piperidinol;
1-[[5-bromo-2-[(4-methylphenyl)methoxy]phenyl]methyl]-4-piperidinol;
1-[[5-bromo-2-[(4-chlorophenyl)methoxy]phenyl]methyl]-4-piperidinol;
1-[[5-bromo-2-[(6-methyl-3-pyridinyl)methoxy]phenyl]methyl]-4-piperidinol;
1-[[4-bromo-2-[(6-methyl-3-pyridinyl)methoxy]phenyl]methyl]-4-(4-bromophenyl)-4-piperidinol;
4-[[4-chloro-2-(4-morpholinylmethyl)phenoxy]methyl]benzonitrile;
4-[[4-chloro-2-(1-pyrrolidinylmethyl)phenoxy]methyl]benzonitrile;
1-[[5-bromo-2-[(4-chlorophenyl)methoxy]phenyl]methyl]-3-piperidinemethanol;
N-[[5-bromo-2-(4-chlorophenylmethoxy)phenyl]methyl]-N-(3-dimethylaminopropyl)-N′-phenylurea, hydrochloride;
4-[[4-bromo-2-[(dimethylamino)methyl]phenoxy]methyl]-N-(3,4-dimethoxyphenylmethyl)benzamide, hydrochloride;
5-bromo-2-[[4-[(6,7-dimethoxy-3,4-dihydro-2(1H)-isoquinolinyl)carbonyl]phenyl]methoxy]-N,N-dimethylbenzenemethanamine, hydrochloride;
4-bromo-2-(bromomethyl)-1-[(4-chlorophenyl)methoxy]benzene;
2-[[[5-bromo-2-[(4-chlorophenyl)methoxy]phenyl]methyl]amino]-1,3-propanediol;
(2R)-1-[[5-bromo-2-[(4-chlorophenyl)methoxy]phenyl]methyl]-2-pyrrolidinemethanol, trifluoroacetic acid salt;
(2S)-1-[[5-bromo-2-[(4-chlorophenyl)methoxy]phenyl]methyl]-2-pyrrolidinemethanol, trifluoroacetic acid salt;
(2R)-1-[[5-bromo-2-[(4-chlorophenyl)methoxy]phenyl]methyl]-3-pyrrolidinol, trifluoroacetic acid salt;
N¹-[[5-bromo-2-[(4-chlorophenyl)methoxy]phenyl]methyl]-N¹-[2-(diethylamino)ethyl]-N²,N²-diethyl-1,2-ethanediamine;
1-[[5-bromo-2-[(4-chlorophenyl)methoxy]phenyl]methyl]-4-piperidinone;
1-[[5-bromo-2-[(4-chlorophenyl)methoxy]phenyl]methyl]-4-(4-bromophenyl)-4-piperidinol;
1-[[5-bromo-2-[(4-chlorophenyl)methoxy]phenyl]methyl]-4-piperidinol;
[1-[[5-bromo-2-[(4-chlorophenyl)methoxy]phenyl]methyl]-4-piperidinyl]-carbamic acid, 1,1-dimethylethyl ester;
1-[[5-bromo-2-[(4-chlorophenyl)methoxy]phenyl]methyl]-4-ethoxy-piperidine.
8-[[5-bromo-2-[(4-chlorophenyl)methoxy]phenyl]methyl]-1,4-dioxa-8-azaspiro[4.5]decane,
[[1-[[5-bromo-2-[(4-chlorophenyl)methoxy]phenyl]methyl]-4-piperidinyl]methyl]-carbamic acid, 1,1 -dimethylethyl ester;
5-bromo-2-[(4-chlorophenyl)methoxy]benzenemethanamine;
1-[[5-bromo-2-[(4-chlorophenyl)methoxy]phenyl]methyl]pyridinium bromide;
1-[[5-bromo-2-[(4-chlorophenyl)methoxy]phenyl]methyl]-4-piperidinecarboxylic acid, ethyl ester;
2-[[[5-bromo-2-[(4-chlorophenyl)methoxy]phenyl]methyl]amino]ethanol;
2-[[[5-bromo-2-[(4-chlorophenyl)methoxy]phenyl]methyl](methyl)amino]-ethanol;
1-[[5-bromo-2-[(4-chlorophenyl)methoxy]phenyl]methyl]-3-piperidinol;
1-[[5-bromo-2-[(4-chlorophenyl)methoxy]phenyl]methyl]-3-pyrrolidinol,
(1S,2S)-2-[[[5-bromo-2-[(4-chlorophenyl)methoxy]phenyl]methyl]amino]-1-(4-nitrophenyl)-1,3-propanediol;
5-bromo-2-[(4-chlorophenyl)methoxy]-N,N,N-trimethyl-benzenemethanaminium iodide;
(3R,4S)-1-[[5-bromo-2-[(4-chlorophenyl)methoxy]phenyl]methyl]-3,4-pyrrolidinediol;
1-[[5-bromo-2-[(4-chlorophenyl)methoxy]phenyl]methyl]-4-piperidinecarboxylic acid;
1-[[5-bromo-2-[(4-chlorophenyl)methoxy]phenyl]methyl]-4-piperidinamine;
1-[[5-bromo-2-[(4-chlorophenyl)methoxy]phenyl]methyl]-4-piperidinemethanamine;
1-[[5-bromo-2-[(4-chlorophenyl)methoxy]phenyl]methyl]-N-methyl-4-piperidinemethanamine
[1-[[5-bromo-2-[(4-chlorophenyl)methoxy]phenyl]methyl]-4-piperidinyl](ethyl)carbamic acid, 1,1-dimethylethyl ester;
[1-[[5-bromo-2-[(4-chlorophenyl)methoxy]phenyl]methyl]-4-piperidinyl](methyl)carbamic acid, 1,1-dimethylethyl ester;
1-[[5-bromo-2-[(4-chlorophenyl)methoxy]phenyl]methyl]-N,N-diethyl-4-piperidinamine;
N-[1-[[5-bromo-2-[(4-chlorophenyl)methoxy]phenyl]methyl]-4-piperidinyl]-N′-(4-fluorophenyl)-urea;
N-[1-[[5-bromo-2-[(4-chlorophenyl)methoxy]phenyl]methyl]-3-pyrrolidinyl]-N′-(4-fluorophenyl)-urea;
N-[[1-[[5-bromo-2-[(4-chlorophenyl)methoxy]phenyl]methyl]-4-piperidinyl]methyl]-N′-(4-fluorophenyl)-N-methyl-urea;
N-[[1-[[5-bromo-2-[(4-chlorophenyl)methoxy]phenyl]methyl]-4-piperidinyl]methyl]-N′-[(4-fluorophenyl)methyl]-N-methyl-urea;
N-[1-[[5-bromo-2-[(4-chlorophenyl)methoxy]phenyl]methyl]-3-pyrrolidinyl]-2-chloroacetamide;
N-[[1-[[5-bromo-2-[(4-chlorophenyl)methoxy]phenyl]methyl]-4-piperidinyl]methyl]acetamide, trifluoroacetic acid salt;
N-[1-[[5-bromo-2-[(4-chlorophenyl)methoxy]phenyl]methyl]-4-piperidinyl]-acetamide, Trifluoroacetic acid salt;
N-[1-[[5-bromo-2-[(4-chlorophenyl)methoxy]phenyl]methyl]-3-pyrrolidinyl]-N-methyl-2-pyrazinecarboxamide;
N-[[1-[[5-bromo-2-[(4-chlorophenyl)methoxy]phenyl]methyl]-4-piperidinyl]methyl]-N,4-dimethyl-3-pyridinecarboxamide;
[1-[[5-bromo-2-[(4-chlorophenyl)methoxy]phenyl]methyl]-4-piperidinyl]carbamic acid, methyl ester;
4-[[4-bromo-2-[(diethylamino)methyl]phenoxy]methyl]benzoic acid;
5-bromo-N,N-diethyl-2-[[4-[[4-(phenylmethyl)-1-piperazinyl]carbonyl]phenyl]methoxy]benzenemethanamine;
N-(1,3-Benzodioxo-5-ylmethyl)-4-[[4-bromo-2-[(diethylamino)methyl]phenoxy]methyl]benzamide;
4-[[4-bromo-2-[(diethylamino)methyl]phenoxy]methyl]-N-[(4-methoxyphenyl)methyl]benzamide;
4-[[4-bromo-2-[(diethylamino)methyl]phenoxy]methyl]-N-methyl-N-(2-phenylethyl)benzamide;
4-[[4-bromo-2-[(diethylamino)methyl]phenoxy]methyl]-N-[2-(4-bromo-phenyl)ethyl]benzamide;
4-[4-[[4-bromo-2-[(diethylamino)methyl]phenoxy]methyl]benzoyl]-N-octyl-1-piperazincarboxamide;
5-bromo-N,N-didiethyl-2-[[4-[[4-[[3-nitrophenyl)sulfonyl]-1-piperazinyl]carbonyl]phenyl]methoxy]benzenemethanamine;
5-bromo-N,N-diethyl-2-[[4-[[4-(2-furanylcarbonyl)-1-piperazinyl]carbonyl]phenyl]methoxy]benzenemethanamine;
5-bromo-2-[[4-[[4-(2,6-dichlorobenzoyl)-1-piperazinyl]carbonyl]phenyl]methoxy]-N,N-diethylbenzenemethanamine;
N-[[5-[[4-[4-[[4-bromo-2-[(diethylamino)methyl]phenoxy]methyl]benzoyl]-1-piperazinyl]sulfonyl]-2-thienyl]methyl]benzamide;
1-[(5-bromo-2-propoxyphenyl)methyl]-4-(4-fluorophenyl)-4-piperidinol;
[4-bromo-2-[[4-(4-bromophenyl)-4-hydroxy-1-piperidinyl]methyl]phenoxy]-O-ethyloxime-ethanal;
1-[(5-bromo-2-propoxyphenyl)methyl]-4-(4-chlorophenyl)-4-piperidinol;
1-[[5-bromo-2-(pentyloxy)phenyl]methyl]-4-(4-bromophenyl)-4-piperidinol;
1-[[5-bromo-2-(hexyloxy)phenyl]methyl]-4-(4-bromophenyl)-4-piperidinol;
1-[(5-bromo-2-methoxyphenyl)methyl]-4-(4-bromophenyl)-4-piperidinol;
1-[[5-bromo-2-(1,3-dioxolan-2-ylmethoxy)phenyl]methyl]-4-(4-bromophenyl)-4-piper
1-[(5 -bromo-2-hydroxyphenyl)methyl]-4-(4-bromophenyl)-4-piperidinol;
1-[[5-bromo-2-(2-methylpropoxy)phenyl]methyl]-4-(4-bromophenyl)-4-piperidinol;
1-[[5-bromo-2-(heptyloxy)phenyl]methyl]-4-(4-bromophenyl)-4-piperidinol, trifluoroacetic acid;
1-[[5-bromo-2-(cyclopropylmethoxy)phenyl]methyl]-4-(4-bromophenyl)-4-piperidinol, trifluoroacetic acid;
1-[(5-bromo-2-butoxyphenyl)methyl]-4-(4-bromophenyl)-4-piperidinol, trifluoroacetic acid;
1-[[5-bromo-2-(2-methoxyethoxy)phenyl]methyl]-4-(4-bromophenyl)-4-piperidinol, trifluoroacetic acid;
4-(4-bromophenyl)-1-[(5-bromo-2-propoxyphenyl)methyl]-4-piperidinol, trifluoroacetic acid;
1-[(5-bromo-2-ethoxyphenyl)methyl]-4-(4-bromophenyl)-4-piperidinol, trifluoroacetic acid;
4-(4-bromophenyl)-1-[[5-bromo-2-(2-propenyloxy)phenyl]methyl]-4-piperidinol, trifluoroacetic acid;
[5-bromo-2-[[4-(4-bromophenyl)-4-hydroxy-1-piperidinyl]methyl]phenoxy]-acetonitrile, trifluoroacetic acid;
N-[2-[4-bromo-2-[[4-(4-bromophenyl)-4-hydroxy-1-piperidinyl]methyl]phenoxy]ethyl]-N′-ethyl-urea;
1-[[2-(2-aminoethoxy)-5-bromophenyl]methyl]-4-(4-bromophenyl)-4-piperidinol: 2-bromo-1-[5 -bromo-2-[(4-chlorophenyl)methoxy]phenyl]-ethanone;
4-[[4-bromo-2-(bromoacetyl)phenoxy]methyl]benzoic acid, methyl ester;
1-[2-([1,1′-biphenyl]-4-ylmethoxy)-5-bromophenyl]-2-bromoethanone;
3-[[4-[4-[[4-bromo-2-(bromoacetyl)phenoxy]methyl]benzoyl]-1-piperazinyl]sulfonyl]-N-hydroxy-N-oxo-benzenaminium;
2-bromo-1-[5-bromo-2-[(4-chlorophenyl)methoxy]phenyl]-1-propanone;
1-[5-bromo-2-[(4-chlorophenyl)methoxy]phenyl]-2-(dimethylamino)-ethanone;
1-[2-([1,1′-biphenyl]-4-ylmethoxy)-5-bromophenyl]-2-(dimethylamino)-ethanone;
1-[2-([1,1′-biphenyl]-4-ylmethoxy)-5-bromophenyl]-2-bromoethanone;
5-bromo-2-[(4-chlorophenyl)methoxy]-α-[[(2-hydroxyethyl)(methyl)amino]methyl]benzenemethanol, trifluoroacetic acid salt;
α-[5-bromo-2-[(4-chlorophenyl)methoxy]phenyl]-3-hydroxy-1-piperidineethanol, trifluoroacetic acid salt;
α-[5-bromo-2-[(4-chlorophenyl)methoxy]phenyl]-3-hydroxy-1-pyrrolidineethanol, trifluoroacetic acid salt;
(2S, 4R)-1-[2-[5-bromo-2-[(4-chlorophenyl)methoxy]phenyl]-2-hydroxyethyl]-4-hydroxy-2-pyrrolidinecarboxylic acid, trifluoroacetic acid salt;
5-bromo-2-[(4-chlorophenyl)methoxy]-α-[(dimethylamino)methyl]benzenemethanol, trifluoroacetic acid salt;
2-Amino-α-[5-bromo-2-[(4-chlorophenyl)methoxy]phenyl]-1H-imidazole-1-ethanol;
α-[5-bromo-2-[(4-chlorophenyl)methoxy]phenyl]-4-hydroxy-1-piperidineethanol;
4-[[4-bromo-2-[1-hydroxy-2-(4-hydroxy-1-piperidinyl)ethyl]phenoxy]methyl]benzoic acid, methyl ester, trifluoroacetic acid salt.;
4-[[4-bromo-2-[1-hydroxy-2-(3-hydroxy-1-piperidinyl)ethyl]phenoxy]methyl]benzoic acid, methyl ester, trifluoroacetic acid salt;
4-[[4-bromo-2-[2-[4-[[(1,1-dimethylethoxy)carbonyl]amino]-1-piperidinyl]-1-hydroxyethyl]phenoxy]methyl]benzoic acid, methyl ester;
2-([1,1′-biphenyl]-4-ylmethoxy)-5-bromo-α-[(dimethylamino)methyl]-benzenemethanol, trifluoroacetic acid salt;
4-[[4-chloro-2-[1-hydroxy-2-(4-hydroxy-1-piperidinyl)ethyl]phenoxy]methyl]benzoic acid;
1-[5-bromo-2-[(4-chlorophenyl)methoxy]phenyl]-2-(dimethylamino)-1-propanone;
5-chloro-2-[(4-chlorophenyl)methoxy]-α-[1-(dimethylamino)ethyl]benzenemethanol;
α-[5-chloro-2-[(4-chlorophenyl)methoxy]phenyl]-β-methyl-1H-imidazole-1-ethanol;
α-[5-chloro-2-[(4-chlorophenyl)methoxy]phenyl]-4-(4-chlorophenyl)-4-hydroxy-β-methyl-1-piperidineethanol;
α-[5-chloro-2-[(4-chlorophenyl)methoxy]phenyl]-4-hydroxy-β-methyl-4-(phenylmethyl)-1-piperidineethanol;
α-[5-chloro-2-[(4-chlorophenyl)methoxy]phenyl]-4-(4-fluorophenyl)-4-hydroxy-β-methyl-1-piperidineethanol;
5-chloro-2-[(4-chlorophenyl)methoxy]-α-[1-(diethylamino)ethyl-benzenemethanol;
α-[5-bromo-2-[[4-[[4-[(3-nitrophenyl)sulfonyl]-1-piperazinyl]carbonyl]phenyl]methoxy]phenyl]-3-hydroxy-1-piperidineethanol;
α-5-bromo-2-[[4-[[4-[(3-nitrophenyl)sulfonyl]-1-piperazinyl]carbonyl]phenyl]methoxy]phenyl]-4-hydroxy-1-piperidineethanol;
α-[5-bromo-2-[[4-[[4-[(3-nitrophenyl)sulfonyl]-1-piperazinyl]carbonyl]phenyl]methoxy]phenyl]-3-hydroxy-1-pyrrolidineethanol;
5-bromo-α-[(diethylamino)methyl]-2-[[4-[[4-[(3-nitrophenyl)sulfonyl]-1-piperazinyl]carbonyl]phenyl]methoxy]benzenemethanol;
α-[5-bromo-2-[(4-chlorophenyl)methoxy]phenyl]-1-piperazineethanol;
α-[5-bromo-2-[(4-chlorophenyl)methoxy]phenyl]-4-(3-pyridinylcarbonyl)-1-piperazineethanol;
α-[5-bromo-2-[(4-chlorophenyl)methoxy]phenyl]-4-[(4-methyl-3-pyridinyl)carbonyl]-1-piperazineethanol.;
4-[[4-bromo-2-[1-hydroxy-2-[4-[[(phenylmethoxy)carbonyl]amino]-1-piperidinyl]ethyl]phenoxy]methyl]benzoic acid, methyl ester;
4-[[4-bromo-2-[1-hydroxy-2-(4-hydroxy-1-piperidinyl)ethyl]phenoxy]methyl]-N-(4-pyridinyl)benzamide;
4-[[4-chloro-2-[1-hydroxy-2-(4-hydroxy-1-piperidinyl)ethyl]phenoxy]methyl]-N-(3-hydroxypropyl)benzamide;
2-[5-bromo-2-[(4-chlorophenyl)methoxy]phenyl]oxirane;
1-(2S)-α-[5-bromo-2-[(4-chlorophenyl)methoxy]phenyl]-2-(hydroxymethyl)-1-pyrrolidineethanol, trifluoroacetic acid salt;
(2R)-α-[5-bromo-2-[(4-chlorophenyl)methoxy]phenyl]-2-(hydroxymethyl)-1-pyrrolidineethanol, trifluoroacetic acid salt;
(3R)-α-[5-bromo-2-[(4-chlorophenyl)methoxy]phenyl]-3-hydroxy-1-pyrrolidineethanol, trifluoroacetic acid salt;
5-bromo-2-[(4-chlorophenyl)methoxy]-α-[[[2-(diethylamino)ethyl]ethylamino]methyl]-benzenemethanol, trifluoroacetic acid salt;
α-[5-bromo-2-[(4-chlorophenyl)methoxy]phenyl]-1,4-piperidinediethanol, trifluoroacetic acid salt.;
α-[5-bromo-2-[(4-chlorophenyl)methoxy]phenyl]-4-(piperidyl)-1-piperidineethanol;
5-bromo-2-[(4-chlorophenyl)methoxy]-α-[(dipropylamino)methyl]-benzenemethanol, trifluoroacetic acid salt;
α-[5-bromo-2-[(4-chlorophenyl)methoxy]phenyl]-4-(phenylmethyl)-1-piperidineethanol, trifluoroacetic acid salt;
5-bromo-2-[(4-chlorophenyl)methoxy]-α-[(dibutylamino)methyl]-benzenemethanol, trifluoroacetic acid salt;
5-bromo-α-[(butylethylamino)methyl]-2-[(4-chlorophenyl)methoxy]-benzenemethanol;
5-bromo-2-[(4-chlorophenyl)methoxy]-α-[[ethyl(2-hydroxyethyl)amino]methyl]benzenemethanol, trifluoroacetic acid salt;
5-bromo-2-[(4-chlorophenyl)methoxy]-α-[[(2-hydroxyethyl)propylamino]methyl]benzenemethanol trifluoroacetic acid salt;
1-[2-[5-bromo-2-[(4-chlorophenyl)methoxy]phenyl]-2-hydroxyethyl]-N,N-diethyl-3-piperidinecarboxamide, trifluoroacetic acid salt;
α-[5-bromo-2-[(4-chlorophenyl)methoxy]phenyl]-4-(4-bromophenyl)-4-hydroxy-1-piperidineethanol, trifluoroacetic acid salt;
1-[1-[2-[5-bromo-2-[(4-chlorophenyl)methoxy]phenyl]-2-hydroxyethyl]-4-piperidinyl]-1,3-dihydro-H-benzimidazol-2-one, trifluoroacetic acid salt;
1-[2-[5-bromo-2-[(4-chlorophenyl)methoxy]phenyl]-2-hydroxyethyl]-4-phenyl-4-piperidinecarbonitrile, trifluoroacetic acid salt.;
α-[5-bromo-2-[(4-chlorophenyl)methoxy]phenyl]-1,4-dioxa-8-azaspiro[4.5]decane-8-ethanol, trifluoroacetic acid salt;
5-bromo-2-[(4-chlorophenyl)methoxy]-α-[[(2-hydroxyethyl)(phenylmethyl)amino]methyl]-benzenemethanol, trifluoroacetic acid salt;
5-bromo-2-[(4-chlorophenyl)methoxy]-α-[[[2-(dimethylamino)ethyl]ethylamino]methyl]benzenemethanol, trifluoroacetic acid salt;
α-[5-bromo-2-[(4-chlorophenyl)methoxy]phenyl]-1,2,3,4-tetrahydro-1-quinolineethanol, trifluoroacetic acid salt;
1-[2-[5-bromo-2-[(4-chlorophenyl)methoxy]phenyl]-2-hydroxyethyl]-3,4-pyrrolidinediol, trifluoroacetic acid salt.;
5-bromo-2-[(4-chlorophenyl)methoxy]-α-[(methylamino)methyl]-benzenemethanol, trifluoroacetic acid salt;
2-[[2-[5-bromo-2-[(4-chlorophenyl)methoxy]phenyl]-2-hydroxyethyl]amino]-1,3-propaned trifluoroacetic acid salt;
5-bromo-2-[(4-chlorophenyl)methoxy]-α-[(diethylamino)methyl]-benzenemethanol, trifluoroacetic acid salt;
2-[[2-[5-bromo-2-[(4-chlorophenyl)methoxy]phenyl]-2-hydroxyethyl]amino]-2-(hydroxymethyl)-1,3-propanediol;
α-[5-bromo-2-[(4-chlorophenyl)methoxy]phenyl]-1-pyrrolidineethanol;
α-[5-bromo-2-[(4-chlorophenyl)methoxy]phenyl]-1-piperidineethanol;
5-bromo-2-[(4-chlorophenyl)methoxy]-α-[[(3-hydroxyphenyl)amino]methyl]benzenemethanol;
5-bromo-2-[(4-chlorophenyl)methoxy]-α-[[(cyclopropylmethyl)amino]methyl]benzenemethanol;
5-bromo-α-[[[2-(3-chlorophenyl)ethyl]amino]methyl]-2-[(4-chlorophenyl)methoxy]benzenemethanol;
α-[5-bromo-2-[(4-chlorophenyl)methoxy]phenyl]-1-azetidineethanol;
5-bromo-2-[(4-chlorophenyl)methoxy]-α-[(ethylmethylamino)methyl]benzenemethanol;
5-bromo-2-[(4-chlorophenyl)methoxy]-α-[(cyclopropylamino)methyl]benzenemethanol;
5-bromo-2-[(4-chlorophenyl)methoxy]-α-[[(cyclopropylmethyl)methylamino]methyl]benzenemethanol;
α-[5-bromo-2-[(4-chlorophenyl)methoxy]phenyl]-4-thiomorpholineethanol;
α-(Aminomethyl)-5-bromo-2-[(4-chlorophenyl)methoxy]benzenemethanol;
5-bromo-2-[(4-chlorophenyl)methoxy]-α-[(cyclopropylmethylamino)methyl]benzenemethanol;
(αS)-5-bromo-2-[(4-chlorophenyl)methoxy]-α-[(diethylamino)methyl]benzenemethanol;
(αR)-5-bromo-2-[(4-chlorophenyl)methoxy]-α-[(diethylamino)methyl]-benzenemethanol;
α-[[bis(2-hydroxyethyl)amino]methyl]-5-bromo-2-[(4-chlorophenyl)methoxy]benzenemethanol;
α-[5-bromo-2-[(4-chlorophenyl)methoxy]phenyl]-4-methyl-1-piperazineethanol;
5-bromo-2-[(4-chlorophenyl)methoxy]-α-[[(1-methylethyl)amino]methyl]-benzenemethanol;
α-[5-bromo-2-[(4-chlorophenyl)methoxy]phenyl]-4-morpholineethanol;
5-bromo-2-[(4-chlorophenyl)methoxy]-α-[[(2-hydroxyethyl)amino]methyl]-benzenemethanol;
5-bromo-2-[(4-chlorophenyl)methoxy]-α-[[(2-hydroxyethyl)amino]methyl]-benzenemethanol;
5-bromo-2-[(4-chlorophenyl)methoxy]-α-ethoxy-N,N-diethylbenzeneethanamine;
5-bromo-2-[(4-chlorophenyl)methoxy]-N,N-diethyl-α-(2-pyridinyloxy)benzeneethanamine;
5-bromo-2-[(4-chlorophenyl)methoxy]-α-(methylamino)benzeneethanol;
1-[5-bromo-2-[(4-chlorophenyl)methoxy]phenyl]-2-propen-1-one;
(3R)-α-[5-bromo-2-[(4-chlorophenyl)methoxy]phenyl]-3-hydroxy-1-pyrrolidinepropanol;
5-bromo-2-[(4-chlorophenyl)methoxy]-α-[2-(dimethylamino)ethyl]-benzenemethanol:
α-[5-bromo-2-[(4-chlorophenyl)methoxy]phenyl]-4-hydroxy-1-piperidinepropanol;
5-bromo-2-[(4-chlorophenyl)methoxy]-α-[2-(dipropylamino)ethyl]-benzenemethanol;
5-bromo-2-[(4-chlorophenyl)methoxy]-α-[2-(diethylamino)ethyl]-benzenemethanol;
5-chloro-2-[(4-fluorophenyl)methoxy]benzeneethanamine;
N-[[2-[5-chloro-2-[(4-fluorophenyl)methoxy]-phenyl]ethyl]-4-pyridinemethanamine;
5-chloro-2-[(4-fluorophenyl)methoxy]-N,N,α-trimethylbenzeneethanamine;
4-[[[2-[5-chloro-2-[(4-fluorophenyl)methoxy]phenyl]ethyl]amino]methyl]benzonitrile;
N-[2-[5-chloro-2-[[4-fluorophenyl)methoxy]phenyl]ethyl]-N-(1H-imidazol-5-ylmethyl)-1H-imidazole-4-methanamine;
5-chloro-α-ethyl-2-[(4-fluorophenyl)methoxy]-N-[(4-fluorophenyl)methyl]benzeneethanamine;
5-chloro-α-ethyl-2-[(4-fluorophenyl)methoxy]-N-[(3-methyl-4-methoxyphenyl)methyl]benzeneethanamine;
1-[[5-bromo-2-[(4-chlorophenyl)methoxy]phenyl]methyl]-4-hydroxy-4-piperidinecarboxylic acid, methyl ester;
1-[[5-bromo-2-[(4-chlorophenyl)methoxy]phenyl]methyl]-4-hydroxy-4-piperidinecarboxylic acid;
4-[[1-[[5-bromo-2-[(4-chlorophenyl)methoxy]phenyl]methyl]-4-hydroxy-4-piperidinyl]carbonyl]-1-piperazineethanol;
1-[[5-bromo-2-[(4-chlorophenyl)methoxy]phenyl]methyl]-4-(1-piperazinylcarbonyl)-4-piperidinol;
1-[[5-bromo-2-[(4-chlorophenyl)methoxy]phenyl]methyl]-4-[[(3R)-3-methylpiperazinyl]carbonyl]-4-piperidinol;
4-[1-[[5-bromo-2-[(4-chlorophenyl)methoxy]phenyl]methyl]-4-piperidinyl]-1-piperazinecarboxylic acid, 1,1-dimethylethyl ester;
1-[1-[[5-bromo-2-[(4-chlorophenyl)methoxy]phenyl]methyl]-4-piperidinyl]piperazine;
1-[1-[[5-bromo-2-[(4-chlorophenyl)methoxy]phenyl]methyl]-4-piperidinyl]-4-[(2,4-dimethyl-3-pyridinyl)carbonyl]piperazine;
1-[[5-bromo-2-[(4-chlorophenyl)methoxy]phenyl]methyl]-3-methyl-4-piperidinone;
1-[[5-bromo-2-[(4-chlorophenyl)methoxy]phenyl]methyl]-3-methyl-4-piperidinol;
1-[[5-bromo-2-[(4-chlorophenyl)methoxy]phenyl]methyl]-4,4-difluoropiperidine;
8-[[5-bromo-2-[(4-chlorophenyl)methoxy]phenyl]methyl]-1,3,8-triazaspiro[4.5]decane-2,4-dione;
1-[[5-bromo-2-[(4-chlorophenyl)methoxy]phenyl]methyl]-4-phenyl-4-piperidinol;
1-[[5-bromo-2-[(4-chlorophenyl)methoxy]phenyl]methyl]-4-ethyl-4-piperidinol;
1-[[5-bromo-2-[(4-chlorophenyl)methoxy]phenyl]methyl]-4-(trifluoromethyl)-4-piperdinol;
1-[[5-bromo-2-[(4-chlorophenyl)methoxy]phenyl]methyl]-4-piperidinone-oxime;
1-[[5-bromo-2-[[4-(trifluoromethyl)phenyl]methoxy]phenyl]methyl]-4-fluoropiperidine;
1-[[5-bromo-2-[(4-chlorophenyl)methoxy]phenyl]methyl]-4-(2-pyridinyloxy)piperidine;
2-[[1-[[5-bromo-2-[(4-chlorophenyl)methoxy]phenyl]methyl]-4-piperidinyl]oxy]pyrimidine;
1-[[5-bromo-2-[(4-chlorophenyl)methoxy]phenyl]methyl]-N-ethyl-4-piperidinamine;
6-[[5-bromo-2-[(4-chlorophenyl)methoxy]phenyl]methyl]-1-oxa-6-azaspiro[2.5]octane;
4-(aminomethyl)-1-[[5-bromo-2-[(4-chlorophenyl)methoxy]phenyl]methyl]-4-piperidinol;
1-[[5-bromo-2-[(4-chlorophenyl)methoxy]phenyl]methyl]-4-[[[[1-[[5-bromo-2-[(4-chlorophenyl)methoxy]phenyl]methyl]-4-hydroxy-4-piperidinyl]methyl]amino]methyl]-4-piperidinol;
4-[[1-[[5-bromo-2-[(4-chlorophenyl)methoxy]phenyl]methyl]-4-hydroxy-4-piperidinyl]methyl]-1-piperazinecarboxylic acid, 1,1-dimethylethyl ester;
4-[[1-[[5-bromo-2-[(4-chlorophenyl)methoxy]phenyl]methyl]-4-hydroxy-4-piperidinyl]methyl]hexahydro-1H-1,4-diazepine-1-carboxylic acid, 1,1-dimethylethyl ester;
1-[[5-bromo-2-[(4-chlorophenyl)methoxy]phenyl]methyl]-4-[[4-(2-pyridinyl)-1-piperazinyl]methyl]-4-piperidinol;
1-[[5-bromo-2-[(4-chlorophenyl)methoxy]phenyl]methyl]-4-[[4-(2-pyrimidinyl)-1-piperazinyl]methyl]-4-piperidinol.;
1-[[5-bromo-2-[(4-chlorophenyl)methoxy]phenyl]methyl]-4-(1-piperazinylmethyl)-4-piperidinol;
1-[[5-bromo-2-[(4-chlorophenyl)methoxy]phenyl]methyl]-4-[(hexahydro-1H-1,4-diazepin-1-yl)methyl]-4-piperidinol;
1-[[5-bromo-2-[(4-chlorophenyl)methoxy]phenyl]methyl]-4-[[(4-methylphenyl)amino]methyl]-4-piperidinol;
1-[[5-bromo-2-[(4-chlorophenyl)methoxy]phenyl]methyl]-4-[[(4-methoxyphenyl)amino]methyl]-4-piperidinol;
1-[[5-bromo-2-[(4-chlorophenyl)methoxy]phenyl]methyl]-4-[[(3 S)-3-methylpiperazinyl]methyl]-4-piperidinol;
1-[[5-bromo-2-[(4-chlorophenyl)methoxy]phenyl]methyl]-4-[(2,5-dimethyl-1-piperazinyl)methyl]-4-piperidinol;
4-[[(3-Aminopropyl)amino]methyl]-1-[[5-bromo-2-[(4-chlorophenyl)methoxy]phenyl]methyl]-4-piperidinol;
1-[[5-bromo-2-[(4-chlorophenyl)methoxy]phenyl]methyl]-4-[[[2-(1-piperidinyl)ethyl]amino]methyl]-4-piperidinol;
2-[[[[1-[[5-bromo-2-[(4-chlorophenyl)methoxy]phenyl]methyl]-4-hydroxy-4-piperidinyl]methyl]amino]methyl]-1-pyrrolidinecarboxylic acid, 1,1-dimethylethyl ester;
1-[[5-bromo-2-[(4-chlorophenyl)methoxy]phenyl]methyl]-4-[[(2-pyrrolidinylmethyl)amino]methyl]-4-piperidinol;
1-[[5-bromo-2-[(4-chlorophenyl)methoxy]phenyl]methyl]-4-[[4-[(2,4-dimethyl-3-pyridinyl)carbonyl]-1-piperazinyl]methyl]-4-piperidinol;
4-[[1-[[5-bromo-2-[(4-chlorophenyl)methoxy]phenyl]methyl]-4-hydroxy-4-piperidinyl]methyl]-1-piperazinecarboxylic acid, ethyl ester;
4-[(4-Acetyl-1-piperazinyl)methyl]-1-[[5-bromo-2-[(4-chlorophenyl)methoxy]phenyl]methyl]-4-piperidinol;
1-[[5-bromo-2-[(4-chlorophenyl)methoxy]phenyl]methyl]-4-[(1-piperazinylamino)methyl]-4-piperidinol;
4-[[1-[[5-bromo-2-[(4-chlorophenyl)methoxy]phenyl]methyl]-4-hydroxy-4-piperidinyl]methyl]-1-piperazineethanol;
4-[[1-[[5-bromo-2-[(4-chlorophenyl)methoxy]phenyl]methyl]-4-hydroxy-4-piperidinyl]methyl]-1-piperazinecarboxaldehyde;
4-[[1-[[5-bromo-2-[(4-chlorophenyl)methoxy]phenyl]methyl]-4-hydroxy-4-piperidinyl]methyl]-1-piperazinecarboxylic acid, phenylmethyl ester;
1-[[5-bromo-2-[(4-chlorophenyl)methoxy]phenyl]methyl]-4-[[4-(phenylmethyl)-1-piperazinyl]methyl]-4-piperidinol;
1-[[5-bromo-2-[(4-chlorophenyl)methoxy]phenyl]methyl]-4-[[(2-methylphenyl)amino]methyl]-4-piperidinol;
1-[[1-[[5-bromo-2-[(4-chlorophenyl)methoxy]phenyl]methyl]-4-hydroxy-4-piperidinyl]methyl]-4-piperidinecarboxamide;
1-[[5-bromo-2-[(4-chlorophenyl)methoxy]phenyl]methyl]-4-[[(3S)-3-methylpiperazinyl]methyl]-4-piperidinol;
4-[[1-[[5-bromo-2-[(4-chlorophenyl)methoxy]phenyl]methyl]-4-hydroxy-4-piperidinyl]methyl]-2-piperazinone;
1-[[5-bromo-2-[(4-chlorophenyl)methoxy]phenyl]methyl]-4-[[(3S)-3-methylpiperazinyl]methyl]-4-piperidinol;
1-[[5-bromo-2-[(4-chlorophenyl)methoxy]phenyl]methyl]-4-[(3,5-dimethyl-1-piperazinyl)methyl]-4-piperidinol;
1-[[5-bromo-2-[(4-chlorophenyl)methoxy]phenyl]methyl]-4-(2,5-diazabicyclo[2.2.1]hept-2-ylmethyl)-4-piperidinol;
[1-[[1-[[5-bromo-2-[(4-chlorophenyl)methoxy]phenyl]methyl]-4-hydroxy-4-piperidinyl]methyl]-3-pyrrolidinyl]-carbamic acid, 1,1-dimethylethyl ester;
4-[(3-Amino-1-pyrrolidinyl)methyl]-1-[[5-bromo-2-[(4-chlorophenyl)methoxy]phenyl]methyl]-4-piperidinol;
1-[[5-bromo-2-[(4-chlorophenyl)methoxy]phenyl]methyl]-4-[[4-[2-(dimethylamino)ethyl]-1-piperazinyl]methyl]-4-piperidinol;
1-[[5-bromo-2-[(4-chlorophenyl)methoxy]phenyl]methyl]-4-[[4-[2-(4-morpholinyl)-2-oxoethyl]-1-piperazinyl]methyl]-4-piperidinol;
1-[[5-bromo-2-[(4-chlorophenyl)methoxy]phenyl]methyl]-4-[[4-[3-(4-morpholinyl)propyl]-1-piperazinyl]methyl]-4-piperidinol;
1-[[5-bromo-2-[(4-chlorophenyl)methoxy]phenyl]methyl]-4-[[4-[2-(4-morpholinyl)ethyl]-1-piperazinyl]methyl]-4-piperidinol;
1-[[5-bromo-2-[(4-chlorophenyl)methoxy]phenyl]methyl]-4-[(dimethylamino)methyl]-4-piperidinol;
1-[[5-bromo-2-[(4-chlorophenyl)methoxy]phenyl]methyl]-4-[(diethylamino)methyl]-4-piperidinol;
1-[[5-bromo-2-[(4-chlorophenyl)methoxy]phenyl]methyl]-4-[[(1-methylethyl)amino]methyl]-1-4-piperidinol;
1-[[5-bromo-2-[(4-chlorophenyl)methoxy]phenyl]methyl]-4-[(4-hydroxy-1-piperidinyl)methyl]-4-piperidinol;
1-[[5-bromo-2-[(4-chlorophenyl)methoxy]phenyl]methyl]-4-[[(3R)-3-hydroxypyrrolidinyl]methyl]-4-piperidinol;
1-[[5-bromo-2-[(4-chlorophenyl)methoxy]phenyl]methyl]-4-[[[(4-fluorophenyl)methyl]amino]methyl]-4-piperidinol;
1-[[5-bromo-2-[(4-chlorophenyl)methoxy]phenyl]methyl]-4-(1H-imidazol-1-ylmethyl)-4-piperidinol;
1-[[5-bromo-2-[(4-chlorophenyl)methoxy]phenyl]methyl]-4-[(phenylamino)methyl]-4-piperidinol;
1-[[5-bromo-2-[(4-chlorophenyl)methoxy]phenyl]methyl]-4-[(4-pyridinylamino)methyl]-4-piperidinol;
1-[[5-bromo-2-[(4-chlorophenyl)methoxy]phenyl]methyl]-4-[[(2-hydroxyethyl)methylamino]methyl]-4-piperidinol;
1-[[5-bromo-2-[(4-chlorophenyl)methoxy]phenyl]methyl]-4-[(4-methyl-1-piperazinyl)methyl]-4-piperidinol;
1-[[5-bromo-2-[(4-chlorophenyl)methoxy]phenyl]methyl]-4-[(dipropylamino)methyl]-4-piperidinol;
1-[[1-[[5-bromo-2-[(4-chlorophenyl)methoxy]phenyl]methyl]-4-hydroxy-4-piperidinyl]methyl]-N,N-diethyl-3-piperidinecarboxamide;
1-[[5-bromo-2-[(4-chlorophenyl)methoxy]phenyl]methyl]-4-[[(2,2,2-trifluoroethyl)amino]methyl]-4-piperidinol;
1-[[5-bromo-2-[(4-chlorophenyl)methoxy]phenyl]methyl]-4-[[(3-methylphenyl)amino]methyl]-4-piperidinol;
1-[[5-bromo-2-[(4-chlorophenyl)methoxy]phenyl]methyl]-4-[[[(1R)-1-phenylethyl]amino]methyl]-4-piperidinol;
4-[[1-[[5-bromo-2-[(4-chlorophenyl)methoxy]phenyl]methyl]-4-hydroxy-4-piperidinyl]methyl]-N-ethyl-1-piperazinecarboxamide;
N-[[1-[[5-bromo-2-[(4-chlorophenyl)methoxy]phenyl]methyl]-4-hydroxy-4-piperidinyl]methyl]-N′-(2,6-difluorophenyl)urea;
N-[[1-[[5-bromo-2-[(4-chlorophenyl)methoxy]phenyl]methyl]-4-hydroxy-4-piperidinyl]methyl]-N′-(2,6-dimethoxyphenyl)urea;
N-[[1-[[5-bromo-2-[(4-chlorophenyl)methoxy]phenyl]methyl]-4-hydroxy-4-piperidinyl]methyl]-N′-(2,6-diethylphenyl)urea;
N-[[1-[[5-bromo-2-[(4-chlorophenyl)methoxy]phenyl]methyl]-4-hydroxy-4-piperidinyl]methyl]-N′-(2,4,6-trichlorophenyl)urea.;
N-[[1-[[5-bromo-2-[(4-chlorophenyl)methoxy]phenyl]methyl]-4-hydroxy-4-piperidinyl]methyl]-N′-(2,6-dichlorophenyl)urea;
N-[[1-[[5-bromo-2-[(4-chlorophenyl)methoxy]phenyl]methyl]-4-hydroxy-4-piperidinyl]methyl]-N′-(2,6-dimethylphenyl)urea;
N-[[1-[[5-bromo-2-[(4-chlorophenyl)methoxy]phenyl]methyl]-4-hydroxy-4-piperidinyl]methyl]-N′-(2,6-dibromophenyl)urea;
N-[[1-[[5-bromo-2-[(4-chlorophenyl)methoxy]phenyl]methyl]-4-hydroxy-4-piperidinyl]methyl]-N′-(4-bromo-2,6-dimethylphenyl)urea;
N-[2,6-Bis(1-methylethyl)phenyl]-N′-[[1-[[5-bromo-2-[(4-chlorophenyl)methoxy]phenyl]methyl]-4-hydroxy-4-piperidinyl]methyl]urea;
N-[[1-[[5-bromo-2-[(4-chlorophenyl)methoxy]phenyl]methyl]-4-hydroxy-4-piperidinyl]methyl]-N′-(4-fluorophenyl)urea;
2-amino-N-[[1-[[5-bromo-2-[(4-chlorophenyl)methoxy]phenyl]methyl]-4-hydroxy-4-piperidinyl]methyl]acetamide;
N-[2-[[[1-[[5-bromo-2-[(4-chlorophenyl)methoxy]phenyl]methyl]-4-hydroxy-4-piperidinyl]methyl]amino]-2-oxoethyl]-2,6-difluorobenzamide;
N-[[1-[[5-bromo-2-[(4-chlorophenyl)methoxy]phenyl]methyl]-4-hydroxy-4-piperidinyl]methyl]benzamide;
N-[[1-[[5-bromo-2-[(4-chlorophenyl)methoxy]phenyl]methyl]-4-hydroxy-4-piperidinyl]methyl]-4-chlorobenzamide;
3-[[[[1-[[5-bromo-2-[(4-chlorophenyl)methoxy]phenyl]methyl]-4-hydroxy-4-piperidinyl]methyl]amino]carbonyl]-1-hydroxy-2,4-dimethylpyridinium;
N-[[1-[[5-bromo-2-[(4-chlorophenyl)methoxy]phenyl]methyl]-4-hydroxy-4-piperidinyl]methyl]acetamide;
2-(acetylamino)-N-[[1-[[5-bromo-2-[(4-chlorophenyl)methoxy]phenyl]methyl]-4-hydroxy-4-piperidinyl]methyl]acetamide;
[2-[[[1-[[5-bromo-2-[(4-chlorophenyl)methoxy]phenyl]methyl]-4-hydroxy-4-piperidinyl]methyl]amino]-2-oxoethyl]carbamic acid, phenylmethyl ester;
(αS)-α-amino-N-[[1-[[5-bromo-2-[(4-chlorophenyl)methoxy]phenyl]methyl]-4-hydroxy-4-piperidinyl]methyl]benzeneacetamide;
N-[[1-[[5-bromo-2-[(4-chlorophenyl)methoxy]phenyl]methyl]-4-hydroxy-4-piperidinyl]methyl]-2-chloroacetamide;
N-[[1-[[5-bromo-2-[(4-chlorophenyl)methoxy]phenyl]methyl]-4-hydroxy-4-piperidinyl]methyl]-N-methylacetamide
   or pharmaceutically acceptable salts thereof, wherein these compounds can be in the form of individual optical isomers or mixtures thereof such as diastereomeric mixtures or racemic mixtures.

The term “alkyl” is used herein at all occurrences (as a group per se or a part of a group) to mean straight or branched chain alkyl groups of 1 to 6 carbon atoms, unless the chain length is otherwise indicated, including, but not limited to methyl, ethyl, n-propyl, isopropyl, n-butyl, sec-butyl, isobutyl, tert-butyl, and the like. Alkyl groups may also be substituted one or more times by halogen, aryl, substituted aryl, hydroxy, methoxy, amino, substituted amino, nitro, carboxy, or cyano.

The term “cycloalkyl” as used herein by itself or as part of another group refers to saturated and partially unsaturated (containing 1 or 2 double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 7 carbons, forming the ring. The rings of multi-ring cycloalkyls may be either fused, bridged and/or joined through one or more spiro union to 1 or 2 aromatic, cycloalkyl or heterocyclo rings. Exemplary cycloalkyl groups include cyclopropyl, cyclobutyl, cyclopentyl, cyclohexyl, cycloheptyl, cyclooctyl, cyclodecyl, cyclododecyl, cyclopentenyl, cyclohexenyl, cycloheptenyl, cyclooctenyl, cyclohexadienyl, cycloheptadienyl, 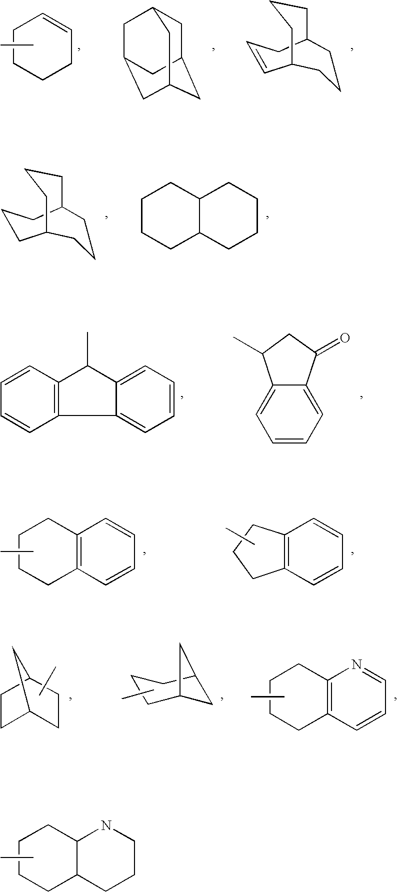
and the like.

Alkoxy group means alkyl-O— groups in which the alkyl portion (substituted or unsubstituted) is in accordance with the previous definition. Suitable alkoxy groups include methoxy, ethoxy, propoxy and butoxy.

The term “cyclic ether” means a cyclic ring of carbon atoms containing an O heteroatom (e.g., epoxide). Rings typically have 3-7 ring atoms and 1 or 2 O atoms.

Alkenyl represents C₂-C₆ carbon chains having one or two unsaturated bonds, provided that two unsaturated bonds are not adjacent to each other.

The term “allyl” means a hydrocarbon radical of 3 to 8 or more carbon atoms, containing a double bond between carbons 2 and 3, and includes, for example, propenyl, 2-butenyl, cinnamyl, and the like.

Suitable substituents on the amino groups herein can be the same or different and include alkyl (optionally substituted), and cycloalkyl, e.g, C₃₋₇ cycloalkyl (optionally substituted e.g., as for alkyl alone). Typical substituents include OH, and C₁₋₆ alkoxy.

The terms “halo” or “halogen” are used interchangeably herein at all occurrences to mean radicals derived from the elements chlorine, fluorine, iodine or bromine. “Halogenated” is analogous and refers to a degree of halogen substitutions from single to full (per) substitution. Fluoro-(C₁-C₆)-alkyl represents a straight or branched alkyl chain, substituted by 1 to 5 fluoro atoms, which can be attached to the same or different carbon atoms, e.g., —CH₂F, —CHF₂, —CF₃, F₃CCH₂— and —CF₂CF₃.

The term “heteroaryl” as used herein by itself or as part of another group refers to monocyclic and bicyclic aromatic rings containing from 5 to 10 atoms, which includes 1 to 4 hetero atoms such as nitrogen, oxygen or sulfur, and such rings fused to an aryl, cycloalkyl, heteroaryl or heterocyclo ring, where the nitrogen and sulfur heteroatoms may optionally be oxidized and the nitrogen heteroatoms may optionally be quaternized. Examples of heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl, indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridyl, dihydroisoindolyl, tetrahydroquinolinyl, carbazolyl, benzidolyl, phenanthrollinyl, acridinyl, phenanthridinyl, xanthenyl 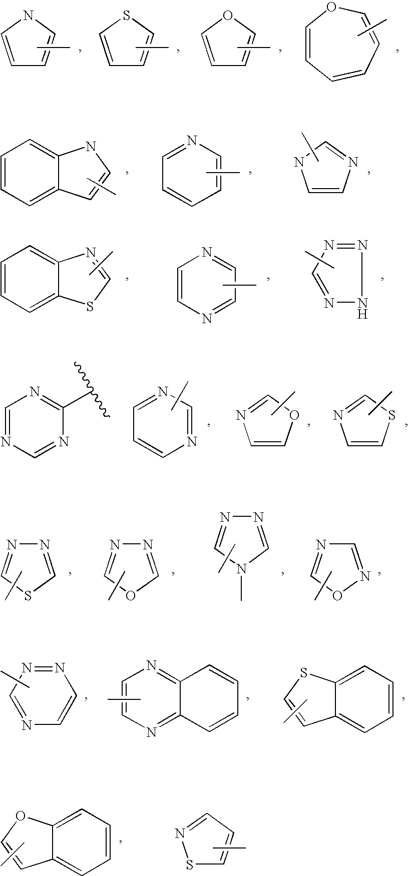
and the like.

The terms “heterocyclic” or “heterocyclo” as used herein by itself or as part of another group refer to optionally substituted, fully saturated or partially unsaturated cyclic groups (for example, 3 to 13 member monocyclic, 7 to 17 member bicyclic, or 10 to 20 member tricyclic ring systems, preferably containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valance allows. The rings of multi-ring heterocycles may be either fused, bridged and/or joined through one or more spiro unions to 1 or 2 aromatic, heteroaryl or cycloalkyl rings. Exemplary heterocyclic groups include azetidinyl, pyrrolidinyl, oxetanyl, imidazolinyl , oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, 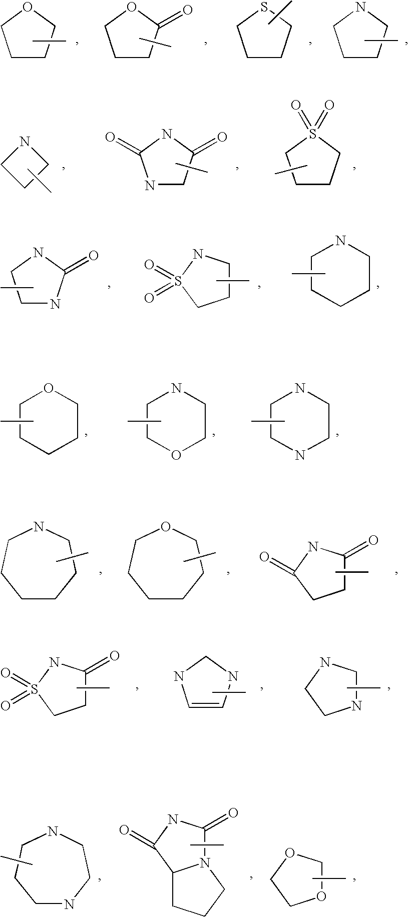 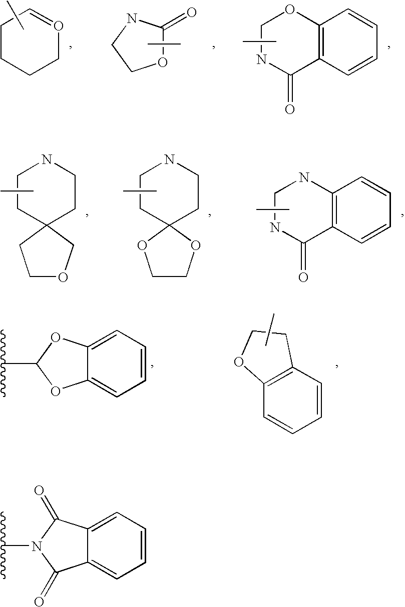
and the like.

The terms “ar” or “aryl” as used herein by itself or as part of another group refer to aromatic homocyclic (i.e., hydrocarbon) monocyclic, bicyclic or tricyclic aromatic groups containing 6 to 14 carbons in the ring portion (such as phenyl, biphenyl, naphthyl (including 1-naphthyl and 2-naphthyl) and antracenyl) and may optionally include one to three additional rings (either cycloalkyl, heterocyclo or heteroaryl) fused thereto. Examples include: 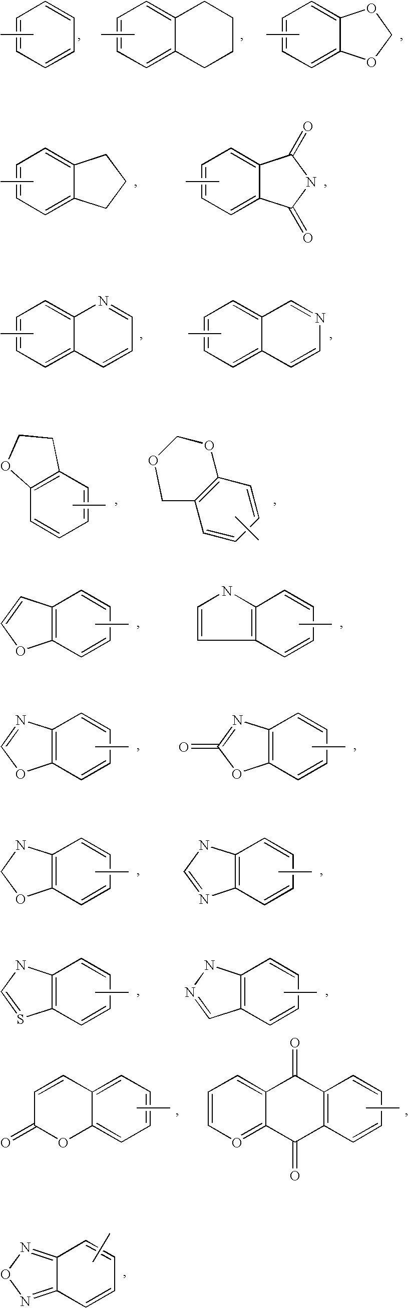
and the like.

The term “arylalkyl”, “aralkyl”, “(aryl)alkyl” or “(ar)alkyl” refers to a residue in which an aryl moiety is attached to the parent structure via an alkyl residue, wherein the aryl and alkyl portions are in accordance with the descriptions above. Similarly, terms such as “(heteroaryl)alkyl”, “(heterocyclo)alkyl”, and “(cycloalkyl)alkyl” refer respectively to heteroaryl, heterocyclo and cycloalkyl moeities that are attached to the parent structure via an alkyl residue.

The term “acyl” or “Ac” refers for example to alkanoyl radicals having 1 to 6 carbon atoms in which the alkyl portion can be substituted as defined above.

It will be understood throughout that the optional substituents are selected independently from one another.

Some of the compounds of Formula I and related compounds are capable of forming both pharmaceutically acceptable acid addition and/or base salts. All of these forms are within the scope of the present invention, as are separated diastereomers and enantiomers.

The optical isomers can be obtained by resolution of the racemic mixtures according to conventional processes, for example, by the formation of diastereoisomeric salts using an optically active acid or base, or by formation of covalent diastereomers. Examples of appropriate optically active acids are tartaric, diacetyltartaric, dibenzoyltartaric, ditoyluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids may then be liberated from the separated diastereomeric salts. A different process for separation of optical isomers involves use of chiral chromatography (e.g., chiral HPLC columns), with or without conventional derivitization, optimally chosen to maximize the separation of the enantiomers. Suitable chiral HPLC columns are manufactured by Diacel, e.g., Chiracel OD and Chiracel OJ, among many others, all routinely selectable. Enzymatic separations, with or without derivitization, are also useful. The optically active compounds of formula I can likewise be obtained by utilizing optically active starting materials.

Pharmaceutically acceptable acid addition salts of the compounds of Formula I include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, 2-phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al., “Pharmaceutical Salts,” J. Pharma. Sci., 1977;66:1).

The acid addition salts of basic compounds of formula I can be prepared by contacting the free base form with a sufficient amount of the desired acid to produce the salt in the conventional manner. The free base form may be regenerated by contacting the salt form with a base and isolating the free base in the conventional manner. The free base forms can differ from their respective salt forms somewhat in certain physical properties such as solubility in polar solvents.

Pharmaceutically acceptable base addition salts of the compounds of formula I can be formed with metals or amines, such as alkali and alkaline earth metals or organic amines. Examples of such metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see Berge, Supra, 1977).

The base addition salts of acidic compounds of formula I can be prepared by contacting the free acid form with a sufficient amount of the desired base to produce the salt in the conventional manner. The free acid form may be regenerated by contacting the salt form with an acid and isolating the free acid in the conventional manner. The free acid forms can differ from their respective salt forms somewhat in certain physical properties such as solubility in polar solvents.

Certain of the compounds of the present invention can exist in unsolvated forms as well as solvated forms, including hydrated forms. Solvated and unsolvated forms are intended to be encompassed within the scope of the present invention.

Certain of the compounds of the present invention possess one or more chiral centers and each center may exist in the R(D) or S(L) configuration. The present invention includes all diastereomeric, enantiomeric and epimeric forms as well as all mixtures thereof such as racemic mixtures.

The activity of compounds of the present invention can be assessed using suitable assays, such as receptor binding assays and chemotaxis assays. For example, as described in the example section, antagonist compounds of the present invention have been identified utilizing a CCR-5 Receptor MIP1α SPA binding assay and have been found to exhibit IC₅₀ values ranging from 0.01 μM to 38 μM. Such values are indicative of the intrinsic activity of the compounds in use as modulators of chemokine receptor activity. There are numerous other such screening assays known to those skilled in the art which may be used to determine the CCR-5 receptor antagonistic activity of the compounds of the present invention. One such screening technique is described in PCT WO 92/01810. Another assay, for example, maybe employed for screening a receptor antagonist by contacting melanophore cells which encode the CCR-5 receptor with both the RANTES and a compound to be screened. Inhibition of the signal generated by the ligand indicates that a compound is an antagonist for the receptor, i.e., inhibits activation of the receptor.

Other screening techniques include the use of cells which express the CCR-5 receptor (for example, transfected CHO cells, RBL-2 cells or other mammalian cells) in a system which measures extracellular pH changes caused by receptor activation, for example, as described in Science, volume 246, pages 181-296 (October 1989), herein incorporated by reference. Potential antagonists may be contacted with a cell which expresses the CCR-5 receptor and a second messenger response, e.g. signal transduction or pH changes, or making use of a reporter gene system, for example luciferase, may be measured to determine whether the potential antagonist is effective.

Another such screening technique involves introducing mRNA encoding the CCR-5 receptor into Xenopus oocytes, RBL-2 or other mammalian cells to transiently express the receptor. The cells with the expressed receptor may then be contacted in the case of antagonist screening with RANTES and a compound to be screened, followed by detection of inhibition of a calcium or cAMP signal.

Another screening technique involves expressing the CCR-5 receptor in which the receptor is linked to a phospholipase C or D. As representative examples of such cells, there may be mentioned endothelial cells, smooth muscle cells, embryonic kidney cells, etc. The screening for an antagonist may be accomplished as herein above described by detecting inhibition of activation of the receptor from the phospholipase second signal.

Another method involves screening for CCR-5 receptor inhibitors by determining inhibition of binding of labeled RANTES to cells or membranes which have the receptor on the surface thereof. Such a method involves transfecting a eukaryotic cell, such as CHO or RBL-2 cell, with DNA encoding the CCR-5 receptor such that the cell expresses the receptor on its surface and contacting the cell with a potential antagonist in the presence of a labeled form of RANTES. The RANTES can be labeled, e.g., by radioactivity. The amount of labeled ligand bound to the receptors is measured, e.g., by measuring radioactivity associated with transfected cells or membrane from these cells. If the potential antagonist binds to the receptor, as determined by a reduction of labeled ligand which binds to the receptors, the binding of labeled ligand to the receptor is inhibited.

Another method involves screening for CCR-5 inhibitors by determining inhibition or stimulation of CCR-5-mediated cAMP and/or adenylate cyclase accumulation or diminution. Such a method involves transfecting a eukaryotic cell, such as CHO or RBL-2 cell, with CCR-5 receptor to express the receptor on the cell surface. The cell is then exposed to potential antagonists in the presence of RANTES. The amount of cAMP accumulation is then measured. If the potential antagonist binds the receptor, and thus inhibits CCR-5 binding, the levels of CCR-5-mediated cAMP, or adenylate cyclase, activity will be reduced or increased.

Another such screening technique is described in U.S. Pat. No. 5,928,881, which provides a method for determining whether a ligand not known to be capable of binding to the CCR-5 receptor can bind to such receptor which comprises contacting a mammalian cell which expresses the CCR-5 receptor with RANTES under conditions permitting binding of ligands to the CCR-5 receptor, detecting the presence of a ligand which binds to the receptor and thereby determining whether the ligand binds to the CCR-5 receptor.

A review of the role of chemokines in allergic inflammation is provided by Kita, H., et al., J. Exp. Med. 183, 2421-2426 (1996) suggesting that agents which modulate chemokine receptors would be useful in allergic inflammatory disorders and diseases. Compounds which modulate chemokine receptors are especially useful in the treatment and prevention of atopic conditions including allergic rhinitis, dermatitis, conjunctivitis, and particularly bronchial asthma.

Migration of leukocytes from blood vessels into diseased tissues is important to the initiation of normal disease-fighting inflammatory responses. But this process, known as leukocyte recruitment, is also involved in the onset and progression of debilitating and life-threatening chronic inflammatory, allergic inflammatory and autoimmune diseases. Thus, compounds which block leukocyte recruitment to target tissues in inflammatory and autoimmune disease would be a highly effective therapeutic intervention.

It has been recognized that for efficient entry into target cells, human immunodeficiency viruses require chemokine receptors, such as CCR-5 or CXCR4, as well as the primary receptor CD4 (Levy, N. Engl. J. Med., 335(20), 1528-1530 (Nov. 14, 1996). The principal cofactor for entry mediated by the envelope glycoproteins of certain strains of HIV-1 is CCR-5, a receptor for the chemokines RANTES, MIP-1α and MIP-10 (Deng, et al., Nature, 381, 661666 (1996)). Accordingly, an agent which could block chemokine receptors in humans who possess normal chemokine receptors will prevent infection in healthy individuals and slow or halt viral progression in infected patients. Inhibition of chemokine receptors presents a viable method for the prevention or treatment of infection by HIV and the prevention or treatment of AIDS.

Small molecule antagonists of the interaction between C—C chemokine receptors and their ligands, including RANTES and MIP-1α, provide compounds useful for blocking chemokine receptors and inhibiting harmful inflammatory processes “triggered” by receptor ligand interaction, as well as valuable tools for the investigation of receptor-ligand interactions. The selective inhibition of a CCR-5 receptor by treatment with the receptor antagonists of the invention represents a novel therapeutic and/or preventative approach to the treatment of a broad spectrum of inflammatory and autoimmune diseases or conditions, in particular for the treatment of inflammatory diseases or conditions, atherosclerosis, restenosis, and autoimmune disorders such as arthritis and transplant rejection.

In a preferred embodiment, the disease or condition is one which is associated with lymphocyte and/or monocyte infiltration of tissues (including recruitment and/or accumulation in tissues), such as arthritis (e.g., rheumatoid arthritis), inflammatory bowel diseases (e.g., Crohn's disease, ulcerative colitis), multiple sclerosis, idiopathic pulmonary fibrosis, and graft rejection (e.g., in transplantation), including allograft rejection or graft-versus-host disease. In addition, diseases characterized by basophil activation and/or eosinophil recruitment, including allergic hypersensitivity disorders such as psoriasis, asthma and allergic rhinitis can be treated according to the present invention. Other diseases that may be treated with the compounds of Formula I are: chronic contact dermatitis, sarcoidosis, dermatomyositis, skin phemphigoid and related diseases (e.g., pemphigus vulgaris, p. foliacious, p. erythematosus), glomerulonephritides, vasculitides (e.g., necrotizing, cutaneous, and hypersensitivity vasculitis), hepatitis, diabetes, systemic lupus erythematosus and myasthenia gravis. In addition to psoriasis, other inflammatory dermatoses such as dermatitis, eczema, atopic dermatitis, allergic contact dermatitis, urticaria and reperfusion injury can also be treated.

The antagonists of the present invention bind to the CCR-5 receptor, making it inaccessible to ligands such that normal biological activity is prevented. They may be administered to a mammal in need of treatment of CCR-5 mediated disease states. Thus, the active ingredient may be administered in the mammal using conventional course of treatment determination tests.

The term “CCR-5 mediated disease state” is used herein at all occurrences to mean any disease state which is affected or modulated by CCR-5.

The subject treated in the methods above is preferably a mammal, preferably a human being, male or female, in whom modulation of chemokine receptor activity is desired. “Modulation” as used herein is intended to encompass antagonism, agonism, partial antagonism, inverse agonism and/or partial agonism. In a preferred aspect of the present invention, modulation refers to antagonism of chemokine receptor activity, since the compounds of the invention are antagonists.

Combined therapy to modulate chemokine receptor activity and thereby prevent and treat the above-noted conditions illustrated by the combination of the compounds of this invention and other compounds which are known for such utilities.

For example, in the treatment or prevention of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, epinephrine, naphazoline, xylometazoline, propylhexedrine, or levo-desoxy-ephedrine; an antitussive such as codeine, hydrocodone, caramiphen, carbetapentane, or dextromethorphan; a diuretic; and a sedating or non-sedating antihistamine. Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred.

Accordingly, the pharmaceutical compositions of the present invention include those that also contain one or more other active ingredients, in addition to a compound of the present invention. Examples of other active ingredients that may be combined with a compound of the present invention, either administered separately or in the same pharmaceutical compositions, include, but are not limited to: (a) VLA-4 antagonists such as those described in U.S. Pat. No. 5,510,332, (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as .beta.2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, caiprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CXCRA, CCR-1, CCR-2, CCR-3 and CCR-5; (j) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (metformin), .α.-glucosidase inhibitors (acarbose) and glitazones (troglitazone and pioglitazone); (l) preparations of interferon beta (interferon-beta-lac, interferon-beta-1.beta.); (m) other compounds such as 5-aminosalicylic acid and prodrugs thereof, antimetabolites such as azathioprine and 6-mercaptopurine, and cytotoxic cancer chemotherapeutic agents.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. The compounds of the invention are effective for use in primates, such as humans, as well as for the treatment of warm-blooded animals such as mice, rats, horses, cattle, sheep, dogs, cats, monkeys, guinea pigs, other bovine, ovine, equine, canine, feline, rodent or murine species. However, the compounds of the invention are also effective for use in other species, such as avian species (e.g., chickens).

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.) The pharmaceutical composition and method of the present invention may further comprise other therapeutically active compounds as noted herein which are usually applied in the treatment of the above mentioned pathological conditions.

The compounds of the invention, or their pharmaceutically acceptable salts, are administered in a therapeutically effective amount which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disease-states; and the host undergoing therapy. Generally, a therapeutically effective daily dose is from about 0.14 mg to about 14.3 mg/kg of body weight per day of a compound of the invention, or a pharmaceutically acceptable salt thereof; preferably, from about 0.7 mg to about 10 mg/kg of body weight per day; and most preferably, from about 1.4 mg to about 7.2 mg/kg of body weight per day. For example, for administration to a 70 kg person, the dosage range would be from about 10 mg to about 1.0 gram per day of a compound of the invention, or a pharmaceutically acceptable salt thereof, preferably from about 50 mg to about 700 mg per day, and most preferably from about 100 mg to about 500 mg per day. The compounds may be administered on a regimen of 1 to 4 times per day, preferably once or twice per day.

In the foregoing and in the following examples, all temperatures are set forth uncorrected in degrees Celsius; and, unless otherwise indicated, all parts and percentages are by weight.

Compounds of the invention can be made by procedures known in the art, such as those disclosed in WO 00/66559; WO00/66558; WO 02/079157, and WO 02/079194. With respect to identified subgenuses and procedures of making, applicants incorporate by reference the entire disclosures of WO 00/66559; WO00/66558; WO 02/079157; and WO 02/079194, as if fully set forth herein. Furthermore, the entire disclosures of all applications, patents and publications, cited above or below, are hereby incorporated by reference.

Compounds of the invention can also be prepared as described in the following reaction schemes and by the methods described in the examples below.

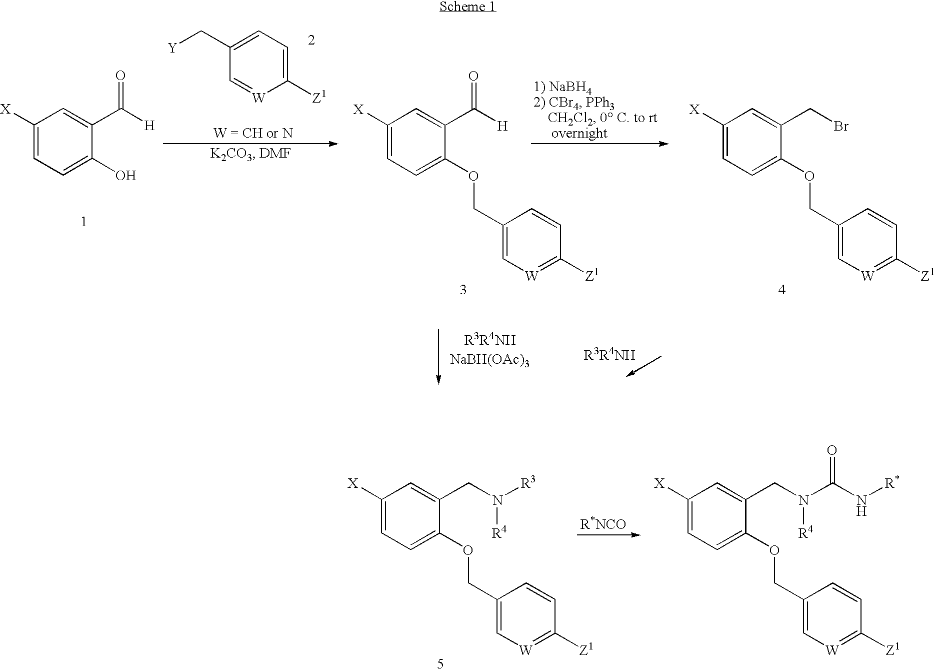

5- Substituted-2-hydroxybenzaldehyde 1 is reacted with an arylmethyl bromide or substituted pyridylmethyl bromide 2 in the presence of base, such as K₂CO₃, in DMF at ambient temperature to afford 3. The transformation of 3 to 5may be achieved via two different ways:
a) Reductive amination of 3 with amines (R³R⁴NH) using a reducing reagent such as NaBH(OAc)₃ to afford 5;
b) Reduction of 3 with NaBH₄, followed by bromination with CBr₄ and PPh₃ afforded 4. Further reaction of 4 with amines (R³R⁴NH) to afford 5.
   Further reaction of 5 (R³═H) with isocyanate (R⁹NCO) affords 6.

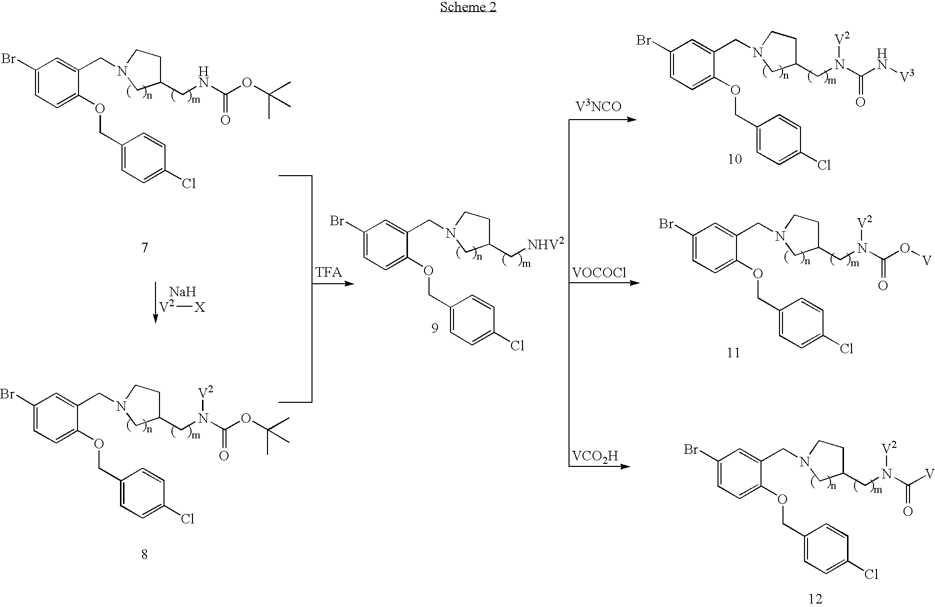

Deprotonation of 7 (prepared according to Scheme 1) with NaH, followed by alkylation with halides V²—X affords 8. Deprotection of 7 or 8 with TFA, followed by reaction with isocyanates (V³NCO), chloroformates (VOCOCl), and acids (VCO₂H) affords the corresponding product 10, 11 or 12, respectively.

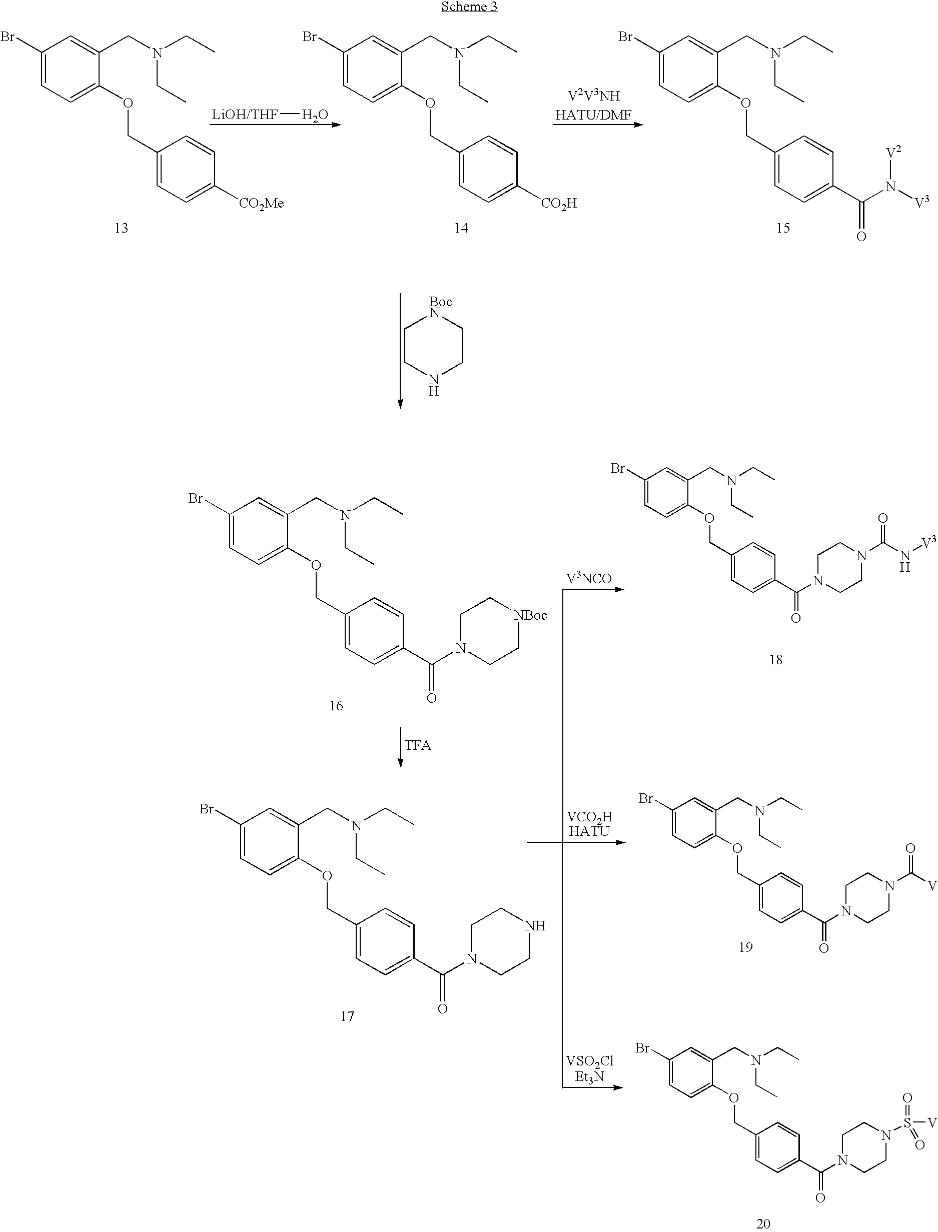

Hydrolysis of 13 (prepared according to Scheme 1), followed by coupling with amines (V²V³NH) affords 15. Amidation of 14 with Boc-piperazine, followed by deprotection and coupling with isocyanates (V³NCO), acids (VCO₂H) or sulfonyl chlorides (VSO₂Cl) affords 18, 19, or 20, respectively.

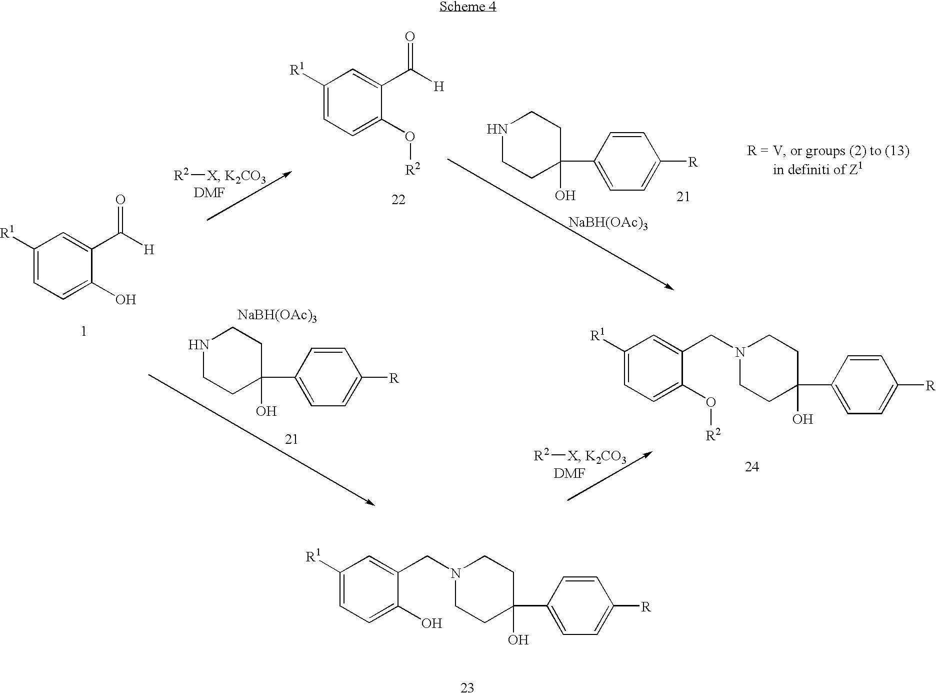

Compound 24 may be synthesized by the following two methods:
a) Alkylation of 1 with alkyl halides (R²—X), followed by reductive amination with 21 affords 24;
b) Reductive amination of 1 with 21, followed by alkylation with halides (R²—X) affords 24.

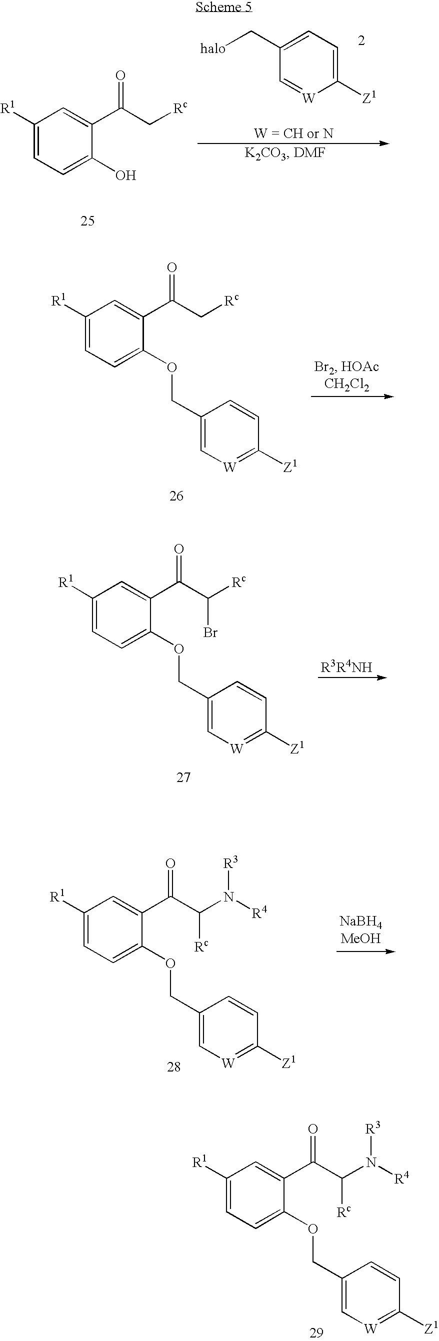

Phenol 25 reacts with arylmethyl bromide or substituted pyridylmethyl halides 2 in the presence of base, such as K₂CO₃, in DMF at ambient temperature to afford 26. Bromination of 26, followed by reaction with amines (R³R⁴NH) affords 27. Reduction of 28 with a reducing agent such as NaBH₄ affords 29. 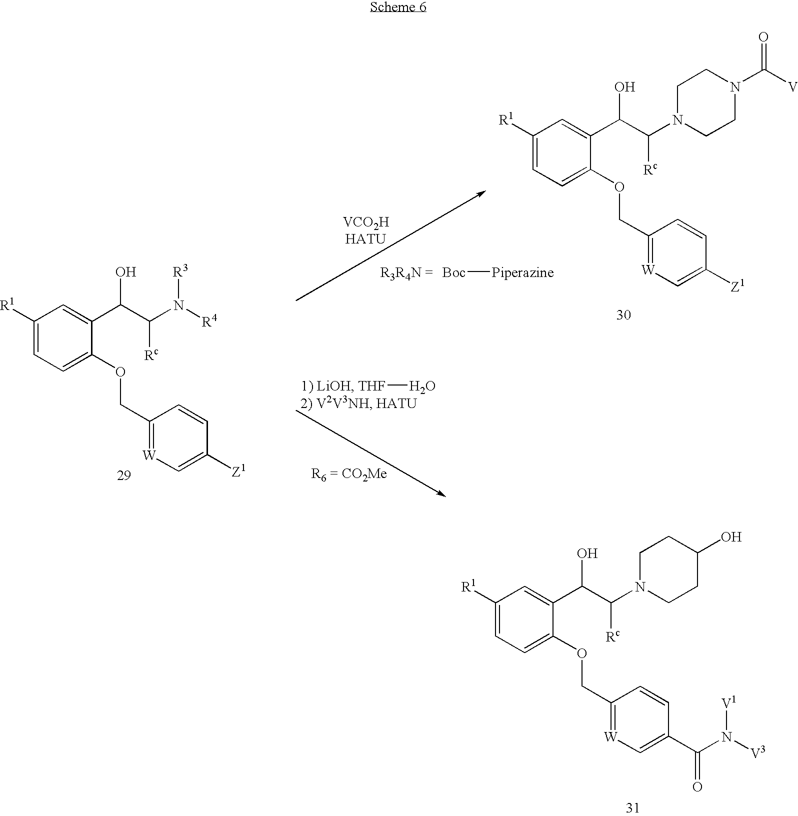
1) For compound 29 with R³R⁴N=Boc-piperazine: de-protection of 29 with an acid such as TFA, followed by reaction with an activated acid (VCO₂H) affords 30;
2) For compound 29 with Z¹═—CO₂Me: reaction of 29 with an acid or base, followed by reaction with V²V³NH, affords 31.

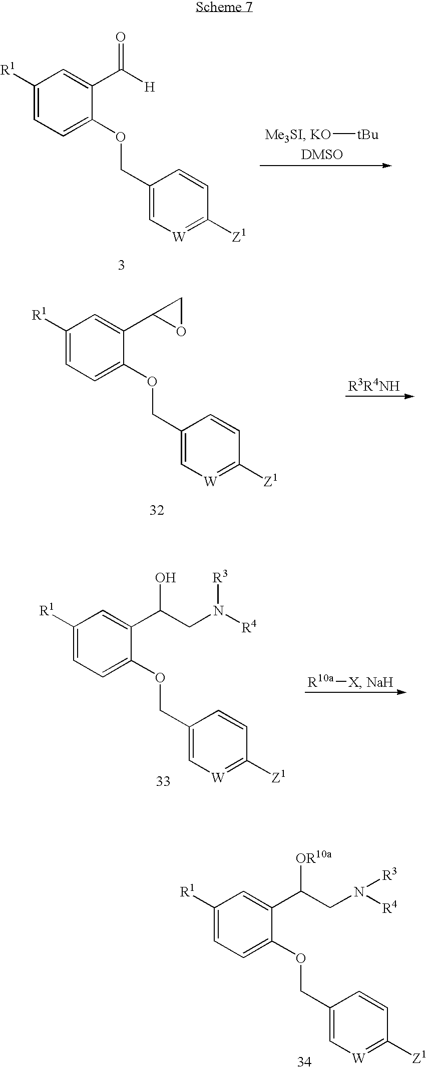

Compound 3 (prepared according to Scheme 1) reacts with Me₃S⁺I⁻ using KOtBu as a base to afford epoxide 32. Ring opening of epoxide 32 with amines (R³R⁴NH) affords 33. Alkylation of the alcohol of 33 with alkyl halides (R^{10a}—X) using a base such as NaH affords 34.

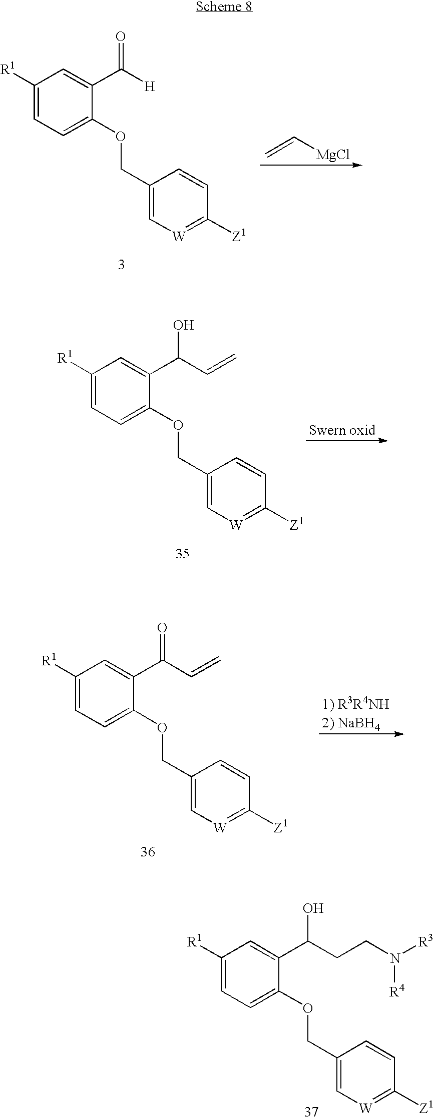

Reaction of aldehyde 3 with ethylenemagnesium chloride affords 35, which is converted to 36 by oxidation under conditions such as those described by Swern. Michael addition of amines (R³R⁴NH) to 36 followed by reduction with a reagent such as NaBH₄ affords 37.

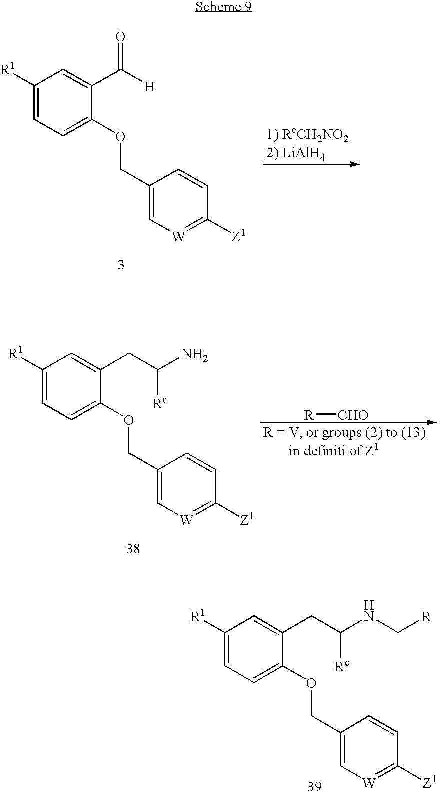

Condensation of 3 with R^{c}CH₂NO₂, followed by reaction with a reducing agent such as LiAlH₄ affords 38. Reductive-amination of 38 with aldehydes (R—CHO) affords 39.

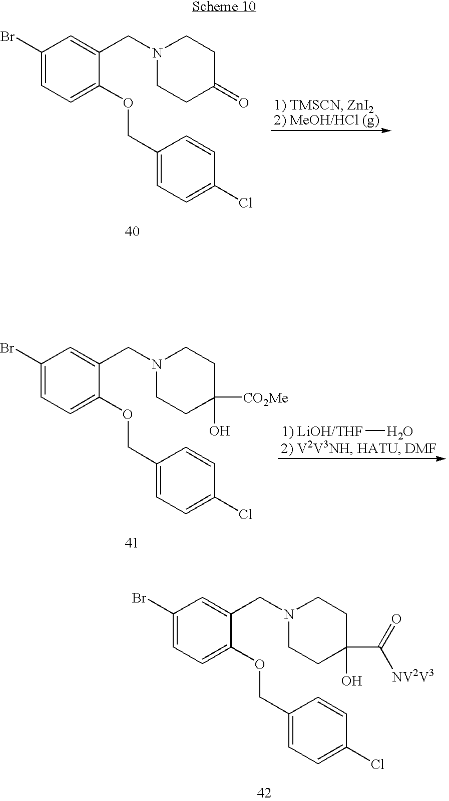

Treatment of 40 (prepared according to Scheme 1) with TMSCN and ZnI₂, followed by reaction with MeOH/HCl affords methyl ester 41. Hydrolysis of 41 to the acid, and amidation with amines (V²V³NH) affords 42. 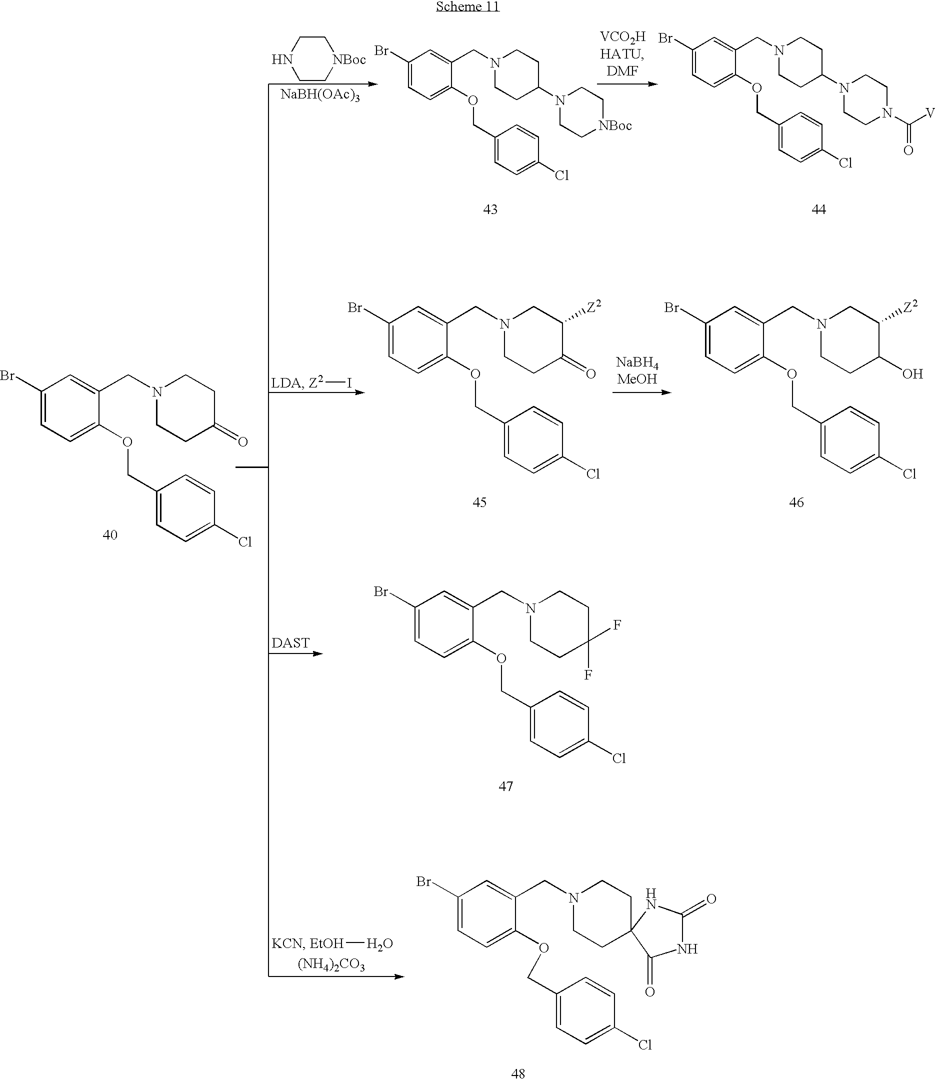
a) Reductive amination of 40 with N-Boc-piperazine affords 43. Removal of the Boc group under standard conditions and coupling with an activated acid (VCO₂H) affords 44.
b) De-protonation of 40, followed by treatment with halide (Z²—I) affords 45. Reduction of 45 affords 46.
c) Treatment of 40 with DAST affords 47.
d) Treatment of 40 with KCN and (NH₄)₂CO₃ affords 48. 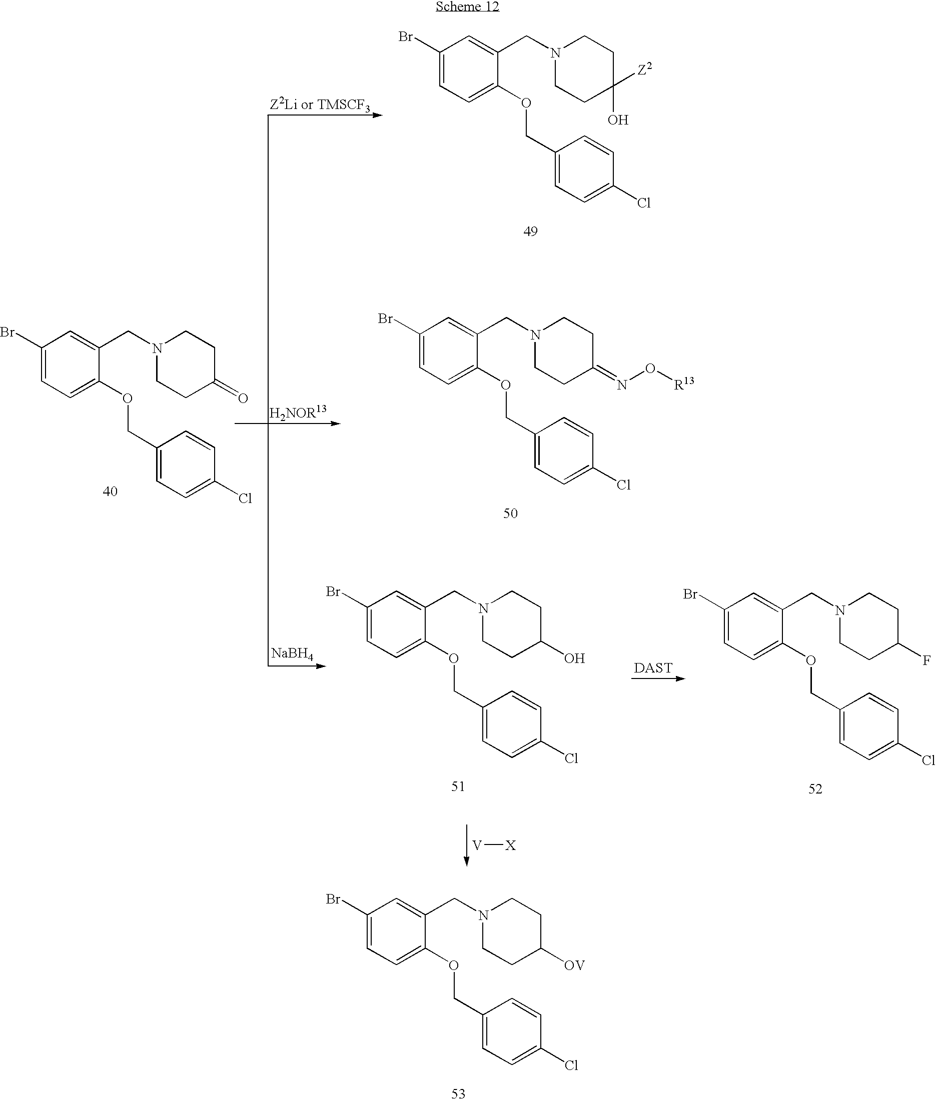
a) Treatment of 40 with a nucleophile such as an aryl or alkyl lithium reagent or TMSCF₃ affords 49.
b) Treatment of 40 with H₂NOR¹³ affords 50.
c) Reduction of 40 with a reagent such as NaBH₄, followed by treatment with DAST affords 52.
d) Reaction of 51 with alkyl halides (V—X) affords 53.

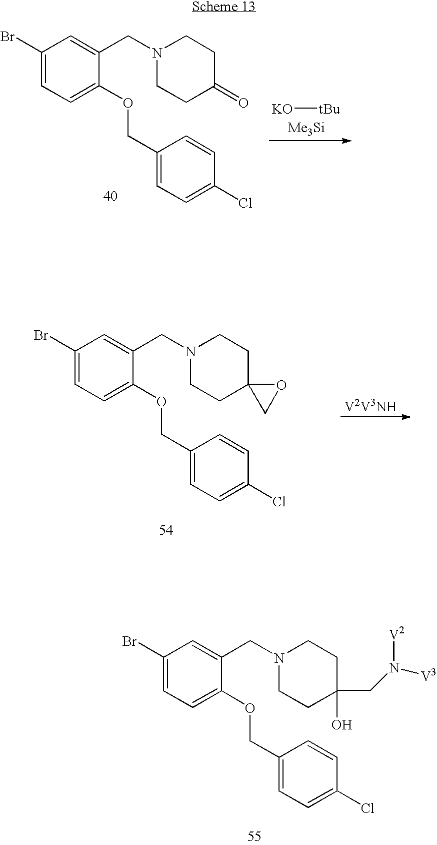

Compound 40, prepared according to Scheme 1, reacts with Me₃S⁺I⁻ to afford epoxide 54. Ring opening of epoxide 54 with amines (V²V³NH) affords 55.

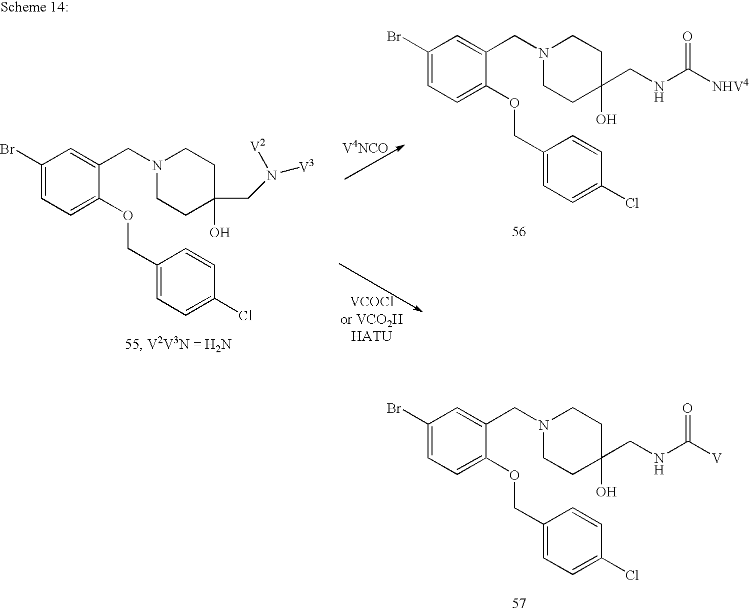

Coupling of 55 (V²═V³═H) with isocyanates (V⁴NCO) and acid chlorides (VCOCl) affords 56 and 57, respectively.

### EXAMPLES

### Example 1

### 5-Bromo-2-(4-chlorophenylmethoxy)benzaldehyde

To a stirred solution of 5-bromo-2-hydroxybenzaldehyde (100 g, 0.5 mol) in DMF (600 mL) was added K₂CO₃ (206 g, 1.49 mol) at rt. After 30 min, 4-chlorobenzylchloride (78 g, 0.48 mol) was added. The reaction mixture was kept at 65° C. overnight, then cooled to rt, and poured into an ice-cooled mixture of EtOAc/water (1:1, 2 L). The solid was collected by filtration, washed with water, and dried under vacuum for 20 h to give the desired product (160 g, 100%). ¹H NMR (400 MHz, CDCl₃): δ 5.18 (s, 2H), 6.92 (d, 1H), 7.37 (m, 4H), 7.61 (d, 1H), 7.98 (s, 1H), 10.4 (s, 1).

The following compounds were prepared in a similar manner:
4-[(4-Chloro-2-formylphenoxy)methyl]benzonitrile
5-Bromo-2-[[4-(trifluoromethyl)phenyl]methoxy]benzaldehyde
5-Bromo-2-[(4-iodophenyl)methoxy]benzaldehyde
5-Bromo-2-[(6-methyl-3-pyridinyl)methoxy]benzaldehyde
5-Bromo-2-[(4-methylphenyl)methoxy]benzaldehyde
4-[[4-Bromo-2-(bromomethyl)phenoxy]methyl]benzoic acid, methyl ester

### Example 2

### N-[[5-Bromo-2-(4-chlorophenylmethoxy)phenyl]methyl]morpholineethanamine, dihydrochloride

To methylene chloride (50 mL) was added 4-(2-aminoethyl)morpholine (1 mL, 6.8 mmol), 5-bromo-2-(4-chlorophenylmethyl)benzaldehyde (1 g, 3.1 mmol), and sodium triacetoxyborohydride (1 g, 4.7 mmol). The reaction was stirred for 18 h and washed with 2 N aqueous KOH. The organic layer was dried (MgSO₄) and the solvent was removed in vacuo. The residue was crystallized from petroleum ether. The solids were dissolved in ethanol (100 mL) and acidified with concentrated HCl. The solvent was removed in vacuo and the residue was triturated with acetone to give the title compound as a white solid. ¹H NMR (DMSO-d₆, 400 MHz): δ 3.1 (br, 2H), 3.45 (br, 6H), 3.8 (br, 2H), 3.9 (br, 2H), 4.2 (s, 2H), 7.05 (d, 1H), 7.45 (d, 2H), 7.55 (m, 3H), 7.8 (s,1 H), 9.8 (br, 2H), 11.6 (br, 1H).

The following compounds were prepared in a similar manner
5-Bromo-2-(4-chlorophenylmethoxy)-N,N-diethylbenzenemethanamine, Hydrochloride. ¹H NMR (DMSO-d_{6,} 400 MHz): δ 1.15 (t, 6H), 3.0 (m, 4H), 4.2 (s, 2H), 5.15 (s, 2H), 7.15 (d, 1H), 7.45 (d, 2H), 7.55 (m, 3H), 7.85 (s,1 H), 10.3 (br, 1H).
1-[[[5-Bromo-2-(4-chlorophenylmethoxy)phenyl]methyl]amino]-2-propanol, Hydrochloride.

¹H NMR (DMSO-d₆, 400 MHz): δ 1.05 (d, 3H), 2.65 (m, 1H), 2.85 (m, 1H), 3.95 (m, 1H), 4.1 (m, 2H), 5.15 (s, 2H), 5.4 (br, 1H), 7.15 (d, 1H), 7.45 (d, 2H), 7.55 (m, 3H), 7.75 (s,1 H), 8.95 (br, 1H), 9.4 (br, 1H).
1-[[5-Bromo-2-(4-chlorophenylmethoxy)phenyl]methyl]-4-ethylpiperazine, Dihydrochloride.

¹H NMR (DMSO-d₆, 400 MHz): δ 1.15 (m, 3H), 3.0-3.8 (m, 12H), 5.15 (s, 2H), 7.1 (d, 1H), 7.45 (d, 2H), 7.55 (m, 3H), 7.75 (br, 1 H).
N-[[5-Bromo-2-(4-chlorophenylmethoxy)phenyl]methyl]-N′,N′-dimethylpropanediamine, Dihydrochloride. ¹H NMR (DMSO-d₆, 400 MHz): δ 2.15 (m, 2H), 2.7 (s, 6H), 3.0 (m, 2H), 3.1 (m, 2H), 4.1 (s, 1H), 5.15 (s, 2H), 7.05 (d, 1H), 7.45 (d, 2H), 7.55 (m, 3H), 7.75 (s,1 H), 9.6 (br, 2H), 10.9 (br, 1H).
3-[[4-Bromo-2-[(diethylamino)methyl]phenoxy]methyl]benzoic acid, methyl ester, hydrochloride. ¹H NMR (DMSO-d₆, 400 MHz): δ 1.15 (t, 3H), 3.0 (m, 4H), 3.8 (s, 3H), 4.2 (s, 2H), 5.25 (s, 2H), 7.15 (d, 1H), 7.55 (m, 2H), 7.8 (m, 1H), 7.85 (m,1 H), 7.9 (m,1H), 8.05 (s,1H), 10.15 (br, 1H).
4-[[5-Bromo-2-[(4-chlorophenyl)methoxy]phenyl]methyl]thiomorpholine. ¹H NMR (DMSO-d₆/TFA): δ 2.75˜3.00 (m, 4H), 3.20 (m, 2H), 3.51 (m, 2H), 4.30 (br.s, 1H), 5.20 (s, 2H), 7.18 (d, 1H), 7.44˜7.54 (m, 4H), 7.62 (dd, 1H), 7.73 (d, 1H), 9.50 (br.s, 1H).
5-Bromo-2-[(4-chlorophenyl)methoxy]-N-methyl-N-(phenylmethyl)benzenemethanamine. ¹H NMR (DMSO-d₆/TFA): δ 2.54 (s, 3H), 4.10˜4.44 (m, 4H), 5.12 (q, 2H), 7.13 (d, 1H), 7.36˜7.50 (m, 9H), 7.56˜7.62 (m, 1H), 7.66 (d, 1H), 7.60 (br.s, 1H).
5-Bromo-2-[(4-chlorophenyl)methoxy]-N-ethylbenzenemethanamine

¹H NMR (CDCl₃): δ 1.25 (t, 3H), 2.86 (q, 2H), 3.95 (s, 2H), 5.15 (s, 2H), 6.82 (d, 1H), 7.34˜7.48 (m, 5H), 7.67 (d, 1H).
4-[[5-Bromo-2-[(4-chlorophenyl)methoxy]phenyl]methyl]morpholine

¹H NMR (CDCl₃-D₂O): δ 3.92 (dd, 4H), 4.20 (s, 2H), 4.80 (s, 2H), 5.06 (s, 2H), 6.88 (d, 1H), 7.30˜7.42 (m, 4H), 7.50 (m, 1H), 7.58 (d, 1H).
5-Bromo-2-[(4-chlorophenyl)methoxy]-N-(phenylmethyl)benzenemethanamine. ¹H NMR (DMSO-d₆/TFA): δ 4.10 (s, 2H), 4.17 (s, 2H), 5.12 (s, 2H), 7.09 (d, 1H), 7.36˜7.46 (m, 9H), 7.72 (dd, 1H), 7.68 (d, 1H), 9.20 (br.s, 2H).
5-Bromo-2-[(4-chlorophenyl)methoxy]-N,N-dimethylbenzenemethanamine,

¹H NMR (DMSO-d₆/TFA): δ 2.70 (s, 6H), 4.25 (s, 2H), 5.17 (s, 2H), 7.12 (d, 1H), 7.42˜7.54 (m, 4H), 7.60 (dd, 1H), 7.68 (d, 1H), 9.20 (br.s, 1H).
[1-[[5-bromo-2-[(4-chlorophenyl)methoxy]phenyl]methyl]-3-pyrrolidinyl]carbamic acid, 1,1-dimethylethyl ester ¹H NMR (400 MHz, DMSO-d₆): δ 1.42 (s, 9H), 1.58 (m, 1H), 2.3 (m, 2H), 2.6 (m, 2H), 2.8 (m, 1H), 3.61 (dd, 2H), 4.2 (br. s, 1H), 4.9 (br. s, 1H), 5.0 (s, 2H), 6.75 (d, 1H), 7.29 (d, 1H), 7.35 (dd, 4), 7.47 (d, 1H).
3-[[4-Bromo-2-[(diethylamino)methyl]phenoxy]methyl]benzoic acid, methyl ester, hydrochloride. Prepared in a similar manner as example 2, starting with 3-[(4-bromo-2-formylphenoxy)methyl]-benzoic acid methyl ester and diethylamine. ¹H NMR (DMSO-d₆, 400 MHz): δ 1.15 (t, 3H), 3.0 (m, 4H), 3.8 (s, 3H), 4.2 (s, 2H), 5.25 (s, 2H), 7.15 (d, 1H), 7.55 (m, 2H), 7.8 (m, 1H), 7.85 (m,1 H), 7.9 (m,1H), 8.05 (s,1H), 10.15 (br, 1H).
1-[[5-Bromo-2-[(4-iodophenyl)methoxy]phenyl]methyl]-4-piperidinol. Prepared in a similar manner as example 2, starting with 5-bromo-2-[(4-iodophenyl)methoxy]benzaldehyde and 4-hydroxylpiperidine. ¹H NMR (400 MHz, DMSO-d₆): δ1.38 (m, 2H), 1.62 (m, 2H), 2.1 (t, 2H), 2.6 (br., d, 2H), 4.57 (br. s, 1H), 5.04 (s, 2H), 6.97 (d, 1H), 7.21 (d, 2H), 7.37 (d, 1H), 7.4 (s, 1H), 7.7 (d, 2H).
1-[[5-Bromo-2-[(4-methylphenyl)methoxy]phenyl]methyl]-4-piperidinol. Prepared in a similar manner as example 2, starting with 5-bromo-2-[(4-methylphenyl)methoxy]benzaldehyde and 4-hydroxylpiperidine. ¹H NMR (400 MHz, DMSO-d₆): δ1.38 (m, 2H), 1.67 (m, 2H), 2.0 (t, 2H), 2.24 (s, 3H), 2.6 (m, 2H), 3.4 (m, 4H), 4.5 (br. s, 1H), 5.02 (s, 2H), 7.0 (d, 1H), 7.16 (d, 2H), 7.3 (d, 2H), 7.34 (dd, 1H), 7.4 (d, 1H).
1-[[5-Bromo-2-[(4-chlorophenyl)methoxy]phenyl]methyl]-4-piperidinol. Prepared in a similar manner as example 2, starting with 5-bromo-2-[[4-(trifluoromethyl)phenyl]methoxy]benzaldehyde and 4-hydroxylpiperidine. ¹H NMR (400 MHz, DMSO-d₆): δ 1.38 (br. s, 2H), 1.7 (br. s, 2H), 1.7 (m, 2H), 2.7 (br. s, 2H), 3.1 (m, 1H), 3.5 (m, 4H), 5.21 (s, 2H), 7.0 (dd, 1H), 7.4 (d, 1H), 7.43 (br.s, 1H), 7.62 (d, 2H), 7.74 (d, 2).
1-[[5-Bromo-2-[(6-methyl-3-pyridinyl)methoxy]phenyl]methyl]-4-piperidinol. Prepared in a similar manner as example 2, starting with 5-bromo-2-[(6-methyl-3-pyridinyl)methoxy]benzaldehyde and 4-hydroxylpiperidine. ¹H NMR (CDCl₃): δ 1.60 (m, 2H), 1.88 (m, 2H), 2.19 (m, 2H), 2.58 (s, 3H), 2.76 (m, 2H), 3.51 (s, 2H), 3.70 (m, 1H), 5.02 (s, 2H), 6.79 (d, 1H), 7.18 (d, 1H),7.32 (dd, 1H), 7.51 (d, 1H), 7.64 (dd, 1H), 8.56 (d, 1H).
1-[[4-Bromo-2-[(6-methyl-3-pyridinyl)methoxy]phenyl]methyl]-4-(4-bromophenyl)-4-piperidinol. Prepared in a similar manner as example 2, starting with 5-bromo-2-[(6-methyl-3-pyridinyl)methoxy]benzaldehyde and 4-(4-bromophenyl)-4-hydroxylpiperidine. ¹H NMR (DMSO-d₆/TFA): δ 1.75 (br.d, 2H), 2.15 (m, 2H), 2.54 (s, 3H), 3.20˜3.40 (m, 4H), 4.45 (d, 2H), 5.45 (s, 2H), 7.22 (d, 1H), 7.14 (m, 2H), 7.52 (m, 2H), 7.64 (dd, 1H), 7.78 (d, 1H), 7.95 (br.d, 1H), 8.59 (br.d, 1H), 8.96 (d, 1H).

The following compounds were prepared in a similar manner starting with 4-[(4-chloro-2-formylphenoxy)methyl]benzonitrile
4-[[4-Chloro-2-(4-morpholinylmethyl)phenoxy]methyl]benzonitrile. ¹H NMR (CDCl₃, 400 MHz) δ2.50 (m, 4H), 3.55 (s, 2H), 3.75 (m, 4H), 5.10 (s, 2H), 6.80 (d, 1H), 7.18 (d, 1H), 7.40 (s, 1H), 7.55 (d, 2H), 7.70 (d, 2H).
4[[4-Chloro-2-(1-pyrrolidinylmethyl)phenoxy]methyl]benzonitrile

¹H NMR (CDCl₃, 400 MHz) δ1.80 (m, 4H), 2.60 (m, 4H), 3.70 (s, 2H), 5.10 (s, 2H), 6.80 (d, 1H), 7.15 (d, 1H), 7.40 (s, 1H), 7.55, (d, 2H), 7.70 (d, 2H).
1-[[5-Bromo-2-[(4-chlorophenyl)methoxy]phenyl]methyl]-3-piperidinemethanol. ¹H NMR (CD₃OD, 400 MHz) δ 1.15 (m, 1H), 1.90 (m, 5H), 2.20 (m, 1H), 3.05 (m, 1H), 3.20 (m, 1H), 3.60 (m, 2H), 3.75 (s, 2H), 5.30 (s, 2H), 7.15 (d, 1H), 7.60 (m, 6H).

### Example 3

### N-[[5-Bromo-2-(4-chlorophenylmethoxy)phenyl]methyl]-N-(3-dimethylaminopropyl)-N′-phenylurea, hydrochloride

To methylene chloride (50 mL) was added phenyl isocyanate (0.5 mL, 3.8 mmol) and N-[[5-bromo-2-(4-chlorophenylmethoxy)phenyl]methylamino]-N′,N′-dimethylpropanediamine (0.8 g, 1.9 mmol). After stirring for 24 h the solvent was removed in vacuo. The residue was dissolved in ethanol (50 mL) and acidified with concentrated HCl. The solvent was removed in vacuo and the residue was triturated with ether to give the title compound as a yellow foam.

1H NMR (DMSO-d₆, 400 MHz) δ 1.9 (m, 2H), 2.65 (s, 6H), 3.0 (m, 2H), 3.35 (m, 2H), 4.6 (s, 1H), 5.15 (s, 2H), 6.9 (t, 1H), 7.05 (d, 1H), 7.2 (M, 3H), 7.45 (d, 4H), 7.55 (m, 3H), 8.5 (s, 2H), 10.2 (br, 1H).

### Example 4

### 4-[[4-Bromo-2-[(dimethylamino)methyl]phenoxy]methyl]-N-(3,4-dimethoxyphenylmethyl)benzamide, hydrochloride

To methylene chloride (600 mL) was added 4-(chloromethyl)benzoyl chloride (30 g, 159 mmol), veratrylamine (25 g, 150 mmol), and diisopropylethylamine (26 mL, 150 mmol) at −5° C. After warming to ambient temperature, the reaction was washed with 1N HCl and 10% K₂CO₃, dried (MgSO₄), and the solvent was removed in vacuo. Crystallization from ether gave 44 g of a tan solid. The solid was dissolved in DMSO (300 mL) and 5-bromosalicylaldehyde (31 g, 150 mmol) and K₂CO₃ (24 g, 170 mmol) were added. After stirring at 35° C. for 20 the reaction was poured into 10% K₂CO₃. The solid was collected by filtration and washed with acetonitrile and ether to give 56 g of an off-white solid. A portion of the solid (2 g, 4.1 mmol) was dissolved in methylene chloride (200 mL) and dimethylamine (10 mL of a 2M solution in THF, 20 mmol) and sodium triacetoxyborohydride (1.5 g, 7.0 mmol) was added. The reaction was stirred for 18 h and washed with 10% aqueous KOH. The organic layer was dried (MgSO₄) and the solvent was removed in vacuo. The solids were dissolved in acetone and acidified with HCl (g). The solid was filtered and washed with acetone and ether to give the title compound. 1H NMR (DMSO-d₆, 400 MHz): δ 3.65 (s, 6H), 3.7 (s, 6H), 4.25 (s, 2H), 4.4 (s, 2H), 5.25 (s, 2H), 6.8 (n, 2H), 6.9 (s, 1H), 7.1 (d, 1H), 7.55 (m, 3H), 7.8 (s,1 H), 7.9 (m, 2H), 9.0 (t, 1H), 10.6 (br, 1H).

The following compound was prepared in a similar manner:

5-Bromo-2-[[4-[(6,7-dimethoxy-3,4-dihydro-2(1H)-isoquinolinyl)carbonyl]phenyl]methoxy]-N,N-dimethylbenzenemethanamine, Hydrochloride. ¹H NMR (DMSO-d₆, 400 MHz): δ 2.65 (s, 6H), 2.7 (br, 2H), 3.6 (m, 2H), 2.7 (s, 6H), 4.25 (s, 2H), 4.5 (m, 2H), 5.2 (s, 2H), 6.7 (s, 1H), 6.8 (br, 1H), 7.15 (d, 1H), 7.45 (d, 2H), 7.55 (m, 3H), 7.85 (s, 1H), 10.75 (br, 1H).

### Example 5

### 4-Bromo-2-(bromomethyl)-1-[(4-chlorophenyl)methoxy]benzene

To a stirred solution of 5-bromo-2-(4-chlorophenylmethyl)benzaldehyde (30 g, 81.5 mmol) in MeOH/CH₂Cl₂ (300 mL/300 mL) at 0° C. was added NaBH₄ (3.8 g, 97.8 mmol) in three portions. After 30 min at 0° C. and 1 h at rt, the solvents were removed under vacuum, and the resulting residue was diluted with water (200 mL). The mixture was extracted with EtOAc (3×150 mL), washed with brine (2×100 mL), dried over Na₂SO₄, and concentrated to give crude product. This crude product was re-dissolved in CH₂Cl₂ (800 mL), and PPh₃ (23.6 g, 90 mmol) was added. After cooling to 0° C., CBr₄ (29.7 g, 90 mmol) was added with caution. The mixture was stirred at rt overnight, and concentrated to remove solvents. The resulting mixture was purified directly by flash chromatography to afford the title compound (29.3 g, 92% in two steps). ¹H NMR (400 MHz, CDCl₃): δ 4.52 (s, 2H), 5.11 (s, 2H), 6.76 (d, 1H), 7.4 (m, 5H), 7.52 (s, 1).

### Example 6A

### 2-[[[5-Bromo-2-[(4-chlorophenyl)methoxy]phenyl]methyl]amino]-1,3-propanediol

To a stirred solution of 4-bromo-2-(bromomethyl)-1-[(4-chlorophenyl)methoxy]benzene (300 mg, 0.77 mmol) in DMF (3 mL) was added 2-amino-1,3-propanediol (300 mg, 3.3 mmol) at room temperature. After 2 h, the reaction mixture was purified directly by HPLC to afford the title compound as a trifluoroacetic acid salt. ¹H NMR (400 MHz, DMSO-d₆): δ 3.1 (br. s, 1H), 3.4 (br., s, 2H), 3.62 (m, 4H), 4.23 (s, 2H), 5.2 (s, 2H), 5.36 (br. s, 1H), 7.1 (d, 1H), 7.43 (dd, 2H), 7.56 (m, 3H), 7.67(s, 1H), 8.63 (br. 1H).

The following compounds were prepared in a similar manner:

(2R)-1-[[5-Bromo-2-[(4-chlorophenyl)methoxy]phenyl]methyl]-2-pyrrolidinemethanol, trifluoroacetic Acid salt, ¹H NMR (400 MHz, DMSO-d₆): δ 1.78 (m, 2H), 1.92 (m, 1H), 2.0 (m, 1H), 3.18 (m, 1H), 3.31 (m, 1H), 3.61 (m, 2H), 4.22 (m, 2H), 4.58 (m, 2H) 5.2 (s, 2H), 7.19 (d, 1H), 7.43 (d, 2H), 7.52 (d, 2H), 7.59 (dd, 1H), 7.68 (d, 1), 9.21 (br. s, 1H).

(2S)-1-[[5-Bromo-2-[(4-chlorophenyl)methoxy]phenyl]methyl]-2-pyrrolidinemethanol, trifluoroacetic Acid salt, ¹H NMR (400 MHz, DMSO-d₆): δ 1.76 (m, 2H), 1.95 (m, 1H), 2.0 (m, 1H), 3.18 (m, 1H), 3.31 (m, 1H), 3.61 (m, 2H), 4.22 (m, 2H), 4.58 (m, 2H), 5.2 (s, 2H), 7.19 (d, 1H), 7.43 (d, 2H), 7.52 (d, 2H), 7.57 (dd, 1H), 7.68 (d, 1), 9.21 (br. s, 1H).

(2R)-1-[[5-Bromo-2-[(4-chlorophenyl)methoxy]phenyl]methyl]-3-pyrrolidinol, trifluoroacetic acid salt, ¹H NMR (400 MHz, DMSO-d₆): δ 1.92 (m, 1H), 3.18 (br. s, 1H), 3.5 (m, 1H), 3.61 (m, 2H), 4.22 (m, 2H), 4.51 (m, 2H), 5.2 (d, 2H), 7.19 (dd, 1H), 7.43 (dd, 2H), 5.2 (s, 2H), 7.58 (dd, 1H), 7.71 (d, 1), 10.01 (br. d, 1H).

N¹-[[5-Bromo-2-[(4-chlorophenyl)methoxy]phenyl]methyl]-N¹-[2-(diethylamino)ethyl]-N²,N²-diethyl-1,2-ethanediamine.

¹H NMR (DMSO-d₆, 400 MHz) δ 0.90 (t, 12H), 2.40 (m, 16H), 3.60 (s, 2H), 5.10 (s, 1H), 6.95 (d, 1H), 7.30 (d, 1H), 7.45 (m, 4H), 7.60 (s, 1H).

1-[[5-Bromo-2-[(4-chlorophenyl)methoxy]phenyl]methyl]-4-piperidinone.

¹H NMR (DMSO-d₆): δ 2.49˜2.50 (m, 2H), 2.75 (m, 2H), 3.35˜3.51 (m, 4H), 4.35 (br.s, 2H), 5.15 (s, 2H), 7.16 (br.d, 1H), 7.42˜7.54 (m, 4H), 7.60 (br.d, 1H), 7.86 (br.s, 1H).

1-[[5-bromo-2-[(4-chlorophenyl)methoxy]phenyl]methyl]-4-(4-bromophenyl)-4-piperidinol.

¹H NMR (DMSO-d₆/TFA): δ 1.75 (br.d, 1H), 2.10 (m, 1H), 2.40˜2.70 (m, 2H), 3.20˜3.60 (m, 4H), 4.30˜4.35 (m, 2H), 5.15 (m, 2H), 7.16 (m, 1H), 7.32˜7.54 (m, 8H), 7.60 (m, 1H), 7.75 (m, 1H).

1-[[5-bromo-2-[(4-chlorophenyl)methoxy]phenyl]methyl]-4-piperidinol. ¹H NMR (DMSO-d₆/TFA): δ 1.54˜1.72 (m, 2H), 1.80˜1.92 (m, 2H), 2.94 (m, 1H), 3.04˜3.16 (m, 2H), 3.24˜3.34 (m, 1H), 3.60 and 3.86 (each m, 1H), 4.18˜4.24 (m, 2H), 5.14˜5.17 (m, 2H), 7.10˜7.16 (m, 1H), 7.40˜7.50 (m, 4H), 7.56 (m, 1H), 7.76 (m, 1H), 9.70 (br.s, 1H).

[1-[[5-bromo-2-[(4-chlorophenyl)methoxy]phenyl]methyl]-4-piperidinyl]carbamic acid, 1,1-dimethylethyl ester. ¹H NMR (CDCl₃): δ 1.40-1.60 (m, 11H), 1.90 (br.d, 2H), 2.15 (br.d, 2H), 2.70 (br.d, 2H), 3.48 and 4.45 (each br.s, 1H), 3.50 (s, 2H), 5.00 (s, 2H), 6.74 (d, 1H), 7.29 (dd, 1H), 7.32˜7.38 (m, 4H), 7.49 (d, 1H).

1-[[5-bromo-2-[(4-chlorophenyl)methoxy]phenyl]methyl]-4-ethoxypiperidine. ¹H NMR (DMSO-d₆/TFA): δ 1.00˜1.14 (m, 3H), 1.44˜2.08 (m, 4H), 2.90˜3.60 (m, 7H), 4.20 and 4.40 (each s, 2H), 5.12 (s, 2H), 7.12 (m, 1H), 7.34˜7.50 (m, 4H), 7.52˜7.58 (m, 1H), 7.66˜7.70 (m, 1H), 9.20 (br.s, 1H).

8-[[5-bromo-2-[(4-chlorophenyl)methoxy]phenyl]methyl]-1,4-dioxa-8-azaspiro[4.5]decane. ¹H NMR (DMSO-d₆/TFA): δ 1.92˜2.08 (m, 4H), 3.20 (m, 2H), 3.48˜3.56 (m, 2H), 4.03 (br.s, 4H), 4.44 (s, 2H), 5.32 (s, 2H), 7.30 (dd, 1H), 7.54˜7.66 (m, 4H), 7.70˜7.76 (m, 1H), 7.86 (d, 1H), 9.60 (br.s, 1H).

[[1-[[5-bromo-2-[(4-chlorophenyl)methoxy]phenyl]methyl]-4-piperidinyl]methyl]carbamic acid, 1,1-dimethylethyl ester. ¹H NMR (DMSO-d₆/TFA): δ 1.30˜1.80 (m, 14H), 2.80˜3.40 (m, 6H), 4.22 and 4.32 (each d, 2H), 5.18 (s, 2H), 6.92 (br.s, 1H), 7.14˜7.20 (m, 1H), 7.44˜7.54 (m, 4H), 7.60 (m, 1H), 7.68 and 7.74 (each d, 1H), 9.10˜9.30 (m, 1H).

5-bromo-2-[(4-chlorophenyl)methoxy]benzenemethanamine. ¹H NMR (DMSO-d₆): δ 3.66 (s, 2H), 5.10 (s, 2H), 6.90˜6.98 (m, 1H), 7.30 (br.dd, 1H), 7.40˜7.48 (m, 4H), 7.50 (br.dd, 1H),

1-[[5-bromo-2-[(4-chlorophenyl)methoxy]phenyl]methyl]pyridinium bromide. ¹H NMR (DMSO-d₆/TFA): δ 3.28 (s, 2H), 5.07 (s, 2H), 5.78 (s, 2H), 7.10 (d, 1H), 7.22˜7.44 (m, 4H), 7.60 (dd, 1H), 7.80 (d, 1H), 8.03 (dd, 2H), 8.56 (m, 1H), 8.96 (dd, 2H).

1-[[5-bromo-2-[(4-chlorophenyl)methoxy]phenyl]methyl]-4-piperidinecarboxylic acid, ethyl ester. ¹H NMR (DMSO-d₆/TFA): δ 1.44 (t, 3H), 1.62˜2.06 (m, 4H), 2.54˜2.80 (m, 1H), 2.92˜3.06 (m, 2H), 3.18˜3.42 (m, 2H), 4.00 (m, 2H), 4.18˜4.28 (m, 2H), 5.16 (br.s, 2H), 7.16 (d, 1H), 7.40˜7.54 (m, 4H), 7.58˜7.63 (m, 1H), 7.68˜7.74 (m, 1H), 9.30˜9.60 (m, 1H).

2-[[[5 -bromo-2-[(4-chlorophenyl)methoxy]phenyl]methyl]amino]ethanol.

¹H NMR (DMSO-d₆/TFA): δ 2.95 (br.s, 2H), 3.65 (br.t, 2H), 4.10˜4.20 (m, 2H), 5.15 (s, 2H), 7.09 (d, 1H), 7.40˜7.58 (m, 5H), 7.64 (d, 1H), 8.84 (br.s, 2H).

2-[[[5-bromo-2-[(4-chlorophenyl)methoxy]phenyl]methyl](methyl)amino]-ethanol. ¹H NMR (DMSO-d₆/TFA): δ 2.70 (s, 3H), 3.05˜3.25 (m, 2H), 3.70 (t, 2H), 4.15˜4.45 (m, 2H), 5.15 (s, 2H), 7.14 (d, 1H), 7.40˜7.54 (m, 4H), 7.59 (dd, 1H), 7.70 (d, 1H), 9.40 (br.s, 1H).

1-[[5-bromo-2-[(4-chlorophenyl)methoxy]phenyl]methyl]-3-piperidinol. ¹H NMR (DMSO-d₆/TFA): δ 1.20˜2.10 (m, 4H), 2.55˜3.30 (m, 4H), 3.65˜4.00 (m, 1H), 4.10˜4.40 (m, 2H), 5.15 (dd, 2H), 7.12˜7.18 (m, 1H), 7.40˜7.52 (m, 4H), 7.56˜7.61 (m, 1H), 7.68˜7.74 (m, 1H), 9.20 and 9.70 (each br.s, 1H).
1-[[5-bromo-2-[(4-chlorophenyl)methoxy]phenyl]methyl]-3-pyrrolidinol. ¹H NMR (DMSO-d₆/TFA): δ 1.72˜2.26 (m, 2H), 3.02˜3.58 (m, 4H), 4.30 (m, 1H), 4.32˜4.44 (m, 2H), 5.14˜5.22 (m, 2H), 7.10˜7.16 (m, 1H), 7.40˜7.52 (m, 4H), 7.54˜7.60 (m, 1H), 7.70˜7.72 (m, 1H), 10.00˜10.20 (m, 1H).

(1S,2S)-2-[[[5-bromo-2-[(4-chlorophenyl)methoxy]phenyl]methyl]amino]-1-(4-nitrophenyl)-1,3-propanediol. ¹H NMR (DMSO-d₆/TFA): δ 3.28 (m, 2H), 3.58 (m, 1H), 4.30 (m, 2H), 4.96 (br.d, 1H), 5.16 (m, 2H), 7.08 (d, 1H), 7.40˜7.44 (m, 2H), 7.48˜7.54 (m, 3H), 7.58˜7.64 (m, 3H), 8.18˜8.22 (m, 2H), 8.50 (br.s, 1H), 9.00 (br.s, 1H).

### Example 6B

### 5-Bromo-2-[(4-chlorophenyl)methoxy]-N,N,N-trimethyl-benzenemethanaminium iodide

To a stirred solution of 4-bromo-2-(bromomethyl)-1-[(4-chlorophenyl)methoxy]benzene (200 mg, 0.51 mmol) in DMF (5 mL), was added NHMe₂HCl (217 mg), followed by Cs₂CO₃ (1.17 g, 0.51 mmol) at rt. After 4 h, Cs₂CO₃ was filtered off and DMF was removed in vacuo. The residue was diluted with ethyl acetate, washed with water, brine, and dried (Na₂SO₄). After filtering off solid, the resulting organic solution was concentrated in vacuo to give 5-bromo-2-[(4-chlorophenyl)methoxy]-N,N-dimethylbenzenemethanamine (104 mg, 61%). To a stirred solution of 5-bromo-2-[(4-chlorophenyl)methoxy]-N,N-dimethylbenzenemethanamine (100 mg) in toluene (5 mL), was added MeI (0.19 mL, 5.1 mmol) at rt. The mixture was heated at 50° C. overnight. After cooling to rt, the solid was separated by filtration and washed with toluene to afford the title compound. ¹H NMR (DMSO-d₆, 400 MHz): δ (3.0 (s, 9H), 4.45 (s, 2H), 5.20 (s, 2H), 7.50(m, 4H), 7.70 (m, 2H).

### Example 7

### (3R,4S)-1-[[5-Bromo-2-[(4-chlorophenyl)methoxy]phenyl]methyl]-3,4-pyrrolidinediol

A mixture of 1-[[5-bromo-2-[(4-chlorophenyl)methoxy]phenyl]methyl]-2,5-dihydro-1H-pyrrole (400 mg, 1.1 mmol), AD-mix-b(1.7 g, 1.21 mmol), and MeSO₂NH₂ (103 mg, 1.1 mmol) was stirred in t-BuOH-H₂O (20 mL, 1:1, v/v) at rt for 2 days. The reaction was quenched by addition (in portion) of Na₂S₂O₅ (1 g). The mixture was partitioned with EtOAc and washed with brine. The organic phase was dried over Na₂SO₄, and concentrated. The residue was purified by HPLC to afford the title compound as a light yellow powder. ¹H NMR (DMSO-d₆/TFA): δ 3.10˜3.25 (m, 2H), 3.30˜3.40 (m, 1H), 3.45˜3.55 (m, 1H), 4.08 (m, 1H), 4.22 (m, 1H), 4.30 (m, 1H), 4.44 (m, 1H), 5.17 (d, 2H), 7.13 (dd, 1H), 7.40˜7.60 (m, 5H), 7.72 (m, 1H),

### Example 8

### 1-[[5-Bromo-2-[(4-chlorophenyl)methoxy]phenyl]methyl]-4-piperidinecarboxylic acid

A mixture of 1-[[5-bromo-2-[(4-chlorophenyl)methoxy]phenyl]methyl]-4-piperidinecarboxylic acid, ethyl ester (320 mg, 0.69 mmol), LiOH.H₂O(200 mg) in THF-H₂O (5 ml, 4:1, v/v) was stirred at rt under N₂ for 2 days. The mixture was acidified by addition of 1.0 HCl (aq.). After removing THF and H₂O under reduced vacuum, the residue was purified by HPLC to afford the product as a white powder. ¹H NMR (DMSO-d₆/TFA): δ 1.60˜2.10 (m, 4H), 2.40˜3.40 (m, 5H), 4.15˜4.30 (m, 2H), 5.16 (s, 2H), 7.15 (d, 1H), 7.40˜7.52 (m, 4H), 7.59 (dd, 1H), 7.69 (br.dd, 1H), 9.40˜9.60 (m, 1H).

### Example 9

### 1-[[5-bromo-2-[(4-chlorophenyl)methoxy]phenyl]methyl]-4-piperidinamine

To a solution of [1-[[5-bromo-2-[(4-chlorophenyl)methoxy]phenyl]methyl]-4-piperidinyl]-carbamic acid, 1,1-dimethylethyl ester (12 mmol) in CH₂Cl₂ (20 mL) was added TFA (10 mL) at rt. After stirring overnight, solvent was removed under vacuum, and the mixture was diluted with EtOAc, washed with NaHCO₃ (sat.) and brine, and dried over Na₂SO₄. After concentration; the residue was purified by recrystallization to afford product as white crystals.

¹H NMR (DMSO-d₆/TFA): δ 1.70(m, 2H), 1.95˜2.05(m, 2H), 2.95˜3.45(m, 5H), 4.18˜4.25(m, 2H), 5.15 (s, 2H), 7.14 (d, 1H), 7.38˜7.50 (m, 4H), 7.57 (dd, 1H), 7.68 (d, 1H), 8.04 (br.s, 3H), 9.70 (br.s, 1H).

The following compounds were prepared in a similar manner:

1-[[5-Bromo-2-[(4-chlorophenyl)methoxy]phenyl]methyl]-4-piperidinemethanamine. ¹H NMR (DMSO-d₆/TFA): δ 1.26˜1.96 (m, 5H), 2.64˜3.42 (m, 6H), 4.18˜4.30 (m, 2H), 5.12˜5.16 (m, 2H), 7.08˜7.14 (m, 1H), 7.38˜7.50 (m, 4H), 7.53˜7.58 (m, 1H), 7.68˜7.70 (m, 1H), 7.76 (br.s, 3H), 9.20˜9.40 (br.s, 1H).

1-[[5-Bromo-2-[(4-chlorophenyl)methoxy]phenyl]methyl]-N-methyl-4-piperidinemethanamine. ¹H NMR (DMSO-d₆/TFA): δ 1.30˜1.90 (m, 5H), 2.60˜3.40 (m, 6H), 4.16˜4.24 (each s, 2H), 5.06 (br.s, 2H), 7.00 (m, 1H), 7.28˜7.40 (m, 4H), 7.47 (m, 1H), 7.60˜7.76 (m, 1H), 8.36 (br.s, 1H), 9.20˜9.30 (m, 1H).

### Example 10

### [1-[[5-Bromo-2-[(4-chlorophenyl)methoxy]phenyl]methyl]-4-piperidinyl](ethyl)carbamic acid, 1,1-dimethylethyl ester

To a suspension of NaH (220 mg, 95%, 12.7 mmol) in DMF (5 mL) was added a solution of [1-[[5-bromo-2-[(4-chlorophenyl)methoxy]phenyl]methyl]-4-piperidinyl]carbamic acid, 1,1-dimethylethyl ester (4.2 g, 8.3 mmol) in DMF (15 mL) at rt under N₂. After 3 h at rt, EtI (0.75 mL, 9.4 mmol) was added. The resulting mixture was stirred at rt under N₂ for 24 h, and was poured into ice-water while stirring vigorously. The solid was collected by filtration, and was re-dissolved in CH₂Cl₂. The organic solution was washed with ice-water, dried over Na₂SO₄, and concentrated. The residue was purified by flash chromatography to afford the title compound as a white powder. ¹H NMR (DMSO-d₆/TFA): δ 0.92˜1.04(m, 3H), 1.38 (br.s, 9H), 1.70˜1.80 (m, 2H), 1.92˜2.04 (m, 2H), 2.96˜3.22 (m, 4H), 3.40 (m, 2H), 3.92 (m, 1H), 4.24 and 4.37 (each m, 2H), 5.13 and 5.18 (each s, 2H), 7.16˜7.21 (m, 1H), 7.42˜7.56 (m, 4H), 7.59˜7.65 (m, 1H), 7.72 and 7.81 (each d, 1H); 9.20 and 9.50 (each br.s, 1H).

The following compound was prepared in a similar manner:

[1-[[5-Bromo-2-[(4-chlorophenyl)methoxy]phenyl]methyl]-4-piperidinyl](methyl)carbamic acid, 1,1-dimethylethyl ester. ¹H NMR (DMSO-d₆/TFA): δ 1.24˜1.70 (m, 12H), 2.02 (m, 2H), 2.85 (s, 3H), 2.90 (m, 2H), 3.10 (m, 2H), 3.55 (s, 2H), 5.00 (s, 2H), 6.75 (d, 1H), 7.29 (dd, 1H). 7.35 (br.s, 4H), 7.50 (d, 1H).

### Example 11

### 1-[[5-Bromo-2-[(4-chlorophenyl)methoxy]phenyl]methyl]-N,N-diethyl-4-piperidinamine

To a stirred solution of 1-[[5-bromo-2-[(4-chlorophenyl)methoxy]phenyl]methyl]-4-piperidinamine (1.5 g, 3.66 mmol) in CH₂Cl₂ (30 mL), was added acetaldehyde (0.3 mL, 5.3 mmol), followed by NaBH(OAc)₃. After 12 h, the solvent was removed, and the resulting residue was diluted with EtOAc. The organic phase was washed with brine, and dried over Na₂SO₄. Concentration, followed by purification by flash chromatography afforded the title compound as a white powder. ¹H NMR (CDCl₃): δ 1.34 (t, 6H), 1.82 (br.ddd, 2H), 1.98 (br.d, 2H), 2.12 (br.dd, 2H), 3.00 (br.d, 2H), 3.08 (q, 4H), 3.17 (m, 1H), 3.52 (s, 2H), 5.00 (s, 2H), 6.76 (d, 1H), 7.28˜7.38 (m, 5H), 7.45 (d, 1H).

### Example 12

### N-[1-[[5-Bromo-2-[(4-chlorophenyl)methoxy]phenyl]methyl]-4-piperidinyl]-N′-(4-fluorophenyl)-urea

To a stirred solution of 1-[[5-bromo-2-[(4-chlorophenyl)methoxy]phenyl]methyl]-4-piperidinamine (544 mg, 1.33 mol) in CH₂Cl₂ (5 mL) was added 4-fluorophenyl isocyanate (200 mg, 1.46 mmol). The reaction was stirred at room temperature for 3 h, then purified directly by column chromatography to afford the title compound. ¹H NMR (400 MHz, DMSO-d₆): 1.4 (m, 2H), 1.76 (m, 2H), 2.1 (m, 2H), 2.7 (m, 1H), 3.3 (d, 1H), 3.5 (br. s, 2H), 5.1 (s, 2H), 6.1 (br. s, 1H), 7.0 (m, 3H), 7.4 (m, 3H), 7.46 (m, 5H), 8.4 (s, 1H).

The following compounds were prepared in a similar manner:

N-[1-[[5-Bromo-2-[(4-chlorophenyl)methoxy]phenyl]methyl]-3-pyrrolidinyl]-N′-(4-fluorophenyl)-urea. Starting with 1-[[5-bromo-2-[(4-chlorophenyl)methoxy]phenyl]methyl]-3-pyrrolidinamine and 4-fluorophenyl isocyanate. ¹H NMR (400 MHz, DMSO-d₆): δ 1.5 (m, 1H), 2.16 (m, 1), 2.34 (m, 1H), 2.4 (m, 1H), 2.6 (m, 1H), 2.7 (m, 1H), 3.3 (br. s, 1H), 3.6 (s, 2H), 4.1 (br. s, 1H), 5.1 (s, 2H), 7.0 (m, 3H), 7.46 (m, 3H), 7.43 (m, 5H), 8.36 (s, 1).

N-[[1-[[5-Bromo-2-[(4-chlorophenyl)methoxy]phenyl]methyl]-4-piperidinyl]methyl]-N′-(4-fluorophenyl)-N-methyl-urea. Starting with 1-[[5-bromo-2-[(4-chlorophenyl)methoxy]phenyl]methyl]-N-methyl-4-piperidinemethanamine and 4-fluorophenyl isocyanate. ¹H NMR (DMSO-d₆/TFA): δ 1.28˜2.00 (m, 5H), 2.86˜3.40 (m, 9H), 4.20 and 4.28 (each d, 2H), 5.12 (s, 2H), 6.22˜6.99 (m, 2H), 7.08˜7.12(m, 1H), 7.32˜7.50 (m, 6H), 7.54 (dd, 1H), 7.66 and 7.72 (each d, 1H), 8.20 (br.s, 1H), 9.00˜9.20 (m, 1H).

N-[[1-[[5-Bromo-2-[(4-chlorophenyl)methoxy]phenyl]methyl]-4-piperidinyl]methyl]-N′-[(4-fluorophenyl)methyl]-N-methyl-urea. Starting with 1-[[5-bromo-2-[(4-chlorophenyl)methoxy]phenyl]methyl]-N-methyl-4-piperidinemethanami and 4-fluorophenymethyll isocyanate. ¹H NMR (DMSO-d₆/TFA): δ 1.08˜1.90 (m, 5H), 2.75 (s, 3H), 2.80˜3.40 (m, 6H), 4.10˜4.26 (m, 4H), 5.10 (s, 2H), 7.00 (br.dd, 1H), 7.10 (m, 1H), 7.19 (br.dd, 2H), 7.34˜7.48 (m, 4H), 7.53 (br.dd, 1H), 7.62˜7.72 (m, 1H), 9.00˜9.20 (m, 1H).

### Example 14

### N-[1-[[5-Bromo-2-[(4-chlorophenyl)methoxy]phenyl]methyl]-3-pyrrolidinyl]-2-chloroacetamide

To a stirred solution of 1-[[5-bromo-2-[(4-chlorophenyl)methoxy]phenyl]methyl]-3-pyrrolidinamine (238 mg, 0.6 mmol) in CH₂Cl₂ (5 mL) at 0° C., was added Et₃N (0.4 mL) followed by 2-chloroacetylchloride (102 mg, 0.9 mmol). After addition, the reaction mixture was stirred at room temperature for 3 h, and then was quenched with NaHCO₃ (sat. 10 mL). The reaction mixture was extracted with EtOAc (3×25 mL), washed with brine (1×15 mL), and dried over Na₂SO₄. Concentration followed by purification by column chromatography afforded the title compound. ¹H NMR (400 MHz, DMSO-d₆): δ 2.2 (m, 1H), 2.5 (m, 1H), 3.0 (m, 1H), 3.2 (m, 1H), 3.8 (m, 1H), 4.0 (s, 2H), 4.25 (dd, 2H), 4.8 (m, 1H), 5.1 (s, 2H), 6.9 (d, 1H), 7.37 (dd, 4H), 7.5 (dd, 1H), 7.63 (d, 1H). 8.6 (br.s, 1H).

The following compounds were prepared in a similar manner starting with different amines and acetyl chlorides:

N-[[1-[[5-Bromo-2-[(4-chlorophenyl)methoxy]phenyl]methyl]-4-piperidinyl]methyl]acetamide, Trifluoroacetic acid salt. Started with 1-[[5-bromo-2-[(4-chlorophenyl)methoxy]phenyl]methyl]4-piperidinemethanamine and acetic anhydride. ¹H NMR (DMSO-d₆/TFA): δ 1.26˜1.84 (m, 8H), 2.88˜3.40 (m, 6H), 4.20˜4.34 (m, 2H), 5.16˜5.20 (m, 2H), 7.14˜7.18 (m, 1H), 7.44˜7.54 (m, 4H), 7.60 (dd, 1H), 7.70˜7.74 (m, 1H), 7.95(br.t, 1H), 9.10˜9.30 (m, 1H).

N-[1-[[5-Bromo-2-[(4-chlorophenyl)methoxy]phenyl]methyl]-4-piperidinyl]-acetamide, Trifluoroacetic acid salt. Starting with 1-[[5-bromo-2-[(4-chlorophenyl)methoxy]phenyl]methyl]-4-piperidinamine and acetyl chloride. ¹H NMR (DMSO-d₆/TFA): δ 1.46˜1.92(m, 7H), 3.00˜3.40(m, 4H), 3.65˜3.90(m, 1H), 4.15˜4.25 (m, 2H), 5.10˜5.20 (m, 2H), 7.12˜7.18 (m, 1H), 7.40˜7.54 (m, 4H), 7.60 (m, 1H), 7.66˜7.74 (m, 1H), 7.94 (m, 1H), 9.34 (br.s, 1H).

### Example 14

### N-[1-[[5-Bromo-2-[(4-chlorophenyl)methoxy]phenyl]methyl]-3-pyrrolidinyl]-N-methyl-2-pyrazinecarboxamide

To a stirred solution of 1-[[5-bromo-2-[(4-chlorophenyl)methoxy]phenyl]methyl]-N-methyl-3-pyrrolidinamine (234 mg, 0.59 mmol) in DMF (5 mL), was added Et₃N (237.4 mg, 2.35 mmol), followed by 2-pyrazinecarboxylic acid (88 mg, 0.71 mmol) and HATU (305 mg, 0.8 mmol) at rt. After 12 h, the reaction was quenched with NaHCO₃ (sat. 10 mL), and extracted with EtOAc (3×15 mL). The organic phase was washed with brine (1×15 mL), and dried over Na₂SO₄. Concentration, followed by purification through column chromatography afforded the title compound. ¹H NMR (400 MHz, DMSO-d₆): δ 1.9 (m, 1H), 2.38 (m, 1H), 2.63 (m, 1H), 2.8 (s, 2H), 3.1 (d, 3H), 3.4 (m, 1H), 3.6 (dd, 2H), 5.0 (s, 2H), 6.8 (d, 1H), 7.3 (dd, 1H), 7.35 (dd, 4H), 7.47 dd, 1H), 8.53 (br. s, 1H), 8.62 (d, 1H), 8.9 (dd, 1H). 7.54 (m, 6H), 7.60 (m, 1H), 7.68˜7.76 (m, 1H), 8.54˜8.64 (m, 1H), 9.24˜9.40 (m, 1H).

The following compounds were prepared in a similar manner:

N-[[1-[[5-Bromo-2-[(4-chlorophenyl)methoxy]phenyl]methyl]-4-piperidinyl]methyl]-N,4-dimethyl-3-pyridinecarboxamide. Starting with 1-[[5-bromo-2-[(4-chlorophenyl)methoxy]phenyl]methyl]-N-methyl-4-piperidinemethanamine and 4-methyl-3-pyridinecarboxylic acid. ¹H NMR (DMSO-d₆/TFA): δ 1.00˜2.26 (m, 5H), 2.44˜2.52 (m, 3H), 2.80˜3.54 (m, 9H), 4.20˜4.44 (m, 2H), 5.14 (m, 2H), 7.14˜7.20 (m, 1H), 7.40˜7.78 (m, 6H), 7.96˜8.04 (m, 1H), 8.80˜8.96 (m, 2H), 9.20˜9.30 (m, 1H).

### Example 15

### [1-[[5-Bromo-2-[(4-chlorophenyl)methoxy]phenyl]methyl]-4-piperidinyl]carbamic acid, methyl ester

To a stirred solution of 1-[[5-bromo-2-[(4-chlorophenyl)methoxy]phenyl]methyl]-4-piperidinamine (246 mg, 0.6 mmol) in CH₂Cl₂ (5 mL) at 0° C., was added Et₃N (0.4 mL) followed by methyl chloroformate (68 mg, 0.72 mmol). After addition, the reaction mixture was stirred at room temperature for 1 h, and quenched with NaHCO₃ (sat. 3 mL). The reaction mixture was extracted with EtOAc (3×25 mL), washed with brine (1×15 mL), and dried over Na₂SO₄. Concentration followed by purification through column chromatography afforded the title compound. ¹H NMR (DMSO-d₆/TFA): δ 1.50˜1.94 (m, 4H), 3.00˜3.70 (m, 8H), 4.16˜4.22 (m, 2H), 5.14˜5.18 (m, 2H), 7.13˜7.16 (m, 1H), 7.40˜7.52 (m, 4H), 7.57˜7.61 (m, 1H), 7.66˜7.71 (m, 1H).

### Example 16

### 4-[[4-Bromo-2-[(diethylamino)methyl]phenoxy]methyl]benzoic acid

To a stirred solution of 5-bromo-2-hydroxybenzaldehyde (15 g, 75 mmol) in DMF (250 mL) was added K₂CO₃ (20.7 g, 150 mmol) at rt. After 30 min, 4-bromomethylbenzoic acid, methyl ester (17.2 g, 75 mmol) was added. The reaction mixture was kept at rt overnight, then poured into an ice-cooled mixture of EtOAc/water (1:1, 2 L). The solid was collected by filtration, washed with water, and dried under vacuum for 20 h to give 4-[[4-bromo-2-(bromomethyl)phenoxy]methyl]benzoic acid, methyl ester (23 g, 88%). To a stirred solution of 4-[[4-bromo-2-(bromomethyl)phenoxy]methyl]benzoic acid, methyl ester (10 g, 28.6 mmol) in EtOAc (200 mL) was added diethylamine (2.2 g, 76 mmol) and HOAc (2 mL), followed by NaBH(OAc)₃ (19 g, 90 mmol) at rt. After 12 h, the mixture was diluted with EtOAc (250 mL), washed with brine (2×120 mL), and dried over Na₂SO₄. Concentration followed by purification by flash chromatography afforded the ester (10.1 g, 87%). This ester (10.1 g, 24.8 mmol) was hydrolyzed with LiOH.H₂O (1.32 g, 32 mmol) in THF (150 mL) and H₂O (100 mL) at rt for 10 h to afford the acid (9.4 g, 97%). ¹H NMR (400 MHz, MDSO): δ 0.99 (t, 6H), 2.46 (q, 4H), 3.61 (s, 2H), 4.85 (s, 2H), 6.72 (d, 1H), 7.0-7.2 (m, 4H), 8.1 (d, 2H).

### Example 17

### 5-Bromo-N,N-diethyl-2-[[4-[[4-(phenylmethyl)-1-piperazinyl]carbonyl]phenyl]methoxy]benzenemethanamine

To a stirred solution of 4-[[4-bromo-2-[(diethylamino)methyl]phenoxy]methyl]benzoic acid (196 mg, 0.5 mmol) in THF (5 mL), was added EDC (115 mg, 0.6 mmol) and HOBT (81 mg, 0.6 mmol) successively. After 15 min, 1-benzylpiperazine (0.091 mL, 0.53 mmol) was added. The reaction was stirred at rt overnight, and solvent was concentrated in vacuo. The residue was diluted with ethyl acetate, washed with a 1M solution of NaHSO₄, saturated NaHCO₃, and brine, and dried over Na₂SO₄. Concentration followed by purification by flash chromatography afforded the title compound. ¹H NMR (CD₃OD, 400 MHz) δ (TMS) 1.05 (m, 6H), 2.55 (m, 8H), 3.45 (m, 2H), 3.55 (s, 2H), 3.70 (m, 4H), 5.15 (s, 2H), 6.95 (d, 1H), 7.30 (m, 6H), 7.40 (m, 2H), 7.50 (m, 1H), 7.54 (m, 2H).

The following compounds were prepared in a similar manner:

N-(1,3-Benzodioxo-5-ylmethyl)-4-[[4-bromo-2-[(diethylamino)methyl]phenoxy]methyl]benzamide. ¹H NMR (CDCl₃, 400 MHz) δ (TMS) 1.05 (t, 6H), 2.58 (q, 4H), 3.60 (s, 2H), 4.55 (d, 2H), 5.10 (s, 2H), 5.95 (s, 2H), 6.35 (m, 1H), 6.80 (m, 4H), 7.25 (m, 1H), 7.50(d, 2H), 7.60 (s, 1H), 7.80 (s, 2H).

4-[[4-Bromo-2-[(diethylamino)methyl]phenoxy]methyl]-N-[(4-methoxyphenyl)methyl]benzamide. ¹H NMR (CDCl₃, 400 MHz) δ (TMS) 1.05 (t, 6H), 2.58 (m, 4H), 3.60 (s, 2H), 3.80 (s, 3H), 4.60 (d, 2H), 5.10 (s, 2H), 6.30 (m, 1H), 6.70 (d, 1H), 6.90 (d, 2H), 7.30 (m, 3H), 7.48 (d, 2H), 7.60 (s, 1H), 7.80 (d, 2H).

4-[[4-Bromo-2-[(diethylamino)methyl]phenoxy]methyl]-N-methyl-N-(2-phenylethyl)benzamide. ¹H NMR (CDCl₃, 400 MHz) δ (TMS) 1.05 (t, 6H), 2.55 (m, 4H), 2.85 (s, 3H), 3.0 (m, 1H), 3.16 (m, 1H), 3.50 (m, 1H), 3.60 (s, 2H), 3.80 (m, 1H), 5.05 (s, 2H), 6.75 (d, 1H), 6.95 (m, 1H), 7.10 (m, 1H), 7.30 (m, 7H), 7.60 (s, 1H).

4-[[4-Bromo-2-[(diethylamino)methyl]phenoxy]methyl]-N-[2-(4-bromo-phenyl)ethyl]benzamide. ¹H NMR (CDCl₃, 400 MHz): δ (TMS) 1.05 (t, 6H), 2.58 (q, 4H), 2.90 (t, 2H), 3.60 (s, 1H), 3.70 (q, 2H), 5.10 (s, 2H), 6.20 (m, 1H), 6.70 (d, 1H), 7.10 (d, 2H), 7.25 (m, 1H), 7.45 (m, 4H), 7.60 (s, 1H), 7.70 (d, 2H).

### Example 18

### 4-[4-[[4-Bromo-2-[(diethylamino)methyl]phenoxy]methyl]benzoyl]-N-octyl-1-piperazincarboxamide

5-Bromo-N,N,diethyl-2-[[4-(1-piperazinylcarbonyl)phenyl]methoxy]-benzenemethanamine (99 mg, 0.23 mmol) was dissolved in CH₂Cl₂ (5 mL), and octylisocyanate (50 mg, 0.32 mmol) was added. The reaction mixture was stirred at rt for 2 h. Solvent was concentrated in vacuo. Flash chromatography afforded the title compound. ¹H NMR (CDCl₃, 400 MHz) δ (TMS) 0.90 (t, 3H), 1.05 (m, 6H), 1.30 (m, 10), 1.50 (m, 2), 2.60 (m, 4H), 3.25 (m, 2H), 3.45 (m, 6H), 3.60 (s, 2H), 3.78 (m, 2H), 4.45 (m, 1H), 5.10 (s, 2H), 6.78 (d, 1H), 7.30 (m, 1H), 7.45 (m, 4H), 7.65 (s, 1H).

### Example 19

### 5-Bromo-N,N-didiethyl-2-[[4-[[4-[[3-nitrophenyl)sulfonyl]-1-piperazinyl]carbonyl]phenyl]methoxy]benzenemethanamine

5-Bromo-N,N,diethyl-2-[[4-(1-piperazinylcarbonyl)phenyl]methoxy]-benzenemethanamine (91 mg, 0.21 mmol) was dissolved in CH₂Cl₂ (5 mL), and 3-nitrobenzenesulfonyl chloride (50 mg, 0.23 mmol) was added followed by triethylamine (0.043 mL, 0.34 mmol) and DMAP (2 mg). After 2 h at rt, the solvent was removed in vacuo. Flash column chromatography on silica gel with a gradient of 1% to 3% MeOH in CH₂Cl₂ afforded the title compound. ¹H NMR (DMSO-d₆, 400 MHz) δ (TMS) 0.95 (t, 6H), 2.40 (m, 4H), 3.0 (m, 4H), 3.40˜3.65 (m, 4H), 3.50 (s, 2H), 5.10 (s, 2H), 6.95 (d, 1H), 7.35 (m, 3H), 7.45 (m, 3H), 7.95 (t, 1H), 8.15 (d, 1H), 8.30 (s, 1H), 8.55 (d, 1H).

The following compounds were prepared in a similar manner:

5-Bromo-N,N-diethyl-2-[[4-[[4-(2-furanylcarbonyl)-1-piperazinyl]carbonyl]phenyl]methoxy]benzenemethanamine. 1H NMR (CDCl₃, 400 MHz) δ (TMS) 1.05 (t, 6H), 2.60 (q, 4H), 3.50-3.90 (m, 8H), 3.60 (s, 2H), 3.80 (s, 2H), 5.10 (s, 2H), 6.50 (s, 1H), 6.75 (d, 1H), 7.05 (d, 1H), 7.30 (m, 1H), 7.50 (m, 5H), 7.60 (s, 1H).

5-Bromo-2-[[4-[[4-(2,6-dichlorobenzoyl)-1-piperazinyl]carbonyl]phenyl]methoxy]-N,N-diethylbenzenemethanamine: MS: 634 (M+1). ¹H NMR (CDCl₃, 400 MHz): δ (TMS) 1.05 (m, 6H), 2.55 (m, 4H), 3.30 (m, 2H), 3.60 (s, 2H), 3.850 (m, 6H), 5.08 (s, 2H), 6.70 (d, 1H), 7.25 to 7.50 (m, 8H), 7.60 (s, 1H).

N-[[5-[[4-[4-[[4-Bromo-2-[(diethylamino)methyl]phenoxy]methyl]benzoyl]-1-piperazinyl]sulfonyl]-2-thienyl]methyl]benzamide. MS: 740 (M+1). ¹H NMR (CDCl₃, 400 MHz): δ (TMS) 1.05 (t, 6H), 2.55 (q, 4H), 3.10 (m, 4H), 3.55-3.90 (m, 4H), 3.60 (s, 2H), 4.80 (d, 2H), 5.06 (s, 2H), 6.70 (d, 1H), 6.85 (m, 1H), 7.05 (d, 1H), 7.25 (m, 1H), 7.36 (m, 3H), 7.45 (m, 4H), 7.54 (m, 1H), 7.60 (s, 1H), 7.80 (m, 2H).

### Example 20

### 1-[(5-bromo-2-propoxyphenyl)methyl]-4-(4-fluorophenyl)-4-piperidinol

To a stirred mixture of 5-bromo-2-propoxy-benzaldehyde (200 mg, 0.82 mmol) and 4-(4-fluorophenyl)-4-piperidinol (177 mg, 0.9 mmol) in EtOAc (10 mL) was added HOAc (70 mg, 1.2 mmol), followed by NaBH(OAc)₃ (262 mg, 1.2 mmol) at room temperature. After 16 h, the reaction was worked-up as usual, and purified by column chromatography to afford the title compound as a white solid. ¹H NMR (DMSO-d₆, 400 MHz): δ 0.8-1.1 (t, 3H), 1.6-1.9 (m, 5H), 2.0-2.2 (m, 2H), 3.0-3.6 (m, 2H), 3.9-4.1 (m, 2H), 4.2-4.4 (m, 2H), 5.4-5.6 (m, 1H), 7.0-7.3 (m, 3H), 7.34-7.5 (m, 2H), 7.54-7.64 (m, 1H), 7.7-7.8 (m, 1H).

The following compounds were prepared in a similar manner:

[4-Bromo-2-[[4-(4-bromophenyl)-4-hydroxy-1-piperidinyl]methyl]phenoxy]-O-ethyloxime-ethanal. ¹H NMR (CDCl₃, 400 MHz): δ 1.2-1.4 (m, 3H), 1.4-1.8 (m, 4H), 2.0-2.3 (m, 2H), 2.4-2.6 (m, 2H), 2.7-2.9 (m, 2H), 3.5-3.68 (d, 2H), 4.1-4.3 (m, 2H), 4.6 (d, 1H), 4.8 (m, 1H), 6.6-7.0 (m, 1H), 7.24-7.6 (m, 6H).

1-[(5-Bromo-2-propoxyphenyl)methyl]-4-(4-chlorophenyl)-4-piperidinol

¹H NMR (DMSO-d₆, 400 MHz) δ 0.8-1.1 (t, 3H), 1.4-1.6 (m, 2H), 1.6-1.8 (m, 4H), 1.8-2.0 (m, 2H), 2.3-2.7 (m, 2H), 3.4-3.6 (m, 2H), 3.8-4.0 (m, 2H), 4.8-5.0 (s, 1H), 6.8-7.0 (m, 1H), 7.3-7.4 (m, 3H), 7.4-7.6 (m, 3H).

1-[[5-Bromo-2-(pentyloxy)phenyl]methyl]-4-(4-bromophenyl)-4-piperidinol

¹H NMR (DMSO-d₆, 400 MHz) δ 0.8-1.0 (t, 3H), 1.0-1.2 (m, 2H), 1.2-1.5 (m, 4H), 1.6-1.84 (m, 3H), 2.0-2.3 (m, 1 H), 3.0-3.6 (m, 4H), 3.9-4.1 (m, 2H), 4.2-4.4 (m, 2H), 5.5-5.7 (m, 1H), 7.0-7.2 (m, 1H), 7.3-7.4 (m, 2H), 7.5-7.7 (m, 3H), 7.7-7.8 (m, 1H).

1-[[5-Bromo-2-(hexyloxy)phenyl]methyl]-4-(4-bromophenyl)-4-piperidinol

¹H NMR (DMSO-d₆, 400 MHz): δ 0.7-1.0 (t, 3H), 1.0-1.5 (m, 6H), 1.6-1.9 (m, 4H), 2.0-2.2 (m, 2H), 3.0-3.6 (m, 4H),3.9-4.1 (m, 2H), 4.2-4.4 (m, 2H), 5.5-5.7 (m, 1H), 7.0-7.2 (m, 1H), 7.3-7.4 (m, 2H),7.5-7.7 (m,3H), 7.7-7.8 (m,1H).

1-[(5-Bromo-2-methoxyphenyl)methyl]-4-(4-bromophenyl)-4-piperidinol.

¹H NMR (DMSO-d₆/TFA): δ 1.70˜1.80 (m, 2H), 2.15 (m, 2H), 3.30 (m, 4H), 3.85 (s, 3H), 4.30 and 4.45 (each d, 2H), 7.06˜7.10 (m, 1H), 7.32˜7.54 (m, 4H), 7.60 (dd, 1H), 7.68˜7.77 (m, 1H), 9.30 (br.s, 1H).

1-[[5-Bromo-2-(1,3-dioxolan-2-ylmethoxy)phenyl]methyl]-4-(4-bromophenyl)-4-piperidinol.

¹H NMR (CDCl₃): δ 1.73 (m, 2H), 2.24 (ddd, 2H), 2.70 (ddd, 2H), 2.95 (m, 2H), 3.76 (s, 2H), 3.94˜4.07 (m, 6H), 5.29 (dd, 1H), 6.76 (d, 1H), 7.35 (dd, 1H), 7.39 (m, 2H), 7.47 (m, 2H), 7.55 (d, 1H).

### Example 21

### 1-[(5-Bromo-2-hydroxyphenyl)methyl]-4-(4-bromophenyl)-4-piperidinol

To a stirred mixture of 5-bromo-2-hydroxybenzaldehyde (7.06 g, 35 mmol), 4-(4-bromophenyl)-4-piperidinol (10 g, 39 mmol), and HOAc (6 mL) in EtOAc (250 mL) was added NaBH(OAc)₃ (7.4 g, 35 mmol) at rt. The mixture was stirred at rt overnight, and diluted with EtOAc (300 mL), washed with brine (2×100 mL), and dried over Na₂SO₄. Concentration followed by purification through flash chromatography afford the title compound. ¹H NMR (400 MHz, DMSO-d₆): δ 1.4-1.7 (m, 2H), 1.8-2.0 (m, 2H), 2.3-2.6 (m, 2H), 2.6-2.8 (m, 2H), 3.5-3.8 (m, 2H), 6.6-6.8 (m, 1H), 7.2 (m, 2H), 7.2-5 (m, 4H).

### Example 22

### 1-[[5-Bromo-2-(2-methylpropoxy)phenyl]methyl]-4-(4-bromophenyl)-4-piperidinol

A mixture of 1-[(5-bromo-2-hydroxyphenyl)methyl]-4-(4-bromophenyl)-4-piperidinol (150 mg, 0.34 mmol), Cs₂CO₃ (200 mg, 0.61 mmol) in DMF (5 mL) in a sealed tube was stirred at rt for 5 min, and isobutyl iodide (0.05 mL, 0.43 mmol) was added. The reaction was stirred at 50° C. for 0.5 h, then 75° C. overnight (the reaction was checked by HPLC). The reaction mixture was poured into ice-water, and the crude product was obtained by filtration. Th crude solid was re-dissolved in CH₂Cl₂, and dried over Na₂SO₄. Concentration, followed by purification by HPLC afforded the title compound as a white powder, trifluoroacetic acid salt. ¹H NMR (DMSO-d₆/TFA): δ 0.90 (d, 6H), 1.77(br. d, 2H), 2.00˜2.20(m, 3H), 3.32(m, 4H), 3.80(d, 2H), 4.30(d, 2H), 7.08(d, 1H), 7.34(m, 2H), 7.52(m, 2H), 7.58 (dd, 1H), 7.73(d, 1H).

The following compounds were prepared in a similar manner:

1-[[5-Bromo-2-(heptyloxy)phenyl]methyl]-4-(4-bromophenyl)-4-piperidinol, trifluoroacetic acid. ¹H NMR (DMSO-d₆/TFA): δ 0.83 (t, 3H), 1.11˜1.44 (m, 8H), 1.68˜1.80 (m, 4H), 2.14 (m, 2H), 3.30 (m, 4H), 4.02 (t, 2H), 4.28 (d, 2H), 7.08 (d, 1H), 7.34 (m, 2H), 7.52 (m, 2H), 7.59 (dd, 1H), 7.71(d, 1H).

1-[[5-Bromo-2-(cyclopropylmethoxy)phenyl]methyl]-4-(4-bromophenyl)-4-piperidinol, trifluoroacetic acid. ¹H NMR (DMSO-d₆/TFA): δ 0.93 (m, 1H), 1.48 (br.d, 2H), 1.84 (br.ddd, 2H), 3.06 (m, 4H), 3.60 (d, 2H), 4.00 (d, 2H), 6.76 (d, 1H), 7.05 (m, 2H), 7.23 (m, 2H), 7.27 (dd, 1H), 7.42 (d, 1H).

1-[(5-Bromo-2-butoxyphenyl)methyl]-4-(4-bromophenyl)-4-piperidinol, trifluoroacetic acid. ¹H NMR (DMSO-d₆/TFA): δ 0.93 (t, 3H), 1.42 (m, 2H), 1.64˜1.82 (m, 4H), 2.14 (m, 2H), 3.30 (m, 4H), 4.02 (t, 2H), 4.28 (d, 2H), 7.09 (m, 1H), 7.36 (m, 2H), 7.53 (m, 2H), 7.58 (m, 1H), 7.72 (d, 1H).

1-[[5-Bromo-2-(2-methoxyethoxy)phenyl]methyl]-4-(4-bromophenyl)-4-piperidinol, trifluoroacetic acid. ¹H NMR (DMSO-d₆/TFA): δ 1.50 (br.d, 2H), 1.83 (br.ddd, 2H), 3.00 (s, 3H), 3.05 (m, 4H), 3.40 (m, 2H), 3.90 (m, 2H), 4.00 (d, 2H), 6.84 (m, 1H), 7.07 (m, 2H), 7.24 (m, 2H), 7.32 (m, 1H), 7.45 (d, 1H).

4-(4-Bromophenyl)-1-[(5-bromo-2-propoxyphenyl)methyl]-4-piperidinol, trifluoroacetic acid.

¹H NMR (DMSO-d₆/TFA): δ 0.97 (t, 3H), 1.68˜1.84 (m, 4H), 2.09˜2.18 (m, 2H), 3.30 (m, 4H), 3.98 (m, 2H), 4.29 (d, 2H), 7.07 (m, 1H), 7.34 (m, 2H), 7.52 (m, 2H), 7.58 (m, 1H), 7.71 (d, 1H).

1-[(5-Bromo-2-ethoxyphenyl)methyl]-4-(4-bromophenyl)-4-piperidinol, trifluoroacetic acid.

¹H NMR (DMSO-d₆/TFA): δ 1.34 (t, 3H), 1.77 (m, 2H), 2.14 (m, 2H), 3.30 (m, 4H), 4.08 (q, 2H), 4.28 (m, 2H), 7.07 (m, 1H), 7.34 (m, 2H), 7.52 (m, 2H), 7.58 (m, 1H), 7.71 (d, 1H).

4-(4-Bromophenyl)-1-[[5-bromo-2-(2-propenyloxy)phenyl]methyl]-4-piperidinol, trifluoroacetic acid. ¹H NMR (DMSO-d₆/TFA): δ 1.75(m, 2H), 2.15(m, 2H), 3.30 (m, 4H), 4.30 (d, 2H), 4.53 (d, 2H), 5.25 (d, 1H), 5.40 (d, 1H), 6.01 (m, 1H), 7.08 (m, 1H), 7.34 (m, 2H), 7.52 (m, 2H), 7.59 (m, 1H), 7.72 (d, 1H).

[5 -Bromo-2-[[4-(4-bromophenyl)-4-hydroxy-1-piperidinyl]methyl]phenoxy]-acetonitrile, trifluoroacetic acid. ¹H NMR (DMSO-d₆/TFA): δ 1.85 (br.d, 2H), 2.20 (m, 2H), 3.40 (m, 4H), 4.40 (d, 2H), 5.35 (s, 2H), 7.32 (d, 1H), 7.42 (m, 2H), 7.60 (m, 2H), 7.79 (m, 1H), 7.88 (d, 1H).

### Example 23

### N-[2-[4-Bromo-2-[[4-(4-bromophenyl)-4-hydroxy-1-piperidinyl]methyl]phenoxy]ethyl]-N′-ethyl-urea

To a suspension of LiAlH₄ (90 mg, 2.5 mmol) in Et₂O (10 mL, anhydrous) was added dropwise a solution of [4-bromo-2-[[4-(4-bromophenyl)-4-hydroxy-1-piperidinyl]methyl]phenoxy]acetonitrile (1.0 mmol) in Et₂O (10 mL) at 0° C. After addition, the mixture was stirred at rt for 2 h, and then quenched by addition of 15% NaOH (90 mL), and water (0.3 mL). MgSO₄ (8 g) was added to the resulting mixture and stirred vigorously for 0.5 h. After removal of solvent, a crude product was obtained which was purified by HPLC to afford 1-[[2-(2-aminoethoxy)-5-bromophenyl]methyl]-4-(4-bromophenyl)-4-piperidinol as a white solid. ¹H NMR (DMSO-d₆/TFA): δ 1.50(d, 2H), 2.10 (m, 2H), 3.15˜3.30(m, 6H), 4.12(t, 2H), 4.35(d, 2H), 7.02(d, 1H), 7.28(m, 2H), 7.45(m, 2H), 7.54(m, 1H), 7.69(d, 1H). To a solution of 1-[[2-(2-aminoethoxy)-5-bromophenyl]methyl]-4-(4-bromophenyl)-4-piperidinol (100 mg, 0.21 mmol), and Et₃N(0.15 mL, 1.1 mmol) in CH₂Cl₂ (5 mL) was added EtNCO(0.03 mL, 0.4 mmol) at rt. The mixture was stirred at rt under N₂ overnight. After concentrating, the residue was purified by HPLC to afford the title compound as a trifluoroacetic acid salt (light yellow syrup). ¹H NMR (DMSO-d₆/TFA): δ 0.95 (t, 3H), 1.80 (br.d, 2H), 2.20 (m, 2H), 2.90 (q, 2H), 3.30 (m, 4H), 3.40 (t, 2H), 4.00 (t, 2H), 4.30 (d, 2H), 7.06 (d, 1H), 7.35 (m, 2H), 7.53 (m, 2H), 7.58 (m, 1H), 7.68 (d, 1H).

### Example 24

### 2-Bromo-1-[5-bromo-2-[(4-chlorophenyl)methoxy]phenyl]-ethanone

To a stirred solution of 5-bromo-2-hydroxy-acetophenone (54 g, 0.25 mol) in DMF (500 mL) was added K₂CO₃ (100 g, 0.23 mol) at rt. After 30 min, 4-chlorobenzyl chloride (36.6 g, 0.23 mmol) was added in one portion, and the mixture was kept at 70° C. for 4 h. The mixture was cooled to rt, and poured into an ice-cooled mixture of EtOAc/water (1:1, 2 L). The solid was collected by filtration, washed with water, and dried under vacuum for 20 h to give 2-[5-bromo-2-[(4-chlorophenyl)methoxy]phenyl]ethanone (65 g, 83%). To a suspension of 2-[5-bromo-2-[(4-chlorophenyl)methoxy]phenyl]ethanone (20 g, 59 mmol) in CH₂Cl₂ (140 mL)-HOAc (5 mL) was added dropwise a solution of Br₂/CH₂Cl₂ (14%, 26 mL, 84 mmol) at rt under N₂. After 3 h, the mixture was concentrated to the minimum volume, and the product precipitated. The solid was purified by recrystallization to afford product (11 g) as dark orange crystals. ¹H NMR (400 MHz, DMSO-d₆): δ 3.3 (s, 2H), 4.72 (s, 2H), 5.14 (s, 2H), 7.21 (d, 1H), 7.41 (d, 2H), 7.5 (d, 2H), 7.72 (dd, 1H), 7.76 (d, 1H).

The following compounds were prepared in a similar manner:

4-[[4-Bromo-2-(bromoacetyl)phenoxy]methyl]benzoic acid, methyl ester

1-[2-([1,1′-Biphenyl]-4-ylmethoxy)-5-bromophenyl]-2-bromoethanone

3-[[4-[4-[[4-Bromo-2-(bromoacetyl)phenoxy]methyl]benzoyl]-1-piperazinyl]sulfonyl]-N-hydroxy-N-oxo-benzenaminium

2-Bromo-1-[5-Bromo-2-[(4-chlorophenyl)methoxy]phenyl]-1-propanone

### Example 25

### 1-[5-Bromo-2-[(4-chlorophenyl)methoxy]phenyl]-2-(dimethylamino)-ethanone

To a solution of 2-bromo-1-[5-bromo-2-[(4-chlorophenyl)methoxy]phenyl]ethanone (1 g, 2.6 mmol), in DMF (5 mL) was added Me₂NH.HCl (420 mg, 5.2 mmol) and K₂CO₃ (1 g) while cooled with an ice-water bath. The mixture was stirred under the same conditions for 4 h. The mixture was poured into ice-water while stirring vigorously. Filtered, the solid was dissolved in EtOAc; washed with brine; dried over Na₂SO₄, concentrated, and the residue was purified by flash chromatography to afford the title compound as an off-white powder. ¹H NMR (DMSO-d₆): δ 2.78 (s, 6H), 4.70 (s, 2H), 5.42(s, 2H), 7.27(d, 1H), 7.45˜7.60(m, 4H), 7.83(dd, 1H), 7.93 (d, 1H).

The following compound was prepared in a similar manner

1-[2-([1,1′-Biphenyl]-4-ylmethoxy)-5-bromophenyl]-2-(dimethylamino)-ethanone, started with 1-[2-([1,1′-biphenyl]-4-ylmethoxy)-5-bromophenyl]-2-bromoethanone, ¹H NMR (400 MHz, DMSO-d₆): 2.8 (s, 6H), 4.78 (s, 2H), 5.4 (s, 2H), 7.32 (d, 1H), 7.36 (dt, 1H), 7.45 9t, 2H), 7.74 (m, 3H), 7.7 (dt, 2H), 7.85 (d, 1H), 7.95 (d, 1H), 9.8 (Br. s, 1H).

### Example 26

### 5-Bromo-2-[(4-chlorophenyl)methoxy]-α-[[(2-hydroxyethyl)(methyl)amino]methyl]benzenemethanol, trifluoroacetic acid salt

To a stirred solution of 2-bromo-1-[5-bromo-2-[(4-chlorophenyl)methoxy]phenyl]ethanone (300 mg, 0.72 mmol) in DMF (5 mL) was added MeNHCH₂CH₂OH (0.3 mL, 3.7 mmo) at 0° C. After 10 min, the mixture was poured into ice-water. The solid was collected by filtration, and then re-dissolved in MeOH (10 mL). To the solution was added NaBH₄ (˜100 mg) and the reaction stirred at rt for 1 h. After removal of MeOH, the residue was diluted with EtOAc, dried over Na₂SO₄, and concentrated. The residue was purified by HPLC to afford the product as a white powder. ¹H NMR (DMSO-d₆/TFA): δ 2.75˜2.90 (m, 3H), 3.05˜3.35 (m, 4H), 3.65˜3.75 (m, 2H), 5.15 (dd, 2H), 5.27 (m, 1H), 7.02˜7.07 (m, 1H), 7.42˜7.53 (m, 5H), 7.56 (br.s, 1H), 9.20˜9.40 (m, 1 H).

The following compounds were prepared in a similar manner:

α-[5-Bromo-2-[(4-chlorophenyl)methoxy]phenyl]-3-hydroxy-1-piperidineethanol, trifluoroacetic acid salt. ¹H NMR (DMSO-d₆/TFA): δ 1.20˜2.10 (m, 4H), 2.50˜4.00 (m, 7H), 5.10˜5.18 (m, 2H), 5.26˜5.34 (m, 1H), 7.02˜7.07 (m, 1H), 7.42˜7.52 (m, 5H), 7.52˜7.56 (m, 1H), 9.00 and 9.60 (each m, 1H).

α-[5-Bromo-2-[(4-chlorophenyl)methoxy]phenyl]-3-hydroxy-1-pyrrolidineethanol, trifluoroacetic acid salt. ¹H NMR (DMSO-d₆/TFA): δ 1.68˜2.20 (m, 2H), 2.90˜3.70 (m, 6H), 4.34˜4.42 (m, 1H), 5.10˜5.24 (m, 3H), 7.04˜7.08 (m, 1H), 7.45-7.56 (m, 6H), 9.80˜10.00 (m, 1H).

(2S,4R)-1-[2-[5-Bromo-2-[(4-chlorophenyl)methoxy]phenyl]-2-hydroxyethyl]-4-hydroxy-2-pyrrolidinecarboxylic acid, trifluoroacetic acid salt. ¹H NMR (DMSO-d₆/TFA): δ 2.02˜2.28 (m, 2H), 3.06˜3.54 (m, 3H), 3.78˜3.94 (m, 1H), 4.34˜4.64 (m, 1H), 4.50˜4.66 (m, 1H), 5.10˜5.28 (m, 3H), 7.01˜7.05 (m, 1H), 7.40˜7.50 (m, 5H), 7.54˜7.59 (m, 1H).

5-Bromo-2-[(4-chlorophenyl)methoxy]-α-[(dimethylamino)methyl]benzenemethanol,

trifluoroacetic acid salt. ¹H NMR (DMSO-d₆/TFA): δ 2.72˜2.80(m, 6H), 3.12˜3.18(m, 2H), 5.14 (dd, 2H), 5.22 (br.dd, 1H), 7.05 (d, 1H), 7.43˜7.53 (m, 5H), 7.55 (d, 1H), 9.50 (br.s, 1H).

2-Amino-α-[5-Bromo-2-[(4-chlorophenyl)methoxy]phenyl]-1H-imidazole-1-ethanol. ¹H NMR (DMSO-d₆/TFA): δ 3.96 (d, 2H), 5.10˜5.16 (m, 3H), 6.57 (d, 1H), 6.87 (d, 1H), 7.02 (d, 1H), 7.40˜7.50 (m, 7H), 7.54 (d, 1H), 12.20 (s, 1H).

α-[5-Bromo-2-[(4-chlorophenyl)methoxy]phenyl]-4-hydroxy-1-piperidineethanol, ¹H NMR (DMSO-d₆/TFA): δ1.48˜2.02 (m, 4H), 2.76˜3.50 (m, 6H), 3.60˜3.88 (each m, 1H), 5.10˜5.18 (m, 2H), 5.22˜5.32 (m, 1H), 7.04 (d, 1H), 7.40˜7.52 (m, 5H), 7.54˜7.58 (m, 1H), 9.40 (br.s, 1H).

4-[[4-Bromo-2-[1-hydroxy-2-(4-hydroxy-1-piperidinyl)ethyl]phenoxy]methyl]benzoic acid, methyl ester, trifluoroacetic acid salt. Started with 4-[[4-bromo-2-(bromoacetyl)phenoxy]methyl]benzoic acid, methyl ester and 4-hydroxypiperidine. ¹H NMR (DMSO-d₆/TFA): δ 1.50˜2.00 (m, 4H), 2.80˜3.70 (m, 7H), 3.82 (s, 3H), 5.24 (br.s, 2H), 5.28˜5.37 (m, 1H), 7.10 (d, 1H), 7.32˜7.36 (m, 1H), 7.44 (dd, 1H), 7.58˜7.63 (m, 2H), 7.95˜8.00 (m, 2H), 9.30 (br.s, 1H).

4-[[4-bromo-2-[1-hydroxy-2-(3-hydroxy-1-piperidinyl)ethyl]phenoxy]methyl]benzoic acid, methyl ester, trifluoroacetic acid salt. Started with 4-[[4-bromo-2-(bromoacetyl)phenoxy]methyl]benzoic acid, methyl ester and 3-hydroxy-piperidine. ¹H NMR (DMSO-d₆/TFA): δ 1.20˜2.10 (m, 4H), 2.50˜4.00 (m, 10H), 5.20˜5.30 (m, 2H), 5.30˜5.40 (m, 1H), 7.06˜7.12 (m, 1H), 7.30˜7.36 (m, 1H), 7.42˜7.46 (m, 1H), 7.58˜7.64 (m, 2H), 7.96˜8.02 (m, 2H), 9.00˜9.60 (m, 1H).

4-[[4-Bromo-2-[2-[4-[[(1,1-dimethylethoxy)carbonyl]amino]-1-piperidinyl]-1-hydroxyethyl]phenoxy]methyl]benzoic acid, methyl ester. Started with 4-[[4-bromo-2-(bromoacetyl)phenoxy]methyl]benzoic acid, methyl ester and 4-[[(1,1-dimethylethoxy)carbonyl]amino]-1-piperidinyl. ¹H NMR (400 MHz, DMSO): 1.4 (s, 9H), 1.9 (m, 2H, 2.1 (m, 1H), 2.4 (m, 2H), 2.7 (m, 2H), 3.1 (m, 1H), 3.5 (br. s, 1H), 3.9 (s, 3H), 4.5 (br. s, 1H), 5.2 9 m, 3H), 6.8 (d, 1H), 7.2 (d, 1H), 7.42 (d, 2H), 7.6 (s, 1H), 8.1 (d, 2H).

2-([1,1′-Biphenyl]-4-ylmethoxy)-5-bromo-′-[(dimethylamino)methyl]-benzenemethanol, trifluoroacetic acid salt. Started with 1-[2-([1,1′-biphenyl]-4-ylmethoxy)-5-bromophenyl]-2-bromoethanone and dimethylamine. ¹H NMR (400 MHz, DMSO): 2.8 (m, 6H), 3.2 (m, 2H), 5.2 (s, 2H), 5.23 (m, 1), 7.1 (d, 1H), 7.36 (tt, 1H), 7.46 (m, 3), 7.55 (m, 2H), 7.67 (m, 3H), 9.42 (br. s, 1H).

### Example 27

### 4-[[4-Chloro-2-[1-hydroxy-2-(4-hydroxy-1-piperidinyl)ethyl]phenoxy]methyl]benzoic acid,

To a stirred solution of 4-[[4-bromo-2-[2-[4-[[(1,1-dimethylethoxy)carbonyl]amino]-1-piperidinyl]-1-hydroxyethyl]phenoxy]methyl]benzoic acid, methyl ester (3.2 g, 7.88 mmol) in THF (25 mL) and MeOH (25 mL), was added LiOH (495 mg, 11.83 mmol) in water (25 mL) at rt. After 10 h, the reaction was neutralized with 0.1 N HCl to pH 6, and extracted with EtOAc (3×60 mL). The organic phase was washed with brine (50 mL), and dried over Na₂SO₄. Concentration in vacuo followed by purification through column chromatography afforded the title product. ¹H NMR (400 MHz, DMSO-d₆): 1.75 (m, 4H), 2 (m, 1H), 3.08 (m, 3H), 3.24 (m, 1H), 3.4 (m, 1H), 5.2 s, 2H), 5.4 (dd, 1H), 7.1 (d, 1H), 7.3 (dd, 1H), 7.41 (dd, 1H), 7.52 (d, 2H), 7.83 (d, 2H), 10.6 (br. s, 1H).

### Example 28

### 1-[5-Bromo-2-[(4-chlorophenyl)methoxy]phenyl]2-(dimethylamino)-1-propanone

To a stirred solution of 2-bromo-1-[5-bromo-2-[(4-fluorophenyl)methoxy]phenyl-1-propanone (216 mg, 0.5 mmol) in DMF (3 ml) was added dimethylamine .HCl salt (108 mg), and Cs₂CO₃ (494 mg). The reaction mixture was stirred for 2.5 h at rt, and an additional 2.5 eq. of amine was added. After an additional hour, DMF was concentrated in vacuo. The resulting residue was diluted with EtOAc, washed with water and brine, and dried over Na₂SO₄. Concentration in vacuo, followed by purification through flash column chromatography afforded the title compound.¹H NMR (CDCl₃, 400 MHz): d 1.15 (d,3H), 2.25 (s, 6H), 4.10 (q, 1H), 5.05 (s, 2H), 6.80 (d, 1H), 7.38 (m, 4H), 7.45 (d, 1H), 7.60 (s, 1H).

### Example 29

### 5-Bromo-2-[(4-chlorophenyl)methoxy]-α-[1-(dimethylamino)ethyl]benzenemethanol

To the mixture of NaBH₄ (15.4 mg) in ethanol (4 mL) was added a solution of 1-[5-bromo-2-[(4-chlorophenyl)methoxy]phenyl]2-(dimethylamino)-1-propanone (123 mg, 0.31 mmol) in ethanol (3 mL). The reaction mixture was stirred at rt overnight. Solvent was concentrated in vacuo, and the residue was diluted with ethyl acetate, washed with water, and extracted with ethyl acetate (2×). The combined organic phase was washed with brine, dried over Na₂SO₄. Concentration in vacuo, followed by purification with flash column chromatography afforded the title compound. ¹H NMR (CDCl₃, 400 MHz) δ 0.70 (d, 3H), 2.30 (s, 6H), 2.60 (m, 1H), 4.80 (d, 1H), 5.00 (m,2H), 6.75 (d, 1H), 7.30 (m, 5H), 7.65 (s, 1H).

The following compounds were prepared in a similar manner:
5-Chloro-2-[(4-chlorophenyl)methoxy]-α-[1-(dimethylamino)ethyl]-benzenemethanol, ¹H NMR (CD₃OD, 400 MHz) δ (TMS) 0.95 (d, 3H), 2.40 (s, 6H), 2.90 (m, 1H), 5.05 (m, 2H), 7.00 (d, 1H), 7.20 (d, 1H), 7.40 (m, 5H).
α-[5-Chloro-2-[(4-chlorophenyl)methoxy]phenyl]-β-methyl-1H-imidazole-1-ethanol, ¹H NMR(CD₃OD, 400 MHz) δ (TMS) 1.3 (d,3H), 4.50 (m, 1H), 5.10 (m, 3H), 6.80 (m, 1H), 7.00 (m, 2H), 7.20 (m, 2H), 7.45 (m, 5H).
-[5-Chloro-2-[(4-chlorophenyl)methoxy]phenyl]-4-(4-chlorophenyl)-4-hydroxy-β-methyl-1-piperidineethanol, MS:520 (M+1). ¹H NMR (CD₃OD, 400 MHz) δ (TMS) 1.0 (d, 3H), 1.50 (m, 1H), 1.65 (m, 1H), 1.95(m, 2H), 2.50 (m, 1H), 2.90 (m, 4H), 5.05 (m, 2H), 5.40 (m, 1H), 7.00 (d, 1H), 7.20 (m, 1H), 7.30 (d, 2H), 7.45 (m, 7H).
α-[5-Chloro-2-[(4-chlorophenyl)methoxy]phenyl]-4-hydroxy-β-methyl-4-(phenylmethyl)-1-piperidineethanol, ¹H NMR (CD₃OD, 400 MHz) δ (TMS) 1.0 (d, 3H), 1.45 (m, 1H), 1.70 (m, 3H), 2.70 ( s, 2H), 2.90 (m, 1H), 3.10 (m,1H), 3.30(m,3H), 5.0 (m, 2H), 5.40 (s, 1H), 6.90 (d, 1H), 7.20 (m, 6H), 7.40 (m, 5H).
α-[5-Chloro-2-[(4-chlorophenyl)methoxy]phenyl]-4-(4-fluorophenyl)-4-hydroxy-β-methyl-1-piperidineethanol. ¹H NMR (CDCl₃, 400 MHz): δ (TMS) 0.85 (d, 3H), 1.75 (m, 2H), 2.05 (m, 2H), 2.65 (m, 2H), 2.80 (m,1H), 2.90 (m, 1H), 3.0(m,1H), 5.05 (m, 2H), 5.26 (m, 1H), 6.80 (d, 1H), 7.05 (t, 2H), 7.20 (d, 1H), 7.35 (m, 4H), 7.45 (m, 2H), 7.50 (s, 1H).
5-Chloro-2-[(4-chlorophenyl)methoxy]-α-[1-(diethylamino)ethyl-benzenemethanol. ¹H NMR (CDCl₃, 400 MHz) δ (TMS) 0.85 (d,3H), 0.95 (t, 6H), 2.50 (m, 4H), 3.05 (m, 1H), 5.00 (m,2H), 5.05 (m, 1H), 6.78 (d, 1H), 7.15 (d, 1H), 7.35 (m, 4H), 7.50 (s, 1H).

### Example 30

### α-[5-bromo-2-[[4-[[4-[(3-nitrophenyl)sulfonyl]-1-piperazinyl]carbonyl]phenyl]methoxy]phenyl]-3-hydroxy-1-piperidineethanol

To a solution of 2-bromo-1-[5-bromo-2-[[4-[[4-[(3-nitrophenyl)sulfonyl]-1-piperazinyl]carbonyl]phenyl]methoxy]phenyl]ethanone (300 mg, 0.47 mmol) in DMF (5 mL), 3-hydroxypiperidine.HCl (330 mg, 2.4 mmol) and K₂CO₃ (300 mg) were added at rt. After 1 h, the mixture was poured into ice-water. The solid was collected by filtration, and re-dissolved in MeOH (15 mL). To this solution was added NaBH₄ (100 mg) and the mixture was kept at rt overnight. After removal of MeOH, the residue was diluted with EtOAc, washed with brine; dried over Na₂SO₄, and concentrated. The residue was purified by HPLC to afford the product as a white powder. ¹H NMR (DMSO-d₆/TFA): δ 1.10˜2.00 (m, 4H), 2.60˜4.00 (m, 15H), 5.10˜5.20 (m, 2H), 5.25˜5.35 (m, 1H), 7.04˜7.14 (m, 1H), 7.26˜7.56 (m, 6H), 7.86˜7.98 (m, 1H), 8.10˜8.18 (m, 1H), 8.32˜8.38 (m, 1H), 8.48˜8.60 (m, 1H).

The following compounds were prepared in a similar manner:
α-[5-Bromo-2-[[4-[[4-[(3-nitrophenyl)sulfonyl]-1-piperazinyl]carbonyl]phenyl]methoxy]phenyl]-4-hydroxy-1-piperidineethanol.

¹H NMR (DMSO-d₆/TFA): δ 1.45˜2.10 (m, 4H), 2.70˜3.90 (m, 15H), 5.08˜5.32 (m, 3H), 7.04˜7.14 (m, 1H), 7.20˜7.56 (m, 6H), 7.88˜7.98 (m, 1H), 8.12˜8.18 (m, 1H), 8.34˜8.38 (m, 1H), 8.50˜8.58 (m, 1H).
α-[5-Bromo-2-[[4-[[4-[(3-nitrophenyl)sulfonyl]-1-piperazinyl]carbonyl]phenyl]methoxy]phenyl]-3-hydroxy-1-pyrrolidineethanol

¹H NMR (DMSO-d₆/TFA): δ 1.70˜2.20 (m, 2H), 2.90˜4.40 (m, 15H), 5.10˜5.25 (m, 3H), 7.06˜7.12 (m, 1H), 7.28˜7.52 (m, 6H), 7.92 (m, 1H), 8.14 (br.d, 1H), 8.33 (br.s, 1H), 8.54 (br.d, 1H), 9.70 and 10.00 (each br.s, 1H).
5-Bromo-α-[(diethylamino)methyl]-2-[[4-[[4-[(3-nitrophenyl)sulfonyl]-1-piperazinyl]carbonyl]phenyl]methoxy]benzenemethanol, ¹H NMR (DMSO-d₆/TFA): δ 0.90 (t, 3H), 1.10 (t, 3H), 3.00˜3.80 (m, 15H), 5.05˜5.20 (m, 3H), 7.08˜7.14 (m, 1H), 7.30˜7.52 (m, 6H), 7.88˜7.96 (m, 1H), 8.10˜8.18 (m, 1H), 8.30˜8.38 (m, 1H), 8.50˜8.56 (m, 1H), 9.10 (br.s, 1H).

### Example 31

### α-[5-Bromo-2-[(4-chlorophenyl)methoxy]phenyl]-1-piperazineethanol

To a stirred solution of 2-bromo-1-[5-bromo-2-[(4-chlorophenyl)methoxy]phenyl]-ethanone (7.0 g, 17.5 mmol) in DMF (50 mL) was added N-Boc-piperazine (4.9 g, 26.3 mmol), followed by NaHCO₃ (10 g). After 14 h, the reaction mixture was poured into ice-water (150 mL). The reaction mixture was extracted with EtOAc (3×80 mL), washed with brine (100 mL), and dried over Na₂SO₄. Concentration in vacuo afforded crude ketone-amine (6.1 g). To a stirred solution of the ketone-amine in MeOH (80 mL) at 0° C. was added NaBH₄ (796 mg, 21 mmol). After 30 min, methanol was removed in vacuo, and the resulting reaction mixture was quenched with brine (80 mL) and extracted with EtOAc (3×80 mL). The organic phase was washed with brine (100 mL), and dried over Na₂SO₄. Concentration in vacuo followed by purification through column chromatography afforded product (7.21 g, 78%). To a stirred solution of the product (7 g, 13.3 mmol) in CH₂Cl₂ (60 mL) at room temperature was added trifluoroacetic acid (30 mL).

After 2 h, the reaction mixture was concentrated in vacuo, and re-dissolved in CH₂Cl₂ (200 mL). The solution was washed with 10% NaOH (3×25 mL) and brine (2×20 mL), dried (Na₂SO₄), and concentrated to give the title product.

### Example 32

### α-[5-Bromo-2-[(4-chlorophenyl)methoxy]phenyl]-4-(3-pyridinylcarbonyl)-1-piperazineethanol

To a stirred solution of α-[5-bromo-2-[(4-chlorophenyl)methoxy]phenyl]-1-piperazineethanol (371 mg, 0.87 mmol) in DMF (5 mL) was added 3-pyridinecarboxylic acid (107 mg, 0.87 mmol), followed by HATU (306 mg, 0.8 mmol) and Et₃N (271 mg, 2.68 mmol) at rt. After 4 hr, the reaction mixture was worked up as usual, and purified by column chromatography to afford the title compound. ¹H NMR (400 MHz, DMSO-d₆): 3.2 (br.s, 4H), 3.3 (dd, 2H), 3.56 (br. s, 4H), 5.0 (m, 1H), 5.06 (s, 2), 6.98 (d, 1H), 7.34 (dd, 1H), 7.4 (d, 2H), 7.44 (d, 2H), 7.48 (dd, 2H), 7.76 (m, 1H), 8.55 (d, 1H), 8.61 (dd, 1H).

The following compounds were prepared in a similar manner:
α-[5-Bromo-2-[(4-chlorophenyl)methoxy]phenyl]-4-[(2-methyl-3-pyridinyl)carbonyl]-1-piperazineethanol. ¹H NMR (400 MHz, DMSO-d₆): 2.64 (s, 3H), 3.0 (m, 2H), 3.37 (m, 4H), 3.6 (m, 4H), 4.94 (m, 1H), 5.04 (s, 2H), 6.98 (d, 1H), 7.26 (dd, 1H), 7.37 (dd, 1H), 7.43 (d, 2H), 7.52 (dd, 2), 8.44 (d, 1H).
α-[5-Bromo-2-[(4-chlorophenyl)methoxy]phenyl]-4-[(4-methyl-3-pyridinyl)carbonyl]-1-piperazineethanol. ¹H NMR (400 MHz, DMSO-d₆): 2.57 (s, 3H), 3.1 (m, 1H), 3.2 (m, 2H), 3.3 (m, 4H), 3.6 (m, 4H), 5 (m, 1H), 5.1 (s, 2H), 6.98 (d, 1H), 7.3 (dd, 1H), 7.35 (dd, 1H), 7.4 (d, 2H), 7.52 (dd, 2), 7.64 (d, 1H), 8.41 (d, 1H).

### Example 33

### 4-[[4-Bromo-2-[1-hydroxy-2-[4-[[(phenylmethoxy)carbonyl]amino]-1-piperidinyl]ethyl]phenoxy]methyl]benzoic acid, methyl ester

To a stirred solution of 4-[[4-bromo-2-[2-[4-[[(1,1-dimethylethoxy)carbonyl]amino]-1-piperidinyl]-1-hydroxyethyl]phenoxy]methyl]benzoic acid, methyl ester (450 mg, 0.8 mmol) in CH₂Cl₂ (5 mL) was added TFA (2 mL) at room temperature. After 2 h, the reaction was concentrated in vacuo, and dried under vacuum for 2 h. This crude de-protected product was reacted with benzyloxycarbonylchloride (178.2 mg, 1.05 mmol) at 0° C. in the presence of Et₃N (440 mg) for 1 h. After regular work up, the crude product was purified by column chromatography to afford the title product. ¹H NMR (400 MHz, DMSO-d₆): 1.4 (m, 2H), 1.8 (m, 2H), 2.0 (m, 1H), 2.3 (m, 2H), 2.6 (m, 2H), 2.96 (m, 1H), 3.5 (m, 1H, 3.81 (s, 3H), 4.6 (s, 2H), 4.8-5.5 (m, 5H), 6.73 (d, 1H), 7.3 (dd, 1), 7.18-7.35 (m, 7H), 7.4 (d, 2H), 7.57 (d, 1H), 8.1 (d, 2H).

### Example 35

### 4-[[4-Bromo-2-[1-hydroxy-2-(4-hydroxy-1-piperidinyl)ethyl]phenoxy]methyl]-N-(4-pyridinyl)benzamide

To a stirred solution of 4-[[4-bromo-2-[1-hydroxy-2-(4-hydroxy-1-piperidinyl)ethyl]phenoxy]methyl]benzoic acid (300 mg, 0.62 mmol) in DMF (5 mL), 4-pyridinamine (87 mg, 0.92 mmol) was added, followed by HATU (351 mg, 0.92 mmol) and Et₃N (187 mg, 1.85 mmol) at room temperature. After 14 h, the reaction was purified by HPLC to afford the title product as a trifluoroacetic acid salt. ¹H NMR (400 MHz, DMSO-d₆): δ 1.7 (m, 2H), 1.9 (m, 2H), 3.2 (m, 3H), 3.4 (m, 1H), 3.6 (m, 1H), 5.22 (s, 2H), 5.36 (m, 1H), 7.1 (d, 1H), 7.34 (dd, 1H), 7.46 (d, 1H), 68 (d, 2H), 8.05 (d, 2H), 8.27 (d, 2H), 8.75 (d, 2H), 9.5 (br. s, 1 H), 11.6 (d, 1 H).

The follow compound was prepared in a similar manner:
4-[[4-Chloro-2-[1-hydroxy-2-(4-hydroxy-1-piperidinyl)ethyl]phenoxy]methyl]-N-(3-hydroxypropyl)benzamide. ¹H NMR (400 MHz, DMSO-d₆): 0.8 (t, 2H), 1.22 (m, 2H), 1.61 (m, 1H), 1.82 (m, 1H), 3.0-3.6 (m, 10H), 5.2 (s, 2H), 5.3 (m, 1H), 7.1 (d, 1), 7.36 (dd, 1H0, 7.43 (t, 1H), 7.58 (dd, 2H), 7.84 (dd, 2H), 9.23 (br. s, 1H).

### Example 36A

### 2-[5-Bromo-2-[(4-chlorophenyl)methoxy]phenyl]oxirane

To a homogeneous solution of (Me)₃SI (13.8 g, 67.6 mmol) in DMSO (300 mL) was added 5-bromo-2-(4-chlorophenylmethyl)benzaldehyde (20.0 g, 61.4 mmol), followed by KO-tBu (8.27 g, 73.7 mmol) at room temperature. The reaction was kept at room temperature overnight, and was poured into ice-water (300 mL). The reaction mixture was extracted with EtOAc (3×200 mL), washed with brine (150 mL), and dried (Na₂SO₄). Concentration, followed by purification through column chromatography afforded the title compound. ¹H NMR (400 MHz, DMSO): δ 1.53 (m, 2H), 1.84 (m, 2H), 2.6 (m, 6H), 3.6 (s, 2H), 5.02 (s, 2H), 6.78 (d, 1H), 7.32 (d, 1H), 7.4 (s, 4H), 7.54 (s, 1H).

### Example 36B

### 1(2S)-α-[5-Bromo-2-[(4-chlorophenyl)methoxy]phenyl]-2-(hydroxymethyl)-1-pyrrolidineethanol, trifluoroacetic acid salt

To a stirred solution of 2-[5-bromo-2-[(4-chlorophenyl)methoxy]phenyl]oxirane (340 mg, 1 mmol) in DMF (3 mL) was added (2R)-pyrrolidinemethanol (152 mg, 1.5 mmol) at room temperature. The reaction mixture was kept at 110° C. for 8 h, and was cooled down to room temperature. The reaction was purified by HPLC to afford the title compound as a trifluoroacetic acid salt. ¹H NMR (400 MHz, DMSO-d₆): 1.63 (m, 1H), 1.7-2.1 (m, 3H), 2.9 (m, 1H), 3.24 (m, 2H), 3.3-3.7 (m, 9H), 5.12 (s, 2H), 7.04 (t,1H), 7.46 (m, 6H).

The following compounds were prepared in a similar manner:
(2R)-α-[5-Bromo-2-[(4-chlorophenyl)methoxy]phenyl]-2-(hydroxymethyl)-1-pyrrolidineethanol, trifluoroacetic acid salt. ¹H NMR (400 MHz, DMSO-d₆): 1.63 (m, 1H), 1.8-2.1 (m, 3H), 2.9 (m, 1H), 3.2 (m, 2H), 3.3-3.7 (m, 10H), 5.12 (s, 2H), 7.0 (t, 1H), 7.4-7.6 (m, 6H).
(3R)-α-[5-Bromo-2-[(4-chlorophenyl)methoxy]phenyl]-3-hydroxy-1-pyrrolidineethanol, trifluoroacetic acid salt. ¹H NMR (400 MHz, DMSO-d₆): 1.6-1.9 (m, 2H), 3.0-3.7 (m, 6H), 4.4 (m, 1H), 5.1 (s, 2H), 5.2 (m, 1H), 7.0 (d, 1H), 7.3-7.6 (m, 6H).
5-Bromo-2-[(4-Chlorophenyl)methoxy]-α-[[[2-(diethylamino)ethyl]ethylamino]methyl]-benzenemethanol, trifluoroacetic acid salt. ¹H NMR (DMSO-d₆/TFA): δ 1.00˜1.20(m, 9H), 3.20˜3.51 (m, 12H), 5.13 (s, 2H), 5.25 (br.s, 1H), 7.08 (br.d, 1H), 7.40˜7.53(m, 5H), 7.62(d, 1H), 9.70 (br.s, 2H).
α-[5-Bromo-2-[(4-chlorophenyl)methoxy]phenyl]-1,4-piperidinediethanol, trifluoroacetic acid salt. ¹H NMR (DMSO-d₆/TFA): δ 1.10˜1.87 (m, 7H), 2.68˜3.60 (m, 8H), 5.10˜5.35 (m, 3H), 7.00˜7.10 (m, 1H), 7.40˜7.55 (m, 5H), 7.57˜7.61 (m, 1H), 9.20˜9.40 (m, 1H).
α-[5-Bromo-2-[(4-chlorophenyl)methoxy]phenyl]-4-(piperidyl)-1-piperidineethanol ¹H NMR (DMSO-d₆/TFA): δ 1.20˜2.15 (m, 10H), 2.75˜3.70 (m, 11H), 5.00 (br.s, 2H), 5.20 (m, 1H), 6.90 (br.dd, 1H), 7.30˜7.40 (m, 5H), 7.47 (br.dd, 1H), 9.30˜9.80 (m, 2H).
5-Bromo-2-[(4-Chlorophenyl)methoxy]-α-[(dipropylamino)methyl]-benzenemethanol, trifluoroacetic acid salt. ¹H NMR (DMSO-d₆/TFA): δ 0.65 (t, 3H), 0.80 (t, 3H), 1.35˜1.65 (m, 4H), 2.90˜3.15 (m, 6H), 5.05 (dd, 2H), 5.10 (dd, 1H), 7.05 (d, 1H), 7.40˜7.50 (m, 5H), 7.60 (d, 1H), 9.18 (br.s, 1H).
α-[5-Bromo-2-[(4-chlorophenyl)methoxy]phenyl]-4-(phenylmethyl)-1-piperidineethanol, trifluoroacetic acid salt. ¹H NMR (DMSO-d₆/TFA): δ 1.20˜1.85 (m, 5H), 2.60˜3.55 (m, 8H), 5.00˜5.30 (m, 3H), 7.00˜7.30 (m, 6H), 7.35˜7.60 (m, 6H), 9.20˜9.40 (m, 1H).
5-Bromo-2-[(4-chlorophenyl)methoxy]-α-[(dibutylamino)methyl]-benzenemethanol, trifluoroacetic acid salt. ¹H NMR (DMSO-d₆/TFA): δ 0.70 (t, 3H), 0.85 (t, 3H), 1.10 (m, 2H), 1.23 (m, 2H), 1.35˜1.65 (m, 4H), 2.90˜3.20 (m, 6H), 5.10 (m, 2H), 5.18 (m, 1H), 7.10 (d, 1H), 7.40˜7.51 (m, 5H), 7.60 (d, 1H), 9.30 (br.s, 1H).
5-Bromo-α-[(butylethylamino)methyl]-2-[(4-chlorophenyl)methoxy]-benzenemethanol, ¹H NMR (DMSO-d₆/TFA): δ 0.70˜1.60 (m, 10H), 2.90˜3.20 (m, 6H), 5.05˜5.20 (m, 3H), 7.10 (m, 1H), 7.40˜7.55 (m, 5H), 7.62 (s, 1H), 9.10 (br.s, 1H).
5-Bromo-2-[(4-chlorophenyl)methoxy]-α-[[ethyl(2-hydroxyethyl)amino]methyl]benzenemethanol, trifluoroacetic acid salt. ¹H NMR (DMSO-d₆/TFA): δ 0.90˜1.15 (m, 3H), 3.00˜3.30 (m, 6H), 3.65 (m, 2H), 5.00 (br.s, 1H), 5.20 (m, 1H), 6.92 (br.s, 1H), 7.30˜7.45 (m, 5H), 7.58 (s, 1H), 9.00 (br.s, 1H).
5-Bromo-2-[(4-chlorophenyl)methoxy]-α-[[(2-hydroxyethyl)propylamino]methyl]benzenemethanol trifluoroacetic acid salt. ¹H NMR (DMSO-d₆/TFA): δ 0.64˜0.76 (each t, 3H), 1.32˜1.62 (m, 2H), 2.90˜3.30 (m, 6H), 3.55˜3.70 (m, 2H), 5.00˜5.30 (m, 3H), 7.02˜7.10 (m, 1H), 7.40˜7.52 (m, 5H), 7.57(d, 1H), 8.90˜9.10(m, 1H).
1-[2-[5-Bromo-2-[(4-chlorophenyl)methoxy]phenyl]-2-hydroxyethyl]-N,N-diethyl-3-piperidinecarboxamide, trifluoroacetic acid salt. ¹H NMR (DMSO-d₆/TFA): δ 0.90˜1.15 (m, 6H), 1.40˜2.00 (m, 4H), 2.70˜3.70 (m, 11H), 5.10 (m, 2H), 5.28˜5.38 (m, 1H), 7.00 (m, 1H), 7.36˜7.50 (m, 5H), 7.60 (m, 1H), 9.10˜9.50 (m, 1H).
α-[5-Bromo-2-[(4-chlorophenyl)methoxy]phenyl]-4-(4-bromophenyl)-4-hydroxy-1-piperidineethanol, trifluoroacetic acid salt. ¹H NMR (DMSO-d₆/TFA): δ 1.54˜1.84 (m, 2H), 2.16˜2.42 (m, 2H), 3.00˜3.70 (m, 6H), 5.02˜5.18 (m, 2H), 5.20˜5.28 (m, 1H), 7.00˜7.18 (m, 1H), 7.30˜7.56 (m, 9H), 7.58˜7.62 (m, 1H), 9.30˜9.50 (m, 1H).
1-[1-[2-[5-Bromo-2-[(4-chlorophenyl)methoxy]phenyl]-2-hydroxyethyl]-4-piperidinyl]-1,3-dihydro-2H-benzimidazol-2-one, trifluoroacetic acid salt. ¹H NMR (DMSO-d₆/TFA): δ 1.82(m, 2H), 2.60˜2.86 (m, 2H), 3.00˜3.40 (m, 4H), 3.54 (m, 1H), 3.70 (m, 1H), 4.52 (m, 1H), 4.98˜5.14 (m, 2H), 5.18˜5.36 (m, 1H), 6.88˜7.00 (m, 4H), 7.34˜7.46 (m, 6H), 7.58˜7.62 (m, 1H), 9.60 (br.s, 1H), 10.80 (m, 1H).
1-[2-[5-Bromo-2-[(4-chlorophenyl)methoxy]phenyl]-2-hydroxyethyl]-4-phenyl-4-piperidinecarbonitrile, trifluoroacetic acid salt. ¹H NMR (DMSO-d₆/TFA): δ 2.10˜2.54 (m, 4H), 3.04 (m, 1H), 3.24˜3.48 (m, 3H), 3.68 (m, 1H), 3.84 (m, 1H), 5.04˜5.18 (m, 2H), 5.36 (m, 1H), 7.06 (d, 1H), 7.36˜7.56 (m, 10H), 7.59 (d, 1H), 9.70˜9.90 (m, 1H).
α-[5-Bromo-2-[(4-chlorophenyl)methoxy]phenyl]-1,4-dioxa-8-azaspiro[4.5]decane-8-ethanol, trifluoroacetic acid salt. ¹H NMR (DMSO-d₆/TFA): δ 1.72˜2.10 (m, 4H), 2.88 (m, 1H), 3.08˜3.30 (m, 3H), 3.40˜3.58 (m, 2H), 3.92 (m, 4H), 5.14 (s, 2H), 5.28 (m, 1H), 7.06 (d, 1H), 7.42˜7.54 (m, 5H), 7.58 (d, 1H), 9.58 (br.s, 1H).

5-Bromo-2-[(4-chlorophenyl)methoxy]-α-[[(2-hydroxyethyl)(phenylmethyl)amino]methyl]-benzenemethanol, trifluoroacetic acid salt. ¹H NMR (DMSO-d₆/TFA): δ 3.30˜3.32 (m, 4H), 3.72 (br.s, 2H), 4.30˜4.48 (m, 2H), 5.08 (br.s, 2H), 5.28 (m, 1H), 6.97 (br.s, 1H), 7.30˜7.56 (m, 11H), 9.30 (br.s, 1H).
5-Bromo-2-[(4-chlorophenyl)methoxy]-α-[[[2-(dimethylamino)ethyl]ethylamino]methyl]benzenemethanol, trifluoroacetic acid salt. ¹H NMR (DMSO-d₆/TFA): δ 1.00˜1.25 (m, 3H), 2.75˜2.90 (m, 6H), 3.10˜3.60 (m, 8H), 5.15 (br.s, 2H), 5.25 (br.s, 1H), 7.10 (m, 1H), 7.45˜7.55 (m, 5H), 7.65 (d, 1H).
α-[5-Bromo-2-[(4-chlorophenyl)methoxy]phenyl]-1,2,3,4-tetrahydro-1-quinolineethanol, trifluoroacetic acid salt. ¹H NMR (DMSO-d₆/TFA): δ 3.30˜3.50 (m, 5H), 3.55˜3.75 (m, 1H), 4.10˜4.40 (m, 1H), 4.50(m, 1H), 5.00˜5.20 (m, 2H), 5.28˜5.45 (m, 1H), 7.00˜7.26 (m, 5H), 7.36˜7.53 (m, 5H), 7.59 (br.s, 1H), 10.00˜10.20 (m, 1H).
1-[2-[5-Bromo-2-[(4-chlorophenyl)methoxy]phenyl]-2-hydroxyethyl]-3,4-pyrrolidinediol, trifluoroacetic acid salt. ¹H NMR (DMSO-d₆/TFA): δ 3.30˜3.70 (m, 6H), 4.00˜4.25 (m, 2H), 5.10˜5.30 (m, 3H), 7.02˜7.10 (m, 1H), 7.44˜7.54 (m, 5H), 7.54˜7.58 (m, 1H), 9.70 (br.s, 1H).
5-Bromo-2-[(4-chlorophenyl)methoxy]-α-[(methylamino)methyl]-benzenemethanol, trifluoroacetic acid salt. ¹H NMR (DMSO-d₆/TFA): δ 2.52 (m, 3H), 2.90˜3.10 (m,2H), 5.10˜5.18 (m, 3H), 7.02 (d, 1H), 7.42˜7.50 (m, 5H), 7.17 (d, 1H), 8.50 (br.s, 1H), 8.70 (br.s, 1H).
2-[[2-[5-Bromo-2-[(4-chlorophenyl)methoxy]phenyl]-2-hydroxyethyl]amino]-1,3-propanediol, trifluoroacetic acid salt. ¹H NMR (DMSO-d₆/TFA): δ 2.90˜3.00 (m, 1H), 3.16 (m, 1H), 3.26 (m, 1H), 3.54˜3.66 (m, 4H), 5.13 (dd, 2H), 5.22 (dd, 1H), 7.04 (d, 1H), 7.40˜7.50 (m, 5H), 7.77 (d, 1H), 8.20 (br.s, 1H), 8.70 (br.s, 1H).
5-Bromo-2-[(4-chlorophenyl)methoxy]-α-[(diethylamino)methyl]-benzenemethanol, trifluoroacetic acid salt. ¹H NMR (DMSO-d₆/TFA): δ 0.96 (t, 3H), 1.12 (t, 3H), 2.98˜3.16 (m, 6H), 5.11 (dd, 2H), 5.16 (dd, 1H), 7.08 (d, 1H), 7.42˜7.52 (m, 5H), 7.58 (d, 1H), 9.20 (br.s, 1H).
2-[[2-[5-Bromo-2-[(4-chlorophenyl)methoxy]phenyl]-2-hydroxyethyl]amino]-2-(hydroxymethyl)-1,3-propanediol. ¹H NMR (DMSO-d₆/TFA): δ 2.97 (m, 1H), 3.34 (m, 1H), 3.56(br.s, 6H), 5.14 (dd, 2H), 5.23 (dd, 1H), 7.15 (d, 1H), 7.40˜7.52 (m, 5H), 7.55 (d, 1H), 8.70 (br.s, 1H).
α-[5-Bromo-2-[(4-chlorophenyl)methoxy]phenyl]-1-pyrrolidineethanol.

¹H NMR (DMSO-d₆/TFA): δ 1.80˜1.90 (m, 4H), 2.85 (m, 1H), 3.05 (m, 1H), 3.20 (m, 2H), 3.50 (m, 2H), 5.10˜5.20 (m, 3H), 7.25 (d, 1H), 7.43˜7.52 (m, 5H), 7.55 (d, 1H), 9.70 (br.s, 1H).

α-[5-Bromo-2-[(4-chlorophenyl)methoxy]phenyl]-1-piperidineethanol.

¹H NMR (DMSO-d₆/TFA): δ 1.32 (m, 1H), 1.54˜1.80 (m, 5H), 2.74 (m, 1H), 2.94 (m, 1H), 3.02˜3.18 (m, 2H), 3.30˜3.46 (m, 2H), 5.13 (dd, 2H), 5.26 (br.dd, 1H), 7.05 (d, 1H), 7.43˜7.53 (m, 5H), 7.55 (d, 1H), 9.30 (br.s, 1H).
5-Bromo-2-[(4-chlorophenyl)methoxy]-α-[[(3-hydroxyphenyl)amino]methyl]benzenemethanol. ¹H NMR (DMSO-d₆/TFA): δ 3.22˜3.38 (m, 2H), 5.00˜5.10 (m, 3H), 6.70 (br.dd, 1H), 6.75 (br.dd, 1H), 6.80 (m, 1H), 7.02 (d, 1H), 7.14 (dd, 1H), 7.28˜7.44 (m, 5H), 7.60 (d, 1H).
5-Bromo-2-[(4-chlorophenyl)methoxy]-α-[[(cyclopropylmethyl)amino]methyl]benzenemethanol. ¹H NMR (DMSO-d₆/TFA): δ 0.00(m, 2H), 0.20 (m, 2H), 0.67 (m, 1H), 2.40˜2.70 (m, 3H), 2.88 (m, 1H), 4.84˜4.96 (m, 3H), 6.76˜6.84 (m, 1H), 7.12˜7.24 (m, 5H), 7.28˜7.34 (m, 1H), 8.30˜8.46 (m, 2H).
5-Bromo-[[[2-(3-chlorophenyl)ethyl]amino]methyl]-2-[(4-chlorophenyl)methoxy]benzenemethanol. ¹H NMR (DMSO-d₆/TFA): δ 2.84˜3.20 (m, 6H), 5.12 (s, 2H), 5.20 (dd, 1H), 7.04 (m, 1H), 7.15 (m, 1H), 7.26˜7.48 (m, 8H), 7.57 (d, 1H), 8.70 (br.s, 1H), 8.90 (br.s, 1H).
α-[5-Bromo-2-[(4-chlorophenyl)methoxy]phenyl]-1-azetidineethanol. ¹H NMR (DMSO-d₆/TFA): δ 2.18 (m, 1H), 2.38 (m, 1H), 3.18 (m, 1H), 3.30 (m, 1H), 3.83 (m, 1H), 3.96˜4.12 (m, 3H), 5.00 (dd, 1H), 5.10˜5.20 (m, 2H), 7.04 (dd, 1H), 7.40˜7.54 (m, 6H), 9.90 (br.s, 1H).
5-Bromo-2-[(4-chlorophenyl)methoxy]-α-[(ethylmethylamino)methyl]benzenemethanol. ¹H NMR (DMSO-d₆/TFA): δ 1.00˜1.20 (m, 3H), 2.68˜2.76 (m, 3H), 3.00˜3.22 (m, 4H), 5.08˜5.24 (m, 3H), 7.02˜7.08 (m, 1H), 7.38˜7.52 (m, 5H), 7.56˜7.58 (m, 1H), 9.20 and 9.40 (each br.s, 1H).
5-Bromo-2-[(4-chlorophenyl)methoxy]-α-[(cyclopropylamino)methyl]benzenemethanol. ¹H NMR (DMSO-d₆/TFA): δ 0.60˜0.80 (m, 4H), 2.50˜2.80 (m, 2H), 3.00 (m, 1H), 3.15 (m, 1H), 5.10 (dd, 2H), 5.18 (dd, 1H), 7.02˜7.08 (m, 1H), 7.40˜7.51 (m, 5H), 7.55˜758 (m, 1H), 8.00 (br.s, 1H), 8.80 (br.s, 1H).
5-Bromo-2-[(4-chlorophenyl)methoxy]-α-[[(cyclopropylmethyl)methylamino]methyl]benzenemethanol. ¹H NMR (DMSO-d₆/TFA): δ 0.00˜0.30 (m, 4H), 0.50˜0.80 (m, 1H), 2.50˜3.05 (m, 7H), 4.80˜4.92 (m, 2H), 4.96˜5.02 (m, 1H), 6.78˜6.84 (m, 1H), 7.14˜7.28 (m, 5H), 7.32˜7.35 (m, 1H), 9.00˜9.20 (m, 1H).
α-[5-Bromo-2-[(4-chlorophenyl)methoxy]phenyl]-4-thiomorpholineethanol.

¹H NMR (DMSO-d₆/TFA): δ 2.76 (m, 2H), 2.90˜3.30 (m, 6H), 3.56˜3.64 (m, 1H), 3.70˜3.78 (m, 1H), 5.12 (br.s, 2H), 5.30 (br.dd, 1H), 7.00˜7.05 (m, 1H), 7.40˜7.52 (m, 5H), 7.56 (d, 1H), 9.60 (br.s, 1H).
α-(Aminomethyl)-5-bromo-2-[(4-chlorophenyl)methoxy]benzenemethanol.

¹H NMR (DMSO-d₆/TFA): δ 2.75 (m, 1H), 3.00 (m, 1H), 5.05˜5.15 (m, 3H), 7.00˜7.08 (m, 1H), 7.38˜7.50 (m, 5H), 7.54 and 7.60 (each d, 1H), 7.86 and 8.28 (each br.s, 3H).
5-Bromo-2-[(4-chlorophenyl)methoxy]-α-[(cyclopropylmethylamino)methyl]benzenemethanol. ¹H NMR (DMSO-d₆/TFA): δ0.65˜1.10 (m, 4H), 2.55˜3.40 (m, 6H), 5.00˜5.10 (m, 2H), 5.10˜5.50 (m, 1H), 7.00˜7.10 (m, 1H), 7.40˜7.60 (m, 6H), 8.89˜9.10 (m, 1H).
(αS)-5-Bromo-2-[(4-chlorophenyl)methoxy]-α-[(diethylamino)methyl]benzenemethanol. ¹H NMR (DMSO-d₆/TFA): δ 1.10(t, 3H), 1.13(t, 3H), 3.15˜3.30 (m,6H), 5.25 (dd, 2H), 5.30 (dd, 1H), 7.21˜7.25 (m, 1H), 7.56˜7.67 (m, 5H), 7.72 (d, 1H), 9.20 (br.s, 1H).
(αR)-5-Bromo-2-[(4-chlorophenyl)methoxy]-α-[(diethylamino)methyl]-benzenemethanol. ¹H NMR (DMSO-d₆/TFA): δ 0.95 (t, 3H), 1.10 (t, 3H), 3.00˜3.15 (m, 6H), 5.10 (dd, 2H), 5.15 (dd, 1H), 7.04˜7.08 (m, 1H), 7.40˜7.50 (m, 5H), 7.58 (d, 1H), 9.10 (br.s, 1H).
α-[[Bis(2-hydroxyethyl)amino]methyl]-5-bromo-2-[(4-chlorophenyl)methoxy]benzenemethanol. ¹H NMR (DMSO-d₆/TFA): δ 3.20˜3.38 (m, 6H), 3.62˜3.76 (m, 4H), 5.13 (dd, 2H), 5.26 (dd, 1H), 7.03 (d, 1H), 7.40˜7.50 (m, 5H), 7.85 (d, 1H), 8.65 (br.s, 1H).
α-[5-Bromo-2-[(4-chlorophenyl)methoxy]phenyl]-4-methyl-1-piperazineethanol. ¹H NMR (DMSO-d₆/TFA): δ 2.74 (s, 3H), 2.80˜4.20 (m, 10H), 5.08˜5.20 (m, 3H), 7.02 (d, 1H), 7.40˜7.50 (m, 5H), 7.53 (d, 1H).
5-Bromo-2-[(4-chlorophenyl)methoxy]-α-[[(1-methylethyl)amino]methyl]-benzenemethanol.

¹H NMR (DMSO-d₆/TFA): δ 1.02 (d, 3H), 1.03 (d, 3H), 2.85 (m, 1H), 3.05 (m, 1H), 3.25 (m, 1H), 5.10˜5.16 (m, 3H), 7.06 (d, 1H), 7.42˜7.50 (m, 5H), 7.57 (d, 1H), 8.40˜8.70 (m, 2H).
α-[5-Bromo-2-[(4-chlorophenyl)methoxy]phenyl]-4-morpholineethanol. ¹H NMR (DMSO-d₆/TFA): δ 2.90˜3.90 (m, 10H), 5.15 (dd, 2H), 5.30 (m, 1H), 7.04 (d, 1H), 7.43˜7.53 (m, 5H), 7.55 (d, 1H), 10.00 br.s, 1H).
5-Bromo-2-[(4-chlorophenyl)methoxy]-α-[[(2-hydroxyethyl)amino]methyl]-benzenemethanol. ¹H NMR (DMSO-d₆/TFA): δ 2.88˜3.02 (m, 3H), 3.12˜3.20 (m, 1H), 3.58˜3.64 (m, 2H), 5.10˜5.24 (m, 3H), 7.04 (d, 1H), 7.42˜7.50 (m, 5H), 7.54 (d, 1H), 8.50 (br.s, 1H), 8.70 (br.s, 1H).
5-Bromo-2-[(4-chlorophenyl)methoxy]-α-[[(2-hydroxyethyl)amino]methyl]-benzenemethanol.

¹H NMR (DMSO-d₆/TFA): δ 2.88˜3.02(m, 3H), 3.12˜3.20(m, 1H), 3.58˜3.64(m, 2H), 5.10˜5.24(m, 3H), 7.04(d, 1H), 7.42˜7.50(m, 5H), 7.54(d, 1H), 8.50(br.s, 1H), 8.70(br.s, 1H).

### Example 37

### 5-Bromo-2-[(4-chlorophenyl)methoxy]-β-ethoxy-N,N-diethylbenzeneethanamine.

To a suspension of NaH (95%, 20 mg, 0.83 mmol, 1.3 eq.) in DMF (3 mL) was added 5-bromo-2-[(4-chlorophenyl)methoxy]-α-[(diethylamino)methyl]benzenemethanol (270 mg, 0.65 mmol) at rt. After stirring for 1 h, EtI (0.07 mL, 0.95 mmol) was added, and the resulting mixture was stirred at rt under N₂ overnight. The mixture was poured into ice-water, kept in the refrigerator overnight, and filtered. The solid was re-dissolved in EtOAc, and dried over Na₂SO₄. Concentration in vacuo, followed by purification by HPLC afforded the title compound as a colorless syrup. ¹H NMR (DMSO-d₆/TFA): δ 1.00˜1.22 (m, 9H), 3.00˜3.90 (m, 8H), 4.80˜5.00 (m, 1H), 5.10˜5.20 (m, 2H), 7.08˜7.20 (m, 1H), 7.40˜7.60 (m, 6H), 9.10 (br.s, 1H),

The following compound was prepared in a similar manner:
5-bromo-2-[(4-chlorophenyl)methoxy]-N,N-diethyl-β-(2-pyridinyloxy)benzeneethanamine. ¹H NMR (DMSO-d₆/TFA): δ 1.02 (t, 3H), 1.18 (t, 3H), 2.80˜3.20 (m, 4H), 3.35 (m, 1H), 3.65 (m, 1H), 4.70˜5.25 (m, 3H), 6.58 and 6.80 (each m, 1H), 6.96˜7.18 (m, 3H), 7.36˜7.60 (m, 5H), 7.74 and 7.89 (each m, 1H), 8.04˜8.14 (m, 1H), 9.32 (br.s, 1H).

### Example 38

### 5-Bromo-2-[(4-chlorophenyl)methoxy]-β-(methylamino)-benzeneethanol

A mixture of 2-[5-bromo-2-[(4-chlorophenyl)methoxy]phenyl]oxirane (500 mg, 1.47 mmol), TMSCN (0.4 mL, 3.0 mmol), and ZnI₂ (cat.) in CH₂Cl₂ (5 mL) in a sealed tube was stirred at 60° C. for 4 h. After cooling to rt, the reaction mixture was concentrated, and the resulting residue was diluted with EtOAc, washed with brine, and dried over Na₂SO₄. After removal of the solvent, the crude product was obtained. To a suspension of LiAIH₄ (100 mg, 2.8 mmol) in Et₂O (10 mL, anhydrous) was added dropwise a solution of the crude intermediate in Et₂O (10 mL) at rt. After 4 h, the reaction was quenched by addition of 15% NaOH (0.1 mL). After filtering off the solid, the filtrate was concentrated. The residue was purified by HPLC to afford the title compound as a white powder. ¹H NMR (DMSO-d₆/TFA): δ 2.46 (m, 3H), 3.71 (dd, 1H), 3.82(dd, 1H), 4.54 (m, 1H), 5.12˜5.20 (m, 2H), 7.06˜7.14 (m, 1H), 7.38˜7.58 (m, 5H), 7.64˜7.70 (m, 1H), 8.85 (br.s, 1H), 8.95 (br.s, 1H).

### Example 39

### 1-[5-Bromo-2-[(4-chlorophenyl)methoxy]phenyl]-2-propen-1-one

To a solution of vinylmagnasium bromide (1.0M in THF, 80 mL, 80 mmol) was added 5-bromo-2-(4-chlorophenylmethyl)benzaldehyde (21.5 g, 66 mmol) in THF (200 mL) at 0° C. After addition, the mixture was stirred at rt for 2 h, then poured into a chilled 10% HCl solution(150 mL). The mixture was extracted with EtOAc (3×1 50 mL), washed with brine (2×60 mL), and dried over Na₂SO₄. Concentration, followed by recrystallization from CH₂Cl₂-Hexane-EtOAc afforded the product allyl alcohol as a light yellow solid. To a stirred solution of the alcohol (3.8 g, 10.7 mmol) in CH₂Cl₂ (100 mL) was added Dess-Martin iodanane (5 g, 12 mmol) at rt. After 2 h, the reaction was quenched by adding Na₂S₂O₃ (10 g) and NaHCO₃ (sat., 20 mL). After removal of solvent, the residue was extracted with EtOAc, washed with brine, and dried over Na₂SO₄. Concentration followed by purification by flash chromatography afforded the title compound as white needles. ¹H NMR (400 MHz, DMSO-d₆): δ 5.07 s, 2H), 5.8 (d, 1H), 6.22 (d, 1H), 6.9 (m, 2H), 7.2˜7.4 (m, 4H), 7.5 (d, 1H), 7.7 (s, 1H).

### Example 40

### (3R)-α-[5-Bromo-2-[(4-chlorophenyl)methoxy]phenyl]-3-hydroxy-1-pyrrolidinepropanol

To a solution of 1-[5-bromo-2-[(4-chlorophenyl)methoxy]phenyl]-2-propen-1-one (300 mg, 0.85 mmol) in 10 mL MeOH—CH₂Cl₂(1:1, v/v) was added 3-(R)-hydroxypyrrolidine (0.1 mL, 1.25 mmol) at rt under N₂. After 30 min, the resulted mixture was added to a stirred solution of NaBH₄ in MeOH—CH₂Cl₂ (10 mL, 1:1, v/v) at rt. The mixture was stirred at rt for 0.5 h. After removing solvents, the residue was diluted with EtOAc, washed with brine, and dried over Na₂SO₄. Concentration followed by purification by HPLC afforded the title compound as a white powder. ¹H NMR (DMSO-d₆/TFA): δ 1.70˜2.22 (m, 4H), 2.84˜3.62 (m, 6H), 4.28˜4.40 (m, 1H), 5.06˜5.14 (m, 2H), 6.94˜7.00 (m, 1H), 7.32˜7.46 (m, 5H), 7.50˜7.53 (m, 1H), 9.50˜9.80 (m, 1H).

The following compounds were prepared in a similar manner:
5-Bromo-2-[(4-chlorophenyl)methoxy]-α-[2-(dimethylamino)ethyl]-benzenemethanol. ¹H NMR (DMSO-d₆/TFA): δ 1.78˜1.88 (m, 1H), 1.96˜2.06 (m, 1H), 2.70˜2.78 (m, 6H), 3.14 (m, 2H), 4.95 (br.dd, 1H), 5.10˜5.19 (m, 2H), 7.02 (d, 1H), 7.39 (dd, 1H), 7.43˜7.49 (m, 4H), 7.56 (d, 1H), 9.30 (br.s, 1H).
α-[5-Bromo-2-[(4-chlorophenyl)methoxy]phenyl]-4-hydroxy-1-piperidinepropanol. ¹H NMR (DMSO-d₆/TFA): δ 1.42˜2.10 (m, 6H), 2.88 (m, 1H), 3.04˜3.28 (m, 4H), 3.40 (br.t, 1H), 3.60 and 3.90 (each m, 1H), 4.93 (br.dd, 1H), 5.10˜5.19 (m, 2H), 7.03 (d, 1H), 7.40 (dd, 1H), 7.44˜7.49 (m, 4H), 7.55 (br.t, 1H), 9.06 (br.s, 1H).
5-Bromo-2-[(4-chlorophenyl)methoxy]-α-[2-(dipropylamino)ethyl]-benzenemethanol. ¹H NMR (DMSO-d₆/TFA): δ 0.83 (br.q, 6H), 1.54 (m, 4H), 1.80 (m, 1H), 2.00 (m, 1H), 2.94 (m, 4H), 3.14 (m, 2H), 4.93 (m, 1H), 5.08˜5.18 (m, 2H), 6.98˜7.04 (m, 1H), 7.36˜7.50 (m, 5H), 7.57 (d, 1H), 9.10 (br.s, 1H).

5-Bromo-2-[(4-chlorophenyl)methoxy]-α-[2-(diethylamino)ethyl]-benzenemethanol. ¹H NMR (DMSO-d₆/TFA): δ 1.07 (br.q, 6H), 1.76 (m, 1H), 1.96 (m, 1H), 2.95˜3.15 (m, 6H), 4.90 (dd, 1H), 5.10 (dd, 2H), 6.99 (br.dd, 1H), 7.34˜7.48 (m, 5H), 7.54 (d, 1H), 9.05 (br.s, 1H),

### Example 41

### 5-Chloro-2-[(4-fluorophenyl)methoxy]benzeneethanamine

A mixture of 5-bromo-2-(4-chlorophenylmethyl)benzaldehyde (5.0 g), nitromethane (2.23 mL, 41 mmol), and NH₄OAc (1.86 g, 23.4 mmol) in HOAc (25 mL) was kept at reflux for 2 h. After cooling to rt, the product was obtained by filtration (3.7 g). To a suspension of LiAIH₄ (1.6 g, 40 mmol) in THF (40 mL) at 0° C. was added a solution of the above product (3.5 g, 11.4 mmol) in THF (10 mL) dropwise. After addition, the mixture was kept at reflux for 1.5 h, cooled to rt, and quenched with concentrated NaOH (3 mL). The solid was filtered off, and the solution was concentrated to afford the desired amine. ¹H NMR (400 MHz, DMSO-d,): δ 2.7 (t, 2H), 2.94 (t, 2H), 5.04 (s, 2H), 6.8 (d, 11H), 7.0-7.24 (m, 4H), 7.4 (m,

### Example 42

### N-[[2-[5-Chloro-2-[(4-fluorophenyl)methoxy]-phenyl]ethyl]-4-pyridinemethanamine

To 5-chloro-2-[(4-fluorophenyl)methoxy]benzenethanamine (280 mg, 1 mmol) in MeOH (5 mL) was added 4-pyridinecarboxaldehyde (93.5 μL) and BH₃Py (0.19 mL, 8 M). The reaction mixture was stirred at rt overnight. Solvent was removed in vacuo, and the resulting residue was diluted with methylene chloride, washed with water and brine, and dried over Na₂SO₄. Concentration in vacuo, followed by purification by flash column chromatography afforded the title product. ¹H NMR (CDCl₃, 400 MHz) δ 2.80 (m, 4H), 3.78 (s, 2H), 5.0 (s, 2H), 6.80 (d, 1H), 7.05 (t, 2H), 7.15 (m, 4H), 7.35 (m, 2H), 8.50 (s, 1H).

The following compounds were prepared in a similar manner:
5-Chloro-2-[(4-fluorophenyl)methoxy]-N,N,α-trimethylbenzeneethanamine. ¹H NMR (DMSO-d₆, 400 MHz) d 0.80 (d, 3H), 2.10 (s, 6H), 2.36 (m, 1H), 2.80 (m, 2H), 5.05 (s, 2H), 6.80 (d, 1H), 7.0 (d, 1H), 7.20 (m, 4H), 7.50 (m, 2H).
4-[[[2-[5-Chloro-2-[(4-fluorophenyl)methoxy]phenyl]ethyl]amino]methyl]benzonitrile. ¹H NMR (DMSO-d₆, 400 MHz) δ (TMS) 2.95 (m, 2H), 3.10 (m, 2H), 4.25 (m, 2H), 5.10 (s, 2H), 7.05 (d, 1H), 7.20 (t, 2H), 7.25 (m, 2H), 7.45 (m, 2H), 7.65 (m, 2H), 7.90 (d, 2H).
N-[2-[5-chloro-2-[[4-fluorophenyl)methoxy]phenyl]ethyl]-N-(1H-imidazol-5-ylmethyl)-1H-imidazole-4-methanamine. ¹H NMR(CDCl₃, 400 MHz) δ (TMS) 2.65 (m, 2H), 2.90 (m, 2H), 3.60 (m, 4H), 5.0 (s, 2H), 6.80 (d, 1H), 6.90 (s, 2H), 7.05 (t, 2H), 7.10 (m, 2H), 7.35 (m, 2H), 7.60 (d, 2H).
5-Chloro-α-ethyl-2-[(4-fluorophenyl)methoxy]-N-[(4-fluorophenyl)methyl]benzeneethanamine. ¹H NMR (DMSO-d₆, 400 MHz) δ (TMS) 0.75 (t, 3H), 2.65 (m, 1H), 2.78 (m, 2H), 3.70 (m, 2H), 5.0 (m, 2H), 7.05 (m, 3H), 7.20 (m, 6H), 7.40 (m, 2H).
5-Chloro-α-ethyl-2-[(4-fluorophenyl)methoxy]-N-[(3-methyl-4-methoxyphenyl)methyl]benzeneethanamine. ¹H NMR (CDCl₃, 400 MHz) δ (TMS) 0.85 (t, 3H), 1.65 (m, 2H), 2.0 (s, 2H), 2.10 (s, 3H), 2.95 (m, 1H), 3.05(m, 2H), 3.75 (s, 3H), 3.80 (m, 2H), 4.90 (m, 2H), 6.70 (d, 1H), 6.80 (d, 1H), 6.95 (s, 1H), 7.05 (t, 2H), 7.20 (m, 5H).

### Example 43

### 1-[[5-Bromo-2-[(4-chlorophenyl)methoxy]phenyl]methyl]-4-hydroxy-4-piperidinecarboxylic acid, methyl ester

To a stirred solution of 1-[[5-bromo-2-[(4-chlorophenyl)methoxy]phenyl]methyl]-4-piperidinone (5 g, 12.2 mmol) and ZnI₂ (500 mg, 1.6 mmol) in CH₂Cl₂ (10 mL) was added TMSCN (2 mL, 15 mmol). The reaction mixture was stirred at 70° C. in a sealed tube for 5 h. After removing CH₂Cl₂, the residue was purified by flash chromatography to afford the intermediate cyanohydrin as a white solid. A solution of the above intermediate (1.8 g, 3.4 mmol) in MeOH (20 mL) was saturated with HCl (g) at 0° C. The solution was warmed to rt and stirred overnight. The reaction mixture was poured into cooled Et₂O (400 mL), and product precipitated. ¹H NMR (CDCl₃): δ 1.58˜1.68 (m, 2H), 2.13 (ddd, 2H), 2.46 (ddd, 2H), 2.70˜2.78 (m, 2H), 2.90 (br.s, 1H), 3.58 (s, 2H), 3.80 (s, 3H), 5.02 (s, 2H), 6.75 (d, 1H), 7.29 (dd, 1H), 7.36 (br.s, 4H), 7.53 (br.d, 1H).

### Example 44

### 1-[[5-Bromo-2-[(4-chlorophenyl)methoxy]phenyl]methyl]-4-hydroxy-4-piperidinecarboxylic acid

A mixture of 1-[[5-bromo-2-[(4-chlorophenyl)methoxy]phenyl]methyl]-4-hydroxy-4-piperidinecarboxylic acid, methyl ester (Ig) and LiOH (1.5 g)in 25 ml THF-H₂O (4:1, v/v) was stirred at rt overnight. After removing solvent, the resulting mixture was acidified with 1.0N HCl (aq.) to pH 2˜3. The mixture was extracted with CH₂Cl₂ and dried over Na₂SO₄. Concentration followed by purification through flash chromatography afforded the title compound as a white solid. ¹H NMR (CDCl₃), δ 1.64 (br.d, 2H), 2.50 (br.ddd, 2H), 3.02 (br. dd, 2H), 3.14˜3.24 (m, 2H), 4.30 (br.s, 2H), 5.07 (s, 2H), 6.81 (d, 1H), 7.28˜7.40 (m, 4H), 7.42 (dd, 1H), 7.81 (d, 1H).

### Example 45

### 4-[[1-[[5-Bromo-2-[(4-chlorophenyl)methoxy]phenyl]methyl]-4-hydroxy-4-piperidinyl]carbonyl]-1-piperazineethanol

To a stirred solution of 1-[[5-bromo-2-[(4-chlorophenyl)methoxy]phenyl]methyl]-4-hydroxy-4-piperidinecarboxylic acid (181 mg, 0.4 mmol) in DMF (5 mL) was added 1-(2-hydroxyethyl)piperazine (65 mg, 0.5 mmol), followed by HATU (198 mg, 0.52 mmol) and DIEA (103 mg, 0.8 mmol) at rt. After 4 h, the mixture was poured into ice-water (10 mL) and extracted with EtOAc (3×15 ml). The organic phase was dried over Na₂SO₄, and concentrated. The crude reside was purified by flash chromatography to afford the title compound. ¹H NMR (DMSO-d₆/TFA) δ 1.74˜2.30 (m, 4H), 2.88˜3.76 (m, 14H), 4.24˜4.90 (m, 4H), 5.16(s, 2H), 7.12˜7.18 (m, 1H), 7.40˜7.52 (m, 4H), 7.57˜7.62 (m, 1H), 7.70 (d, 1H), 9.40 (br.s, 1H), 9.80 (br.s, 1H).

The following compounds were prepared in a similar manner:
1-[[5-Bromo-2-[(4-chlorophenyl)methoxy]phenyl]methyl]-4-(1-piperazinylcarbonyl)-4-piperidinol. ¹H NMR (DMSO-d₆/TFA) δ 1.70˜2.30 (m, 4H), 3.00˜3.40 (m, 8H), 3.50˜4.20 (m, 4H), 4.28 (m, 2H), 5.15 (m, 2H), 7.12˜7.18 (m, 1H), 7.40˜7.52 (m, 4H), 7.60 (dd, 1H), 7.68˜7.73 (m, 1H), 8.80 (br.s, 2H), 9.35 (br.s, 1H).
1-[[5-Bromo-2-[(4-chlorophenyl)methoxy]phenyl]methyl]-4-[[(3R)-3-methylpiperazinyl]carbonyl]-4-piperidinol. ¹H NMR (DMSO-d₆/TFA): δ 1.18 (d, 3H), 1.70˜2.30 (m, 4H), 2.70˜3.50 (m, 9H), 4.10˜4.76 (m, 4H), 5.14˜5.20 (m, 2H), 7.12˜7.18 (m, 1H), 7.40˜7.52 (m, 4H), 7.56˜7.62 (m, 1H), 7.68˜7.72 (m, 1H), 8.80 (br.s, 1H), 9.10 (br.s, 1H), 9.40 (br.s, 1H).

### Example 46

### 4-[1-[[5-Bromo-2-[(4-chlorophenyl)methoxy]phenyl]methyl]-4-piperidinyl]-1-piperazinecarboxylic acid, 1,1-dimethylethyl ester

To a stirred solution of 1-[[5-bromo-2-[(4-chlorophenyl)methoxy]phenyl]methyl]-4-piperidinone (600 mg, 1.49 mmol) in EtOAc (10 mL) was added HOAc (98 mg, 1.63 mmol), followed by N-Boc-piperazine (304 mg, 1.63 mmol), and NaBH(OAc)₃ (474 mg, 2.24 mmol) in one portion at room temperature. After 16 h, the reaction was quenched with brine (40 mL), and extracted with EtOAc (3×35 mL). The organic phase was washed with brine (30 mL) and dried over Na₂SO₄. Concentration, followed by purification by column chromatography afforded the title compound. ¹H NMR (400 MHz, DMSO): 1.42 (s, 9H), 1.58 m, 2H), 1.76 (m, 3H), 2.3 (m, 1H), 2.95 (d, 2H), 3.41 (m, 4H), 3.54 (s, 2H), 5 (s, 2H), 6.74 (d, 1H), 7.28 (dd, 1H), 7.36 (dd, 4H), 7.5 (d, 1).

### Example 47

### 1-[1-[[5-Bromo-2-[(4-chlorophenyl)methoxy]phenyl]methyl]-4-piperidinyl]piperazine

To a stirred solution of 4-[1-[[5-bromo-2-[(4-chlorophenyl)methoxy]phenyl]methyl]-4-piperidinyl]-1-piperazinecarboxylic acid, 1,1-dimethylethyl ester (70 mg) in CH₂Cl₂ (1 mL) was added TFA (1 mL) at room temperature. After 2 h, the reaction was concentrated in vacuo to afford the title compound as a trifluoroacetic acid salt. ¹H NMR (400 MHz, DMSO-d₆): δ 1.78 (m, 2H), 2.1 (d, 12H), 3.1 (m, 10H), 3.5 (d, 2H), 4.3 (s, 2H), 5.2 (s, 2H), 7.17 (d, 1H), 7.46 (dd, 4H), 7.6 (dd, 1H), 7.67 (d, 1H), 9.1 (br. s, 1H).

### Example 48

### 1-[1-[[5-Bromo-2-[(4-chlorophenyl)methoxy]phenyl]methyl]-4-piperidinyl]-4-[(2,4-dimethyl-3-pyridinyl)carbonyl]piperazine

To a stirred solution of 1-[1-[[5-bromo-2-[(4-chlorophenyl)methoxy]phenyl]methyl]-4-piperidinyl]piperazine (144 mg, 0.3 mmol) in DMF (5 mL) was added 2,6-dimethyl-3-pyridyl carboxylic acid (60 mg, 0.4 mmol), followed by HATU (198 mg, 0.52 mmol), and DIEA (78 mg, 0.6 mmol) at rt. After 4 h, the mixture was poured into ice-water (10 mL), and extracted with EtOAc (3×15 ml). The organic phase was dried over Na₂SO₄, and concentrated. The crude reside was purified by flash chromatography to afford the title compound. ¹H NMR (DMSO-d₆/TFA): δ 1.82˜2.26 (m, 4H), 2.42 (br,s, 3H), 2.56 (br.s, 3H), 2.85˜3.60 (m, 13H), 4.25 (s, 2H), 5.15 (s, 2H), 7.16 (d, 1H), 7.42˜7.60 (m, 4H), 7.60 (dd, 1H), 7.82 (d, 1H) 8.74 (d, 2H).

### Example 49

### 1-[[5-Bromo-2-[(4-chlorophenyl)methoxy]phenyl]methyl]-3-methyl-4-piperidinone

To a stirred solution of 1-[[5-bromo-2-[(4-chlorophenyl)methoxy]phenyl]methyl]-4-piperidinone (640.5 mg, 1.5 mmol) in THF (10 mL) at −78° C. was added LDA (0.9 mL, 1.8 mmol, 2.0 M in THF/hexane). After 30 min, MeI (320 mg, 2.25 mmol) was added, and the reaction was allowed to warm to room temperature overnight. The reaction was quenched with 0.1 N HCl (10 mL) at 0° C., and extracted with EtOAc (3×30 mL). The organic phase was washed with brine (15 mL) and dried over Na₂SO₄. Concentration followed by purification through column chromatography afforded the title compound. ¹H NMR (400 MHz, DMSO-d₆): δ 0.8 (d, 3H), 2.12 (m, 2H), 2.36 (m, 2H), 3.0 (m, 2H), 3.6 (s, 2H), 5.1 (s, 2H), 7.0 (d, 1H), 7.45 (dd, 4H), 7.52 (d, 1H).

### Example 50

### 1-[[5-Bromo-2-[(4-chlorophenyl)methoxy]phenyl]methyl]-3-methyl-4-piperidinol

To a stirred solution of 1-[[5-bromo-2-[(4-chlorophenyl)methoxy]phenyl]methyl]-3-methyl-4-piperidinone (150 mg, 0.36 mmol) in MeOH (10 mL) at 0° C. was added NaBH₄ (100 mg, 2.63 mmol) in one portion. After 30 min, the reaction mixture was diluted with brine (10 mL), and extracted with EtOAc (3×30 mL). The organic phase was washed with brine (10 mL) and dried over Na₂SO₄. Concentration followed by purification through column chromatography afforded the title compound. ¹H NMR (400 MHz, DMSO-d₆): δ 0.9 (d, 3H), 1.5 (m, 2H), 1.7 (m, 2H), 1.8 (m, 1H), 2.0 (m, 1H), 2.78 (m, 2H), 3.1 (m, 1H), 3.4 (s, 2H), 4.9 (s, 2H), 6.7 (d, 1H), 7.2 (dd, 1H), 7.32 (dd, 4H), 7.52 (d, 1H).

### Example 51

### 1-[[5-Bromo-2-[(4-chlorophenyl)methoxy]phenyl]methyl]-4,4-difluoropiperidine

To a stirred solution of 1-[[5-bromo-2-[(4-chlorophenyl)methoxy]phenyl]methyl-4-piperidinone (500 mg, 1.24 mmol) in CH₂Cl₂ (20 mL) at −78° C. was added DAST (978 mg, 6.19 mmol). The reaction mixture was allowed to warm to room temperature overnight, then re-cooled to 0° C., and quenched with NaOH (10%, 15 mL). The reaction mixture was extracted with EtOAc (3×15 mL), washed with brine (10 mL), and dried over Na₂SO₄. Concentration followed by purification through column chromatography afforded the title compound. ¹H NMR(400 MHz, DMSO-d₆): 1.9 (m, 4H), 3.55 (s, 2H), 5.1 (s, 2H), 7.0 (d, 1H), 7.36 (dd, 1H), 7.42 (m, 5H).

### Example 52

### 8-[[5-Bromo-2-[(4-chlorophenyl)methoxy]phenyl]methyl]-1,3,8-triazaspiro[4.5]decane-2,4-dione

A mixture of 1-[[5-bromo-2-[(4-chlorophenyl)methoxy]phenyl]methyl]-4-piperidinone(2 g, 4.9 mmol), KCN (500 mg, 7.7 mmol) and (NH₄)₂CO₃ (2 g, 19 mmol) in EtOH-H₂O(40 mL, 1:1) in a sealed tube was stirred at 120° C. overnight. After diluting with water, the cooled mixture was acidified with concentrated hydrochloric acid slowly. The crude hydantoin product was recrystallized from CH₂Cl₂-MeOH to afford the product as a white powder. ¹H NMR (DMSO-d₆/TFA): δ 1.82˜2.20 (m, 4H), 3.10˜3.50(m, 4H), 4.25˜4.35 (m, 2H), 5.20 (s, 2H), 7.19 (d, 1H), 7.44˜7.56 (m, 4H), 7.63 (m, 1H), 7.81(br.s, 1H), 8.32 and 8.78 (each s, 1H), 9.90˜10.10 (m, 1H), 10.80˜10.90 (m, 1H).

### Example 53

### 1-[[5-Bromo-2-[(4-chlorophenyl)methoxy]phenyl]methyl]-4-phenyl-4-piperidinol

To a solution of 1-[[5-bromo-2-[(4-chlorophenyl)methoxy]phenyl]methyl]-4-piperidinone (300 mg, 0.73 mmol) in THF (5 mL) was added a solution of phenylmagnesium bromide(0.9 mL, 0.9 mmol, 1.0M in THF) at 0° C. The mixture was warmed to rt and stirred for an additional 1 h. The reaction was quenched with 1.0N HCl (aq.) at 0° C. After removing the THF in vacuo, the residue was diluted with EtOAc, washed with brine, and dried over Na₂SO₄. Concentration followed by purification by HPLC affords the product as white powder. ¹H NMR (DMSO-d₆/TFA): δ 1.62 and 1.78(each br.d, 2H), 2.10˜2.32(m, 2H), 3.02˜3.44(m, 4H), 4.32 and 4.44(each br.d, 2H), 5.12 and 5.20 (each s, 2H), 7.16˜7.26 (m, 2H), 7.30˜7.54 (m, 8H), 7.60˜7.65 (m, 1H), 7.75 and 7.81 (each d, 1H), 9.30˜9.50 (m, 1H).

The follow compound was made in a similar manner:
1-[[5-Bromo-2-[(4-chlorophenyl)methoxy]phenyl]methyl]-4-ethyl-4-piperidinol. ¹H NMR (DMSO-d₆/TFA): δ 0.72 and 0.79 (each t, 3H), 1.30˜1.74 (m, 6H), 2.80˜3.25 (m, 4H), 4.20˜4.30 (m, 2H), 5.13 and 5.17 (each s, 2H), 7.16 (d, 1H), 7.40˜7.54 (m, 4H), 7.57˜7.63 (m, 1H), 7.68˜7.74 (m, 1H).

### Example 54

### 1-[[5-Bromo-2-[(4-chlorophenyl)methoxy]phenyl]methyl]-4-(trifluoromethyl)-4-piperidinol

To a solution of 1-[[5-bromo-2-[(4-chlorophenyl)methoxy]phenyl]methyl]-4-piperidinone (380 mg, 0.93 mmol) in THF(5 mL) was added TMSCF₃ (0.3 mL, 2.03 mmol) and a catalytic amount of TMAF.H₂O at rt. After 1 h, 1 mL of conc HCl was added, and stirred for another 30 min. After removing solvents, the residue was diluted with EtOAc; washed with NaHCO₃ (sat.) and brine, and dried over Na₂SO₄. Concentration followed by purification by HPLC afforded the title compound.

### Example 55

### 1-[[5-Bromo-2-[(4-chlorophenyl)methoxy]phenyl]methyl]-4-piperidinone-oxime

To a solution of 1-[[5-bromo-2-[(4-chlorophenyl)methoxy]phenyl]methyl]-4-piperidinone (300 mg, 0.73 mmol) and HONH₂.HCl (61 mg, 0.88 mmol) in CH₂Cl₂ (5 mL) was added pyridine (0.3 mL, 3.7 mmol) at rt. After 20 h, the solid was collected by filtration, and washed with Hexane-EtOAc to afford the product as a white solid. ¹H NMR (DMSO-d₆/TFA): δ 2.30˜3.50 (m, 8H), 4.26 (br.s, 2H), 5.16 (s, 2H), 7.14 (d, 1H), 7.40˜7.54 (m, 4H), 7.58 (dd, 1H), 7.86 (d, 1H), 10.80˜10.90 (m, 1H).

### Example 56

### 1-[[5-Bromo-2-[[4-(trifluoromethyl)phenyl]methoxy]phenyl]methyl]-4-fluoropiperidine

To a solution of 1-[[5-bromo-2-[[4-(trifluoromethyl)phenyl]methoxy]phenyl]methyl]-4-piperidinol (870 mg, 2.0 mmol) in CH₂Cl₂ (10 mL) was added DAST (0.85 mL, 5.5 mmol) at −78° C. The mixture was warmed to rt overnight. The mixture was re-cooled to −78° C. and quenched with MeOH. After concentration, the residue was purified by recrystallization from hexane-EtOAc (collect the liquid) followed by flash chromatography to afford the product as a colorless syrup. ¹H NMR (DMSO-d₆/TFA): δ 1.78˜2.24 (m, 4H), 3.02˜3.56 (m, 4H), 4.28˜4.36 (m, 2H), 4.70˜5.00 (m, 1H), 5.28 (s, 2H), 7.15 (d, 1H), 7.59 (dd, 1H), 7.66˜7.75 (m, SH), 9.50 (br.s, 1H).

### Example 57

### 1-[[5-Bromo-2-[(4-chlorophenyl)methoxy]phenyl]methyl]-4-(2-pyridinyloxy)piperidine

To a stirred solution of 1-[[5-bromo-2-[(4-chlorophenyl)methoxy]phenyl]methyl]-4-piperidinol (205 mg, 0.5 mmol) in DMF at 0° C. was added NaH (16.1 mg, 0.7 mmol). After 30 min, 2-fluoropyridine (116 mg, 1.2 mmol) was added, and the solution was kept at 100° C. for 5 h. After cooling to rt, the reaction was worked-up as usual, and purified by flash chromatography to afford the title compound as a white powder. ¹H NMR (DMSO-d₆/TFA): δ 1.82 (m, 1H), 1.98˜2.14 (m, 2H), 2.26 (m, 1H), 3.18 (m, 2H), 3.30 (m, 1H), 3.45 (m, 1H), 4.30 (m, 2H), 5.06˜5.30 (m, 3H), 6.84 (m, 1H), 7.00 (m, 1H), 7.16 (m, 1H), 7.36 (m, 1H), 7.40˜7.54 (m, 3H), 7.60 (m, 1H), 7.70˜7.80 (m, 2H), 8.14 (m, 1H), 9.40 (br.s, 1H).

The following compound was prepared in a similar manner:
2-[[1-[[5-Bromo-2-[(4-chlorophenyl)methoxy]phenyl]methyl]-4-piperidinyl]oxy]pyrimidine.

¹H NMR (DMSO-d₆/TFA): δ 1.84 (m, 1H), 2.10 (m, 2H), 2.28 (m, 1H), 3.22 (br.s, 2H), 3.32 (m, 1H), 3.46 (m, 1H), 4.30 (m, 2H), 5.12˜5.30 (m, 3H), 7.16 (m, 1H), 7.36˜7.56 (m, 4H), 7.60 (m, 1H), 7.74 (m, 1H), 8.14˜8.28 (m, 3H), 9.45 (br.s, 1H).

### Example 58

### 1-[[5-Bromo-2-[(4-chlorophenyl)methoxy]phenyl]methyl]-N-ethyl-4-piperidinamine

To a stirred solution of 1-[[5-bromo-2-[(4-chlorophenyl)methoxy]phenyl]methyl]-4-piperidinone (204 mg, 0.5 mmol) in CH₂Cl₂-MeOH (2 mL/2 mL) was added ethylamine (0.2 mL), followed by NaBH(OAc)₃ (170 mg, 0.8 mmol) and HOAc (0.2 mL) at rt. After 10 h, the reaction was worked up as usual, and purified by flash chromatography to afford the title compound as a white powder. ¹H NMR (DMSO-d₆/TFA): δ 1.10˜1.26 (m, 3H), 1.72 (m, 2H), 1.86˜2.22 (m, 2H), 2.86˜3.50 (m, 7H), 4.21 (br.s, 2H), 5.16 (s, 2H), 7.15 (d, 1H), 7.40˜7.52 (m, 4H), 7.59 (dd, 1H), 7.67 (br.d, 1H), 8.90 (br.s, 2H), 10.00 (br.s, 1H).

### Example 59

### 6-[[5-Bromo-2-[(4-chlorophenyl)methoxy]phenyl]methyl]-1-oxa-6-azaspiro[2.5]octane

To a homogeneous solution of (Me)₃SI (6.28 g, 30.7 mmol) in DMSO (150 mL) was added 1-[[5-bromo-2-[(4-chlorophenyl)methoxy]phenyl]methyl]-4-piperidinone (8.0 g, 19.81 mmol), followed by KO-tBu (3.45 g, 30.71 mmol) at rt. The reaction was kept at rt overnight, and was poured into ice-water (150 mL). The reaction mixture was extracted with EtOAc (3×160 mL), washed with brine (150 mL), and dried over Na₂SO₄. Concentration followed by purification through column chromatography afforded the title compound. ¹H NMR (400 MHz, DMSO-d₆): 1.55 (m, 2H), 1.9 (m, 2H), 2.6 m, 2H), 2.65 (s, 2H), 3.6 (s, 2H), 5.02 (s, 2H), 6.78 (d, 1H), 7.3 (dd, 1H), 7.36 (s, 4H), 7.53 (d, 1H).

### Example 60

### 4-(Aminomethyl)-1-[[5-bromo-2-[(4-chlorophenyl)methoxy]phenyl]methyl]-4-piperidinol, and 1-[[5-bromo-2-[(4-chlorophenyl)methoxy]phenyl]methyl]-4-[[[[1-[[5-bromo-2-[(4-chlorophenyl)methoxy]phenyl]methyl]-4-hydroxy-4-piperidinyl]methyl]amino]methyl]-4-piperidinol

A mixture of 6-[[5-bromo-2-[(4-chlorophenyl)methoxy]phenyl]methyl]-1-oxa-6-azaspiro[2.5]octane (4 g), NH₄OH(12 mL, 28˜30% wt) in MeOH (50 mL) in a sealed tube was stirred at 75° C. overnight. After concentration, the residue was purified directly by flash chromatography to afford 4-(aminomethyl)-1-[[5-bromo-2-[(4-chlorophenyl)methoxy]phenyl]methyl]-4-piperidinol as a white solid. ¹H NMR (DMSO-d₆/TFA): δ 1.70˜1.96(m, 4H), 2.80˜3.80 (m, 6H), 4.26˜4.38 (m, 2H), 5.18˜5.22 (m, 2H), 7.14˜7.20 (m, 1H), 7.44˜7.54 (m, 4H), 7.60˜7.64 (m, 1H), 7.70˜7.74 (m, 1H), 7.86 (br.s, 2H), 9.50 (m, 1H), and a by-product: 1-[[5-bromo-2-[(4-chlorophenyl)methoxy]phenyl]methyl]-4-[[[[1-[[5-bromo-2-[(4-chlorophenyl)methoxy]phenyl]methyl]-4-hydroxy-4-piperidinyl]methyl]amino]methyl]-4-piperidinol as light yellow solid. ¹H NMR (DMSO-d₆/TFA): δ 1.68˜2.06 (m, 8H), 3.00˜3.34 (m, 12H), 4.24˜4.34 (m, 4H), 5.14˜5.20 (m, 4H), 7.11˜7.17 (m, 2H), 7.40˜7.51 (m, 8H), 7.56˜7.61 (m, 2H), 7.68˜7.71 (m, 2H), 8.35 (br.s, 2H), 9.60 (m, 2H).

The following compounds were prepared in a similar manner:
4-[[1-[[5-Bromo-2-[(4-chlorophenyl)methoxy]phenyl]methyl]-4-hydroxy-4-piperidinyl]methyl]-1-piperazinecarboxylic acid, 1,1-dimethylethyl ester. ¹H NMR (DMSO-d₆/TFA): δ 1.34˜1.40 (m, 9H), 1.68˜2.10 (m, 4H), 3.00˜4.00 (m, 14H), 4.25 (br.s, 2H), 5.15 (s, 2H), 7.12˜7.17 (m, 1H), 7.40˜7.52 (m, 4H), 7.59 (dd, 1H), 7.70 (d, 1H), 9.50 (br.s, 1H).
4-[[1-[[5-Bromo-2-[(4-chlorophenyl)methoxy]phenyl]methyl]-4-hydroxy-4-piperidinyl]methyl]hexahydro-1H-1,4-diazepine-1-carboxylic acid, 1,1-dimethylethyl ester. ¹H NMR (DMSO-d₆/TFA): δ 1.36˜1.40 (m, 9H), 1.68˜2.16 (m, 6H), 3.10˜3.80 (m, 14H), 4.30 (s, 2H), 5.15 (s, 2H), 7.13˜7.18 (m, 1H), 7.41˜7.52 (m, 4H), 7.59 (dd, 1H), 7.70 (d, 1H), 9.30˜9.60 (m, 1H).
1-[[5-Bromo-2-[(4-chlorophenyl)methoxy]phenyl]methyl]-4-[[4-(2-pyridinyl)-1-piperazinyl]methyl]-4-piperidinol. ¹H NMR (DMSO-d₆/TFA): δ 1.72˜2.12 (m, 4H), 3.10˜4.20 (m, 14H), 4.30 (s, 2H), 5.18 (s, 2H), 6.97 (m, 1H), 7.12˜7.18 (m, 1H), 7.33 (m, 1H), 7.41˜7.52 (m, 4H), 7.60 (m, 1H), 7.70 (m, 1H), 7.97 (m, 1H), 8.10˜8.16 (m, 1H), 9.60 (br.s, 1H).
1-[[5-Bromo-2-[(4-chlorophenyl)methoxy]phenyl]methyl]-4-[[4-(2-pyrimidinyl)-1-piperazinyl]methyl]-4-piperidinol. ¹H NMR (DMSO-d₆/TFA): δ 1.70˜2.10 (m, 4H), 3.10˜3.70 (m, 12H), 4.30 (s, 2H), 4.50 (br.s, 2H), 5.18 (s, 2H), 6.72 (m, 1H), 7.13˜7.18 (m, 1H), 7.41˜7.52 (m, 4H), 7.60 (m, 1H), 7.71 (d, 1H), 8.40 (m, 2H), 9.50 (br.s, 1H).
1-[[5-Bromo-2-[(4-chlorophenyl)methoxy]phenyl]methyl]-4-(1-piperazinylmethyl)-4-piperidinol. ¹H NMR (DMSO-d₆/TFA): δ 1.69˜2.06(m, 4H), 3.10˜3.51(m, 14H), 4.27 (br.s, 2H), 5.14 (br.s, 2H), 7.07˜7.13 (m, 1H), 7.36˜7.48 (m, 4H), 7.54 (m, 1H), 7.69 (m, 1H), 9.10 (br.s, 2H), 9.41 (br.s, 1H).
1-[[5-Bromo-2-[(4-chlorophenyl)methoxy]phenyl]methyl]-4-[(hexahydro-1H-1,4-diazepin-1-yl)methyl]-4-piperidinol. ¹H NMR (DMSO-d₆/TFA): δ 1.48˜1.97 (m, 6H), 2.88˜3.58 (m, 14H), 4.08 (br.s, 2H), 4.96 (br.s, 2H), 6.90˜6.95 (m, 1H), 7.18˜7.30 (m, 4H), 7.37 (m, 1H), 7.50 (d, 1H), 8.70 (br.s, 2H), 9.22 (br.s, 1H).
1-[[5-Bromo-2-[(4-chlorophenyl)methoxy]phenyl]methyl]-4-[[(4-methylphenyl)amino]methyl]-4-piperidinol. ¹H NMR (DMSO-d₆/TFA): δ 1.70˜2.06 (m, 4H), 2.22˜2.24 (m, 3H), 3.10˜3.38 (m, 6H), 4.22˜4.32 (m, 2H), 5.17 (s, 2H), 7.06˜7.24 (m, 5H), 7.38˜7.52 (m, 4H), 7.56˜7.61 (m, 1H), 7.70 (d, 1H), 9.20˜9.40 (m, 1H).
1-[[5-Bromo-2-[(4-chlorophenyl)methoxy]phenyl]methyl]-4-[[(4-methoxyphenyl)amino]methyl]-4-piperidinol. ¹H NMR (DMSO-d₆/TFA): δ 1.72˜2.08 (m, 4H), 3.10˜3.42 (m, 6H), 3.72˜3.76 (m, 3H), 4.26˜4.32 (m, 2H), 5.18 (s, 2H), 6.96˜7.60 (m, 2H), 7.10˜7.20 (m, 1H), 7.28˜7.38 (m, 2H), 7.40˜7.52 (m, 4H), 7.58˜7.62 (m, 1H), 7.70 (d, 1H), 9.30˜9.50 (m, 1H).
1-[[5-Bromo-2-[(4-chlorophenyl)methoxy]phenyl]methyl]-4-[[(3S)-3-methylpiperazinyl]methyl]-4-piperidinol. ¹H NMR (DMSO-d₆/TFA): δ 1.15˜1.40 (m, 3H), 1.70˜2.05 (m, 4H), 2.95˜3.70 (m, 13H), 4.28 (br.s, 2H), 5.18 (br.s, 2H), 7.12˜7.18 (m, 1H), 7.40˜7.52 (m, 4H), 7.58˜7.62 (m, 1H), 7.70 (d, 1H), 9.10˜9.60 (m, 1H).
1-[[5-Bromo-2-[(4-chlorophenyl)methoxy]phenyl]methyl]-4-[(2,5-dimethyl-1-piperazinyl)methyl]-4-piperidinol. ¹H NMR (DMSO-d₆/TFA): δ 1.16˜1.30 (m, 6H), 1.70˜2.08 (m, 4H), 3.00˜3.74 (m, 12H), 4.26˜4.34 (m, 2H), 5.18 (s, 2H), 7.13˜7.17 (m, 1H), 7.41˜7.52 (m, 4H), 7.60 (dd, 1H), 7.71 (d, 1H), 9.50 (br.s, 1H).
4-[[(3-Aminopropyl)amino]methyl]-1-[[5-bromo-2-[(4-chlorophenyl)methoxy]phenyl]methyl]-4-piperidinol. ¹H NMR (DMSO-d₆/TFA): δ 1.66˜2.00 (m, 6H), 2.80˜3.36 (m, 10H), 4.26˜4.34 (m, 2H), 5.18 (m, 2H), 7.11˜7.18 (m, 1H), 7.46 (AB, 4H), 7.60 (m, 1H), 7.70 (m, 1H), 7.82 (br.s, 3H), 8.54 (br.s, 2H), 9.50 (br.s, 1H).
1-[[5-Bromo-2-[(4-chlorophenyl)methoxy]phenyl]methyl]-4-[[[2-(1-piperidinyl)ethyl]amino]methyl]-4-piperidinol. ¹H NMR (DMSO-d₆/TFA): δ 1.30˜2.02 (m, 10H), 2.86˜3.48 (m, 14H), 4.26˜4.32 (m, 2H), 5.16˜5.19 (m, 2H), 7.11˜7.17 (m, 1H), 7.50 (AB, 4H), 7.60 (m, 1H), 7.70 (m, 1H), 8.80 (br.s, 1H), 9.60 (br.s, 1H).
2-[[[[1-[[5-Bromo-2-[(4-chlorophenyl)methoxy]phenyl]methyl]-4-hydroxy-4-piperidinyl]methyl]amino]methyl]-1-pyrrolidinecarboxylic acid, 1,1-dimethylethyl ester. ¹H NMR (DMSO-d₆/TFA): δ 1.38 (m, 9H), 1.64˜2.00 (m, 8H), 2.90˜3.36 (m, 10H), 4.04 (m, 1H), 4.24˜4.30 (m, 2H), 5.18 (br.s, 2H), 7.12˜7.18 (m, 1H), 7.40˜7.52 (m, 4H), 7.60 (m, 1H), 7.68˜7.70 (m, 1H), 8.40˜8.60 (m, 2H), 9.50 (br.s, 1H).
1-[[5-Bromo-2-[(4-chlorophenyl)methoxy]phenyl]methyl]-4-[[(2-pyrrolidinylmethyl)amino]methyl]-4-piperidinol. ¹H NMR (DMSO-d₆/TFA): δ 1.60˜2.06 (m, 7H), 2.12 (m,1H), 2.92˜3.38 (m, 10H), 3.86 (br.s, 1H), 4.26˜4.32 (m, 2H), 5.18˜5.20 (m, 2H), 7.11˜7.17 (m, 1H), 7.40˜7.52 (m, 4H), 7.58˜7.62 (m, 1H), 7.68˜7.72 (m, 1H), 8.60 (br.s, 1H), 8.80 (br.s, 2H), 9.05 (br.s, 1H), 9.60 (br.s, 1H).
1-[[5-Bromo-2-[(4-chlorophenyl)methoxy]phenyl]methyl]-4-[[4-[(2,4-dimethyl-3-pyridinyl)carbonyl]-1-piperazinyl]methyl]-4-piperidinol. ¹H NMR (DMSO-d₆/TFA): δ 1.72˜2.04 (m, 4H), 2.42 (br.s, 3H), 2.56 (br.s, 3H), 2.86˜3.58 (m, 14H), 4.28 (br.s, 2H), 5.18 (s, 2H), 7.12˜7.18 (m, 1H), 7.42˜7.52 (m, 4H), 7.60 (dd, 1H), 7.70 (d, 1H), 7.80˜7.84 (m, 1H), 8.72 (br.d, 1H), 9.60 (br.s, 1H).
4-[[1-[[5-Bromo-2-[(4-chlorophenyl)methoxy]phenyl]methyl]-4-hydroxy-4-piperidinyl]methyl]-1-piperazinecarboxylic acid, ethyl ester. ¹H NMR (DMSO-d₆/TFA): δ 1.14˜1.20(m, 3H), 1.70˜2.06 (m, 4H), 3.10˜3.90 (m, 14H), 4.00˜4.10 (m, 2H), 4.30 (br.s, 2H), 5.17 (s, 2H), 7.12˜7.18 (m, 1H), 7.42˜7.52 (m, 4H), 7.60 (dd, 1H), 7.70 (d, 1H), 9.50 (br.s, 1H).
4-[(4-Acetyl-1-piperazinyl)methyl]-1-[[5-bromo-2-[(4-chlorophenyl)methoxy]phenyl]methyl]-4-piperidinol. ¹H NMR (DMSO-d₆/TFA): δ 1.70˜2.08 (m, 7H), 3.00˜4.00 (m, 14H), 4.30 (br.s, 2H), 5.18 (s, 2H), 7.12˜7.18 (m, 1H), 7.42˜7.52 (m, 4H), 7.60 (dd, 1H), 7.70 (d, 1H), 9.60 (br.s, 1H).
1-[[5-Bromo-2-[(4-chlorophenyl)methoxy]phenyl]methyl]-4-[(1-piperazinylamino)methyl]-4-piperidinol. ¹H NMR (DMSO-d₆/TFA): δ 1.68˜2.00 (m, 4H), 2.78˜3.66 (m, 17H), 4.28 (br.s, 2H), 5.16˜5.20 (m, 2H), 7.11˜7.18 (m, 1H), 7.46 (AB, 4H), 7.60 (br.dd, 1H), 7.70 (d, 1H), 9.55 (br.s, 1H).
4-[[1-[[5-Bromo-2-[(4-chlorophenyl)methoxy]phenyl]methyl]-4-hydroxy-4-piperidinyl]methyl]-1-piperazineethanol. ¹H NMR (DMSO-d₆/TFA): δ 1.70˜2.04 (m, 4H), 3.06˜3.76 (m, 18H), 4.27 (br.s, 2H), 5.17 (s, 2H), 7.12˜7.17 (m, 1H), 7.41˜7.52 (m, 4H), 7.59 (dd, 1H), 7.70 (d, 1H), 9.70 (br.s, 1H).
4-[[1-[[5-Bromo-2-[(4-chlorophenyl)methoxy]phenyl]methyl]-4-hydroxy-4-piperidinyl]methyl]-1-piperazinecarboxaldehyde. ¹H NMR (DMSO-d₆/TFA): δ 1.70˜2.08 (m, 4H), 3.08˜3.76 (m, 14H), 4.28 (br.s, 2H), 5.16 (s, 2H), 7.12˜7.18 (m, 1H), 7.40˜7.52 (m, 4H), 7.56˜7.62 (m, 1H), 7.70 (d, 1H), 8.01˜8.04 (m, 1H), 9.60 (br.s, 1H).
4-[[1-[[5-Bromo-2-[(4-chlorophenyl)methoxy]phenyl]methyl]-4-hydroxy-4-piperidinyl]methyl]-1-piperazinecarboxylic acid, phenylmethyl ester. ¹H NMR (DMSO-d₆/TFA): δ 1.70˜2.10 (m, 4H), 3.05˜4.10 (m, 14H), 4.30 (br.s, 2H), 5.08˜5.10 (m, 2H), 5.17 (s, 2H), 7.12˜7.18 (m, 1H), 7.28˜7.38 (m, 5H), 7.42˜7.52 (m, 4H), 7.60 (dd, 1H), 7.70 (d, 1H), 9.55 (br.s, 1H).
1-[[5-Bromo-2-[(4-chlorophenyl)methoxy]phenyl]methyl]-4-[[4-(phenylmethyl)-1-piperazinyl]methyl]-4-piperidinol. ¹H NMR (DMSO-d₆/TFA): δ1.36˜1.94 (m, 8H), 2.44˜3.50 (m, 12H), 4.16 (br.s, 2H), 5.05 (s, 2H), 7.00˜7.08 (m, 4H), 7.12˜7.18 (m, 2H), 7.28˜7.40 (m, 4H), 7.46˜7.50 (m, 1H), 7.57˜7.60 (m, 1H), 8.60˜9.40 (m, 2H).
1-[[5-Bromo-2-[(4-chlorophenyl)methoxy]phenyl]methyl]-4-[[(2-methylphenyl)amino]methyl]-4-piperidinol. ¹H NMR (DMSO-d₆/TFA): δ 1.70˜2.04 (m, 4H), 2.12 and 2.18 (each s, 3H), 3.08˜3.34 (m, 6H), 4.24˜4.34 (m, 2H), 5.16 (m, 2H), 6.60˜6 6.60˜6.90 (m, 2H), 6.98˜7.18 (m, 3H), 7.38˜7.52 (m, 4H), 7.59 (dd, 1H), 7.69˜7.72 (m, 1H), 9.20˜9.40 (m, 1H).
1-[[1-[[5-Bromo-2-[(4-chlorophenyl)methoxy]phenyl]methyl]-4-hydroxy-4-piperidinyl]methyl]-4-piperidinecarboxamide. ¹H NMR (DMSO-d₆/TFA): δ 1.68˜2.04 (m, 8H), 2.32 (m, 1H), 2.90˜3.65 (m, 10H), 4.30 (m, 2H), 5.18 (s, 2H), 6.88˜6.98 (m, 1H), 7.13˜7.18 (m, 1H), 7.36 (m, 1H), 7.42˜7.52 (m, 1H), 7.60 (dd, 1H), 7.70 (d, 1H), 8.90˜9.60 (m, 2H). LC-MS(API150EX): calcd:550, found:550.
1-[[5-Bromo-2-[(4-chlorophenyl)methoxy]phenyl]methyl]-4-[[(3S)-3-methylpiperazinyl]methyl]-4-piperidinol. ¹H NMR (DMSO-d₆/TFA): δ 1.15˜1.40 (m, 3H), 1.70˜2.05 (m, 4H), 2.95˜3.70 (m, 13H), 4.28 (br.s, 2H), 5.18 (br.s, 2H), 7.12˜7.18 (m, 1H), 7.40˜7.52 (m, 4H), 7.58˜7.62 (m, 1H), 7.70 (d, 1H), 9.10˜9.60 (m, 1H).
4-[[1-[[5-Bromo-2-[(4-chlorophenyl)methoxy]phenyl]methyl]-4-hydroxy-4-piperidinyl]methyl]-2-piperazinone. ¹H NMR (DMSO-d₆/TFA): δ 1.68˜2.06 (m, 4H), 3.10˜3.70 (m, 10H), 3.75 (s, 2H), 4.28 (s, 2H), 5.18 (s, 2H), 7.12˜7.18 (m, 1H), 7.40˜7.52 (m, 4H), 7.58˜7.62 (m, 1H), 7.70 (d, 1H), 8.38˜8.42 (m, 1H), 9.50 (br.s, 1H).
1-[[5-Bromo-2-[(4-chlorophenyl)methoxy]phenyl]methyl]-4-[[(3S)-3-methylpiperazinyl]methyl]-4-piperidinol. ¹H NMR (DMSO-d₆/TFA): δ 1.16˜1.26 (m, 3H), 1.68˜2.04 (m, 4H), 2.95˜3.65 (m, 13H), 4.28 (s, 2H), 5.18 (s, 2H), 7.12˜7.18 (m, 1H), 7.40˜7.52 (m, 4H), 7.59 (dd, 1H), 7.70 (d, 1H), 8.70˜9.60 (m, 2H).
1-[[5-Bromo-2-[(4-chlorophenyl)methoxy]phenyl]methyl]-4-[(3,5-dimethyl-1-piperazinyl)methyl]-4-piperidinol. ¹H NMR (DMSO-d₆/TFA): δ 1.16˜1.24 (m, 6H), 1.68˜2.08 (m, 4H), 2.80˜3.30 (m, 8H), 3.62 (m, 4H), 4.28 (s, 2H), 5.18 (s, 2H), 7.12˜7.18 (m, 1H), 7.40˜7.52 (m, 4H), 7.60 (dd, 1H), 7.70 (d, 1H), 8.90˜9.60 (m, 2H).
1-[[5-Bromo-2-[(4-chlorophenyl)methoxy]phenyl]methyl]-4-(2,5-diazabicyclo[2.2.1]hept-2-ylmethyl)-4-piperidinol. ¹H NMR (DMSO-d₆/TFA): δ 1.66˜2.40 (m, 6H), 3.04˜3.76 (m, 10H), 4.26 (s, 2H), 4.42 (br.s, 1H), 4.58˜4.64 (m, 1H), 5.17 (m, 2H), 7.11˜7.18 (m, 1H), 7.40˜7.52 (m, 4H), 7.56˜7.62 (m, 1H), 7.68˜7.71 (m, 1H), 9.20˜9.60 (m, 2H).
[1-[[1-[[5-Bromo-2-[(4-chlorophenyl)methoxy]phenyl]methyl]-4-hydroxy-4-piperidinyl]methyl]-3-pyrrolidinyl]-carbamic acid, 1,1-dimethylethyl ester. ¹H NMR (DMSO-d₆/TFA): δ 1.35 (br.s, 9H), 1.66˜2.32 m, 6H), 2.85˜4.20 (m, 11H), 4.25 (s, 2H), 5.15 (s, 2H), 7.12˜7.18 (m, 1H), 7.40˜7.52 (m, 4H), 7.57˜7.62 (m, 1H), 7.70 (d, 1H), 9.50 (br.s, 1H).
4-[(3-Amino-1-pyrrolidinyl)methyl]-1-[[5-bromo-2-[(4-chlorophenyl)methoxy]phenyl]methyl]-4-piperidinol. ¹H NMR (DMSO-d₆/TFA): δ 1.60˜2.40 (m, 6H), 3.00˜4.10 (m, 11H), 4.30 (s, 2H), 5.16 (s, 2H), 7.10˜7.18 (m, 1H), 7.40˜7.52 (m, 4H), 7.56˜7.62 (m, 1H), 7.68˜7.71 (m, 1H), 8.20 (br.s, 3H), 9.60 (br.s, 1H).
1-[[5-Bromo-2-[(4-chlorophenyl)methoxy]phenyl]methyl]-4-[[4-[2-(dimethylamino)ethyl]-1-piperazinyl]methyl]-4-piperidinol. ¹H NMR (DMSO-d₆/TFA): δ 1.68˜2.02 (m, 4H), 2.74˜2.80 (m, 6H), 2.80˜3.40 (m, 18H), 4.27 (br.s, 2H), 5.16 (s, 2H), 7.12˜7.18 (m, 1H), 7.40˜7.52 (m, 4H), 7.56˜7.62 (m, 1H), 7.70 (d, 1H), 9.50 (br.s, 1H).
1-[[5-Bromo-2-[(4-chlorophenyl)methoxy]phenyl]methyl]-4-[[4-[2-(4-morpholinyl)-2-oxoethyl]-1-piperazinyl]methyl]-4-piperidinol. ¹H NMR (DMSO-d₆/TFA): δ 1.68˜2.02 (m, 4H), 3.02˜3.64 (m, 22H), 4.26 (br.s, 2H), 4.36 (br.s, 2H), 5.17 (s, 2H), 7.12˜7.18 (m, 1H), 7.40˜7.52 (m, 4H), 7.59 (dd, 1H), 7.70 (d, 1H), 9.55 (br.s, 1H).
1-[[5-Bromo-2-[(4-chlorophenyl)methoxy]phenyl]methyl]-4-[[4-[3-(4-morpholinyl)propyl]-1-piperazinyl]methyl]-4-piperidinol. ¹H NMR (DMSO-d₆/TFA): δ 1.15˜2.10 (m, 6H), 2.95˜4.10 (m, 26H), 4.26 (s, 2H), 5.16 (s, 2H), 7.10˜7.18 (m, 1H), 7.40˜7.50 (m, 4H), 7.56˜7.62 (m, 1H), 7.69 (d, 1H), 9.50 (br.s, 1H).
1-[[5-Bromo-2-[(4-chlorophenyl)methoxy]phenyl]methyl]-4-[[4-[2-(4-morpholinyl]-1-piperazinyl]methyl]-4-piperidinol. ¹H NMR (DMSO-d₆/TFA): δ 1.70˜2.05 (m, 4H), 2.85˜3.50 (m, 22H), 3.80 (br.s, 4H), 4.28 (br.s, 2H), 5.18 (s, 2H), 7.12˜7.18 (m, 1H), 7.40˜7.52 (m, 4H), 7.56˜7.62 (m, 1H), 7.70 (d, 1H), 9.52 (br.s, 1H).
1-[[5-Bromo-2-[(4-chlorophenyl)methoxy]phenyl]methyl]-4-[(dimethylamino)methyl]-4-piperidinol. ¹H NMR (DMSO-d₆/TFA): δ 1.68˜2.20 (m, 4H), 2.83 (m, 6H), 3.06˜3.34 (m, 6H), 4.24˜4.32 (m, 2H), 5.16 (s, 2H), 7.12˜7.18 (m, 1H), 7.40˜7.52 (m, 4H), 7.56˜7.60 (m, 1H), 7.70 (d, 1H), 9.30 (br.s, 1H), 9.60 (br.s, 1H).
1-[[5-Bromo-2-[(4-chlorophenyl)methoxy]phenyl]methyl]-4-[(diethylamino)methyl]-4-piperidinol. ¹H NMR (DMSO-d₆/TFA): δ 1.16˜1.24 (m, 6H), 1.72˜2.08 (m, 4H), 3.06˜3.36 (m, 10H), 4.28˜4.38 (m, 2H), 5.19 (s, 2H), 7.15˜7.20 (m, 1H), 7.42˜7.56 (m, 4H), 7.59˜7.64 (m, 1H), 7.72˜7.76 (m, 1H), 8.80 (br.s, 1H), 9.40 (br.s, 1H).
1-[[5-Bromo-2-[(4-chlorophenyl)methoxy]phenyl]methyl]-4-[[(1-methylethyl)amino]methyl]-4-piperidinol. ¹H NMR (DMSO-d₆/TFA): δ 1.20˜1.26 (m, 6H), 1.70˜2.04 (m, 4H), 2.88˜3.38 (m, 7H), 4.28˜4.34 (m, 2H), 5.20 (s, 2H), 7.14˜7.20 (m, 1H), 7.42˜7.54 (m, 4H), 7.58˜7.64 (m, 1H), 7.72˜7.74 (m, 1H), 8.24 (br.s, 2H), 9.50 (br.s, 1H).
1-[[5-Bromo-2-[(4-chlorophenyl)methoxy]phenyl]methyl]-4-[(4-hydroxy-1-piperidinyl)methyl]-4-piperidinol. ¹H NMR (DMSO-d₆/TFA): δ 1.20˜1.26 (m, 6H), 1.70˜2.04 (m, 4H), 2.88˜3.38 (m, 7H), 4.28˜4.34 (m, 2H), 5.20 (s, 2H), 7.14˜7.20 (m, 1H), 7.42˜7.54 (m, 4H), 7.58˜7.64 (m, 1H), 7.72˜7.74 (m, 1H), 8.24 (br.s, 2H), 9.50 (br.s, 1H).
1-[[5-Bromo-2-[(4-chlorophenyl)methoxy]phenyl]methyl]-4-[[(3R)-3-hydroxypyrrolidinyl]methyl]-4-piperidinol. ¹H NMR (DMSO-d₆/TFA): δ 1.68˜2.28 (m, 6H), 3.04˜3.78 (m, 10H), 4.24˜4.46 (m, 3H), 5.16 (s, 2H), 7.11˜7.17 (m, 1H), 7.50˜7.52 (m, 4H), 7.56˜7.60 (m, 1H), 7.70 (d, 1H), 9.60 (br.s, 1H).
1-[[5-Bromo-2-[(4-chlorophenyl)methoxy]phenyl]methyl]-4-[[[(4-fluorophenyl)methyl]amino]methyl]-4-piperidinol. ¹H NMR (DMSO-d₆/TFA): δ 1.64˜2.00 (m, 4H), 2.80˜3.32 (m, 6H), 4.14 (br.s, 2H), 4.24˜4.30 (m, 2H), 5.16 (m, 2H), 7.11˜7.29 (m, 3H), 7.40˜7.51 (m, 4H), 7.53˜7.61 (m, 3H), 7.67˜7.70 (m, 1H), 8.85 (br.s, 2H), 9.50 (br.s, 1H).
1-[[5-Bromo-2-[(4-chlorophenyl)methoxy]phenyl]methyl]-4-(1H-imidazol-1-ylmethyl)-4-piperidinol. ¹H NMR (DMSO-d₆/TFA): δ 1.46˜1.94 (m, 4H), 3.00˜3.30 m, 4H), 4.16˜4.34 (m, 4H), 5.15˜5.20 (m, 2H), 7.13˜7.17 (m, 1H), 7.40˜7.53 (m, 4H), 7.60 (dd, 1H), 7.64˜7.74 (m, 3H), 8.95 and 9.00 (each s, 1H), 9.50 (br.s, 1H).
1-[[5-Bromo-2-[(4-chlorophenyl)methoxy]phenyl]methyl]-4-[(phenylamino)methyl]-4-piperidinol. ¹H NMR (DMSO-d₆/TFA): δ 1.66˜2.04 (m, 4H), 3.08˜3.30 (m, 6H), 4.24˜4.32 (m, 2H), 5.16 (s, 2H), 6.70˜7.02 (m, 3H), 7.12˜7.28 (m, 3H), 7.38˜7.50 (m, 4H), 7.58 (dd, 1H), 7.68˜7.72 (m, 1H), 9.20˜9.40 (m, 1H).
1-[[5-Bromo-2-[(4-chlorophenyl)methoxy]phenyl]methyl]-4-[(4-pyridinylamino)methyl]-4-piperidinol. ¹H NMR (DMSO-d₆/TFA): δ 1.44˜1.96 (m, 4H), 3.00˜3.30(m, 4H), 4.00˜4.16 (m, 2H), 4.24˜4.34 (m, 2H), 5.14˜5.18 (m, 2H), 6.76˜6.82 (m, 2H), 7.14 (d, 1H), 7.38˜7.54 (m, 4H), 7.56˜7.62 (m, 1H), 7.68˜7.74 (m, 1H), 7.90˜8.02 (m, 2H), 8.10˜8.18 (m, 2H), 9.50 (br.s, 1H).
1-[[5-Bromo-2-[(4-chlorophenyl)methoxy]phenyl]methyl]-4-[[(2-hydroxyethyl)methylamino]methyl]-4-piperidinol. 1H NMR (DMSO-d₆/TFA): δ 1.68˜2.06 (m, 4H), 2.86˜2.94 (m, 3H), 3.04˜3.40 (m, 8H), 3.70˜3.80 (m, 2H), 4.26˜4.32 (m, 2H), 5.17 (s, 2H), 7.12˜7.18 (m, 1H), 7.40˜7.52 (m, 4H), 7.58 (dd, 1H), 7.70 (d, 1H), 8.99 (br.s, 2H), 9.50 (br.s, 1H).
1-[[5-Bromo-2-[(4-chlorophenyl)methoxy]phenyl]methyl]-4-[(4-methyl-1-piperazinyl)methyl]-4-piperidinol. ¹H NMR (DMSO-d₆/TFA): δ 1.70˜2.02 (m, 4H), 2.80˜2.88 (m, 3H), 3.06˜3.78 (m, 14H), 4.28 (br.s, 2H), 5.16 (s, 2H), 7.11˜7.16 (m, 1H), 7.40˜7.52 (m, 4H), 7.56˜7.60 (m, 1H), 7.70 (d, 1H), 9.60 (br.s, 1H).
1-[[5-Bromo-2-[(4-chlorophenyl)methoxy]phenyl]methyl]-4-[(dipropylamino)methyl]-4-piperidinol. ¹H NMR (DMSO-d₆/TFA): δ 0.82˜0.90 (m, 6H), 1.58˜2.08 (m, 8H), 3.00˜3.30 (m, 10H), 4.25˜4.35 (m, 2H), 5.18 (s, 2H), 7.12˜7.18 (m, 1H), 7.40˜7.52 (m, 4H), 7.60 (dd, 1H), 7.69˜7.73 (m, 1H), 8.99 (br.s, 1H), 9.70 (br.s, 1H).
1-[[1-[[5-Bromo-2-[(4-chlorophenyl)methoxy]phenyl]methyl]-4-hydroxy-4-piperidinyl]methyl]-N,N-diethyl-3-piperidinecarboxamide. ¹H NMR (DMSO-d₆/TFA): δ 0.94˜1.16 (m, 6H), 1.40˜2.10 (m, 8H), 3.00˜3.75 (m, 15H), 4.30(br.s, 2H), 5.18 (s, 2H), 7.12˜7.18 (m, 1H), 7.40˜7.52 (m, 4H), 7.59 (dd, 1H), 7.70 (m, 1H), 9.10˜9.30 (m, 1H), 9.50˜9.70 (m, 1H).
1-[[5-Bromo-2-[(4-chlorophenyl)methoxy]phenyl]methyl]-4-[[(2,2,2-trifluoroethyl)amino]methyl]-4-piperidinol. ¹H NMR (DMSO-d₆/TFA): δ 1.70˜2.04(m, 4H), 2.92˜3.32(m, 6H), 3.86(m, 2H), 4.26˜4.34(m, 2H), 5.18(s, 2H), 7.12˜7.18(m, 1H), 7.42˜7.52(m, 4H), 7.60(dd, 1H), 7.70(d, 1H), 9.60(m, 1H).
1-[[5-Bromo-2-[(4-chlorophenyl)methoxy]phenyl]methyl]-4-[[(3-methylphenyl)amino]methyl]-4-piperidinol. ¹H NMR (DMSO-d₆/TFA): δ 1.64˜2.02 (m, 4H), 2.16 and 2.22 (each s, 3H), 3.02˜3.30 (m, 6H), 4.22˜4.34 (m, 2H), 5.16 (br.s, 2H), 6.48˜6.76 (m, 3H), 6.98˜7.18 (m, 2H), 7.38˜7.52 (m, 4H), 7.60 (dd, 1H), 7.68˜7.72 (m, 1H), 9.10˜9.40 (m, 1H).
1-[[5-Bromo-2-[(4-chlorophenyl)methoxy]phenyl]methyl]-4-[[[(1R)-1-phenylethyl]amino]methyl]-4-piperidinol. ¹H NMR (DMSO-d₆/TFA): δ 1.52˜2.00 (m, 7H), 2.50˜3.34 (m, 6H), 4.22˜4.38 (m, 2H), 5.16 (br.s, 2H), 7.10˜7.16 (m, 1H), 7.35˜7.54 (m, 9H), 7.58 (dd, 1H), 7.65˜7.70 (m, 1H), 8.70˜9.60 (m, 3H).
4-[[1-[[5-Bromo-2-[(4-chlorophenyl)methoxy]phenyl]methyl]-4-hydroxy-4-piperidinyl]methyl]-N-ethyl-1-piperazinecarboxamide. ¹H NMR (DMSO-d₆/TFA): δ 0.93˜1.02 (m, 3H), 1.68˜2.06 (m, 4H), 3.00˜4.00 (m, 16H), 4.28 (br.s, 2H), 5.18 (s, 2H), 7.12˜7.17 (m, 1H), 7.41˜7.51 (m, 4H), 7.57˜7.61 (m, 1H), 7.70 (d, 1H), 9.55 (br.s, 1H).

### Example 61

### N-[[1-[[5-Bromo-2-[(4-chlorophenyl)methoxy]phenyl]methyl]-4-hydroxy-4-piperidinyl]methyl]-N′-(2,6-difluorophenyl)urea

To a stirred mixture of 4-(aminomethyl)-1-[[5-bromo-2-[(4-chlorophenyl)methoxy]phenyl]methyl]-4-piperidinol (150 mg, 0.34 mmol) and Et₃N (69 mg, 0.68 mmol) in CH₂Cl₂ (5 mL) was added 2,6-difluorophenyl isocyanate (59 mg, 0.37 mmol) dropwise at 0° C. After addition, the mixture was stirred overnight at rt. The mixture was poured into ice water (10 mL), and extracted with CH₂Cl₂ (3×15 mL). The organic phase was dried over Na₂SO₄, and concentrated in vacuo. The crude product was purified by column chromatography to afforded the title product. ¹H NMR: (DMSO-d₆, 400 MHz) 6:1.1 (s, 1H), 1.3-1.7 (m, 4H), 2.2-2.8 (m, 2H), 2.9-3.2 (m, 2H),3.4-3.6 (s, 2H),4.3-4.5 (s, 1H), 5.0-5.2 (s, 2H), 6.2-6.4 (m, 1H), 6.9-7.1 (m, 3H),7.1-7.3 (m, 1H), 7.3-7.5 (m, 5H), 7.9-8.1 (m, 1H).

The following compounds were prepared in a similar manner:
N-[[1-[[5-Bromo-2-[(4-chlorophenyl)methoxy]phenyl]methyl]-4-hydroxy-4-piperidinyl]methyl]-N′-(2,6-dimethoxyphenyl)urea. ¹H NMR (CDCl₃, 400 MHz): δ: 1.1-1.8 (m, 5H), 2.3-3.0 (m, 4H),3.2-3.3 (m, 2H), 3.5-3.7 (m, 2H), 3.8-3.9 (s, 6H), 5.0 (s, 2H), 0.2-5.4 (s, 1H), 5.8-6.0 (s, 1H),6.58-6.64 (d, 2H), 6.7-6.82 (d, 1H), 7.15-7.22 (m, 1H), 7.35 (s, 4H), 7.50-7.57 (m, 1H).
N-[[1-[[5-Bromo-2-[(4-chlorophenyl)methoxy]phenyl]methyl]-4-hydroxy-4-piperidinyl]methyl]-N′-(2,6-diethylphenyl)urea. ¹H NMR (CDCl₃, 400 MHz) δ:1.0-1.3 (t, 6H), 1.5-2.1 (m, 3H), 2.4-2.8 (m, 4H), 3.0-3.7 (m, 6H), 4.0-4.4 (m, 2H), 4.8-5.3 (m, 3H), 6.8-6.94 (m, 1H), 7.1-7.22 (m, 3H), 7.26-7.33 (m, 2H), 7.33-7.45 (m, 2H), 7.45-7.57 (m, 2H).
N-[[1-[[5-Bromo-2-[(4-chlorophenyl)methoxy]phenyl]methyl]-4-hydroxy-4-piperidinyl]methyl]-N′-(2,4,6-trichlorophenyl)urea. ¹H NMR (CDCl₃, 400 MHz) δ 1.6-1.8 (m, 4H), 2.3-2.5 (m,3 H), 2.5-2.7 (m, 2H), 3.2-3.4 (m, 2H), 3.5-3.7 (s, 2H), 4.9-5.1 (m, 3H), 6.7-6.8 (d, 1H), 7.26-7.31 (m, 1H), 7.32-7.36 (m, 4H), 7.37-7.41 (m, 2H), 7.47-7.52 (m, 1H).
N-[[1-[[5-Bromo-2-[(4-chlorophenyl)methoxy]phenyl]methyl]-4-hydroxy-4-piperidinyl]methyl]-N′-(2,6-dichlorophenyl)urea. ¹H NMR (DMSO, 400 MHz): δ 1.4-1.9 (m, 4H), 2.9-3.4 (m, 6H), 4.1-4.4 (m, 2H), 4.8-5.3 (m, 2H), 6.4-6.6 (m, 1H), 7.06-7.24 (m, 2H), 7.25-7.52 (m, 6H), 7.52-7.62 (m, 1H), 7.63-7.74 (d, 1H).
N-[[1-[[5-Bromo-2-[(4-chlorophenyl)methoxy]phenyl]methyl]-4-hydroxy-4-piperidinyl]methyl]-N′-(2,6-dimethylphenyl)urea. ¹H NMR (DMSO-d₆/TFA): δ 1.56˜1.84 (m, 4H), 2.08˜2.14 (m, 6H), 3.04˜3.28 (m, 6H), 4.22˜4.28 (m, 2H), 5.14 (m, 2H), 6.95˜7.02 (m, 3H), 7.11˜7.18 (m, 1H), 7.34˜7.52 (m, 4H), 7.56˜7.64 (m, 2H), 7.70 (d, 1H), 9.10˜9.30 (m, 1H).
N-[[1-[[5-Bromo-2-[(4-chlorophenyl)methoxy]phenyl]methyl]-4-hydroxy-4-piperidinyl]methyl]-N′-(2,6-dibromophenyl)urea. ¹H NMR (DMSO-d₆): δ 1.38˜1.52 (m, 4H), 2.10 (s, 6H) 2.30˜2.45 (m, 4H), 3.05 (d, 2H), 3.30 (s, 3H), 3.45 (s, 2H), 4.40 (s, 1H), 5.20 (s, 2H), 6.97 (d, 1H), 7.21 (s, 2H), 7.33 (dd, 1H), 7.38˜7.47 (m, 5H), 7.61 (s, 1H).
N-[[1-[[5-Bromo-2-[(4-chlorophenyl)methoxy]phenyl]methyl]-4-hydroxy-4-piperidinyl]methyl]-N′-(4-bromo-2,6-dimethylphenyl)urea. ¹H NMR (DMSO-d₆/TFA): δ 1.52˜1.84 (m, 4H), 2.02˜2.10 (m, 6H), 2.17 (s, 3H), 3.00˜3.30 (m, 6H), 4.24 (m, 2H), 5.12˜5.20 (m, 2H), 6.80 (s, 2H), 7.10˜7.19 (m, 1H), 7.32˜7.64 (m, 6H), 7.70 (d, 1H), 9.10˜9.30 (m, 1H).
N-[2,6-Bis(1-methylethyl)phenyl]-N′-[[1-[[5-bromo-2-[(4-chlorophenyl)methoxy]phenyl]methyl]-4-hydroxy-4-piperidinyl]methyl]urea. ¹H NMR (DMSO-d₆/TFA): δ 1.10 (d, 12H), 1.55˜1.85 (m, 4H), 3.00˜3.30 (m, 8H), 4.26 (br.s, 2H), 5.10˜5.20 (m, 2H), 6.35 (br.s, 1H), 7.02˜7.64 (m, 11H), 7.67˜7.72 (m, 2H), 9.10˜9.30 (m, 1H).
N-[[1-[[5-Bromo-2-[(4-chlorophenyl)methoxy]phenyl]methyl]-4-hydroxy-4-piperidinyl]methyl]-N′-(4-fluorophenyl)urea. ¹H NMR DMSO-d₆/TFA): δ 1.60˜1.95 (m, 4H), 3.10˜3.40 (m, 6H), 4.30˜4.40 (m, 2H), 5.25 (s, 2H), 7.08 (m, 2H), 7.20˜7.24 (m, 1H), 7.39˜7.46 (m, 2H), 7.48˜7.60 (m, 4H), 7.67 (m, 1H), 7.76˜7.80 (m, 1H), 8.70˜8.80 (m, 1H), 9.10˜9.30 (m, 1H).

### Example 62

### 2-Amino-N-[[1-[[5-bromo-2-[(4-chlorophenyl)methoxy]phenyl]methyl]-4-hydroxy-4-piperidinyl]methyl]acetamide

To a solution of 4-(aminomethyl)-1-[[5-bromo-2-[(4-chlorophenyl)methoxy]phenyl]methyl]-4-piperidinol (150 mg, 0.35 mmol), N-Boc-glycine (86 mg, 0.49 mmol), and Et₃N(0.3 mL, 2.1 mmol) in DMF (4 mL) was added HATU(180 mg, 0.48 mmol) at rt. After stirring at rt overnight, the mixture was poured into ice-water, and kept in the refrigerator overnight to precipitate the product. The crude product was collected by filtration, and the solid was re-dissolved in dichloromethane and dried over Na₂SO₄. Concentration in vacuo afforded the crude product, which was used directly in the next step. The residue was dissolved in TFA (5 mL) and CH₂Cl₂ (5 mL) and stirred at rt under N₂ for 1 h. After concentration, the residue was purified by HPLC to afford product as a white solid. ¹H NMR (DMSO-d₆/TFA): δ 1.50˜1.90 (m, 4H), 3.00˜3.30 (m, 6H), 3.50 (m, 2H), 4.20˜4.35 (m, 2H), 5.25 (m, 2H), 7.12˜7.17 (m, 1H), 7.40˜7.52 (m, 4H), 7.56˜7.60 (m, 1H), 7.69 (d, 1H), 7.94 (m, 3H), 8.28˜8.38 (m, 1H), 9.20˜9.30 (m, 1H).

### Example 63

### N-[2-[[[1-[[5-Bromo-2-[(4-chlorophenyl)methoxy]phenyl]methyl]-4-hydroxy-4-piperidinyl]methyl]amino]-2-oxoethyl]-2,6-difluorobenzamide

To a solution of 4-(aminomethyl)-1-[[5-bromo-2-[(4-chlorophenyl)methoxy]phenyl]methyl]-4-piperidinol (150 mg, 0.30 mmol), 2,6-difluorobenzoic acid (57 mg, 0.36 mmol), and Et₃N (0.15 mL, 1.1 mmol) in DMF (5 mL) was added HATU (150 mg, 0.4 mmol) at RT. After stirring at rt under overnight (the reaction was checked by TLC), the reaction mixture was poured into ice-water. The crude product was collected by filtration, and re-dissolved in CH₂Cl₂. The organic phase was dried over Na₂SO₄, and concentrated. The residue was purified by HPLC to afford the title compound as a light yellow powder. ¹H NMR (DMSO-d₆/TFA): δ 1.50˜1.90 (m, 4H), 3.00˜3.28 (m, 6H), 3.90 (m, 2H), 4.20 (m, 2H), 5.15 (s, 2H), 7.04˜7.16 (m, 3H), 7.38˜7.52 (m, 5H), 7.57 (m, 1H), 7.68 (d, 2H), 7.82˜7.94 (m, 1H), 8.86˜8.96 (m, 1H), 9.02˜9.20 (m, 1H).

The following compounds were prepared in a similar manner:
N-[[1-[[5-Bromo-2-[(4-chlorophenyl)methoxy]phenyl]methyl]-4-hydroxy-4-piperidinyl]methyl]benzamide. ¹H NMR (DMSO-d₆/TFA): δ 1.72˜2.10 (m, 4H), 3.30˜3.62 (m, 6H), 4.40˜4.52 (m, 2H), 5.32˜5.36 (m, 2H), 7.30˜7.36 (m, 1H), 7.58˜7.70 (m, 7H), 7.74˜7.78 (m, 1H), 7.84˜7.92 (m, 1H), 8.00˜8.06 (m, 2H), 8.54˜8.68 (m, 1H), 9.20˜9.40 (m, 1H).
N-[[1-[[5-Bromo-2-[(4-chlorophenyl)methoxy]phenyl]methyl]-4-hydroxy-4-piperidinyl]methyl]-4-chlorobenzamide. ¹H NMR (DMSO-d₆/TFA): δ 1.52˜1.90 (m, 4H), 3.00˜3.40 (m, 6H), 4.20˜4.32 (m, 2H), 5.12˜5.16 (m, 2H), 7.09˜7.14 (m, 1H), 7.36˜7.52 (m, 6H), 7.54˜7.58 (m, 1H), 7.64˜7.70 (m, 1H), 7.82˜7.90 (m, 2H), 8.44˜8.56 (m, 1H), 9.00˜9.20 (m, 1H).
3-[[[[1-[[5-Bromo-2-[(4-chlorophenyl)methoxy]phenyl]methyl]-4-hydroxy-4-piperidinyl]methyl]amino]carbonyl]-1-hydroxy-2,4-dimethylpyridinium. ¹H NMR (DMSO-d₆/TFA): δ 1.56˜1.98 (m, 4H), 3.20˜3.46 (m, 6H), 4.22˜4.38 (m, 2H), 5.16˜5.22 (m, 2H), 7.14˜7.20 (m, 1H), 7.42˜7.56 (m, 5H), 7.58˜7.64 (m, 2H), 7.68˜7.76 (m, 1H), 7.80˜7.88 (m, 1H), 7.94˜7.96 (m, 1H), 8.56˜8.68 (m, 1H), 9.00˜9.30 (m, 1H). LC-MS (API150EX): calcd:578, found:578.
N-[[1-[[5-Bromo-2-[(4-chlorophenyl)methoxy]phenyl]methyl]-4-hydroxy-4-piperidinyl]methyl]acetamide. ¹H NMR (DMSO-d₆/TFA): δ 1.50˜1.72 (m, 4H), 1.80 and 1.86(each s, 3H), 3.00˜3.26 (m, 6H), 4.20˜4.30 (m, 2H), 5.16 (m, 2H), 7.12˜7.18 (m, 1H), 7.40˜7.50 (m, 4H), 7.56˜7.62 (m, 1H), 7.68˜7.72 (m, 1H), 7.84˜7.96 (m, 1H), 9.15˜9.30 (m, 1H).

### Example 64

### 2-(Acetylamino)-N-[[1-[[5-bromo-2-[(4-chlorophenyl)methoxy]phenyl]methyl]-4-hydroxy-4-piperidinyl]methyl]acetamide

To a solution of 4-(aminomethyl)-1-[[5-bromo-2-[(4-chlorophenyl)methoxy]phenyl]methyl]-4-piperidinol (100 mg, 0.2mmol) and Et₃N(1 mL, 7 mmol) in CH₂Cl₂ (4 mL) was added Ac₂O (0.5 mL, 5 mmol) at rt. The mixture was stirred at rt overnight, and then concentrated. The residue was purified by HPLC to afford the title compound as a white powder. ¹H NMR (DMSO-d₆/TFA): δ 1.50˜1.68(m, 4H), 1.80˜1.83 (m, 3H), 3.00˜3.24 (m, 6H), 3.62˜3.70 (m, 2H), 4.20˜4.32 (m, 2H), 5.14˜5.18 (m, 2H), 7.10˜7.16 (m, 1H), 7.40˜7.52 (m, 4H), 7.56˜7.60 (m, 1H), 7.66˜7.72 (m, 1H), 7.78˜7.90 (m, 1H), 8.03˜8.15 (m, 1H), 9.00˜9.20 (m, 1H).

The following compounds were prepared in a similar manner:
[2-[[[1-[[5-Bromo-2-[(4-chlorophenyl)methoxy]phenyl]methyl]-4-hydroxy-4-piperidinyl]methyl]amino]-2-oxoethyl]carbamic acid, phenylmethyl ester. ¹H NMR (DMSO-d₆/TFA): δ 1.50˜1.90 (m, 4H), 3.00˜3.25 (m, 6H), 3.85˜3.90 (m, 2H), 4.20˜4.30 (m, 2H), 5.15˜5.20 (m, 2H), 7.10˜7.16 (m, 1H), 7.40˜7.52 (m, 7H), 7.56˜7.62 (m, 1H), 7.69(d, 1H), 7.82˜7.86 (m, 2H), 7.88˜7.89 (m, 1H), 8.66˜8.80 (m, 1H), 9.10˜9.20 (m, 1H).
(αS)-a-Amino-N-[[1-[[5-bromo-2-[(4-chlorophenyl)methoxy]phenyl]methyl]-4-hydroxy-4-piperidinyl]methyl]benzeneacetamide. ¹H NMR (DMSO-d₆/TFA): δ 1.24˜1.84 (m, 4H), 2.86˜3.28 (m, 8H), 4.20 (m, 2H), 4.22˜4.32 (m, 2H), 5.14˜5.20 (m, 2H), 7.12˜7.30 (m, 6H), 7.40˜7.52 (m, 4H), 7.59 (m, 1H), 7.68˜7.70 (m, 1H), 8.10 (br.s, 3H), 8.40˜8.50 (m, 1H), 9.20˜9.30 (m, 1H).
N-[[1-[[5-Bromo-2-[(4-chlorophenyl)methoxy]phenyl]methyl]-4-hydroxy-4-piperidinyl]methyl]-2-chloroacetamide. ¹H NMR (CDCl₃): δ 1.58˜1.74 (m, 4H), 2.44 (m, 2H), 2.64 (m, 2H), 3.37 (d, 2H), 3.57 (s, 2H), 4.10 (s, 2H), 5.02 (s, 2H), 6.75 (d, 1H), 6.95 (m, 1H), 7.29 (dd, 1H), 7.35 (m, 4H), 7.50 (d, 1H).
N-[[1-[[5-Bromo-2-[(4-chlorophenyl)methoxy]phenyl]methyl]-4-hydroxy-4-piperidinyl]methyl]-N-methylacetamide. ¹H NMR (DMSO-d₆/TFA): δ 1.92˜2.40 (m, 4H), 2.36˜2.42 (m, 3H), 3.20˜3.78 (m, 9H), 4.64˜4.72 (m, 2H), 5.56 (m, 2H), 7.53˜7.58 (m, 1H), 7.82˜7.94 (m, 4H), 7.97˜8.20 (m, 1H), 8.09˜8.13 (m, 1H), 9.40˜9.60 (m, 1H).

### Example 65

This Example illustrates the preparation of representative pharmaceutical compositions for oral administration containing a compound of the invention, or a pharmaceutically acceptable salt thereof:

| | | |
|---|---|---|
| A. | Ingredients | % wt./wt. |

| | |
|---|---|
| Compound of the invention | 20.0% |
| Lactose | 79.5% |
| Magnesium stearate | 0.5% |

The above ingredients are mixed and dispensed into hard-shell gelatin capsules containing 100 mg each, one capsule would approximate a total daily dosage.

| | | |
|---|---|---|
| B. | Ingredients | % wt./wt. |

| | |
|---|---|
| Compound of the invention | 20.0% |
| Magnesium stearate | 0.9% |
| Starch | 8.6% |
| Lactose | 69.6% |
| PVP (polyvinylpyrrolidine) | 0.9% |

The above ingredients with the exception of the magnesium stearate are combined and granulated using water as a granulating liquid. The formulation is then dried, mixed with the magnesium stearate and formed into tablets with an appropriate tableting machine.

| | |
|---|---|
| C. | Ingredients |

| | | |
|---|---|---|
| Compound of the invention | 0.1 | g |
| Propylene glycol | 20.0 | g |
| Polyethylene glycol 400 | 20.0 | g |
| Polysorbate 80 | 1.0 | g |
| Water | q.s. 100 | mL |

The compound of the invention is dissolved in propylene glycol, polyethylene glycol 400 and polysorbate 80. A sufficient quantity of water is then added with stirring to provide 100 mL of the solution, which is filtered and bottled.

| | | |
|---|---|---|
| D. | Ingredients | % wt./wt. |

| | |
|---|---|
| Compound of the invention | 20.0% |
| Peanut Oil | 78.0% |
| Span 60 | 2.0% |

The above ingredients are melted, mixed and filled into soft elastic capsules.

| | | |
|---|---|---|
| E. | Ingredients | % wt./wt. |

| | |
|---|---|
| Compound of the invention | 1.0% |
| Methyl or carboxymethyl cellulose | 2.0% |
| 0.9% saline | q.s. 100 mL |

The compound of the invention is dissolved in the cellulose/saline solution, filtered and bottled for use.

### Example 66

This Example illustrates the preparation of a representative pharmaceutical formulation for parenteral administration containing a compound of the invention, or a pharmaceutically acceptable salt thereof:

| Ingredients | | |
|---|---|---|
| Compound of the invention | 0.02 | g |
| Propylene glycol | 20.0 | g |
| Polyethylene glycol 400 | 20.0 | g |
| Polysorbate 80 | 1.0 | g |
| 0.9% Saline solution | q.s. 100 | mL |

The compound of the invention is dissolved in propylene glycol, polyethylene glycol 400 and polysorbate 80. A sufficient quantity of 0.9% saline solution is then added with stirring to provide 100 mL of the I.V. solution, which is filtered through a 0.2 m membrane filter and packaged under sterile conditions.

### Example 67

This Example illustrates the preparation of a representative pharmaceutical composition in suppository form containing a compound of the invention, or a pharmaceutically acceptable salt thereof:

| Ingredients | % wt./wt. |
|---|---|
| Compound of the invention | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

### Example 68

This Example illustrates the preparation of a representative pharmaceutical formulation for insufflation containing a compound of the invention, or a pharmaceutically acceptable salt thereof:

| Ingredients | % wt./wt. |
|---|---|
| Micronized compound of the invention | 1.0% |
| Micronized lactose | 99.0% |

The ingredients are milled, mixed, and packaged in an insufflator equipped with a dosing pump.

### Example 69

This Example illustrates the preparation of a representative pharmaceutical formulation in nebulized form containing a compound of the invention, or a pharmaceutically acceptable salt thereof:

| Ingredients | % wt./wt. |
|---|---|
| Compound of the invention | 0.005% |
| Water | 89.995% |
| Ethanol | 10.000% |

The compound of the invention is dissolved in ethanol and blended with water. The formulation is then packaged in a nebulizer equipped with a dosing pump.

### Example 70

This Example illustrates the preparation of a representative pharmaceutical formulation in aerosol form containing a compound of the invention, or a pharmaceutically acceptable salt thereof:

| Ingredients | % wt./wt. |
|---|---|
| Compound of the invention | 0.10% |
| Propellant 11/12 | 98.90% |
| Oleic acid | 1.00% |

The compound of the invention is dispersed in oleic acid and the propellants. The resulting mixture is then poured into an aerosol container fitted with a metering valve.

### Example 71

CCR-5 Receptor MIP-1a Scintillation Proximity Binding Assay

A) Assay Buffer: 50 mM Hepes, 5 mM MgCl2, 1 mM CaCl2, 30 ug/ml bacitracin, 0.1% BSA, pH 7.4.

B) Ligand: MIP-1a labeled with I-125 at 20,000-25,000 cpm/well. Non specific binding (nsb) was defined as bound cpm in the presence of 100 nM unlabeled MIP-1b.

C) Cells: Human embryonic kidney, (HEK-293) expressing human CCR-5 and CD4 pretreated overnight with 5 mM sodium butyrate. Harvest cells with calcium and magnesium free phosphate buffered saline. Cell number is counted with hemacytometer. Cell number per assay point was selected so the total counts per minute (cpm) bound was approximately 10% of the total cpms I-125-MIP-1a added per assay point.

D) Beads: Use wheatgerm agglutinin coated scintillation proximity assay beads (sold by Amersham Pharmacia Biotech Inc.) hydrated with the assay buffer for at least an hour before use. Final bead concentration was 0.2 mg beads per well.

E) Scintillation Proximity Assay: 100 ul of assay volume: 60 ul of cell/beads mix (premixed for at least 30 minutes), 20 ul of I-125-MIP-1a, 20 ul of assay buffer for total binding value, or 20 ul of 0.5 uM MIP-1b for nsb, or 20 ul of test compound. Shake the 96 well plates for 30 minutes on an orbital shaker, then let them settle for 30 minutes before reading with a scintillation counter.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of formula I 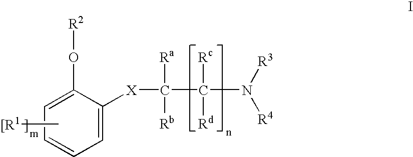
enantiomers, diastereomers, salts and solvates thereof
wherein
X is a bond or oxygen;
m is 0, 1, 2, 3 or 4;
n is 0, 1 or 2;
R¹ is an optional substituent independently selected at each occurrence from halogen, alkyl, haloalkyl, nitro, or —NR⁵R⁶;
R² is
a) hydrogen or
b) alkyl, cycloalkyl, alkenyl, aryl or heteroaryl any of which may be optionally substituted with a group Y;
Y is
a) aryl or heteroaryl either of which may be optionally substituted with one or more Z¹, Z², Z³;
b) cycloalkyl or heterocyclo either of which optionally substituted with one or more Z¹, Z², Z³;
c) —COOR⁷;
d) —NR⁸R⁹;
e) —CHR¹⁰(OR¹¹);
f) —C(═O)—NR⁸R⁹;
g) —NR¹²—(C═O)—NTR⁸R⁹;
h) —CN;
i) —C(═N—OR¹³);
j) alkoxy;
R³ and R⁴ are independently selected from
a) hydrogen;
b) alkyl, cycloalkyl, (cycloalkyl)alkyl, aryl, (aryl)alkyl, heterocyclo, (heterocyclo)alkyl, heteroaryl, or (heteroaryl)alkyl any of which may be optionally substituted with one or more Z¹, Z², Z³; or
c) —C(O)R*, —C(O)OR*, —C(O)NHR* or —SO₂R*;
or R³ and R⁴ together with the nitrogen atom to which they bonded may combine to form a heterocylo or heteroaryl ring optionally substituted with one or more Z¹, Z², Z³;
R⁵ and R⁶ are independently H, —C(O)R*, —SO₂R*, or —C(O)NR^{8a}R^{9a};
R⁷, R⁸, R^{8a}, R⁹, and R^{9a} are independently
a) hydrogen or
b) alkyl, cycloalkyl, (cycloalkyl)alkyl, aryl, (aryl)alkyl, heterocyclo, (heterocyclo)alkyl, heteroaryl, or (heteroaryl)alkyl any of which may be optionally substituted with one or more Z¹, Z², Z³;
R¹⁰ is H, alkyl or —OR*;
R¹¹ and R¹² are independently H or alkyl;
R¹³ is alkyl;
R* at each occurrence is independently alkyl, cycloalkyl, (cycloalkyl)alkyl, aryl, (aryl)alkyl, heterocyclo, (heterocyclo)alkyl, heteroaryl, or (heteroaryl)alkyl any of which may be optionally substituted with one or more Z¹, Z², Z³;
R^{a} and R^{b} are independently hydrogen, —OR^{10a}, alkyl, hydroxyalkyl, or haloalkyl;
or R^{a} and R^{b} may combine to form oxo;
R^{c} and R^{d} at each occurrence are independently H, —OR^{10b}, alkyl or haloalkyl
R^{10a} and R^{10b} are independently hydrogen, alkyl, haloalkyl, aryl, or heteroaryl;
Z¹, Z² and Z³ are optional substituents independently selected from
(1) V, where V is
(i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;
(ii) a group (i) which is itself substituted by one or more of the same or different groups (i); or
(iii) a group (i) or (ii) which is independently substituted by one or more (preferably 1 to 3) of the following groups (2) to (13) of the definition of Z¹,
(2) —OH or —OV,
(3) —SH or —SV,
(4) —C(O)H, —C(O)OH, —C(O)V, —C(O)OV or —O—C(O)V,
(5) —SO₃H, —S(O)ₜV, or S(O)ₜN(V¹)V, where t is 1 or 2,
(6) halo,
(7) cyano,
(8) nitro,
(9) —U¹—NV²V³,
(10) —U¹—N(V¹)—U²—NV²V³,
(11) —U¹—N(V⁴)—U²—V,
(12) —U¹—N(V⁴)—U²—H,
(13) oxo;
U¹ and U² are each independently
(1) a single bond,
(2) —U³—S(O)ₜ—U⁴—,
(3) —U³—C(O)—U⁴—,
(4) —U³—C(S)—U⁴—,
(5) —U³—O—U⁴—,
(6) —U³—S—U⁴—,
(7) —U³—O—C(O)—U⁴—,
(8) —U³—C(O)—O—U⁴—,
(9) —U³—C(═NV^{1a})—U⁴—, or
(10) —U³—C(O)—C(O)—U⁴—;
V¹, V^{1a}, V², V³ and V⁴
(1) are each independently hydrogen or a group provided in the definition of Z¹; or
(2) V² and V³ may together be alkylene or alkenylene, completing a 3- to 8-membered saturated or unsaturated ring together with the atoms to which they are attached, which ring is unsubstituted or substituted with one or more groups listed in the definition of Z¹, or
(3) V² or V³, together with V¹, may be alkylene or alkenylene completing a 3- to 8-membered saturated or unsaturated ring together with the nitrogen atoms to which they are attached, which ring is unsubstituted or substituted with one or more groups listed in the definition of Z¹; and
U³ and U⁴ are each independently
(1) a single bond,
(2) alkylene,
(3) alkenylene, or
(4) alkynylene.

2. A compound of claim 1 wherein
R² is alkyl substituted with Y;
Y is aryl, cycloalkyl, heterocyclo, —CHR¹⁰(OR¹¹), or —NR¹²—(C═O)—NR⁸R⁹ any of which may be optionally substituted with one or more Z¹, Z² and Z³.

3. A compound of claim 2 wherein R² is methyl.

4. A compound of claim 2 wherein Y is phenyl, cyclopropyl or 1,3 dioxolanyl any of which may be optionally substituted with one or more Z¹, Z² and Z³.

5. A compound of claim 4 wherein R² is methyl.

6. A compound of claim 5 wherein Y is phenyl substituted with at least one Z¹ group selected from alkyl, halo, haloalkyl, cyano, —C(O)OH, —C(O)V, —C(O)OV, and —C(O)—NV²V³.

7. A compound of claim 6 wherein R² is a group selected from

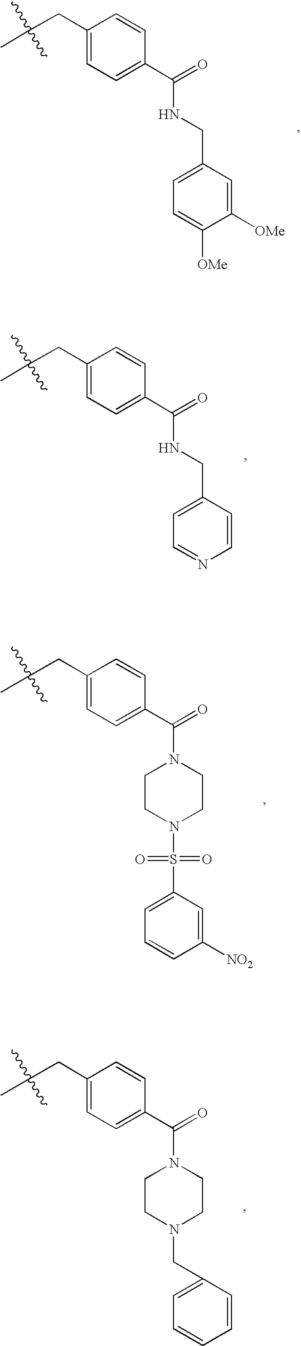

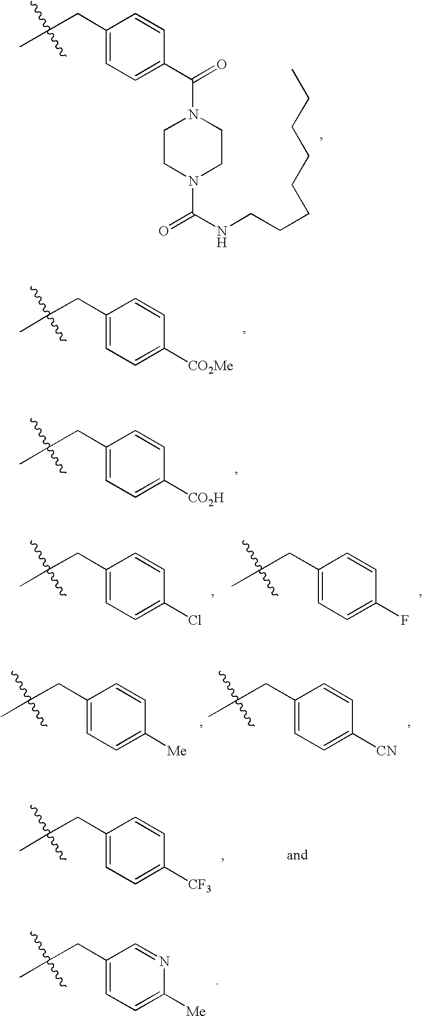

8. A compound of claim 4 wherein
(a) R³ and R⁴ are independently H, alkyl, (hydroxy)alkyl, (heteroaryl)alkyl, (heterocyclo)alkyl or —C(O)NHR* any of which may be optionally substituted with one or more Z¹, Z², Z³; or
(b) R³ and R⁴ together with the nitrogen atom to which they are bonded, combine to form a heterocylo or heteroaryl ring selected from

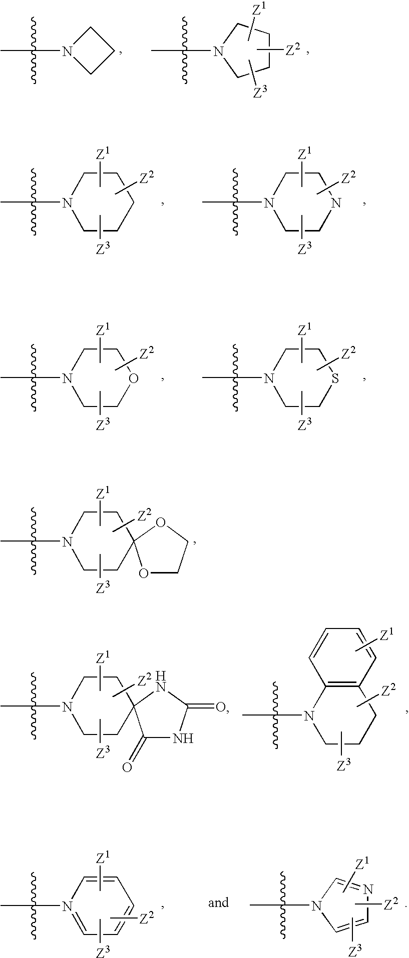

9. A compound of claim 8 wherein —NR³R⁴ is a group selected from

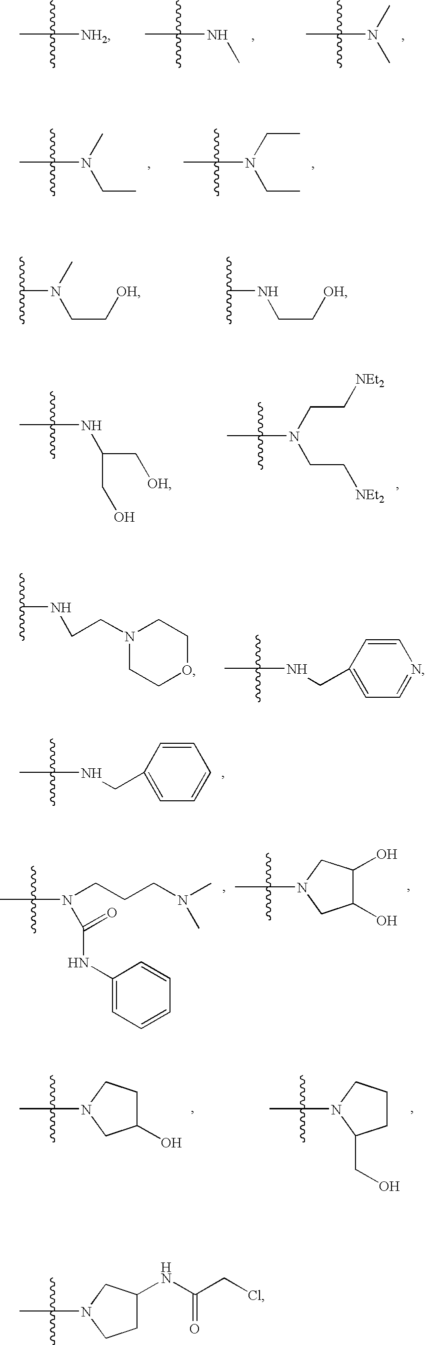

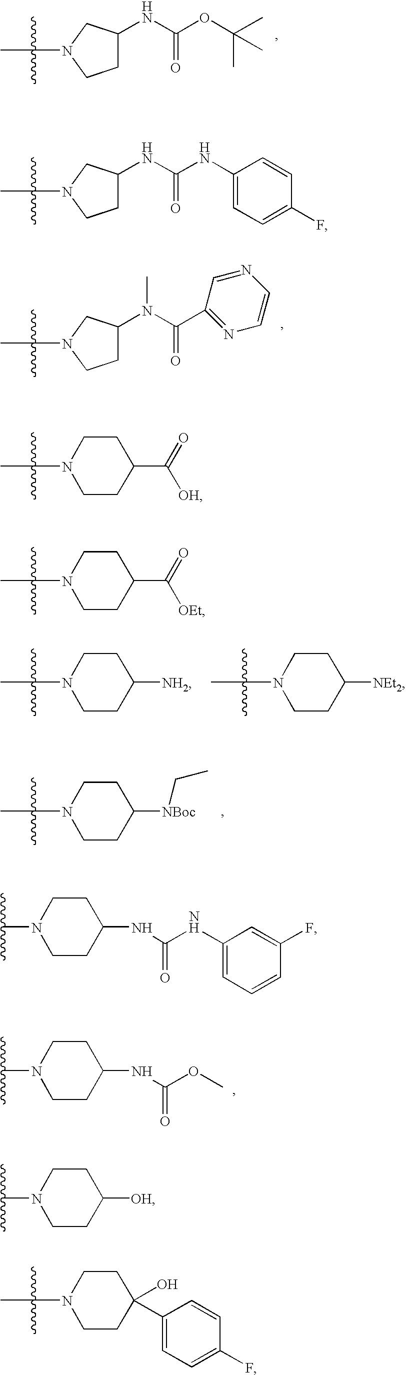

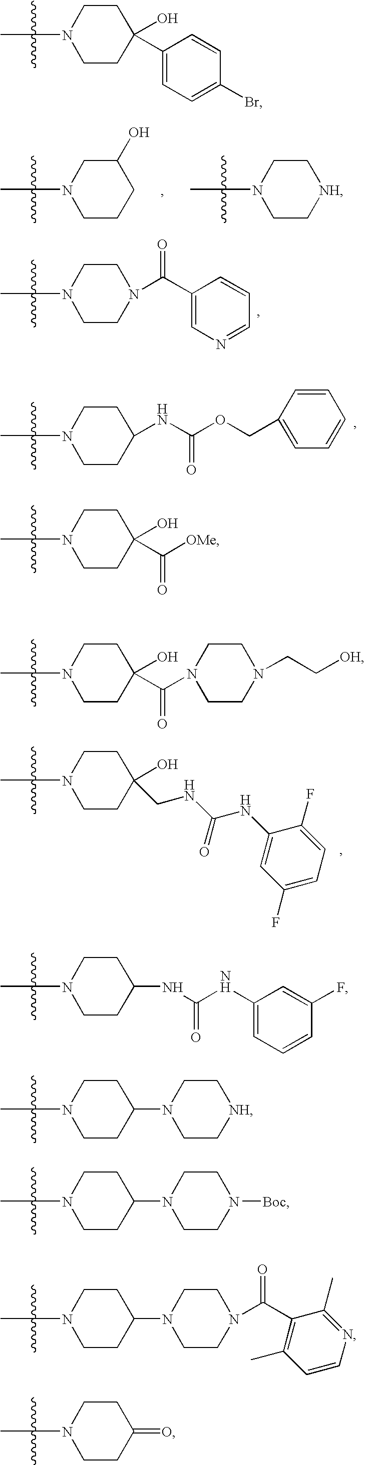

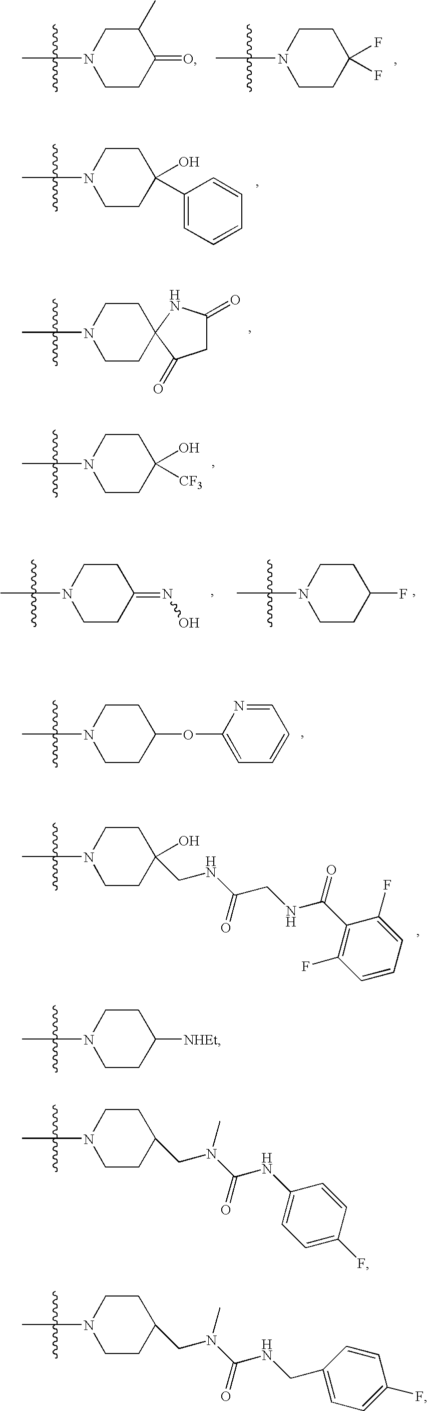

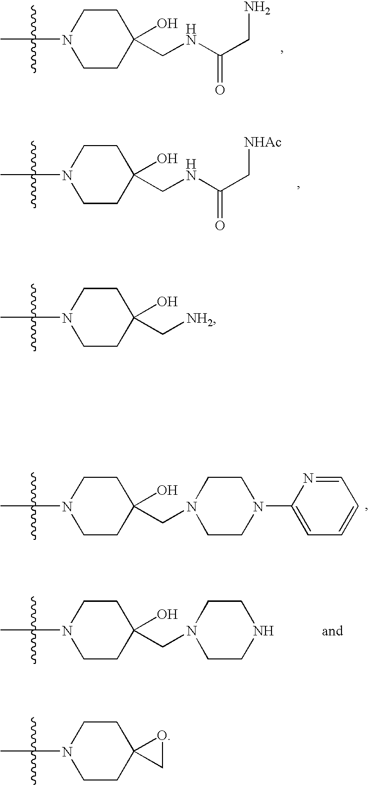

10. A compound of claim 9 wherein R² is a group selected from

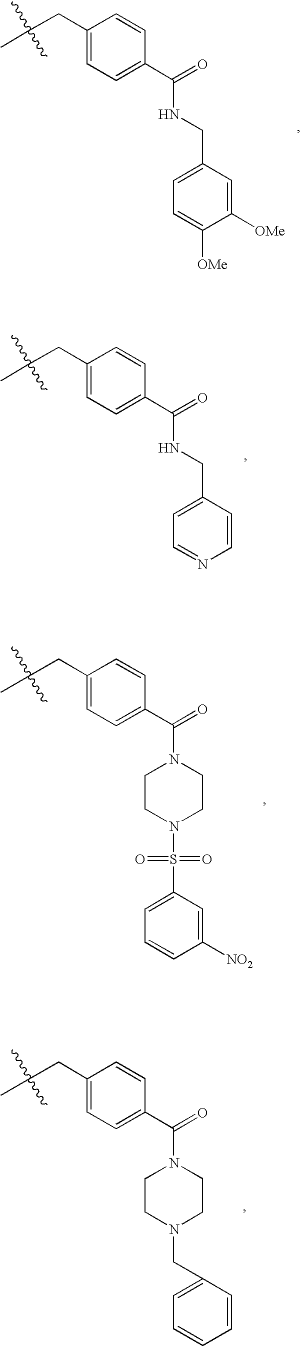

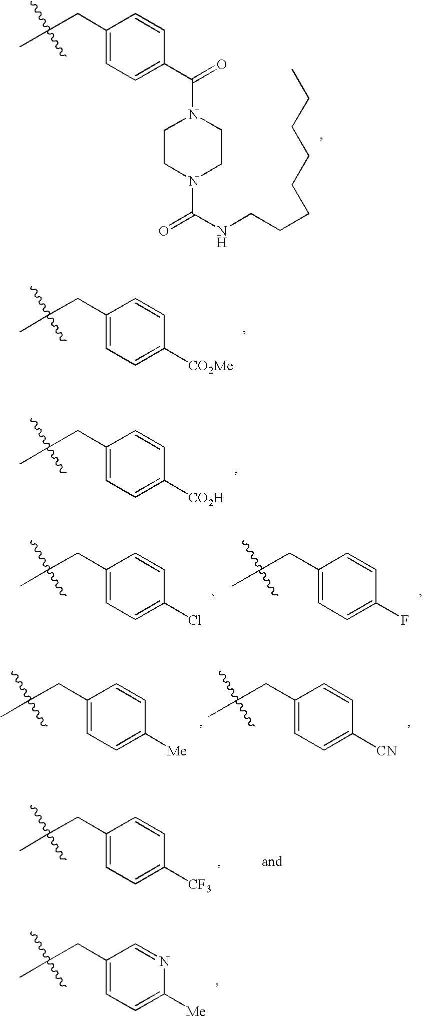

11. A compound of claim 1, 2 or 4 having the following formula II 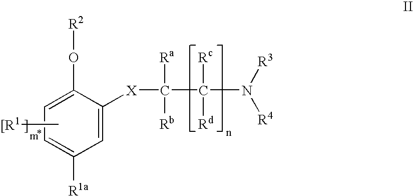
enantiomers, diastereomers, salts and solvates thereof
wherein
m* is 0, 1, 2, or 3; and
R^{1a} is halo.

12. A compound of claim 11 having the following formula III 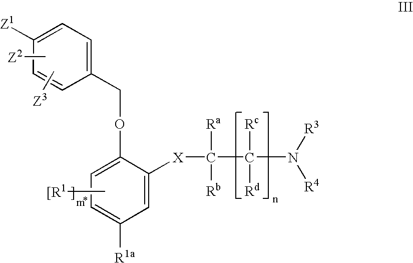
enantiomers, diastereomers, salts and solvates thereof
wherein
Z¹ is halo, cyano, alkyl, haloalkyl, aryl, —C(O)OH, —C(O)V, —C(O)OV, or —U¹—NV²V³.

13. A compound selected from
N-[[5-bromo-2-(4-chlorophenylmethoxy)phenyl]methyl]morpholineethanamine, dihydrochloride
5-bromo-2-(4-chlorophenylmethoxy)-N,N-diethylbenzenemethanamine, hydrochloride;
1-[[[5-bromo-2-(4-chlorophenylmethoxy)phenyl]methyl]amino]-2-propanol, Hydrochloride
1-[[5-bromo-2-(4-chlorophenylmethoxy)phenyl]methyl]-4-ethylpiperazine, dihydrochloride
N-[[5-bromo-2-(4-chlorophenylmethoxy)phenyl]methyl]-N′,N′-dimethylpropanediamine, dihydrochloride;
3-[[4-bromo-2-[(diethylamino)methyl]phenoxy]methyl]benzoic acid, methyl ester, hydrochloride;
4-[[5-bromo-2-[(4-chlorophenyl)methoxy]phenyl]methyl]thiomorpholine;
5-bromo-2-[(4-chlorophenyl)methoxy]-N-methyl-N-(phenylmethyl)benzenemethanamine;
5-bromo-2-[(4-chlorophenyl)methoxy]-N-ethylbenzenemethanamine;
4-[[5-bromo-2-[(4-chlorophenyl)methoxy]phenyl]methyl]morpholine;
5-bromo-2-[(4-chlorophenyl)methoxy]-N-(phenylmethyl)benzenemethanamine;
5-bromo-2-[(4-chlorophenyl)methoxy]-N,N-dimethylbenzenemethanamine;
[1-[[5-bromo-2-[(4-chlorophenyl)methoxy]phenyl]methyl]-3-pyrrolidinyl]-carbamic acid-1,1-dimethylethyl ester;
3-[[4-bromo-2-[(diethylamino)methyl]phenoxy]methyl]benzoic acid, methyl ester, hydrochloride;
1-[[5-bromo-2-[(4-iodophenyl)methoxy]phenyl]methyl]-4-piperidinol;
1-[[5-bromo-2-[(4-methylphenyl)methoxy]phenyl]methyl]-4-piperidinol;
1-[[5-bromo-2-[(4-chlorophenyl)methoxy]phenyl]methyl]-4-piperidinol;
1-[[5-bromo-2-[(6-methyl-3-pyridinyl)methoxy]phenyl]methyl]-4-piperidinol;
1-[[4-bromo-2-[(6-methyl-3-pyridinyl)methoxy]phenyl]methyl]-4-(4-bromophenyl)-4-piperidinol;
4-[[4-chloro-2-(4-morpholinylmethyl)phenoxy]methyl]benzonitrile;
4[[4-chloro-2-(1-pyrrolidinylmethyl)phenoxy]methyl]benzonitrile;
1-[[5-bromo-2-[(4-chlorophenyl)methoxy]phenyl]methyl]-3-piperidinemethanol;
N-[[5-bromo-2-(4-chlorophenylmethoxy)phenyl]methyl]-N-(3-dimethylaminopropyl)-N′-phenylurea, hydrochloride;
4-[[4-bromo-2-[(dimethylamino)methyl]phenoxy]methyl]-N-(3,4-dimethoxyphenylmethyl)benzamide, hydrochloride;
5-bromo-2-[[4-[(6,7-dimethoxy-3,4-dihydro-2(1H)-isoquinolinyl)carbonyl]phenyl]methoxy]-N,N-dimethylbenzenemethanamine, hydrochloride;
4-bromo-2-(bromomethyl)-1-[(4-chlorophenyl)methoxy]benzene;
2-[[[5-bromo-2-[(4-chlorophenyl)methoxy]phenyl]methyl]amino]-1,3-propanediol;
(2R)-1-[[5-bromo-2-[(4-chlorophenyl)methoxy]phenyl]methyl]-2-pyrrolidinemethanol, trifluoroacetic acid salt;
(2S)-1-[[5-bromo-2-[(4-chlorophenyl)methoxy]phenyl]methyl]-2-pyrrolidinemethanol, trifluoroacetic acid salt;
(2R)-1-[[5-bromo-2-[(4-chlorophenyl)methoxy]phenyl]methyl]-3-pyrrolidinol, trifluoroacetic acid salt;
N¹-[[5-bromo-2-[(4-chlorophenyl)methoxy]phenyl]methyl]-N¹-[2-(diethylamino)ethyl]-N²,N²-diethyl-1,2-ethanediamine;
1-[[5-bromo-2-[(4-chlorophenyl)methoxy]phenyl]methyl]-4-piperidinone;
1-[[5-bromo-2-[(4-chlorophenyl)methoxy]phenyl]methyl]-4-(4-bromophenyl)-4-piperidinol;
1-[[5-bromo-2-[(4-chlorophenyl)methoxy]phenyl]methyl]-4-piperidinol;
[1-[[5-bromo-2-[(4-chlorophenyl)methoxy]phenyl]methyl]-4-piperidinyl]-carbamic acid, 1,1-dimethylethyl ester;
1-[[5-bromo-2-[(4-chlorophenyl)methoxy]phenyl]methyl]-4-ethoxy-piperidine.
8-[[5-bromo-2-[(4-chlorophenyl)methoxy]phenyl]methyl]-1,4-dioxa-8-azaspiro[4.5]decane;
[[1-[[5-bromo-2-[(4-chlorophenyl)methoxy]phenyl]methyl]-4-piperidinyl]methyl]-carbamic acid, 1,1-dimethylethyl ester;
5-bromo-2-[(4-chlorophenyl)methoxy]benzenemethanamine;
1-[[5-bromo-2-[(4-chlorophenyl)methoxy]phenyl]methyl]pyridinium bromide;
1-[[5-bromo-2-[(4-chlorophenyl)methoxy]phenyl]methyl]-4-piperidinecarboxylic acid, ethyl ester;
2-[[[5-bromo-2-[(4-chlorophenyl)methoxy]phenyl]methyl]amino]ethanol;
2-[[[5-bromo-2-[(4-chlorophenyl)methoxy]phenyl]methyl](methyl)amino]-ethanol;
1-[[5-bromo-2-[(4-chlorophenyl)methoxy]phenyl]methyl]-3-piperidinol;
1-[[5-bromo-2-[(4-chlorophenyl)methoxy]phenyl]methyl]-3-pyrrolidinol;
(1S,2S)-2-[[[5-bromo-2-[(4-chlorophenyl)methoxy]phenyl]methyl]amino]-1-(4-nitrophenyl)-1,3-propanediol;
5-bromo-2-[(4-chlorophenyl)methoxy]-N,N,N-trimethyl-benzenemethanaminium iodide;
(3R,4S)-1-[[5-bromo-2-[(4-chlorophenyl)methoxy]phenyl]methyl]-3,4-pyrrolidinediol;
1-[[5-bromo-2-[(4-chlorophenyl)methoxy]phenyl]methyl]-4-piperidinecarboxylic acid;
1-[[5-bromo-2-[(4-chlorophenyl)methoxy]phenyl]methyl]-4-piperidinamine;
1-[[5-bromo-2-[(4-chlorophenyl)methoxy]phenyl]methyl]-4-piperidinemethanamine;
1-[[5-bromo-2-[(4-chlorophenyl)methoxy]phenyl]methyl]-N-methyl-4-piperidinemethanamine
[1-[[5-bromo-2-[(4-chlorophenyl)methoxy]phenyl]methyl]-4-piperidinyl](ethyl)carbamic acid, 1,1-dimethylethyl ester;
[1-[[5-bromo-2-[(4-chlorophenyl)methoxy]phenyl]methyl]-4-piperidinyl](methyl)carbamic acid, 1,1-dimethylethyl ester;
1-[[5-bromo-2-[(4-chlorophenyl)methoxy]phenyl]methyl]-N,N-diethyl-4-piperidinamine;
N-[1-[[5-bromo-2-[(4-chlorophenyl)methoxy]phenyl]methyl]-4-piperidinyl]-N′-(4-fluorophenyl)-urea;
N-[1-[[5-bromo-2-[(4-chlorophenyl)methoxy]phenyl]methyl]-3-pyrrolidinyl]-N′-(4-fluorophenyl)-urea;
N-[[1-[[5-bromo-2-[(4-chlorophenyl)methoxy]phenyl]methyl]-4-piperidinyl]methyl]-N′-(4-fluorophenyl)-N-methyl-urea;
N-[[1-[[5-bromo-2-[(4-chlorophenyl)methoxy]phenyl]methyl]-4-piperidinyl]methyl]-N′-[(4-fluorophenyl)methyl]-N-methyl-urea;
N-[1-[[5-bromo-2-[(4-chlorophenyl)methoxy]phenyl]methyl]-3-pyrrolidinyl]-2-chloroacetamide;
N-[[1-[[5-bromo-2-[(4-chlorophenyl)methoxy]phenyl]methyl]-4-piperidinyl]methyl]acetamide, trifluoroacetic acid salt;
N-[1-[[5-bromo-2-[(4-chlorophenyl)methoxy]phenyl]methyl]-4-piperidinyl]-acetamide, Trifluoroacetic acid salt;
N-[1-[[5-bromo-2-[(4-chlorophenyl)methoxy]phenyl]methyl]-3-pyrrolidinyl]-N-methyl-2-pyrazinecarboxamide;
N-[[1-[[5-bromo-2-[(4-chlorophenyl)methoxy]phenyl]methyl]-4-piperidinyl]methyl]-N,4-dimethyl-3-pyridinecarboxamide;
[1-[[5-bromo-2-[(4-chlorophenyl)methoxy]phenyl]methyl]-4-piperidinyl]carbamic acid, methyl ester;
4-[[4-bromo-2-[(diethylamino)methyl]phenoxy]methyl]benzoic acid;
5-bromo-N,N-diethyl-2-[[4-[[4-(phenylmethyl)-1-piperazinyl]carbonyl]phenyl]methoxy]benzenemethanamine;
N-(1,3-Benzodioxo-5-ylmethyl)-4-[[4-bromo-2-[(diethylamino)methyl]phenoxy]methyl]benzamide;
4-[[4-bromo-2-[(diethylamino)methyl]phenoxy]methyl]-N-[(4-methoxyphenyl)methyl]benzamide;
4-[[4-bromo-2-[(diethylamino)methyl]phenoxy]methyl]-N-methyl-N-(2-phenylethyl)benzamide;
4-[[4-bromo-2-[(diethylamino)methyl]phenoxy]methyl]-N-[2-(4-bromo-phenyl)ethyl]benzamide;
4-[4-[[4-bromo-2-[(diethylamino)methyl]phenoxy]methyl]benzoyl]-N-octyl-1-piperazincarboxamide;
5-bromo-N,N-didiethyl-2-[[4-[[4-[[3-nitrophenyl)sulfonyl]-1-piperazinyl]carbonyl]phenyl]methoxy]benzenemethanamine;
5-bromo-N,N-diethyl-2-[[4-[[4-(2-furanylcarbonyl)-1-piperazinyl]carbonyl]phenyl]methoxy]benzenemethanamine;
5-bromo-2-[[4-[[4-(2,6-dichlorobenzoyl)-1-piperazinyl]carbonyl]phenyl]methoxy]-N,N-diethylbenzenemethanamine;
N-[[5-[[4-[4-[[4-bromo-2-[(diethylamino)methyl]phenoxy]methyl]benzoyl]-1-piperazinyl]sulfonyl]-2-thienyl]methyl]benzamide;
1-[(5-bromo-2-propoxyphenyl)methyl]-4-(4-fluorophenyl)-4-piperidinol;
[4-bromo-2-[[4-(4-bromophenyl)-4-hydroxy-1-piperidinyl]methyl]phenoxy]-O-ethyloxime-ethanal;
1-[(5-bromo-2-propoxyphenyl)methyl]-4-(4-chlorophenyl)-4-piperidinol;
1-[[5-bromo-2-(pentyloxy)phenyl]methyl]-4-(4-bromophenyl)-4-piperidinol;
1-[[5-bromo-2-(hexyloxy)phenyl]methyl]-4-(4-bromophenyl)-4-piperidinol;
1-[(5-bromo-2-methoxyphenyl)methyl]-4-(4-bromophenyl)-4-piperidinol;
1-[[5-bromo-2-(1,3-dioxolan-2-ylmethoxy)phenyl]methyl]-4-(4-bromophenyl)-4-piperidinol;
1-[(5-bromo-2-hydroxyphenyl)methyl]-4-(4-bromophenyl)-4-piperidinol;
1-[[5-bromo-2-(2-methylpropoxy)phenyl]methyl]-4-(4-bromophenyl)-4-piperidinol;
1-[[5-bromo-2-(heptyloxy)phenyl]methyl]-4-(4-bromophenyl)-4-piperidinol, trifluoroacetic acid;
1-[[5-bromo-2-(cyclopropylmethoxy)phenyl]methyl]-4-(4-bromophenyl)-4-piperidinol, trifluoroacetic acid;
1-[(5-bromo-2-butoxyphenyl)methyl]-4-(4-bromophenyl)-4-piperidinol, trifluoroacetic acid;
1-[[5-bromo-2-(2-methoxyethoxy)phenyl]methyl]-4-(4-bromophenyl)-4-piperidinol, trifluoroacetic acid;
4-(4-bromophenyl)-1-[(5-bromo-2-propoxyphenyl)methyl]-4-piperidinol, trifluoroacetic acid;
1-[(5-bromo-2-ethoxyphenyl)methyl]-4-(4-bromophenyl)-4-piperidinol, trifluoroacetic acid;
4-(4-bromophenyl)-1-[[5-bromo-2-(2-propenyloxy)phenyl]methyl]-4-piperidinol, trifluoroacetic acid;
[5-bromo-2-[[4-(4-bromophenyl)-4-hydroxy-1-piperidinyl]methyl]phenoxy]-acetonitrile, trifluoroacetic acid.;
N-[2-[4-bromo-2-[[4-(4-bromophenyl)-4-hydroxy-1-piperidinyl]methyl]phenoxy]ethyl]-N′-ethyl-urea;
1-[[2-(2-aminoethoxy)-5-bromophenyl]methyl]-4-(4-bromophenyl)-4-piperidinol: 2-bromo-1-[5-bromo-2-[(4-chlorophenyl)methoxy]phenyl]-ethanone;
4-[[4-bromo-2-(bromoacetyl)phenoxy]methyl]benzoic acid, methyl ester;
1-[2-([1,1′-biphenyl]-4-ylmethoxy)-5-bromophenyl]-2-bromoethanone;
3-[[4-[4-[[4-bromo-2-(bromoacetyl)phenoxy]methyl]benzoyl]-1-piperazinyl]sulfonyl]-N-hydroxy-N-oxo-benzenaminium;
2-bromo-1-[5-bromo-2-[(4-chlorophenyl)methoxy]phenyl]-1-propanone;
1-[5-bromo-2-[(4-chlorophenyl)methoxy]phenyl]-2-(dimethylamino)-ethanone;
1-[2-([1,1′-biphenyl]-4-ylmethoxy)-5-bromophenyl]-2-(dimethylamino)-ethanone;
1-[2-([1,1′-biphenyl]-4-ylmethoxy)-5-bromophenyl]-2-bromoethanone;
5-bromo-2-[(4-chlorophenyl)methoxy]-α-[[(2-hydroxyethyl)(methyl)amino]methyl]benzenemethanol, trifluoroacetic acid salt;
α-[5-bromo-2-[(4-chlorophenyl)methoxy]phenyl]-3-hydroxy-1-piperidineethanol, trifluoroacetic acid salt;
α-[5-bromo-2-[(4-chlorophenyl)methoxy]phenyl]-3-hydroxy-1-pyrrolidineethanol, trifluoroacetic acid salt;
(2S,4R)-1-[2-[5-bromo-2-[(4-chlorophenyl)methoxy]phenyl]-2-hydroxyethyl]-4-hydroxy-2-pyrrolidinecarboxylic acid, trifluoroacetic acid salt;
5-bromo-2-[(4-chlorophenyl)methoxy]-α-[(dimethylamino)methyl]benzenemethanol, trifluoroacetic acid salt;
2-Amino-α-[5-bromo-2-[(4-chlorophenyl)methoxy]phenyl]-1H-imidazole-1-ethanol;
α-[5-bromo-2-[(4-chlorophenyl)methoxy]phenyl]-4-hydroxy-1-piperidineethanol;
4-[[4-bromo-2-[1-hydroxy-2-(4-hydroxy-1-piperidinyl)ethyl]phenoxy]methyl]benzoic acid, methyl ester, trifluoroacetic acid salt.;
4-[[4-bromo-2-[1-hydroxy-2-(3-hydroxy-1-piperidinyl)ethyl]phenoxy]methyl]benzoic acid, methyl ester, trifluoroacetic acid salt;
4-[[4-bromo-2-[2-[4-[[(1,1-dimethylethoxy)carbonyl]amino]-1-piperidinyl]-1-hydroxyethyl]phenoxy]methyl]benzoic acid, methyl ester;
2-([1,1′-biphenyl]-4-ylmethoxy)-5-bromo-α-[(dimethylamino)methyl]-benzenemethanol, trifluoroacetic acid salt;
4-[[4-chloro-2-[1-hydroxy-2-(4-hydroxy-1-piperidinyl)ethyl]phenoxy]methyl]benzoic acid;
1-[5-bromo-2-[(4-chlorophenyl)methoxy]phenyl]-2-(dimethylamino)-1-propanone;
5-chloro-2-[(4-chlorophenyl)methoxy]-α-[1-(dimethylamino)ethyl]benzenemethanol;
α-[5-chloro-2-[(4-chlorophenyl)methoxy]phenyl]-β-methyl-1H-imidazole-1-ethanol;
α-[5-chloro-2-[(4-chlorophenyl)methoxy]phenyl]-4-(4-chlorophenyl)-4-hydroxy-β-methyl-1-piperidineethanol;
α-[5-chloro-2-[(4-chlorophenyl)methoxy]phenyl]-4-hydroxy-β-methyl-4-(phenylmethyl)-1-piperidineethanol;
α-[5-chloro-2-[(4-chlorophenyl)methoxy]phenyl]-4-(4-fluorophenyl)-4-hydroxy-β-methyl-1-piperidineethanol;
5-chloro-2-[(4-chlorophenyl)methoxy]-α-[1-(diethylamino)ethyl-benzenemethanol;
α-[5-bromo-2-[[4-[[4-[(3-nitrophenyl)sulfonyl]-1-piperazinyl]carbonyl]phenyl]methoxy]phenyl]-3-hydroxy-1-piperidineethanol;
α-5-bromo-2-[[4-[[4-[(3-nitrophenyl)sulfonyl]-1-piperazinyl]carbonyl]phenyl]methoxy]phenyl]-4-hydroxy-1-piperidineethanol.;
α-[5-bromo-2-[[4-[[4-[(3-nitrophenyl)sulfonyl]-1-piperazinyl]carbonyl]phenyl]methoxy]phenyl]-3-hydroxy-1-pyrrolidineethanol;
5-bromo-α-[(diethylamino)methyl]-2-[[4-[[4-[(3-nitrophenyl)sulfonyl]-1-piperazinyl]carbonyl]phenyl]methoxy]benzenemethanol;
α-[5-bromo-2-[(4-chlorophenyl)methoxy]phenyl]-1-piperazineethanol;
α-[5-bromo-2-[(4-chlorophenyl)methoxy]phenyl]-4-(3-pyridinylcarbonyl)-1-piperazineethanol;
α-[5-bromo-2-[(4-chlorophenyl)methoxy]phenyl]-4-[(4-methyl-3-pyridinyl)carbonyl]-1-piperazineethanol.;
4-[[4-bromo-2-[1-hydroxy-2-[4-[[(phenylmethoxy)carbonyl]amino]-1-piperidinyl]ethyl]phenoxy]methyl]benzoic acid, methyl ester;
4-[[4-bromo-2-[1-hydroxy-2-(4-hydroxy-1-piperidinyl)ethyl]phenoxy]methyl]-N-pyridinyl)benzamide;
4-[[4-chloro-2-[1-hydroxy-2-(4-hydroxy-1-piperidinyl)ethyl]phenoxy]methyl]-N-(3-hydroxypropyl)benzamide;
2-[5-bromo-2-[(4-chlorophenyl)methoxy]phenyl]oxirane;
1-(2S)-α-[5-bromo-2-[(4-chlorophenyl)methoxy]phenyl]-2-(hydroxymethyl)-1-pyrrolidineethanol, trifluoroacetic acid salt;
(2R)-α-[5-bromo-2-[(4-chlorophenyl)methoxy]phenyl]-2-(hydroxymethyl)-1-pyrrolidineethanol, trifluoroacetic acid salt;
(3R)-α-[5-bromo-2-[(4-chlorophenyl)methoxy]phenyl]-3-hydroxy-1-pyrrolidineethanol, trifluoroacetic acid salt;
5-bromo-2-[(4-chlorophenyl)methoxy]-α-[[[2-(diethylamino)ethyl]ethylamino]methyl]-benzenemethanol, trifluoroacetic acid salt;
α-[5-bromo-2-[(4-chlorophenyl)methoxy]phenyl]-1,4-piperidinediethanol, trifluoroacetic acid salt.;
α-[5-bromo-2-[(4-chlorophenyl)methoxy]phenyl]-4-(piperidyl)-1-piperidineethanol;
5-bromo-2-[(4-chlorophenyl)methoxy]-α-[(dipropylamino)methyl]-benzenemethanol, trifluoroacetic acid salt;
α-[5-bromo-2-[(4-chlorophenyl)methoxy]phenyl]-4-(phenylmethyl)-1-piperidineethanol, trifluoroacetic acid salt;
5-bromo-2-[(4-chlorophenyl)methoxy]-α-[(dibutylamino)methyl]-benzenemethanol, trifluoroacetic acid salt;
5-bromo-α-[(butylethylamino)methyl]-2-[(4-chlorophenyl)methoxy]-benzenemethanol;
5-bromo-2-[(4-chlorophenyl)methoxy]-α-[[ethyl(2-hydroxyethyl)amino]methyl]benzenemethanol, trifluoroacetic acid salt;
5-bromo-2-[(4-chlorophenyl)methoxy]-α-[[(2-hydroxyethyl)propylamino]methyl]benzenemethanol trifluoroacetic acid salt;
1-[2-[5-bromo-2-[(4-chlorophenyl)methoxy]phenyl]-2-hydroxyethyl]-N,N-diethyl-3-piperidinecarboxamide, trifluoroacetic acid salt;
α-[5-bromo-2-[(4-chlorophenyl)methoxy]phenyl]-4-(4-bromophenyl)-4-hydroxy-1-piperidineethanol, trifluoroacetic acid salt;
1-[1-[2-[5-bromo-2-[(4-chlorophenyl)methoxy]phenyl]-2-hydroxyethyl]-4-piperidinyl]-1,3-dihydro-H-benzimidazol-2-one, trifluoroacetic acid salt;
1-[2-[5-bromo-2-[(4-chlorophenyl)methoxy]phenyl]-2-hydroxyethyl]-4-phenyl-4-piperidinecarbonitrile, trifluoroacetic acid salt.;
α-[5-bromo-2-[(4-chlorophenyl)methoxy]phenyl]-1,4-dioxa-8-azaspiro[4.5]decane-8-ethanol, trifluoroacetic acid salt;
5-bromo-2-[(4-chlorophenyl)methoxy]-α-[[(2-hydroxyethyl)(phenylmethyl)amino]methyl]-benzenemethanol, trifluoroacetic acid salt;
5-bromo-2-[(4-chlorophenyl)methoxy]-α-[[[2-(dimethylamino)ethyl]ethylamino]methyl]benzenemethanol, trifluoroacetic acid salt;
α-[5-bromo-2-[(4-chlorophenyl)methoxy]phenyl]-1,2,3,4-tetrahydro-1-quinolineethanol, trifluoroacetic acid salt;
1-[2-[5-bromo-2-[(4-chlorophenyl)methoxy]phenyl]-2-hydroxyethyl]-3,4-pyrrolidinediol, trifluoroacetic acid salt.;
5-bromo-2-[(4-chlorophenyl)methoxy]-α-[(methylamino)methyl]-benzenemethanol, trifluoroacetic acid salt;
2-[[2-[5-bromo-2-[(4-chlorophenyl)methoxy]phenyl]-2-hydroxyethyl]amino]-1,3-propanediol, trifluoroacetic acid salt;
5-bromo-2-[(4-chlorophenyl)methoxy]-α-[(diethylamino)methyl]-benzenemethanol, trifluoroacetic acid salt;
2-[[2-[5-bromo-2-[(4-chlorophenyl)methoxy]phenyl]-2-hydroxyethyl]amino]-2-(hydroxymethyl)-1,3-propanediol;
α-[5-bromo-2-[(4-chlorophenyl)methoxy]phenyl]-1-pyrrolidineethanol;
α-[5-bromo-2-[(4-chlorophenyl)methoxy]phenyl]-1-piperidineethanol;
5-bromo-2-[(4-chlorophenyl)methoxy]-α-[[(3-hydroxyphenyl)amino]methyl]benzenemethanol;
5-bromo-2-[(4-chlorophenyl)methoxy]-α-[[(cyclopropylmethyl)amino]methyl]benzenemethanol;
5-bromo-α-[[[2-(3-chlorophenyl)ethyl]amino]methyl]-2-[(4-chlorophenyl)methoxy]benzenemethanol;
α-[5-bromo-2-[(4-chlorophenyl)methoxy]phenyl]-1-azetidineethanol;
5-bromo-2-[(4-chlorophenyl)methoxy]-α-[(ethylmethylamino)methyl]benzenemethanol;
5-bromo-2-[(4-chlorophenyl)methoxy]-α-[(cyclopropylamino)methyl]benzenemethanol;
5-bromo-2-[(4-chlorophenyl)methoxy]-α-[[(cyclopropylmethyl)methylamino]methyl]benzenemethanol;
α-[5-bromo-2-[(4-chlorophenyl)methoxy]phenyl]-4-thiornorpholineethanol;
α-(Aminomethyl)-5-bromo-2-[(4-chlorophenyl)methoxy]benzenemethanol;
5-bromo-2-[(4-chlorophenyl)methoxy]-α-[(cyclopropylmethylamino)methyl]benzenemethanol;
(αS)-5-bromo-2-[(4-chlorophenyl)methoxy]-α-[(diethylamino)methyl]benzenemethanol;
(αR)-5-bromo-2-[(4-chlorophenyl)methoxy]-α-[(diethylamino)methyl]-benzenemethanol;
α-[[bis(2-hydroxyethyl)amino]methyl]-5-bromo-2-[(4-chlorophenyl)methoxy]benzenemethanol;
α-[5-bromo-2-[(4-chlorophenyl)methoxy]phenyl]-4-methyl-1-piperazineethanol;
5-bromo-2-[(4-chlorophenyl)methoxy]-α-[[(1-methylethyl)amino]methyl]-benzenemethanol;
α-[5-bromo-2-[(4-chlorophenyl)methoxy]phenyl]-4-morpholineethanol;
5-bromo-2-[(4-chlorophenyl)methoxy]-α-[[(2-hydroxyethyl)amino]methyl]-benzenemethanol;
5-bromo-2-[(4-chlorophenyl)methoxy]-α-[[(2-hydroxyethyl)amino]methyl]-benzenemethanol;
5-bromo-2-[(4-chlorophenyl)methoxy]-α-ethoxy-N,N-diethylbenzeneethanamine;
5-bromo-2-[(4-chlorophenyl)methoxy]-N,N-diethyl-α-(2-pyridinyloxy)benzeneethanamine;
5-bromo-2-[(4-chlorophenyl)methoxy]-α-(methylamino)benzeneethanol;
1-[5-bromo-2-[(4-chlorophenyl)methoxy]phenyl]-2-propen-1-one;
(3R)-α-[5-bromo-2-[(4-chlorophenyl)methoxy]phenyl]-3-hydroxy-1-pyrrolidinepropanol;
5-bromo-2-[(4-chlorophenyl)methoxy]-α-[2-(dimethylamino)ethyl]-benzenemethanol;
α-[5-bromo-2-[(4-chlorophenyl)methoxy]phenyl]-4-hydroxy-1-piperidinepropanol;
5-bromo-2-[(4-chlorophenyl)methoxy]-α-[2-(dipropylamino)ethyl]-benzenemethanol;
5-bromo-2-[(4-chlorophenyl)methoxy]-α-[2-(diethylamino)ethyl]-benzenemethanol;
5-chloro-2-[(4-fluorophenyl)methoxy]benzeneethanamine;
N-[[2-[5-chloro-2-[(4-fluorophenyl)methoxy]-phenyl]ethyl]-4-pyridinemethanamine;
5-chloro-2-[(4-fluorophenyl)methoxy]-N,N,α-trimethylbenzeneethanamine;
4-[[[2-[5-chloro-2-[(4-fluorophenyl)methoxy]phenyl]ethyl]amino]methyl]benzonitrile;
N-[2-[5-chloro-2-[[4-fluorophenyl)methoxy]phenyl]ethyl]-N-(1H-imidazol-5-ylmethyl)-1H-imidazole-4-methanamine;
5-chloro-α-ethyl-2-[(4-fluorophenyl)methoxy]-N-[(4-fluorophenyl)methyl]benzeneethanamine;
5-chloro-α-ethyl-2-[(4-fluorophenyl)methoxy]-N-[(3-methyl-4-methoxyphenyl)methyl]benzeneethanamine;
1-[[5-bromo-2-[(4-chlorophenyl)methoxy]phenyl]methyl]-4-hydroxy-4-piperidinecarboxylic acid, methyl ester;
1-[[5-bromo-2-[(4-chlorophenyl)methoxy]phenyl]methyl]-4-hydroxy-4-piperidinecarboxylic acid;
4-[[1-[[5-bromo-2-[(4-chlorophenyl)methoxy]phenyl]methyl]-4-hydroxy-4-piperidinyl]carbonyl]-1-piperazineethanol;
1-[[5-bromo-2-[(4-chlorophenyl)methoxy]phenyl]methyl]-4-(1-piperazinylcarbonyl)-4-piperidinol;
1-[[5-bromo-2-[(4-chlorophenyl)methoxy]phenyl]methyl]-4-[[(3R)-3-methylpiperazinyl]carbonyl]-4-piperidinol;
4-[1-[[5-bromo-2-[(4-chlorophenyl)methoxy]phenyl]methyl]-4-piperidinyl]-1-piperazinecarboxylic acid, 1,1-dimethylethyl ester;
1-[1-[[5-bromo-2-[(4-chlorophenyl)methoxy]phenyl]methyl]-4-piperidinyl]piperazine;
1-[1-[[5-bromo-2-[(4-chlorophenyl)methoxy]phenyl]methyl]-4-piperidinyl]-4-[(2,4-dimethyl-3-pyridinyl)carbonyl]piperazine;
1-[[5-bromo-2-[(4-chlorophenyl)methoxy]phenyl]methyl]-3-methyl-4-piperidinone;
1-[[5-bromo-2-[(4-chlorophenyl)methoxy]phenyl]methyl]-3-methyl-4-piperidinol;
1-[[5-bromo-2-[(4-chlorophenyl)methoxy]phenyl]methyl]-4,4-difluoropiperidine;
8-[[5-bromo-2-[(4-chlorophenyl)methoxy]phenyl]methyl]-1,3,8-triazaspiro[4.5]decane-2,4-dione;
1-[[5-bromo-2-[(4-chlorophenyl)methoxy]phenyl]methyl]-4-phenyl-4-piperidinol;
1-[[5-bromo-2-[(4-chlorophenyl)methoxy]phenyl]methyl]-4-ethyl-4-piperidinol;
1-[[5-bromo-2-[(4-chlorophenyl)methoxy]phenyl]methyl]-4-(trifluoromethyl)-4-piperidinol;
1-[[5-bromo-2-[(4-chlorophenyl)methoxy]phenyl]methyl]-4-piperidinone-oxime;
1-[[5-bromo-2-[[4-(trifluoromethyl)phenyl]methoxy]phenyl]methyl]-4-fluoropiperidine;
1-[[5-bromo-2-[(4-chlorophenyl)methoxy]phenyl]methyl]-4-(2-pyridinyloxy)piperidine;
2-[[1-[[5-bromo-2-[(4-chlorophenyl)methoxy]phenyl]methyl]-4-piperidinyl]oxy]pyrimidine;
1-[[5-bromo-2-[(4-chlorophenyl)methoxy]phenyl]methyl]-N-ethyl-4-piperidinamine;
6-[[5-bromo-2-[(4-chlorophenyl)methoxy]phenyl]methyl]-1-oxa-6-azaspiro[2.5]octane
4-(aminomethyl)-1-[[5-bromo-2-[(4-chlorophenyl)methoxy]phenyl]methyl]-4-piperidinol;
1-[[5-bromo-2-[(4-chlorophenyl)methoxy]phenyl]methyl]-4-[[[[1-[[5-bromo-2-[(4-chlorophenyl)methoxy]phenyl]methyl]-4-hydroxy-4-piperidinyl]methyl]amino]methyl]-4-piperidinol;
4-[[1-[[5-bromo-2-[(4-chlorophenyl)methoxy]phenyl]methyl]-4-hydroxy-4-piperidinyl]methyl]-1-piperazinecarboxylic acid, 1,1-dimethylethyl ester;
4-[[1-[[5-bromo-2-[(4-chlorophenyl)methoxy]phenyl]methyl]-4-hydroxy-4-piperidinyl]methyl]hexahydro-1H-1,4-diazepine-1-carboxylic acid, 1,1-dimethylethyl ester;
1-[[5-bromo-2-[(4-chlorophenyl)methoxy]phenyl]methyl]-4-[[4-(2-pyridinyl)-1-piperazinyl]methyl]-4-piperidinol;
1-[[5-bromo-2-[(4-chlorophenyl)methoxy]phenyl]methyl]-4-[[4-(2-pyrimidinyl)-1-piperazinyl]methyl]-4-piperidinol.;
1-[[5-bromo-2-[(4-chlorophenyl)methoxy]phenyl]methyl]-4-(1-piperazinylmethyl)-4-piperidinol;
1-[[5-bromo-2-[(4-chlorophenyl)methoxy]phenyl]methyl]-4-[(hexahydro-1H-1,4-diazepin-1-yl)methyl]-4-piperidinol;
1-[[5-bromo-2-[(4-chlorophenyl)methoxy]phenyl]methyl]-4-[[(4-methylphenyl)amino]methyl]-4-piperidinol;
1-[[5-bromo-2-[(4-chlorophenyl)methoxy]phenyl]methyl]-4-[[(4-methoxyphenyl)amino]methyl]-4-piperidinol;
1-[[5-bromo-2-[(4-chlorophenyl)methoxy]phenyl]methyl]-4-[[(3S)-3-methylpiperazinyl]methyl]-4-piperidinol;
1-[[5-bromo-2-[(4-chlorophenyl)methoxy]phenyl]methyl]-4-[(2,5-dimethyl-1-piperazinyl)methyl]-4-piperidinol;
4-[[(3-Aminopropyl)amino]methyl]-1-[[5-bromo-2-[(4-chlorophenyl)methoxy]phenyl]methyl]-4-piperidinol;
1-[[5-bromo-2-[(4-chlorophenyl)methoxy]phenyl]methyl]-4-[[[2-(1-piperidinyl)ethyl]amino]methyl]-4-piperidinol;
2-[[[[1-[[5-bromo-2-[(4-chlorophenyl)methoxy]phenyl]methyl]-4-hydroxy-4-piperidinyl]methyl]amino]methyl]-1-pyrrolidinecarboxylic acid, 1,1-dimethylethyl ester;
1-[[5-bromo-2-[(4-chlorophenyl)methoxy]phenyl]methyl]-4-[[(2-pyrrolidinylmethyl)amino]methyl]-4-piperidinol;
1-[[5-bromo-2-[(4-chlorophenyl)methoxy]phenyl]methyl]-4-[[4-[(2,4-dimethyl-3-pyridinyl)carbonyl]-1-piperazinyl]methyl]-4-piperidinol;
4-[[1-[[5-bromo-2-[(4-chlorophenyl)methoxy]phenyl]methyl]-4-hydroxy-4-piperidinyl]methyl]-1-piperazinecarboxylic acid, ethyl ester;
4-[(4-Acetyl-1-piperazinyl)methyl]-1-[[5-bromo-2-[(4-chlorophenyl)methoxy]phenyl]methyl]-4-piperidinol;
1-[[5-bromo-2-[(4-chlorophenyl)methoxy]phenyl]methyl]-4-[(1-piperazinylamino)methyl]-4-piperidinol;
4-[[1-[[5-bromo-2-[(4-chlorophenyl)methoxy]phenyl]methyl]-4-hydroxy-4-piperidinyl]methyl]-1-piperazineethanol;
4-[[1-[[5-bromo-2-[(4-chlorophenyl)methoxy]phenyl]methyl]-4-hydroxy-4-piperidinyl]methyl]-1-piperazinecarboxaldehyde;
4-[[1-[[5-bromo-2-[(4-chlorophenyl)methoxy]phenyl]methyl]-4-hydroxy-4-piperidinyl]methyl]-1-piperazinecarboxylic acid, phenylmethyl ester;
1-[[5-bromo-2-[(4-chlorophenyl)methoxy]phenyl]methyl]-4-[[4-(phenylmethyl)-1-piperazinyl]methyl]-4-piperidinol;
1-[[5-bromo-2-[(4-chlorophenyl)methoxy]phenyl]methyl]-4-[[(2-methylphenyl)amino]methyl]-4-piperidinol;
1-[[1-[[5-bromo-2-[(4-chlorophenyl)methoxy]phenyl]methyl]-4-hydroxy-4-piperidinyl]methyl]-4-piperidinecarboxamide;
1-[[5-bromo-2-[(4-chlorophenyl)methoxy]phenyl]methyl]-4-[[(3S)-3-methylpiperazinyl]methyl]-4-piperidinol;
4-[[1-[[5-bromo-2-[(4-chlorophenyl)methoxy]phenyl]methyl]-4-hydroxy-4-piperidinyl]methyl]-2-piperazinone;
1-[[5-bromo-2-[(4-chlorophenyl)methoxy]phenyl]methyl]-4-[[(3S)-3-methylpiperazinyl]methyl]-4-piperidinol;
1-[[5-bromo-2-[(4-chlorophenyl)methoxy]phenyl]methyl]-4-[(3,5-dimethyl-1-piperazinyl)methyl]-4-piperidinol;
1-[[5-bromo-2-[(4-chlorophenyl)methoxy]phenyl]methyl]-4-(2,5-diazabicyclo[2.2.1]hept-2-ylmethyl)-4-piperidinol;
[1-[[1-[[5-bromo-2-[(4-chlorophenyl)methoxy]phenyl]methyl]-4-hydroxy-4-piperidinyl]methyl]-3-pyrrolidinyl]-carbamic acid, 1,1-dimethylethyl ester;
4-[(3-Amino-1-pyrrolidinyl)methyl]-1-[[5-bromo-2-[(4-chlorophenyl)methoxy]phenyl]methyl]-4-piperidinol;
1-[[5-bromo-2-[(4-chlorophenyl)methoxy]phenyl]methyl]-4-[[4-[2-(dimethylamino)ethyl]-1-piperazinyl]methyl]-4-piperidinol;
1-[[5-bromo-2-[(4-chlorophenyl)methoxy]phenyl]methyl]-4-[[4-[2-(4-morpholinyl)-2-oxoethyl]-1-piperazinyl]methyl]-4-piperidinol;
1-[[5-bromo-2-[(4-chlorophenyl)methoxy]phenyl]methyl]-4-[[4-[3-(4-morpholinyl)propyl]-1-piperazinyl]methyl]-4-piperidinol;
1-[[5-bromo-2-[(4-chlorophenyl)methoxy]phenyl]methyl]-4-[[4-[2-(4-morpholinyl)ethyl]-1-piperazinyl]methyl]-4-piperidinol;
1-[[5-bromo-2-[(4-chlorophenyl)methoxy]phenyl]methyl]-4-[(dimethylamino)methyl]-4-piperidinol;
1-[[5-bromo-2-[(4-chlorophenyl)methoxy]phenyl]methyl]-4-[(diethylamino)methyl]-4-piperidinol;
1-[[5-bromo-2-[(4-chlorophenyl)methoxy]phenyl]methyl]-4-[[(1-methylethyl)amino]methyl]-4-piperidinol;
1-[[5-bromo-2-[(4-chlorophenyl)methoxy]phenyl]methyl]-4-[(4-hydroxy-1-piperidinyl)methyl]-4-piperidinol;
1-[[5-bromo-2-[(4-chlorophenyl)methoxy]phenyl]methyl]-4-[[(3R)-3-hydroxypyrrolidinyl]methyl]-4-piperidinol;
1-[[5-bromo-2-[(4-chlorophenyl)methoxy]phenyl]methyl]-4-[[[(4-fluorophenyl)methyl]amino]methyl]-4-piperidinol;
1-[[5-bromo-2-[(4-chlorophenyl)methoxy]phenyl]methyl]-4-(1H-imidazol-1-ylmethyl)-4-piperidinol;
1-[[5-bromo-2-[(4-chlorophenyl)methoxy]phenyl]methyl]-4-[(phenylamino)methyl]-4-piperidinol;
1-[[5-bromo-2-[(4-chlorophenyl)methoxy]phenyl]methyl]-4-[(4-pyridinylamino)methyl]-4-piperidinol;
1-[[5-bromo-2-[(4-chlorophenyl)methoxy]phenyl]methyl]-4-[[(2-hydroxyethyl)methylamino]methyl]-4-piperidinol;
1-[[5-bromo-2-[(4-chlorophenyl)methoxy]phenyl]methyl]-4-[(4-methyl-1-piperazinyl)methyl]-4-piperidinol;
1-[[5-bromo-2-[(4-chlorophenyl)methoxy]phenyl]methyl]-4-[(dipropylamino)methyl]-4-piperidinol;
1-[[1-[[5-bromo-2-[(4-chlorophenyl)methoxy]phenyl]methyl]-4-hydroxy-4-piperidinyl]methyl]-N,N-diethyl-3-piperidinecarboxamide;
1-[[5-bromo-2-[(4-chlorophenyl)methoxy]phenyl]methyl]-4-[[(2,2,2-trifluoroethyl)amino]methyl]-4-piperidinol;
1-[[5-bromo-2-[(4-chlorophenyl)methoxy]phenyl]methyl]-4-[[(3-methylphenyl)amino]methyl]-4-piperidinol;
1-[[5-bromo-2-[(4-chlorophenyl)methoxy]phenyl]methyl]-4-[[[(1R)-1-phenylethyl]amino]methyl]-4-piperidinol;
4-[[1-[[5-bromo-2-[(4-chlorophenyl)methoxy]phenyl]methyl]-4-hydroxy-4-piperidinyl]methyl]-N-ethyl-1-piperazinecarboxamide;
N-[[1-[[5-bromo-2-[(4-chlorophenyl)methoxy]phenyl]methyl]-4-hydroxy-4-piperidinyl]methyl]-N′-(2,6-difluorophenyl)urea;
N-[[1-[[5-bromo-2-[(4-chlorophenyl)methoxy]phenyl]methyl]-4-hydroxy-4-piperidinyl]methyl]-N′-(2,6-dimethoxyphenyl)urea;
N-[[1-[[5-bromo-2-[(4-chlorophenyl)methoxy]phenyl]methyl]-4-hydroxy-4-piperidinyl]methyl]-N′-(2,6-diethylphenyl)urea;
N-[[1-[[5-bromo-2-[(4-chlorophenyl)methoxy]phenyl]methyl]-4-hydroxy-4-piperidinyl]methyl]-N′-(2,4,6-trichlorophenyl)urea.;
N-[[1-[[5-bromo-2-[(4-chlorophenyl)methoxy]phenyl]methyl]-4-hydroxy-4-piperidinyl]methyl]-N′-(2,6-dichlorophenyl)urea;
N-[[1-[[5-bromo-2-[(4-chlorophenyl)methoxy]phenyl]methyl]-4-hydroxy-4-piperidinyl]methyl]-N′-(2,6-dimethylphenyl)urea;
N-[[1-[[5-bromo-2-[(4-chlorophenyl)methoxy]phenyl]methyl]-4-hydroxy-4-piperidinyl]methyl]-N′-(2,6-dibromophenyl)urea;
N-[[1-[[5-bromo-2-[(4-chlorophenyl)methoxy]phenyl]methyl]-4-hydroxy-4-piperidinyl]methyl]-N′-(4-bromo-2,6-dimethylphenyl)urea;
N-[2,6-Bis(1-methylethyl)phenyl]-N′-[[1-[[5-bromo-2-[(4-chlorophenyl)methoxy]phenyl]methyl]-4-hydroxy-4-piperidinyl]methyl]urea;
N-[[1-[[5-bromo-2-[(4-chlorophenyl)methoxy]phenyl]methyl]-4-hydroxy-4-piperidinyl]methyl]-N′-(4-fluorophenyl)urea;
2-amino-N-[[1-[[5-bromo-2-[(4-chlorophenyl)methoxy]phenyl]methyl]-4-hydroxy-4-piperidinyl]methyl]acetamide;
N-[2-[[[1-[[5-bromo-2-[(4-chlorophenyl)methoxy]phenyl]methyl]-4-hydroxy-4-piperidinyl]methyl]amino]-2-oxoethyl]-2,6-difluorobenzamide;
N-[[1-[[5-bromo-2-[(4-chlorophenyl)methoxy]phenyl]methyl]-4-hydroxy-4-piperidinyl]methyl]benzamide;
N-[[1-[[5-bromo-2-[(4-chlorophenyl)methoxy]phenyl]methyl]-4-hydroxy-4-piperidinyl]methyl]-4-chlorobenzamide;
3-[[[[1-[[5-bromo-2-[(4-chlorophenyl)methoxy]phenyl]methyl]-4-hydroxy-4-piperidinyl]methyl]amino]carbonyl]-1-hydroxy-2,4-dimethylpyridinium;
N-[[1-[[5-bromo-2-[(4-chlorophenyl)methoxy]phenyl]methyl]-4-hydroxy-4-piperidinyl]methyl]acetamide;
2-(acetylamino)-N-[[1-[[5-bromo-2-[(4-chlorophenyl)methoxy]phenyl]methyl]-4-hydroxy-4-piperidinyl]methyl]acetamide;
[2-[[[1-[[5-bromo-2-[(4-chlorophenyl)methoxy]phenyl]methyl]-4-hydroxy-4-piperidinyl]methyl]amino]-2-oxoethyl]carbamic acid, phenylmethyl ester;
(αS)-α-amino-N-[[1-[[5-bromo-2-[(4-chlorophenyl)methoxy]phenyl]methyl]-4-hydroxy-4-piperidinyl]methyl]benzeneacetamide;
N-[[1-[[5-bromo-2-[(4-chlorophenyl)methoxy]phenyl]methyl]-4-hydroxy-4-piperidinyl]methyl]-2-chloroacetamide;
N-[[1-[[5-bromo-2-[(4-chlorophenyl)methoxy]phenyl]methyl]-4-hydroxy-4-piperidinyl]methyl]-N-methylacetamide; and
enantiomers, diastereomers, salts and solvates thereof.

14. A pharmaceutical composition comprising a compound of claim 1 and at least one pharmaceutically acceptable vehicle, carrier, diluent or excipient therefor.

15. A method for treating an inflammatory or immunoregulatory disorder which comprises administering an effective amount of a compound of claim 1 to a patient in need thereof.

16. A method of claim 15 wherein the disorder treated is selected from asthma, allergic rhinitis, dermatitis, conjunctivitis, and atherosclerosis.

17. A method of claim 15 wherein the disorder treated is rheumatoid arthritis.

18. A method of claim 15 wherein the disorder treated is multiple sclerosis.

19. A method of claim 15 wherein the disorder treated is psoriasis.
